(19) United States
(12) Patent Application Publication
(10) Pub. No.: US 2022/0220128 A1
(43) Pub. Date: Jul. 14, 2022

(54) HETEROCYCLIC COMPOUNDS AS KINASE INHIBITORS, COMPOSITIONS COMPRISING THE HETEROCYCLIC COMPOUND, AND METHODS OF USE THEREOF

(71) Applicant: JS Innopharm (Shanghai) Ltd, Shanghai (CN)

(72) Inventors: Qun LI, Sunnyvale, CA (US); Jintao ZHANG, Naperville, IL (US); Shanzhong JIAN, Shanghai (CN); Ao LI, Shanghai (CN); Wen XU, Shanghai (CN)

(21) Appl. No.: 17/613,239

(22) PCT Filed: Jun. 9, 2020
(86) PCT No.: PCT/CN2020/095110

§ 371 Date: Nov. 22, 2021

(30) Foreign Application Priority Data
Jun. 10, 2019 (CN) 201910497783.1

(51) Int. Cl. C07D 519/00; A61K 31/437; A61K 31/444; C07D 471/14; A61K 31/5377; A61K 31/497; A61P 35/00

(52) U.S. Cl. CPC C07D 519/00; A61K 31/437; A61K 31/444; A61P 35/00; A61K 31/5377; A61K 31/497; C07D 471/14

(57) ABSTRACT

(I) Disclosed herein are compounds of formula I, and/or a stereoisomer, a stable isotope, or a pharmaceutically acceptable salt or solvate thereof; and therapeutic uses of these compounds, which are inhibitors of rearranged during transfection (RET), potentially useful in the treatment of RET-associated diseases, such as RET-associated cancers.

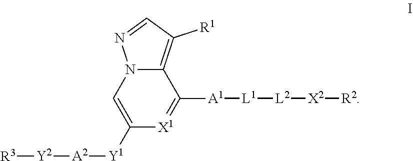

### TECHNICAL BACKGROUND

Disclosed herein are novel heterocyclic compounds that can serve as rearranged during transfection (RET) kinase inhibitors. Further disclosed herein are pharmaceutical compositions, comprising at least one of such compounds, as well as methods of using at least one of such compounds in the treatment of diseases and disorders modulated by RET, such as cancers.

RET is a transmembrane glycoprotein receptor tyrosine kinase (RTK) that is encoded by RET oncogene (Borrello, M. G., et al., Expert Opin. Ther. Targets. 2013, vol. 17, pp. 403-419). Upon homodimerization mediated by the GFL-GFRα complex, RET is activated via trans-autophosphorylation on the tyrosine residues in the intracellular kinase domain. The phosphotyrosine residues of RET serve as docking sites for the SH2 domain of several signaling adaptors which activate several signal transduction cascades involved in cellular proliferation, including the RAS/MARK/ERK, PI3K/Akt/mTOR, and JAK/STAT pathways. There are several major genetic aberrations leading to a dysregulated RET activity in many tumors. RET gene fusions and RET point mutations are RET mutations in many tumors, among others. RET gene fusions are found in a variety of cancers, including 1-2% non-small cell lung cancers (NSCLC), 20-30% of papillary thyroid cancers (PTCs), and less than 1% of other cancers such as pancreatic cancers, salivary gland cancers, spitz tumors, colorectal cancers, ovarian cancers and myeloproliferative cancers. So far at least 12 different fusion variants have been identified, with KIF5B-RET being the most common in NSCLCs, and CCDC6 and NCOA4 being most common in PTCs. RET point mutations occur mostly in sporadic medullary thyroid cancers (MTCs, 30-50%) and hereditary MTCs (100%), with RET M918T, G810R, V804L and V804M and being the most common mutations. Moreover, overexpression of wild-type RET, through its physiological neurotrophic functions, may play a role in the pathogenesis of other tumor types, such as pancreatic cancer.

Therefore, RET is a potential therapeutic target in cancer and other diseases with aberrant RET activity (such as a gastrointestinal disorder such as irritable bowel syndrome). A number of multitargeted kinase inhibitors with RET activity, such as cabozantinib, vandetanib, lenvatinib and alectinib, have been already investigated in clinical trials in cancer patients (Drilon, A. et al. Nat. Rev. Clin. Oncol., 2018, vol. 15, pp. 151-167). Despite showing efficacy in certain tumor types, the clinical activity of such multitargeted agents has been limited due to short duration and severe side effects. Such inhibitors, due to their dose-limiting toxicological liabilities caused by the primary and more potent inhibition of non-RET kinases, such as VEGFR2, have not to date allowed unequivocal demonstration of value of RET per se as a clinically relevant therapeutic target. Therefore, there is a need for more potent and more RET selective inhibitor drugs with better drug-like properties like improved DMPK properties.

### SUMMARY OF THE INVENTION

Disclosed herein are a series of novel potent and selective RET kinase inhibitors and methods for their preparation and use thereof. The compounds disclosed herein can have strong cancer inhibitory effects and can effectively inhibit RET-associated cancers.

Disclosed herein are compounds of Formula I:

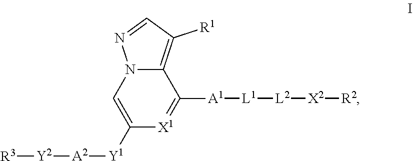

and/or stereoisomers, stable isotopes, or pharmaceutically acceptable salts or solvates thereof, wherein R¹, R², R³, A¹, A², L¹, L², X¹, X², Y¹ and Y² are defined below.
R¹ is selected from H, —CN, ethynyl, halo, —CF₃, —CH₃, —CH₂CH₃, cyclopropyl, —CH₂CN, and —CH(CN)CH₃;
R² is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R² is 1-4 substituents independently selected from R⁴, wherein each R⁴ is independently selected from halo, —OH, NH₂, ═O, —CN, —OC(O)R⁵, —CO₂R⁵, —C(O)N(R^{6a} R^{6b}), —C(═NR⁷)N(R^{6a} R^{6b}), —C(O)R⁵, —S(O)₀₋₂R⁸, —S(O)(═NR⁷)R⁸, —S(O)₁₋₂N(R^{6a} R^{6b}), —N(R^{6a} R^{6b}), —N(R^{6a})C(O)R⁸, —N(R^{6a})C(═NR⁷)R⁸, —N(R^{6a})S(O)₁₋₂R⁸, —N(R^{6c})C(O)N(R^{6a}R^{6b}), —N(R^{6c})C(═NR⁷)N(R^{6a}R^{6b}), —N(R^{6c})S(O)₁₋₂N(R^{6a}R^{6b}), —N(R^{6a})CO₂R⁸, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated and unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from -halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R⁵, R^{6a}, R^{6b} and R^{6c} are independently selected from H, C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; R⁷ is independently selected from H, —CN, —OH, C1-C4 alkyl and C1-C4 alkoxy; R⁸ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; wherein each of R⁵, R^{6a}R^{6b}, R^{6c}, R⁷, and R⁸ is optionally substituted with 1-3 groups independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl;
wherein two substituents on the same or adjacent carbon atoms of R² can optionally be taken together to form a 4-6 membered ring that can be saturated or aromatic and optionally contains 1-2 heteroatoms selected from N, O and S and can optionally be substituted with 1-2 groups independently selected from R⁴;
R³ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, saturated 7-8 membered bridged heterocyclyl containing 1-2 heteroatoms selected from N, O, and S as ring members, saturated 7-11 membered spiroheterocyclyl containing 1-2 heteroatoms selected from N, O, and S as ring members, 5-membered heteroaryl containing 1-3 heteroatoms selected from N, O, and S as ring members; and wherein the optional substituents for R³ is 1-4 substituents independently selected from R⁴;
A¹ is an optionally substituted group selected from para-attached benzene, para-attached 6-membered heteroarene containing 1-2 N as ring members, 2,5-attached thiophene, and 2,5-attached thiazole; wherein the optional substituents are 1-3 substituents selected from F, Cl, CN, CH₃, and CF₃;
A² is a bond or an optionally substituted C1-C6 alkylenyl wherein the optional substituents are 1-3 substituents selected from R⁴;
L¹ is selected from

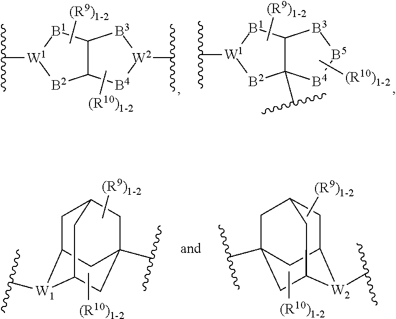

wherein W¹ is N or

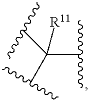

wherein R¹¹ is selected from H, OH, CN, F, and an optionally substituted group selected from C1-C6 alkyl, and C1-C6 alkoxy, and wherein the optional substituents are 1-3 groups independently selected from halo, OH, CN, C1-C3 alkyl, C1-C3 haloalkyl, C1-C3 alkoxy, C1-C3 haloalkoxy, C3-C6 cycloalkyl, and C3-C6 cycloalkyloxy;
wherein W² is N or

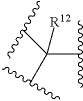

or, wherein R¹² is selected from H, F, OH, —CO₂H, and an optionally substituted group selected from C1-C6 alkyl and C1-C6 alkoxy, and wherein the optional substituents are 1-3 groups independently selected from R⁴;
wherein B¹, B², B³ and B⁴ are independently selected from a bond, —O—, and an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein zero, one, or two of B¹, B², B³ and B⁴ is a bond or —O—; wherein B5 is —O—, or an optionally substituted C1-C3 alkylenyl wherein the optional substituents are 1-3 substituents each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein when B⁵ is —O—, B³ and B⁴ cannot be —O—, or zero or one of B³ and B⁴ is a bond;
wherein R⁹ and R¹⁰ are independently selected from R⁴;
L² is a bond or an optionally substituted C1-C4 alkylenyl wherein the optional substituents are 1-3 groups independently selected from R⁴;
X¹ is —C(H)— or N;
X² is selected from a bond, —O—, —N(R¹³)—, —C(O)—, —C(O)O—, —C(O)N(R¹³)—, —N(R¹³)C(O)—, —N(R¹³)C(O)N(R⁴)—, —N(R¹³)C(O)O—, —S(O)₀₋₂—, —S(O)₁₋₂NR¹³—, —N(R¹³)S(O)₁₋₂—, —S(O)(NR¹⁵)—, —S(O)(NR¹⁵)NR—, —NR¹³S(O)(NR¹⁵)—, —N(R¹³)S(O)₂N(R¹⁴)—, and an optionally substituted group selected from C1-C3 alkylenyl and C3-C6 cycloalkylidenyl; wherein R¹³ and R¹⁴ are independently selected from H and an optionally substituted group independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents are 1-3 groups independently selected from R⁴; R¹⁵ is selected from H, —CN, —OH, and an optionally substituted group selected from C1-C4 alkyl and C1-C4 alkoxy, and the optional substituents are 1-3 groups independently selected from R⁴;
Y¹ is selected from a bond, 0, —N(R¹³)—, and an optionally substituted C1-C3 alkylenyl wherein the optional substituents are 1-3 groups independently selected from R⁴; and
Y² is selected from a bond, —O—, and —N(R¹³)—.

Also disclosed herein is a pharmaceutical composition, comprising a compound of Formula I and/or a stereoisomer, a stable isotope, or a pharmaceutically acceptable salt or solvate thereof disclosed herein and a pharmaceutically acceptable carrier.

Further disclosed herein is a method of inhibiting the activity of RET comprising contacting the protein RET with an effective amount of a compound of Formula I and/or a stereoisomer, a stable isotope, or a pharmaceutically acceptable salt or solvate thereof disclosed herein.

Further disclosed herein is a method of treating a disease treatable by inhibition of ERT in a patient, comprising administering to the patient in recognized need of such treatment, an effective amount of a compound of Formula I and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein and a pharmaceutically acceptable carrier.

Further disclosed herein is a method of treating a cancer in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein, and a pharmaceutically acceptable carrier. In some embodiments, the cancer is selected from lung cancers, thyroid cancers, pancreatic cancers, salivary gland cancers, spitz tumors, colorectal cancers, ovarian cancers, and myeloproliferative cancers.

Further disclosed herein is a use of a compound of Formula I and/or a stereoisomer, a stable isotope, or a pharmaceutically acceptable salt or solvate thereof in preparation of a medication for treating a disease responsive to inhibition of RET, such as a cancer. In some embodiments, the cancer is selected from lung cancers, thyroid cancers, pancreatic cancers, salivary gland cancers, spitz tumors, colorectal cancers, ovarian cancers, and myeloproliferative cancers.

Further disclosed herein are compounds of Formula I and the subgenera of Formula I disclosed herein, as well as pharmaceutically acceptable salts or solvates of these compounds, and all stereoisomers (including diastereoisomers and enantiomers, and isotopically enriched versions thereof (including deuterium substitutions). These compounds can be used to treat conditions responsive to RET inhibition, such as those disclosed herein, and for use in the preparation of a medicament for treating these disorders. The pharmaceutical compositions and methods disclosed herein can also be used with or formulated with a co-therapeutic agent; for example, compounds of Formula I and sub-formula thereof can be used with or formulated with at least one agent selected from inhibitors of and non-RET kinase and other therapeutic agents.

Further disclosed are methods, as well as key intermediate compounds, useful for making the compounds of Formula I as disclosed herein.

As used herein, the following words, phrases and symbols are generally intended to have the meanings as set forth below, except to the extent that the context in which they are used indicates otherwise. The following abbreviations and terms have the indicated meanings throughout.

### DETAILED DESCRIPTION

The following definitions apply unless otherwise provided or apparent from context:

A dash (“—”) that is not between two letters or symbols is used to indicate a point of attachment for a substituent. For example, —CONRₐR_{b} is attached through the carbon atom.

Unless clearly indicated otherwise, use of the terms “a”, “an” and the like refers to one or more.

The term “halogen” or “halo” herein refers to fluorine (F), chlorine (Cl), bromine (Br) or iodine (I). Halogen-substituted groups and moieties, such as alkyl substituted by halogen (haloalkyl) can be mono-, poly-, or per-halogenated. In some embodiments, chloro and fluoro are examples of halo substituents on alkyl or cycloalkyl groups, unless otherwise specified; fluoro, chloro, and bromo are used, for example, on aryl or heteroaryl groups, unless otherwise specified.

The term “heteroatoms” or “hetero atoms” as used herein refers to nitrogen (N) or oxygen (O) or sulfur (S) atoms, such as nitrogen or oxygen, unless otherwise specified.

The term “optional” or “optionally” used herein means that the subsequently described event or circumstance may or may not occur, and that the description includes instances where the event or circumstance occurs and instances in which it does not. For example, “alkyl optionally substituted with X” encompasses both “alkyl without substitution of X” and “alkyl substituted with X.” It will be understood by those skilled in the art, with respect to any group containing one or more substituents, that such groups are not intended to introduce any substitution or substitution patterns that are sterically impractical, synthetically non-feasible and/or inherently unstable in water at room temperature for at least long enough to be administered as a pharmaceutical agent. When multiple substituents are present, the substituents are selected independently unless otherwise indicated, so where 2 or 3 substituents are present, for example, those substituents may be the same or different.

In some embodiments, “substituted with at least one group” refers to one hydrogen on the designated atom or group being replaced with one selection from the indicated group of substituents. In some embodiments, “substituted with at least one group” refers to two hydrogens on the designated atom or group being independently replaced with two selections from the indicated group of substituents. In some embodiments, “substituted with at least one group” refers to three hydrogens on the designated atom or group being independently replaced with three selections from the indicated group of substituents. In some embodiments, “substituted with at least one group” refers to four hydrogens on the designated atom or group being independently replaced with four selections from the indicated group of substituents.

The term “alkyl” herein refers to a hydrocarbon group chosen from linear and branched saturated hydrocarbon groups having up to 18 carbon atoms, such as from 1 to 12, further such as from 1 to 8, even further such as from 1 to 6, carbon atoms. Representative examples of alkyl include, but not limited to, methyl, ethyl, n-propyl, iso-propyl, n-butyl, sec-butyl, iso-butyl, tert-butyl, n-pentyl, iso-pentyl, neopentyl, n-hexyl, 3-methylhexyl, 2,2-dimethylpentyl, 2,3-dimethylpentyl, n-heptyl, n-octyl, n-nonyl, n-decyl, and the like.

Unless indicated specifically, alkyl group can be optionally substituted by one or more substituents in place of hydrogen atoms of the unsubstituted alkyl, such as one, two or three substituents, or 1-4 substituents, up to the number of hydrogens present on the unsubstituted alkyl group. Suitable substituents for alkyl groups, if not otherwise specified, may be selected from halogen, D, CN, oxo, hydroxyl, substituted or unsubstituted C1-C4 alkoxy, substituted or unsubstituted C3-C6 cycloalkyl, substituted or unsubstituted 3-7 membered heterocycloalkyl containing 1 or 2 heteroatoms selected from N, O and S as ring members, substituted or unsubstituted aryl, substituted or unsubstituted heteroaryl containing 1 to 4 heteroatoms selected from N, O and S as ring members, amino, —NH(C1-C4 alkyl), —N(C1-C4 alkyl)₂, —S(═O)₀₋₂(C1-C4 alkyl), —S(═NR)(═O) (C1-C4 alkyl), —C(═O)(C1-C4 alkyl), —C(═NOH)(C1-C4 alkyl), —CO₂H, —CO₂(C1-C4 alkyl), —S(═O)₁₋₂NH₂, —S(═O)₁₋₂NH(C1-C4 alkyl), —S(═O)₁₋₂N(C1-C4 alkyl)₂, —CONH₂, —C(═O)NH(C1-C4 alkyl), —C(═O)N(C1-C4 alkyl)₂, —C(═NOH)NH(C1-C4 alkyl), —OC(═O)(C1-C4 alkyl), —NHC(═O)(C1-C4 alkyl), —NHC(═NOH)(C1-C4 alkyl), —NH(C═O)NH₂, —NHC(═O)O(C1-C4 alkyl), —NHC(═O)NH(C1-C4 alkyl), NHC(═NOH)NH(C1-C4 alkyl), —NHS(═O)₁₋₂(C1-C4 alkyl), —NHS(═O)₁₋₂NH₂, and —NHS(═O)₁₋₂NH(C1-C4 alkyl); wherein the substituents for substituted C1-C4 alkoxy, substituted C3-C6 cycloalkyl, substituted 3-7 membered heterocycloalkyl, substituted aryl, and substituted heteroaryl are up to three groups independently selected from halogen, D, —CN, C1-C4 alkyl, C1-C4 haloalkyl, oxo, hydroxy, C1-C4 alkoxy, amino, —NH(C1-C4 alkyl), and —N(C1-C4 alkyl)₂. In some embodiments, substituents for alkyl groups, unless otherwise specified, are selected, for example, from halogen, CN, oxo, hydroxy, C1-C4 alkoxy, C3-C6 cycloalkyl, phenyl, amino, —NH(C1-C4 alkyl), —N(C1-C4 alkyl)₂, C1-C4 alkylthio, C1-C4 alkylsulfonyl, —C(═O)(C1-C4 alkyl), —CO₂H, —CO₂(C1-C4 alkyl), —OC(═O)(C1-C4 alkyl), —NHC(═O)(C1-C4 alkyl), and —NHC(═O)O(C1-C4 alkyl).

The term “alkoxy” herein refers to a straight or branched alkyl group comprising from 1 to 18 carbon atoms attached through an oxygen bridge such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Typically, alkoxy groups comprise from 1 to 6 carbon atoms, such as 1 to 4 carbon atoms, attached through the oxygen bridge.

Unless indicated specifically, alkoxy group can be optionally substituted by one or more substituents in place of hydrogen atoms of the unsubstituted alkyl portion of the alkoxy, such as one, two or three substituents, or 1-4 substituents, up to the number of hydrogens present on the unsubstituted alkoxy group. Unless otherwise specified, suitable substituents are selected, for example, from the substituents listed above for alkyl groups, except that hydroxyl and amino are not normally present on the carbon that is directly attached to the oxygen of the substituted alkyl-O group.

The term “alkenyl” herein refers to a hydrocarbon group selected from linear and branched hydrocarbon groups, comprising at least one C═C double bond and from 2 to 18, such as from 2 to 6, carbon atoms. Examples of the alkenyl group may be selected from ethenyl or vinyl (—CH═CH₂), prop-1-enyl (—CH═CHCH₃), prop-2-enyl (—CH₂CH═CH₂), 2-methylprop-1-enyl, buta-1-enyl, buta-2-enyl, buta-3-enyl, buta-1,3-dienyl, 2-methylbuta-1,3-diene, hex-1-enyl, hex-2-enyl, hex-3-enyl, hex-4-enyl, and hexa-1,3-dienyl groups. The point of attachment can be on the unsaturated carbon or saturated carbon.

Unless indicated specifically, alkenyl group can be optionally substituted by one or more substituents in place of hydrogen atoms of the unsubstituted alkenyl, such as one, two or three substituents, or 1-4 substituents, up to the number of hydrogens present on the unsubstituted alkenyl group. Unless otherwise specified, suitable substituents are selected, for example, from the substituents listed above for alkyl groups.

The term “alkynyl” herein refers to a hydrocarbon group selected from linear and branched hydrocarbon groups, comprising at least one —C≡C— triple bond and from 2 to 18, such as from 2 to 6 carbon atoms. Examples of the alkynyl group include ethynyl (—C≡CH), 1-propynyl (—C≡CCH₃), 2-propynyl (propargyl, —CH₂C≡CH), 1-butynyl, 2-butynyl, and 3-butynyl groups. The point of attachment can be on the unsaturated carbon or saturated carbon.

Unless indicated specifically, alkynyl group can be optionally substituted by one or more substituents in place of hydrogen atoms of the unsubstituted alkynyl, such as one, two or three substituents, or 1-4 substituents, up to the number of hydrogens present on the unsubstituted alkynyl group. Unless otherwise specified, suitable substituents are selected, for example, from the substituents listed above for alkyl groups.

The term “alkylene” refers to a divalent alkyl group comprising from 1 to 10 carbon atoms, and two open valences to attach to other molecular components. The two molecular components attached to an alkylene can be on the same carbon atom or on different carbon atoms; thus for example propylene is a 3-carbon alkylene that can be 1,1-disubstituted, 1,2-disubstituted or 1,3-disubstituted. Unless otherwise specified, alkylene refers to moieties comprising from 1 to 6 carbon atoms, such as from 1 to 4 carbon atoms. Examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents; unless otherwise specified, suitable substituents are selected, for example, from the substituents listed above for alkyl groups.

Unless indicated specifically, alkylenyl group can be optionally substituted by one or more substituents in place of hydrogen atoms of the unsubstituted alkylenyl, such as one, two or three substituents, or 1-4 substituents, up to the number of hydrogens present on the unsubstituted alkylenyl group. Unless otherwise specified, suitable substituents are selected, for example, from the substituents listed above for alkyl groups.

Similarly, “alkenylene” and “alkynylene” refer to alkylene groups comprising a double bond or a triple bond, respectively; they are, for example, 2-6 such as 2-4 carbon atoms in length, and can be substituted as discussed above for alkylene groups.

The term “haloalkyl” refers to an alkyl as defined herein, which is substituted by one or more halo groups as defined herein. Unless otherwise specified, the alkyl portion of the haloalkyl comprises 1-4 carbon atoms. The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. The polyhaloalkyl comprises, for example, up to 6, or 4, or 3, or 2 halo groups. Examples of haloalkyl include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl. In some embodiments, the haloalkyl groups, unless specified otherwise, include monofluoro-, difluoro- and trifluoro-substituted methyl and ethyl groups, e.g. —CF₃, —CF₂H, —CFH₂ and —CH₂CF₃.

Unless indicated specifically, haloalkyl group can be optionally substituted by one or more substituents in place of hydrogen atoms of the unsubstituted haloalkyl, such as one, two or three substituents, or 1-4 substituents, up to the number of hydrogens present on the unsubstituted haloalkyl group. Unless otherwise specified, suitable substituents are selected, for example, from the substituents listed above for alkyl groups.

As used herein, the term “haloalkoxy” refers to haloalkyl-O—, wherein haloalkyl is defined above. Examples of haloalkoxy include, but are not limited to, fluoromethoxy, difluoromethoxy, trifluoromethoxy, trichloromethoxy, 2-chloroethoxy, 2,2,2-trifluoroethoxy, 1,1,1,3,3,3-hexafluoro-2-propoxy, and the like. In some embodiments, haloalkyloxy groups comprise 1-4 carbon atoms, and up to three halogens, e.g., monofluoro, difluoro and trifluoro substituted methoxy groups and ethoxy groups.

Unless indicated specifically, haloalkoxy group can be optionally substituted by one or more substituents in place of hydrogen atoms of the unsubstituted alkyl portion of the haloalkoxy, such as one, two or three substituents, or 1-4 substituents, up to the number of hydrogens present on the unsubstituted haloalkoxy group. Unless otherwise specified, suitable substituents are selected, for example, from the substituents listed above for alkyl groups, except that hydroxyl and amino are not normally present on the carbon that is directly attached to the oxygen of the substituted haloalkyl-O group.

The term “cycloalkyl” herein refers to a hydrocarbon group selected from saturated and partially unsaturated cyclic hydrocarbon groups comprising from 3 to 20 carbon atoms, such as monocyclic and polycyclic (e.g., bicyclic and tricyclic, adamantanyl and spirocycloalkyl) groups. Monocycloalkyl groups are cyclic hydrocarbon groups comprising from 3 to 20 carbon atoms, such as from 3 to 8 carbon atoms. Examples of monocyclic cycloalkyl include, but are not limited to, cyclopropyl, cyclobutyl, cyclopentyl, cyclohexyl, cycloheptyl, cyclooctyl, cyclodecanyl, cyclodocecanyl, and cyclohexenyl. Bicycloalkyl groups include bridged bicycloalkyl, fused bicycloalkyl and spirocycloalkyls. Bridged bicycloalkyl contains a monocyclic cycloalkyl ring where two non-adjacent carbon atoms of the monocyclic ring are linked by an alkylene bridge of one to three additional carbon atoms (i.e. a bridging group of the form —(CH₂)ₙ—, wherein n is 1, 2, or 3). Examples of bridged bicycloalkyl include, but are not limited to, bicyclo[2.2.1]heptenes, bicyclo[3.1.1]heptanes, bicyclo[2.2.1]heptanes, bicyclo[2.2.2]octane, bicyclo[3.2.2]nonane, bicyclo[3.3.1]nonane, and bicycle[4.2.1]nonane.

Fused bicycloalkyl contains a monocyclic cycloalkyl ring fused to either a phenyl, a monocyclic cycloalkyl, or a monocyclic heteroaryl. Examples of fused bicycloalkyl include, but are not limited to, bicyclo[4.2.0] octa-1,3,5-triene, 2,3-dihydro-1H-indene, 6,7-dihydro-5H-cyclopenta[b]pyridine, 5,6-dihydro-4H-cyclopenta[b]thiophene, and decahydronaphthalene. Spirocycloalkyl contains two monocyclic ring systems that share a carbon atom forming a bicyclic ring system. Examples of spirocycloalkyls include, but are not limited to,

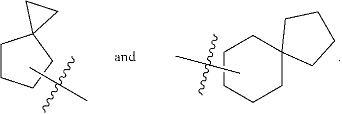

Bicyclic cycloalkyl groups comprise, for example, from 7 to 12 carbon atoms. Monocycloalkyl or bicycloalkyl is attached to the parent molecular moiety through any carbon atom contained within the cycloalkyl ring. Tricycloalkyl groups include bridged tricycloalkyl as used herein referring to 1) a bridged bicycloalkyl ring where two non-adjacent carbon atoms of the bridged bicycloalkyl ring are linked by an alkylene bridge of one to three additional carbon atoms (i.e. a bridging group of the form —(CH₂)ₙ—, wherein n is 1, 2, or 3), or 2) a fused bicycloalkyl ring where two unshared ring atoms on each ring are linked by an alkylene bridge of one to three additional carbon atoms (i.e. a bridging group of the form —(CH₂)ₙ—, wherein n is 1, 2, or 3), wherein “a fused bicycloalkyl ring” refers to a monocycloalkyl ring fused to a monocycloalkyl ring. Examples of bridged tricycloalkyl groups include, but are not limited to, adamantanyl

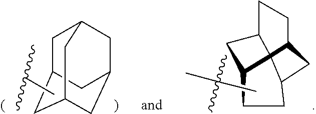

Bridged tricycloalkyl, as used herein, is appended to the parent molecular moiety through any ring atom. The ring atom disclosed herein refers to the carbon atom on the ring skeleton. The cycloalkyl may be saturated or comprise at least one double bond (i.e. partially unsaturated), but is not fully conjugated, and is not aromatic, as aromatic is defined herein. The cycloalkyl may be substituted with at least one hetero atom selected, for example, from O, S, and N.

Unless indicated specifically, cycloalkyl group can be optionally substituted by one or more substituents in place of hydrogen atoms of the unsubstituted cycloalkyl, such as one, two or three substituents, or 1-4 substituents, up to the number of hydrogens present on the unsubstituted cycloalkyl group. In some embodiments, a substituted cycloalkyl comprises 1-4 such as 1-2 substituents. Unless otherwise specified, suitable substituents are selected, for example, from the substituents listed above for alkyl groups.

The term “cycloalkylidenyl” or “cycloalkylidene ring” disclosed herein refers to a divalent cycloalkane ring attached via the same carbon atom of the cycloalkane ring by removal of two hydrogen atoms from the same carbon atoms. Examples of cycloakylidenyl rings include, but are not limited to, cyclopropylidenyl, cyclobutylidenyl, cyclopentylidenyl, and cyclohexylidenyl. It can be represented in illustrative fashion by the following structure in which n is 1, 2, 3, 4, or 5.

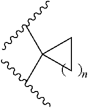

The term “heterocycloalkyl,” “heterocyclyl,” or “heterocyclic” disclosed herein refers to “cycloalkyl” as defined above with at least one ring carbon atom being replaced by a heteroatom independently selected from O, N, and S. Heterocyclyl comprises, for example, 1, 2, 3, or 4 heteroatoms, and the N, C or S can independently be oxidized in the cyclic ring system. The N atom can further be substituted to form tertiary amine or ammonium salts. The point of attachment of heterocyclyl can be on the heteroatom or carbon. “Heterocyclyl” herein also refers to a 5- to 7-membered saturated or partially unsaturated carbocyclic ring comprising at least one heteroatom selected, for example, from N, O, and S (heterocyclic ring) fused with 5-, 6-, and/or 7-membered cycloalkyl, heterocyclic or carbocyclic aromatic ring, provided that the point of attachment is at the heterocyclic ring when the heterocyclic ring is fused with a carbocyclic aromatic ring, and that the point of attachment can be at the cycloalkyl or heterocyclic ring when the heterocylic ring is fused with cycloalkyl. “Heterocyclyl” herein also refers to an aliphatic spirocyclic ring comprising at least one heteroatom selected, for example, from N, O, and S. The rings may be saturated or have at least one double bond (i.e. partially unsaturated). The heterocyclyl may be substituted with, for example, oxo. The point of the attachment may be carbon or heteroatom. A heterocyclyl is not a heteroaryl as defined herein.

Examples of the heterocycle include, but not limited to, pyrrolidinyl, imidazolidinyl, pyrazolidinyl, piperidinyl, piperidinyl, piperazinyl, pyranyl, morpholinyl, oxiranyl, aziridinyl, thiiranyl, azetidinyl, oxetanyl, thietanyl, dithietanyl, dihydropyridinyl, tetrahydropyridinyl, thiomorpholinyl, thioxanyl, homopiperazinyl, homopiperidinyl, azepanyl, oxepanyl, thiepanyl, oxathianyl, dioxepanyl, oxathiepanyl, oxaazepanyldithiepanyl, thiazepanyl and diazepane, dithianyl, azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, indolinyl, dioxanyl, pyrazolinyl, dithianyl, dithiolanyl, pyrazolidinylimidazolinyl, pyrimidinonyl, 1,1-dioxo-thiomorpholinyl, 3-azabicyclo[3.1.0]hexanyl, 3-azabicyclo[4.1.0]heptanyl and azabicyclo[2.2.2]hexanyl. Substituted heterocycles also include ring systems substituted with one or more oxo moieties, such as piperidinyl N-oxide morpholinyl-N-oxide, 1-oxo-1-thiomorpholinyl, 1,1-dioxo-1-thiomorpholinyl,

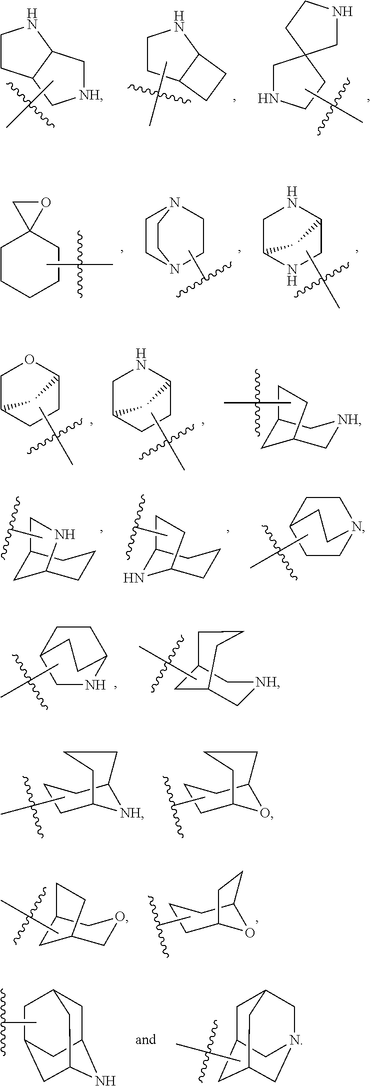

Unless indicated specifically, heterocyclyl group can be optionally substituted by one or more substituents in place of hydrogen atoms of the unsubstituted heterocyclyl, such as one, two or three substituents, or 1-4 substituents, up to the number of hydrogens present on the unsubstituted heterocyclyl group. In some embodiments, a substituted heterocycloalkyl comprises 1-4 such as 1-2 substituents. Unless otherwise specified, suitable substituents are selected, for example, from the substituents listed above for alkyl groups.

The term “aryl” refers to an aromatic hydrocarbon group comprising 5-15 carbon atoms in the ring portion. In some embodiments, aryl refers to a group selected from 5- and 6-membered carbocyclic aromatic rings, for example, phenyl; bicyclic ring systems such as 7 to 12 membered bicyclic ring systems wherein at least one ring is carbocyclic and aromatic, selected, for example, from naphthalene, indane, and 1,2,3,4-tetrahydroquinoline; and tricyclic ring systems such as 10 to 15 membered tricyclic ring systems, wherein at least one ring is carbocyclic and aromatic, for example, fluorene.

In some embodiments, the aryl group is selected from 5- and 6-membered carbocyclic aromatic rings fused to a 5- to 7-membered cycloalkyl or heterocyclic ring (as defined in “heterocyclyl” or “heterocyclic” below) optionally comprising at least one heteroatom selected, for example, from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring (e.g., a heteroaryl as defined below), the resulting ring system is heteroaryl, not aryl, as defined herein.

Unless indicated specifically, aryl group can be optionally substituted by one or more substituents in place of hydrogen atoms of the unsubstituted aryl, such as one, two or three substituents, or 1-4 substituents, up to the number of hydrogens present on the unsubstituted aryl group. In some embodiments, a substituted aryl group comprises 1-5 substituents. Unless otherwise specified, suitable substituents are selected, for example, from the substituents listed above for alkyl groups.

The term “heteroaryl” herein refers to a group selected from 5- to 7-membered aromatic, monocyclic rings comprising at least one heteroatom, for example, from 1 to 4, or, in some embodiments, from 1 to 3, heteroatoms, selected, for example, from N, O, and S, with the remaining ring atoms being carbon; 8- to 12-membered bicyclic rings comprising at least one heteroatom, for example, from 1 to 4, or, in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in the aromatic ring, and with the point of attachment being on any ring and being on either carbon or the heteroatom; and 11- to 14-membered tricyclic rings comprising at least one heteroatom, for example, from 1 to 4, or in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in an aromatic ring, and with the point of attachment being on any ring.

In some embodiments, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

In some embodiments, the heteroaryl group includes a 5- to 7-membered heterocyclic aromatic ring fused to a 5- to 7-membered aryl ring. For such fused, bicyclic heteroaryl ring systems wherein only one of the rings comprises at least one heteroatom, the point of attachment may be at the heteroaromatic ring or at the aryl ring. Non-limiting examples include quinolinyl and quinazolinyl.

In some embodiments, the heteroaryl group includes a 5- to 7-membered heterocyclic aromatic ring fused to another 5- to 7-membered heterocyclic aromatic ring. Non-limiting examples include 1H-pyrazolo[3,4-b]pyridinyl and 1H-pyrrolo[2,3-b]pyridinyl.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, pyridyl, cinnolinyl, pyrazinyl, pyrimidinyl, imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrimidinyl, pyrrolyl, triazolyl, quinolinyl, isoquinolinyl, pyrazolyl, pyrrolopyridinyl (such as 1H-pyrrolo[2,3-b]pyridin-3-yl), pyrazolopyridinyl (such as 1H-pyrazolo[3,4-b]pyridin-3-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

Unless indicated specifically, heteroaryl group can be optionally substituted by one or more substituents in place of hydrogen atoms of the unsubstituted heteroaryl, such as one, two or three substituents, or 1-4 substituents, up to the number of hydrogens present on the unsubstituted heteroaryl group. In some embodiments, a substituted heteroaryl group comprises 1, 2 or 3 substituents. Unless otherwise specified, suitable substituents are selected, for example, from the substituents listed above for alkyl groups.

Compounds disclosed herein may contain an asymmetric center and may thus exist as enantiomers. Where the compounds disclosed herein possess two or more asymmetric centers, they may additionally exist as diastereomers. Enantiomers and diastereomers fall within the broader class of stereoisomers. It is well-known in the art how to prepare optically active forms, such as by resolution of materials or by asymmetric synthesis. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When the compounds disclosed herein contain olefinic double bonds, unless specified otherwise, such double bonds are meant to include both E and Z geometric isomers.

“A pharmaceutically acceptable salt” includes, but is not limited to, salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, aluminum, lithium, and ammonium.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating”, “treat”, “treatment” or “alleviation” refers to administering at least one compound and/or at least one stereoisomer thereof, if any, at least one stable isotope thereof, or at least one pharmaceutically acceptable salt thereof disclosed herein to a subject in recognized need thereof that has, for example, cancer.

The term “effective amount” refers to an amount of at least one compound and/or at least one stereoisomer thereof, if any, at least one stable isotope thereof, or at least one pharmaceutically acceptable salt thereof disclosed herein effective to “treat,” as defined above, a disease or disorder in a subject.

The term “RET-associated disease”, “RET-associated disorder”, “RET-associated cancer”, “diseases and disorders modulated by RET”, or “aberrant RET activity” refers to disease, disorder, or cancer associated with or having a dysregulation of RET gene. The dysregulation of a RET gene is caused by RET gene mutation that consists of, for example, a RET gene translocation resulting in the expression of a fusion protein, a deletion in a RET gene resulting in the expression of a RET protein that includes a deletion of at least one amino acid as compared to the wild-type RET protein, a mutation in a RET gene that results in the expression of a RET protein with one or more mutations, an alternative spliced version of a RET mRNA that results in a RET protein having a deletion of at least one amino acid in the RET protein, or a RET gene amplification that results in overexpression of a RET gene in a cell leading to a pathogenic increase in the activity of a kinase domain of a RET protein or a constitutively active kinase domain of a RET protein in cell. For example, at least 12 different fusion variants have been identified, with KIF5B-RET being the most common in NSCLCs, and CCDC6 and NCOA4 being most common in PTCs Example of RET point mutations are, not limited to, M918T, G810R, V804L and V804M (Drilon, A. et al. Nat. Rev. Clin. Oncol., 2018, 15, 151-167). Examples of RET-associated diseases or disorders include, but are not limited to, cancers and gastrointestinal disorders such as irritable bowel syndrome.

Various embodiments are disclosed herein. It will be recognized that features specified in each embodiment may be combined with other specified features to provide further embodiments of the present disclosure. The following enumerated embodiments are representative of the present disclosure.

Embodiment 1. Disclosed herein is a compound of Formula I:

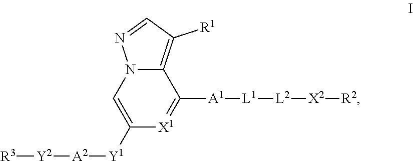

and/or stereoisomers, stable isotopes, or pharmaceutically acceptable salts or solvates thereof, wherein R¹, R², R³, A¹, A², L¹, L², X¹, X², Y¹ and Y² are defined below.
R¹ is selected from H, —CN, ethynyl, halo, —CF₃, —CH₃, —CH₂CH₃, cyclopropyl, —CH₂CN, and —CH(CN)CH₃;
R² is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R² is 1-4 substituents independently selected from R⁴, wherein each R⁴ is independently selected from halo, —OH, NH₂, ═O, —CN, OC(O)R⁵, —CO₂R⁵, —C(O)N(R^{6a}R^{6b}), —C(═NR⁷)N(R^{6a}R^{6b}), —C(O)R⁵, —S(O)₀₋₂R⁸, —S(O)(═NR⁷)R⁸, —S(O)₁₋₂N(R^{6a}R^{b}), —N(R^{6a}R^{6b}), —N(R^{6a})C(O)R⁸, —N(R^{6a})C(═NR⁷)R⁸, —N(R^{6a})S(O)₁₋₂R⁸, —N(R^{6c})C(O)N(R^{6a}R^{6b}), —N(R^{6c})C(═NR⁷)N(R^{6a}R^{6b}), —N(R^{6c})S(O)₁₋₂N(R^{6a}R^{6b}), —N(R^{6a})CO₂R⁸, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated and unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R⁵, R^{6a}, R^{6b} and R^{6c} are independently selected from H, C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; R⁷ is independently selected from H, —CN, —OH, C1-C4 alkyl and C1-C4 alkoxy; R⁸ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; wherein each of R⁵, R^{6a}, R^{6b}, R^{6c}, R⁷, and R⁸ is optionally substituted with 1-3 groups independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl;
wherein two substituents on the same or adjacent carbon atoms of R² can optionally be taken together to form a 4-6 membered ring that can be saturated or aromatic and optionally contains 1-2 heteroatoms selected from N, O, and S and can optionally be substituted with 1-2 groups independently selected from R⁴;
R³ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, saturated and unsaturated, saturated 7-8 membered bridged heterocyclyl containing 1-2 heteroatoms selected from N, O, and S as ring members, saturated 7-11 membered spiroheterocyclyl containing 1-2 heteroatoms selected from N, O, and S as ring members, and 5-membered heteroaryl containing 1-3 heteroatoms selected from N, O, and S as ring members; and wherein the optional substituents for R³ is 1-4 substituents independently selected from R⁴;
A¹ is an optionally substituted group selected from para-attached benzene, para-attached 6-membered heteroarene containing 1-2 N as ring members, 2,5-attached thiophene, and 2,5-attached thiazole, wherein the optional substituents are 1-3 substituents selected from F, Cl, CN, CH₃, and CF₃;
A² is a bond or an optionally substituted C1-C6 alkylenyl wherein the optional substituents are 1-3 substituents selected from R⁴; L¹ is selected from

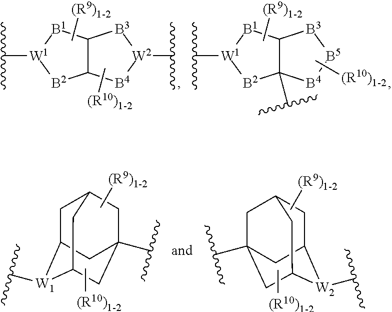

wherein W¹ is N or

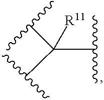

wherein R¹¹ is selected from H, OH, CN, F, and an optionally substituted group selected from C1-C6 alkyl and C1-C6 alkoxy, and wherein the optional substituents are 1-3 groups independently selected from halo, OH, CN, C1-C3 alkyl, C1-C3 haloalkyl, C1-C3 alkoxy, C1-C3 haloalkoxy, C3-C6 cycloalkyl, and C3-C6 cycloalkyloxy;
wherein W² is N or

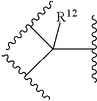

or, wherein R¹² is selected from H, F, OH, —CO₂H, and an optionally substituted group selected from C1-C6 alkyl and C1-C6 alkoxy, and wherein the optional substituents are 1-3 groups independently selected from R⁴; wherein the left wavy line indicates the point of attachment of L¹ to A¹; wherein the right wavy line indicates the point of attachment of L¹ to L²;
wherein B¹, B², B³ and B⁴ are independently selected from a bond, —O—, and an optionally substituted C1-C3 alkylenyl wherein the optional substituents are 1-3 substituents each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein zero, one, or two of B¹, B², B³ and B⁴ is a bond or —O—;
wherein B5 is —O—, or an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein when B⁵ is —O—, B³ and B⁴ cannot be —O—, or zero or one of B³ and B⁴ is a bond;
wherein R⁹ and R¹⁰ are independently selected from R⁴;
L² is a bond or an optionally substituted C1-C4 alkylenyl, wherein the optional substituents are 1-3 groups independently selected from R⁴; wherein L² and W² via R¹² together optionally form 3-6 membered spirocycloalkyl or 4-6 membered spiroheterocycles containing 1-2 heteroatoms independently selected from N, O, and S as ring members;
X¹ is —C(H)— or N;
X² is selected from a bond, —O—, —N(R¹³)—, —C(O)—, —C(O)O—, C(O)N(R¹³)—, —N(R¹³)C(O)—, —N(R¹³)C(O)N(R¹⁴)—, —N(R¹³)C(O)O—, —S(O)₀₋₂—, —S(O)₁₋₂NR¹³—, —N(R¹³)S(O)₁₋₂—, —S(O)(═NR¹⁵)—, —S(O)(═NR¹⁵)NR—, —NR¹³S(O)(═NR¹⁵)—, N(R¹³)S(O)₂N(R¹⁴)—, and an optionally submitted group selected from C1-C3 alkylenyl and C3-C6 cycloalkylidenyl; wherein R¹³ and R¹⁴ are independently selected from H and an optionally substituted group independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members, and the optional substituents are 1-3 groups independently selected from R⁴; R¹⁵ is selected from H, —CN, —OH, and an optionally substituted group selected from C1-C4 alkyl and C1-C4 alkoxy, and the optional substituents are 1-3 groups independently selected from R⁴;
Y¹ is selected from a bond, 0, —N(R¹³)—, and an optionally substituted C1-C3 alkylenyl wherein the optional substituents are 1-3 groups independently selected R⁴; and
Y² is selected from a bond, —O—, and —N(R¹³).

Embodiment 2. The compound of Embodiment 1, and/or a stereoisomer, a stable isotope, or a pharmaceutically acceptable salt or solvate thereof, wherein L¹ is selected from

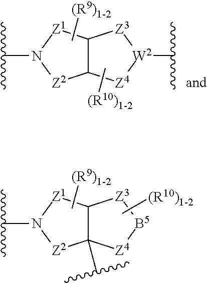

wherein the left wavy line indicates the point of attachment of L¹ to A¹; wherein the right wavy line indicates the point of attachment of L¹ to L²;
wherein Z¹, Z², Z³ and Z⁴ are independently selected from a bond and an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein zero or one of Z¹ and Z² is bond, and zero, one, or two of Z¹, Z², Z³ and Z⁴ are bonds;
wherein B5 is —O—, or an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein when B⁵ is —O—, Z³ and Z⁴ cannot be —O—, or zero or one of Z³ and Z⁴ is a bond; and
wherein R⁹, R¹⁰ and W² are as defined in Embodiment 1.

Embodiment 3. A compound of Embodiment 1, and/or a stereoisomer, a stable isotope, or a pharmaceutically acceptable salt or solvate thereof, wherein L¹ is

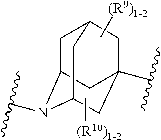

wherein the left wavy line indicates the point of attachment of L¹ to A¹; wherein the right wavy line indicates the point of attachment of L¹ to L²; and
wherein R⁹ and R¹⁰ are as defined in Embodiment 1.

Embodiment 4. The compound of any one of Embodiments 1-2, and/or a stereoisomer, a stable isotope, or a pharmaceutically acceptable salt or solvate thereof, wherein L¹ is

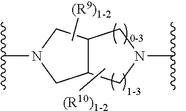

wherein the left wavy line indicates the point of attachment of L¹ to A¹; wherein the right wavy line indicates the point of attachment of L¹ to L²; and
wherein R⁹ and R¹⁰ are as defined in Embodiment 1.

Embodiment 5. The compound of any one of Embodiments 1-2, and/or a stereoisomer, a stable isotope, or a pharmaceutically acceptable salt or solvate thereof, wherein L¹ is selected from

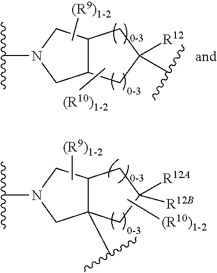

wherein the left wavy line indicates the point of attachment of L¹ to A¹; wherein the right wavy line indicates the point of attachment of L¹ to L²;
wherein R^{12A} and R^{12B} are independently selected from H, F, OH, —CO₂H, and an optionally substituted group selected from C1-C6 alkyl and C1-C6 alkoxy, and wherein the optional substituents are 1-3 groups independently selected from R⁴; and
wherein R⁹, R¹⁰, and R¹² are as defined in Embodiment 1.

Embodiment 6. The compound of any one of Embodiments 1-2, and/or a stereoisomer, a stable isotope, or a pharmaceutically acceptable salt or solvate thereof, wherein L¹ is selected from

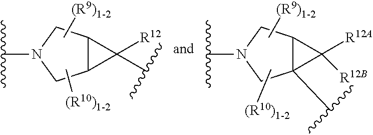

wherein the left wavy line indicates the point of attachment of L¹ to A¹; wherein the right wavy line indicates the point of attachment of L¹ to L²; and
wherein R⁹, R¹⁰, R¹², R^{12A} and R^{12B} are as defined in Embodiment 1 and Embodiment 5.

Embodiment 7. The compound of any one of Embodiments 1-6, and/or a stereoisomer, a stable isotope, or a pharmaceutically acceptable salt or solvate thereof, wherein L² is a bond.

Embodiment 8. The compound of any one of Embodiments 1-6, and/or a stereoisomer, a stable isotope, or a pharmaceutically acceptable salt or solvate thereof, wherein L² is an optionally substituted C1-C4 alkylenyl and wherein the optional substituents are 1-3 groups independently selected R⁴.

Embodiment 9. The compound of any one of Embodiments 1, 2, and 5, and/or a stereoisomer, a stable isotope, or a pharmaceutically acceptable salt or solvate thereof, wherein L¹ and L² together form

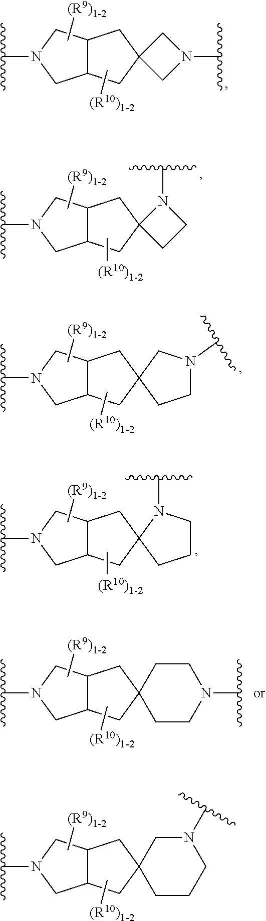

wherein the left wavy line indicates the point of attachment of L¹ to A¹; wherein the right wavy line indicates the point of attachment of L¹ to X²; and wherein R⁹ and R¹⁰ are as defined in Embodiment 1.

Embodiment 10. The compound of any one of Embodiments 1-9, and/or a stereoisomer, a stable isotope, or a pharmaceutically acceptable salt or solvate thereof, wherein A¹ is

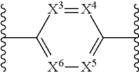

wherein X³, X⁴, X⁵, and X⁶ are independently selected from CH, —C(CH₃)—, CF, and N, wherein zero, one, or two of X³, X⁴, X⁵ and X⁶ is N.

Embodiment 11. The compound of any one of Embodiments 1-10, and/or a stereoisomer, a stable isotope, or a pharmaceutically acceptable salt or solvate thereof, wherein X² is selected from —N(R¹³)C(O)—, C(O)N(R¹³)—, —N(R¹³)C(O)N(R¹⁴)—, —N(R¹³)C(O)O—, —N(R¹³)S(O)₂—, C1-C3 alkylenyl, and C3-C6 cycloalkylidenyl; and
wherein R¹³ and R¹⁴ are as defined in Embodiment 1.

Embodiment 12. The compound of any one of Embodiments 1-3, 5-8, and 10-11, and/or a stereoisomer, a stable isotope, or a pharmaceutically acceptable salt or solvate thereof, wherein -L¹-L²-X²—R² is selected from

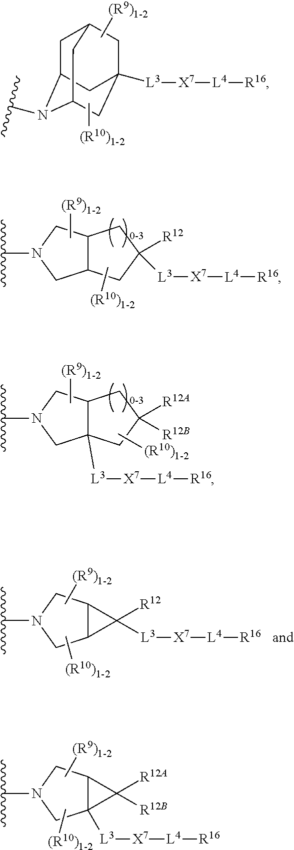

wherein L³ and L⁴ are independently selected from a bond and a C1-C3 alkylenyl group optionally substituted by 1-3 substituents independently selected from R⁴; X⁷ is selected from a bond, —O—, —N(R¹³)—, —N(R¹³)C(O)—, —N(R¹³)S(O) 2-, —C(O)N(R¹³)—, —S(O)₂N(R¹³)—, —N(R¹³)C(O)N(R¹⁴)—, —N(R¹³)C(O)O—, —OC(O)N(R¹³)—, and —N(R¹³)S(O)₂N(R¹⁴)—; R¹⁶ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁶ is 1-4 substituents independently selected from R⁴; and
wherein R⁹, R¹⁰, R¹², R¹³, R¹⁴, R^{12A}, and R^{12B} are as defined in Embodiment 1 and Embodiment 5.

Embodiment 13. The compound of any one of Embodiments 1-2, 4, and 7-11, and/or a stereoisomer, a stable isotope, or a pharmaceutically acceptable salt or solvate thereof, wherein -L¹-L²-X²—R² is selected from

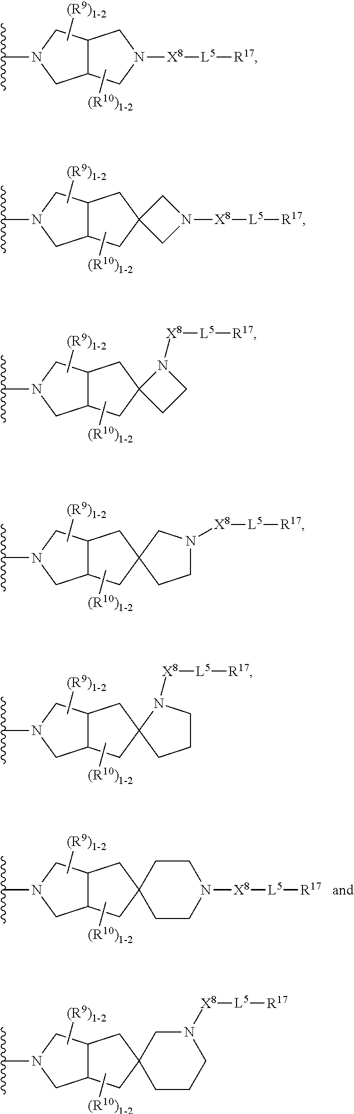

wherein L⁵ is selected from a bond and a C1-C3 alkylenyl group optionally substituted by 1-3 substituents independently selected from R⁴; X⁸ is selected from a bond, —C(O)—, and —S(O)₂—; R¹⁷ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁷ is 1-4 substituents independently selected from R⁴; and
wherein R⁹ and R¹⁰ are as defined in Embodiment 1.

Embodiment 14. The compound of any one of Embodiments 1-13, and/or a stereoisomer, a stable isotope, or a pharmaceutically acceptable salt or solvate thereof, wherein R³ is a saturated or unsaturated 4-7 membered heterocyclyl containing 1-2 heteroatoms selected from N, O, and S as ring members.

Embodiment 15. The compound of any one of Embodiments 1-13, and/or a stereoisomer, a stable isotope, or a pharmaceutically acceptable salt or solvate thereof, wherein R³ is a saturated 7-8 membered bridged heterocyclyl containing 1-2 heteroatoms selected from N, O, and S as ring members.

Embodiment 16. The compound of any one of Embodiments 1 to 13, and/or a stereoisomer, a stable isotope, or a pharmaceutically acceptable salt or solvate thereof, wherein A², Y¹ and Y² are bonds; R³ is an optionally substituted group selected from saturated and unsaturated 4-6 membered heterocyclyl containing 1-2 heteroatoms selected from N, O, and S as ring members, and 5-membered heteroaryl containing 1-4 heteroatoms selected from N, O, and S as ring members; and wherein the optional substituents for R³ is 1-4 substituents independently selected from R⁴.

Embodiment 17. The compound of any one of Embodiments 1 to 13, and/or a stereoisomer, a stable isotope, or a pharmaceutically acceptable salt or solvate thereof, wherein Y¹ is selected from a bond, —O—, and —N(R¹³)—; A² is a an optionally substituted C1-C6 alkylenyl, wherein the optional substituents are 1-3 substituents selected from R⁴; Y² is selected from a bond, —O—, and —N(R¹³)—, and wherein R¹³ is as defined in Embodiment 1.

Embodiment 18. The compound of any one of Embodiments 1 to 13, and 15, and/or a stereoisomer, a stable isotope, or a pharmaceutically acceptable salt or solvate thereof, wherein Y¹ is —O—; A² is a an optionally substituted C1-C6 alkylenyl, wherein the optional substituents are 1-3 substituents selected from R⁴; and Y² is selected from a bond and —O—.

Embodiment 19. The compound of any one of Embodiments 1 to 13, and 15-16, and/or a stereoisomer, a stable isotope, or a pharmaceutically acceptable salt or solvate thereof, wherein R³—Y²-A²-Y¹— is

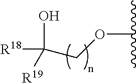

wherein n is 1, 2 or 3; R¹⁸ and R¹⁹ are independently selected from H and an optionally substituted group selected from C1-C6 alkyl, C3-C6 cycloalkyl, and saturated and unsaturated 4-7 membered heterocyclyl containing 1-2 heteroatoms selected from N, O, and S as ring members; and wherein the optional substituents are 1-4 substituents independently selected from R⁴; and wherein R¹⁸ and R¹⁹ together optionally form 3-6 membered cycloalkyl or 4-6 membered heterocycles containing 1-2 heteroatoms independently selected from N, O, and S as ring members.

Embodiment 20. The compound of any one of Embodiments 1-19, and/or a stereoisomer, a stable isotope, or a pharmaceutically acceptable salt or solvate thereof, wherein R¹ is CN; and X¹ is CH.

Embodiment 21. The compound of any one of Embodiments 1-14 and 20, and/or a stereoisomer, a stable isotope, or a pharmaceutically acceptable salt or solvate thereof, wherein Y¹, A² and Y² are bonds; R³ is selected from

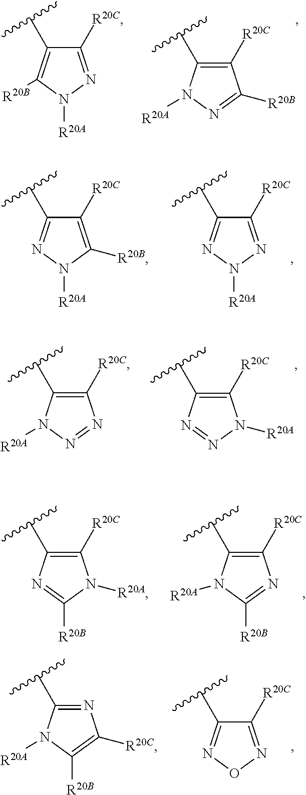

wherein R^{20A} is independently selected from H, Me, Et, propyl, isopropyl, butyl, isobutyl, sec-butyl, t-butyl, —CH₂F, —CF₂H, —CF₃, and cyclopropyl; and R^{20B} and R^{20C} are independently selected from H, Me, Et, propyl, isopropyl, butyl, isobutyl, sec-butyl, t-butyl, —CH₂F, —CF₂H, —CF₃, cyclopropyl, —OMe, —OEt, —OPr, —OⁱPr, —OBu, —OⁱBu, —O^{s}Bu, —O^{t}Bu, —OCF₃, —O(cycloproyl), —CN, Cl, and F.

Embodiment 22. The compound of Embodiment 1, which is of the Formula IA, and/or a stereoisomer, a stable isotope, or a pharmaceutically acceptable salt or solvate thereof:

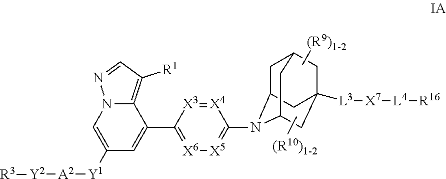

wherein R¹, R³, R⁹, R¹⁶, A², L³, L⁴, X³-X⁷, Y¹, and Y² are as defined previously.

Embodiment 23. The compound of Embodiment 1, which is of the Formula IB, and/or a stereoisomer, a stable isotope, or a pharmaceutically acceptable salt or solvate thereof:

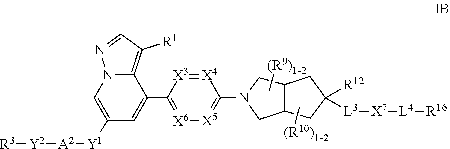

wherein R¹, R³, R⁹, R¹⁰, R¹², R¹⁶, A², L³, L⁴, X³-X⁷, Y¹, and Y² are as defined previously.

Embodiment 24. The compound of Embodiment 1, which is of the Formula IC, and/or a stereoisomer, a stable isotope, or a pharmaceutically acceptable salt or solvate thereof:

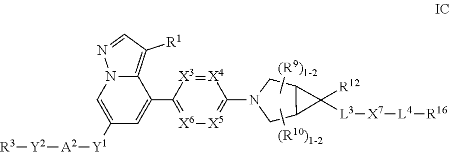

wherein R¹, R³, R⁹, R¹⁰, R¹², R¹⁶, A², L³, L⁴, X³-X⁷, Y¹, and Y² are as defined previously.

Embodiment 25. The compound of Embodiment 1, which is of the Formula ID, and/or a stereoisomer, a stable isotope, or a pharmaceutically acceptable salt or solvate thereof:

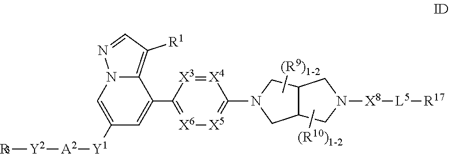

wherein R¹, R³, R⁹, R¹⁰, R⁷, A², L⁵, X³-X⁸, Y¹, and Y² are as defined previously.

Embodiment 26. The compound of Embodiment 1, which is of the Formula IE, and/or a stereoisomer, a stable isotope, or a pharmaceutically acceptable salt or solvate thereof:

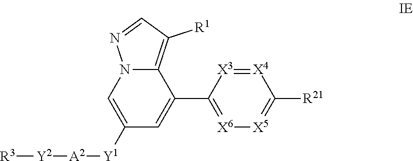

wherein R²¹ is selected from

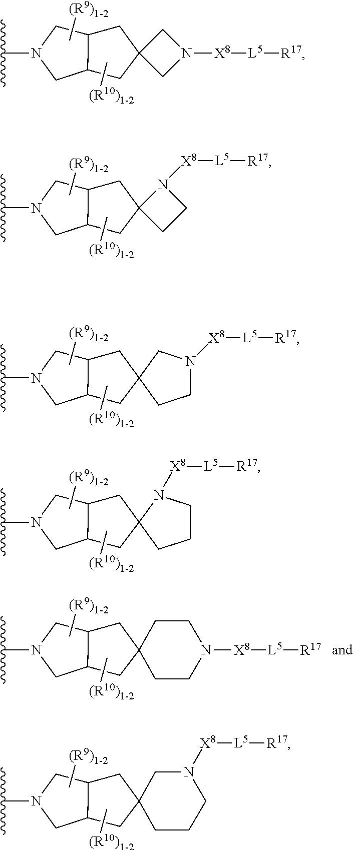

wherein R¹, R³, R⁹, R¹⁰, R⁷, A², L⁵, X³-X⁸, Y¹, and Y² are as defined previously.

Embodiment 27. The compound of Embodiment 1, which is selected from the following compounds, and/or a stereoisomer, a stable isotope, or a pharmaceutically acceptable salt or solvate thereof:
4-(6-((3aR,6aS)-5-(6-methoxynicotinoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-hydroxy-3-methylbutanoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-hydroxy-2-phenylacetyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-chloro-6-fluorobenzoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-ethoxypyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(2-hydroxypropoxy)pyrazolo[1,5-a]pyridine-3-carbonitrile,
4-(6-((3aR,6aS)-5-(2-chloro-6-fluorobenzoyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
4-(6-((3aR,6aS)-5-isobutyrylhexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-chloro-6-fluorophenylsulfonyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-((6-methoxypyridin-3-yl)methyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-((6-methoxypyridin-3-yl)methyl)hexahydropyrrolo[3,4-c]pyrrol-2(1H)-yl)pyridin-3-yl)-6-(1-methyl-1H-pyrazol-3-yl)pyrazolo[1,5-a]pyridine-3-carbonitrile,
6-(2-hydroxy-2-methylpropoxy)-4-(6-((3aR,6aS)-5-((6-methoxypyridin-3-yl)methyl)hexahydropyrrolo[3,4-c]pyrrol-2(1H)-yl)pyridin-3-yl)pyrazolo[1,5-a]pyridine-3-carbonitrile,
N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-6-methoxynicotinamide,
N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-2-hydroxy-3-methylbutanamide,
N-((1R,5S,6r)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-2-hydroxy-3-methylbutanamide,
(R)-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-hydroxy-2-phenylacetamide,
(R)-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-hydroxy-3-methylbutanamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)picolinamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-(trifluoromethyl)picolinamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluoropicolinamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-methylbenzamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-methylbutanamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluoro-2-methylbenzamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-methylpicolinamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluorobenzamide,
3-chloro-N-((1R,5S,6s)-3-(5-(3-cyano-6-(1-methyl-1H-pyrazol-3-yl)pyrazolo[1,5-a]pyridin-4-yl)pyridin-2-yl)-3-azabicyclo[3.1.0]hexan-6-yl)picolinamide,
3-chloro-N-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)picolinamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)isobutyramide,
2-amino-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-phenylacetamide,
4-(6-((1R,5S,6s)-6-(((6-methoxypyridin-3-yl)methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile,
4-(6-((1R,5S,6s)-6-(((6-methoxypyridin-3-yl)methyl)(methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile,
2-chloro-N-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzenesulfonamide,
1-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-phenylurea,
3-chloro-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)picolinamide,
3-chloro-N-((1R,5S,6s)-3-(5-(3-cyano-6-ethoxypyrazolo[1,5-a]pyridin-4-yl)pyridin-2-yl)-3-azabicyclo[3.1.0]hexan-6-yl)picolinamide,
3-chloro-N-((1R,5S,6s)-3-(5-(3-cyano-6-methoxypyrazolo[1,5-a]pyridin-4-yl)pyridin-2-yl)-3-azabicyclo[3.1.0]hexan-6-yl)picolinamide,
(R)-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-2-hydroxy-2-phenylacetamide,
3-chloro-N-((1R,5S,6r)-3-(5-(3-cyano-6-ethoxypyrazolo[1,5-a]pyridin-4-yl)pyrazin-2-yl)-3-azabicyclo[3.1.0]hexan-6-yl)picolinamide,
3-chloro-N-((1R,5S,6s)-3-(5-(3-cyano-6-ethoxypyrazolo[1,5-a]pyridin-4-yl)pyrazin-2-yl)-3-azabicyclo[3.1.0]hexan-6-yl)picolinamide,
3-chloro-N-((1R,5S,6r)-3-(5-(3-cyano-6-ethoxypyrazolo[1,5-a]pyridin-4-yl)pyridin-2-yl)-3-azabicyclo[3.1.0]hexan-6-yl)picolinamide,
3-chloro-N-((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)picolinamide,
1-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-(6-methoxypyridin-3-yl)urea,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzenesulfonamide,
2-chloro-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-6-fluorobenzenesulfonamide,
3-chloro-N-((3aR,5r,6aS)-2-(5-(3-cyano-6-ethoxypyrazolo[1,5-a]pyridin-4-yl)pyridin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyridin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyrazin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-methoxypyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(morpholin-2-ylmethoxy)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)picolinamide,
2-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-methoxynicotinamide,
2-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-fluorobenzenesulfonamide,
3-chloro-N-((3aR,5s,6aS)-2-(5-(3-cyano-6-ethoxypyrazolo[1,5-a]pyridin-4-yl)pyrazin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)picolinamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-3-methylbutanamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-methylbenzamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluoropicolinamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-3-(trifluoromethyl)picolinamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-fluorobenzamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-methylpicolinamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluorobenzamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluoro-2-methylbenzamide,
3-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)picolinamide,
tert-butyl (((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate,
tert-butyl (((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)picolinamide,
2-chloro-N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzenesulfonamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-3-methylbutanamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-2-phenylacetamide,
N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-3-methylbutanamide,
3-chloro-N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)picolinamide,
N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-2-phenylacetamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluorobenzamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluoro-2-methylbenzamide,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylpicolinamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylbenzamide,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluoropicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-(trifluoromethyl)picolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)pivalamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-methylbutanamide. 3-chloro-N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)picolinamide,
(1R,3 S,5s,7s)-N-(3-chloropyridin-2-yl)-2-(5-(3-cyano-6-(1-methyl-1H-pyrazol-4-yl)pyrazolo[1,5-a]pyridin-4-yl)pyridin-2-yl)-2-azaadamantane-5-carboxamide,
N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-3-fluoropicolinamide,
4-(6-((3aR,6aS)-5-((2-chloro-6-fluorophenyl)sulfonyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
2-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-fluorobenzamide,
3-chloro-N-(((1R,5S,6s)-3-(5-(3-cyano-6-ethoxypyrazolo[1,5-a]pyridin-4-yl)pyridin-2-yl)-3-azabicyclo[3.1.0]hexan-6-yl)methyl)picolinamide,
3-chloro-N-(((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)picolinamide,
2-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-fluorobenzamide,
N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)acetamide,
(1R,3 S,5s,7s)-N-(3-chloropyridin-2-yl)-2-(5-(3-cyano-6-(2-hydroxy-2-methylpropoxy)pyrazolo[1,5-a]pyridin-4-yl)pyridin-2-yl)-2-azaadamantane-5-carboxamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-2-hydroxy-3-methylbutanamide,
2-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)-6-fluorobenzamide,
4-(5-((3aR,5s,6aS)-5-(((6-methoxypyridin-3-yl)methyl)amino)-5-methylhexahydrocyclopenta[c]pyrrol-2(1H)-yl)pyrazin-2-yl)-6-(1-methyl-1H-pyrazol-4-yl)pyrazolo[1,5-a]pyridine-3-carbonitrile,
3-chloro-N-((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)picolinamide,
3-chloro-N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)picolinamide,
4-(5-((1R,3 S,5s,7s)-5-hydroxy-2-azaadamantan-2-yl)pyrazin-2-yl)-6-(1-methyl-1H-pyrazol-4-yl)pyrazolo[1,5-a]pyridine-3-carbonitrile,
4-(6-((3aR,5r,6aS)-5-hydroxy-5-(pyridin-2-ylmethyl)hexahydrocyclopenta[c]pyrrol-2(1H)-yl)pyridin-3-yl)-6-(1-methyl-1H-pyrazol-3-yl)pyrazolo[1,5-a]pyridine-3-carbonitrile,
3-chloro-N-((3aR,5r,6aS)-2-(5-(3-cyano-6-(2-hydroxy-2-methylpropoxy)pyrazolo[1,5-a]pyridin-4-yl)pyridin-2-yl)octahydrocyclopenta[c]pyrrol-5-yl)picolinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)formamide,
4-(5-((1R,3 S,5s,7s)-5-amino-2-azaadamantan-2-yl)pyrazin-2-yl)-6-(1-methyl-1H-pyrazol-4-yl)pyrazolo[1,5-a]pyridine-3-carbonitrile,
tert-butyl ((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)carbamate,
N-((3aR,5r,6aS)-2-(5-(3-cyano-6-(2-hydroxy-2-methylpropoxy)pyrazolo[1,5-a]pyridin-4-yl)pyrazin-2-yl)octahydrocyclopenta[c]pyrrol-5-yl)-6-methoxynicotinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)acetamide,
3-chloro-N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)picolinamide,
(3aR,5s,6aS)-2-(5-(3-cyano-6-(1-methyl-1H-pyrazol-3-yl)pyrazolo[1,5-a]pyridin-4-yl)pyridin-2-yl)-N-(6-methoxypyridin-3-yl)-5-methyloctahydrocyclopenta[c]pyrrole-5-carboxamide,
(1R,3 S,5s,7s)-2-(5-(3-cyano-6-(1-methyl-1H-pyrazol-4-yl)pyrazolo[1,5-a]pyridin-4-yl)pyrazin-2-yl)-N-(6-methoxypyridin-3-yl)-2-azaadamantane-5-carboxamide,
3-chloro-N-((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
(3aR,5r,6aS)-2-(5-(3-cyano-6-(2-hydroxy-2-methylpropoxy)pyrazolo[1,5-a]pyridin-4-yl)pyridin-2-yl)-N-(6-methoxypyridin-3-yl)-5-methyloctahydrocyclopenta[c]pyrrole-5-carboxamide,
6-(2-hydroxy-2-methylpropoxy)-4-(6-((3aR,4S,7R,7aS)-8-((6-methoxypyridin-3-yl)methyl)hexahydro-1H-4,7-epiminoisoindol-2(3H)-yl)pyridin-3-yl)pyrazolo[1,5-a]pyridine-3-carbonitrile,
4-(6-((3aR,4S,7R,7aS)-8-((6-methoxypyridin-3-yl)methyl)hexahydro-1H-4,7-epiminoisoindol-2(3H)-yl)pyridin-3-yl)-6-(1-methyl-1H-pyrazol-4-yl)pyrazolo[1,5-a]pyridine-3-carbonitrile,
(1R,5S,6r)-3-(5-(3-cyano-6-(2-hydroxy-2-methylpropoxy)pyrazolo[1,5-a]pyridin-4-yl)pyridin-2-yl)-N-((6-methoxypyridin-3-yl)methyl)-3-azabicyclo[3.1.0]hexane-6-carboxamide,
6-(2-hydroxy-2-methylpropoxy)-4-(5-((3aR,6aS)-5-((6-methoxypyridin-3-yl)methyl)hexahydropyrrolo[3,4-c]pyrrol-2(1H)-yl)pyrazin-2-yl)pyrazolo[1,5-a]pyridine-3-carbonitrile,
3-cyano-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-methoxynicotinamide,
N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-3-fluoropicolinamide,
3-chloro-N-(((1R,5S,6s)-3-(5-(3-cyano-6-(2-hydroxypropoxy)pyrazolo[1,5-a]pyridin-4-yl)pyridin-2-yl)-3-azabicyclo[3.1.0]hexan-6-yl)methyl)picolinamide,
2-chloro-N-(((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)-6-fluorobenzamide,
N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-methoxynicotinamide,
3-cyano-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-3-fluoropicolinamide,
3-chloro-N-(2-((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)propan-2-yl)picolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)acetamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)methanesulfonamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)isobutyramide,
(1R,3 S,5s,7s)-2-(5-(3-cyano-6-(2-hydroxy-2-methylpropoxy)pyrazolo[1,5-a]pyridin-4-yl)pyridin-2-yl)-N-(6-methoxypyridin-3-yl)-2-azaadamantane-5-carboxamide,
(1R,3 S,5s,7s)-2-(5-(3-cyano-6-ethoxypyrazolo[1,5-a]pyridin-4-yl)pyridin-2-yl)-N-(6-methoxypyridin-3-yl)-2-azaadamantane-5-carboxamide,
3-chloro-N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)picolinamide,
3-chloro-N-((1R,3 S,5s,7s)-2-(5-(3-cyano-6-ethoxypyrazolo[1,5-a]pyridin-4-yl)pyridin-2-yl)-2-azaadamantan-5-yl)picolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-3-fluoropicolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-6-methoxynicotinamide,
N-((1R,3 S,5s,7s)-2-(5-(3-cyano-6-ethoxypyrazolo[1,5-a]pyridin-4-yl)pyridin-2-yl)-2-azaadamantan-5-yl)-6-methoxynicotinamide,
N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)-3-fluoropicolinamide,
2-chloro-N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-3-fluoropicolinamide,
3-chloro-N-((3aR,5s,6aS)-2-(5-(3-cyano-6-(2-hydroxy-2-methylpropoxy)pyrazolo[1,5-a]pyridin-4-yl)pyrazin-2-yl)-5-methyloctahydrocyclopenta[c]pyrrol-5-yl)picolinamide,
2-chloro-N-((3aR,5s,6aS)-2-(5-(3-cyano-6-(2-hydroxy-2-methylpropoxy)pyrazolo[1,5-a]pyridin-4-yl)pyrazin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-methoxynicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-methoxypicolinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-2-(trifluoromethyl)isonicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-5-methoxynicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-4-methoxypicolinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-2-methoxyisonicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-3-methoxypicolinamide,
4-(6-((3aR,5s,6aS)-5-(((6-methoxypyridin-3-yl)methyl)amino)-5-methylhexahydrocyclopenta[c]pyrrol-2(1H)-yl)pyridin-3-yl)-6-(1-methyl-1H-pyrazol-4-yl)pyrazolo[1,5-a]pyridine-3-carbonitrile,
3-chloro-N-(((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)methyl)picolinamide,
2-chloro-N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)-6-fluorobenzamide,
N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)-6-methoxynicotinamide,
N-(((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)methyl)-6-methoxynicotinamide,
4-(5-((1R,3 S,5s,7s)-5-hydroxy-2-azaadamantan-2-yl)pyrazin-2-yl)-6-(2-hydroxy-2-methylpropoxy)pyrazolo[1,5-a]pyridine-3-carbonitrile,
4-(6-((3aR,5r,6aS)-5-hydroxy-5-(pyridin-2-ylmethyl)hexahydrocyclopenta[c]pyrrol-2(1H)-yl)pyridin-3-yl)-6-(1-methyl-1H-pyrazol-4-yl)pyrazolo[1,5-a]pyridine-3-carbonitrile,
N-((3aR,5r,6aS)-2-(5-(3-cyano-6-(2-hydroxy-2-methylpropoxy)pyrazolo[1,5-a]pyridin-4-yl)pyridin-2-yl)octahydrocyclopenta[c]pyrrol-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)formamide,
N-((1R,3 S,5s,7s)-2-(5-(3-cyano-6-ethoxypyrazolo[1,5-a]pyridin-4-yl)pyrazin-2-yl)-2-azaadamantan-5-yl)formamide,
4-(5-((1R,3 S,5s,7s)-5-amino-2-azaadamantan-2-yl)pyrazin-2-yl)-6-(2-hydroxy-2-methylpropoxy)pyrazolo[1,5-a]pyridine-3-carbonitrile,
4-(5-((1R,3 S,5s,7s)-5-amino-2-azaadamantan-2-yl)pyrazin-2-yl)-6-ethoxypyrazolo[1,5-a]pyridine-3-carbonitrile,
3-chloro-N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)picolinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-ethoxypyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)-3-fluoropicolinamide,
3-chloro-N-((1R,3 S,5s,7s)-2-(5-(3-cyano-6-ethoxypyrazolo[1,5-a]pyridin-4-yl)pyrazin-2-yl)-2-azaadamantan-5-yl)picolinamide,
(1R,3 S,5s,7s)-2-(5-(3-cyano-6-ethoxypyrazolo[1,5-a]pyridin-4-yl)pyrazin-2-yl)-N-(6-methoxypyridin-3-yl)-2-azaadamantane-5-carboxamide,
(1R,3 S,5s,7s)-2-(5-(3-cyano-6-(2-hydroxy-2-methylpropoxy)pyrazolo[1,5-a]pyridin-4-yl)pyrazin-2-yl)-N-(6-methoxypyridin-3-yl)-2-azaadamantane-5-carboxamide,
N-((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-methoxynicotinamide,
3-chloro-N-((3aR,5r,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
6-(2-hydroxy-2-methylpropoxy)-4-(6-((3aR,4S,7R,7aS)-8-(6-methoxynicotinoyl)hexahydro-1H-4,7-epiminoisoindol-2(3H)-yl)pyridin-3-yl)pyrazolo[1,5-a]pyridine-3-carbonitrile,
(1R,5S,6r)-3-(5-(3-cyano-6-(2-hydroxy-2-methylpropoxy)pyrazolo[1,5-a]pyridin-4-yl)pyridin-2-yl)-N-(6-methoxypyridin-3-yl)-3-azabicyclo[3.1.0]hexane-6-carboxamide,
6-(2-hydroxy-2-methylpropoxy)-4-(5-((3aR,6aS)-5-(1-(6-methoxypyridin-3-yl)ethyl)hexahydropyrrolo[3,4-c]pyrrol-2(1H)-yl)pyrazin-2-yl)pyrazolo[1,5-a]pyridine-3-carbonitrile,
4-(5-((3aR,6aS)-5-((6-cyanopyridin-3-yl)methyl)hexahydropyrrolo[3,4-c]pyrrol-2(1H)-yl)pyrazin-2-yl)-6-(2-hydroxy-2-methylpropoxy)pyrazolo[1,5-a]pyridine-3-carbonitrile,
2-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-fluorobenzenesulfonamide,
4-(6-((3aR,6aS)-5-((2-chloro-6-fluorophenyl)sulfonyl)hexahydropyrrolo[3,4-c]pyrrol-2(1H)-yl)pyridin-3-yl)-6-(1-methyl-1H-pyrazol-3-yl)pyrazolo[1,5-a]pyridine-3-carbonitrile,
tert-butyl ((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)carbamate,
4-(6-((1R,3 S,5s,7s)-5-amino-2-azaadamantan-2-yl)pyridin-3-yl)-6-(1-methyl-1H-pyrazol-4-yl)pyrazolo[1,5-a]pyridine-3-carbonitrile,
4-(5-((3aR,6aS)-5-((2-chloro-6-fluorophenyl)sulfonyl)hexahydropyrrolo[3,4-c]pyrrol-2(1H)-yl)pyrazin-2-yl)-6-(1-methyl-1H-pyrazol-4-yl)pyrazolo[1,5-a]pyridine-3-carbonitrile,
1-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-3-(6-methoxypyridin-3-yl)urea,
2-chloro-N-(((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)-6-fluorobenzenesulfonamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylbenzamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-(trifluoromethyl)picolinamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzamide,
N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)-6-methoxynicotinamide,
(1R,3 S,5s,7s)-2-(5-(3-cyano-6-(1-methyl-1H-pyrazol-4-yl)pyrazolo[1,5-a]pyridin-4-yl)pyridin-2-yl)-N-(6-methoxypyridin-3-yl)-2-azaadamantane-5-carboxamide,
N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-2-(6-methoxypyridin-3-yl)acetamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-methoxynicotinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-4-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-methoxyisonicotinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-(trifluoromethyl)isonicotinamide,
2-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-fluorobenzenesulfonamide,
3-cyano-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)picolinamide, and
3-chloro-N-((3aR,5r,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)picolinamide.

Embodiment 28. A pharmaceutical composition comprising a compound of any one of Embodiments 1-27, and/or a stereoisomer, a stable isotope, or a pharmaceutically acceptable salt or solvate thereof, admixed with at least one pharmaceutically acceptable carrier.

Embodiment 29. The pharmaceutical composition of Embodiment 28, further comprising at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, another RET kinase inhibitor, and kinase inhibitors.

Embodiment 30. The pharmaceutical composition of Embodiment 29, wherein the at least one therapeutic co-agent or co-treatment is combined with the compound in a single dosage form, or the at least one therapeutic co-agent is administered simultaneously or sequentially as separate dosage forms.

Embodiment 31. A method to treat a disease in a patient in need thereof whose disease is a RET-associated disease, comprising administering to the subject in need of such treatment a therapeutically effective amount of a compound of any one of Embodiments 1-27, and/or a stereoisomer, a stable isotope, or a pharmaceutically acceptable salt or solvate thereof, or a pharmaceutical composition of any one of Embodiments 28-30.

Embodiment 32. The method of Embodiment 31, wherein the method comprises determining if the disease in the patient is a RET-associated disease, and administering to a subject in need of such treatment a therapeutically effective RET inhibiting amount of a compound of any one of Embodiments 1-27, and/or a stereoisomer, a stable isotope, or a pharmaceutically acceptable salt or solvate thereof, or a pharmaceutical composition of any one of Embodiments 28-30.

Embodiment 33. The method of any one of Embodiments 31-32, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 34. The method of any one of Embodiments 31-32, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 35. The method of Embodiment 33, wherein the treatment comprises administering at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

Embodiment 36. The method of Embodiment 35, wherein the administering the compound is conducted simultaneously or serially with the administering the therapeutic co-agent.

Embodiment 37. The method of Embodiment 36, wherein the administering the therapeutic co-agent comprises another RET inhibitor, an immunotherapy, or combination thereof.

Embodiment 38. The method of Embodiment 33, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 39. The method of any of one of Embodiments 31-38, wherein the compound of any one of Embodiments 1-27, and/or a stereoisomer, a stable isotope, or a pharmaceutically acceptable salt or solvate thereof, or a pharmaceutical composition of any one of Embodiments 28-30, is orally administered.

Embodiment 40. A use of a compound of any one of Embodiments 1-27, and/or a stereoisomer, a stable isotope, or a pharmaceutically acceptable salt or solvate thereof, or a pharmaceutical composition according to any one of Embodiments 28-30 as a medicament, in the manufacture of a medicament, or in medicine for treatment of a RET-associated disease.

Embodiment 41. The use of Embodiment 40, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 42. The use of Embodiment 41, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 43. The use of any of one of Embodiments 41-42, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 44. The use of any of one of Embodiments 41-43, wherein the medicament is formulated for oral administration.

Embodiment 45. A compound of any one of Embodiments 1-27, and/or a stereoisomer, a stable isotope, or a pharmaceutically acceptable salt or solvate thereof, or a pharmaceutical composition of Embodiments 28-30 for use in treating a RET-associated disease.

Embodiment 46. The compound of Embodiment 45 for use in treating a RET-associated disease, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 47. The compound of Embodiment 46 for use in treating a RET-associated disease, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 48. A compound of Embodiment 46 for use in treating a RET-associated disease, wherein the RET-associated disease is a RET-associated cancer, and the use comprises determining if the cancer in a patient is RET-associated cancer, and administering to the patient in need of such treatment a therapeutically effective amount of the compound.

Embodiment 49. The compound of any of one of Embodiments 46 to 48, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 50. A method of inhibiting RET kinase activity in vitro or in vivo for a RET-associated cancer cell having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein, with a compound of any one of Embodiments 1-27, and/or a stereoisomer, a stable isotope, or a pharmaceutically acceptable salt or solvate thereof.

Embodiment 51. A method of treating RET-associated cancer in a patient who has developed resistance to a RET inhibitor, comprising administering to a subject in need of such treatment a therapeutically effective RET inhibiting amount of a compound that is active against the RET kinase with RET mutations resistant to the prior treatment of any one of Embodiments 1-27, and/or a stereoisomer, a stable isotope, or a pharmaceutically acceptable salt or solvate thereof, or a pharmaceutical composition of any one of Embodiments 28-30.

Embodiment 52. The method of Embodiment 51, wherein the method comprises (a) determining the RET-mutations of a cancer cell in a sample from a patient who developed resistance to prior treatment of a RET inhibitor; and (b) administering a compound that is active against the RET kinase with RET mutations resistant to the prior treatment of any one of Embodiments 1-27, and/or a stereoisomer, a stable isotope, or a pharmaceutically acceptable salt or solvate thereof, or a pharmaceutical composition of any one of Embodiments 28-30.

Embodiment 53. The method of any one of Embodiments 51-52, wherein the treatment comprises administering at least one therapeutic co-agent or co-treatment selected from chemotherapeutics or other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

Embodiment 54. The method of Embodiment 53, wherein administering the therapeutic co-agent comprises another RET inhibitor, an immunotherapy, or combination thereof.

Embodiment 55. A kit comprising a compound of any of Embodiments 1-27 or a pharmaceutically acceptable salt thereof, or a pharmaceutical composition according to any of Embodiments 28-30, and a therapeutic co-agent.

Embodiment 56. A process for preparing compounds of Formula 22, wherein Z³ is Cl, Br, OTf, OMe, or OR; wherein R is H or an optionally substituted C1-C3 alkyl, wherein the optional substituents are 1-3 groups independently selected from H, halogen, C1-C3 alkoxy, C1-C3 alkanoyloxy, and aryl; X³ and X⁶ are independently —CH— or N; R⁹ is H, OH, F, CF₃, —OCF₃, CN, or an optionally substituted group selected from C1-C3 alkyl, C1-C3 alkoxy, C3-C6 cycloalkyl, and C3-C6 cycloalkoxy; and P is an amino protecting group.

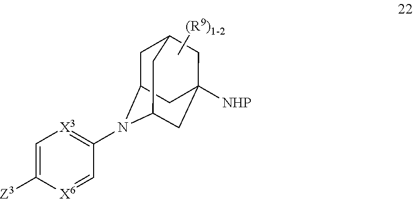

In some embodiments, the compound of Formula I (such as Formulae IA, IB, IC, ID, and IE), has the chiral configuration shown in excess over its enantiomer, so the compound is optically active. For example, such compounds disclosed herein are substantially free of the opposite enantiomer, i.e., at least 95% of the compound has the chirality shown above.

Also disclosed herein is a pharmaceutical composition comprising a compound of Formula I (such as Formulae IA, IB, IC, ID, and IE), and/or a stereoisomer, a stable isotope, or a pharmaceutically acceptable salt or solate thereof, and a pharmaceutically acceptable carrier.

Further disclosed herein is a method of inhibiting the activity of RET comprising contacting the protein RET with an effective amount of a compound of Formula I (such as Formulae IA, IB, IC, ID, and IE), and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a compound of Formula I (such as Formulae IA, IB, IC, ID, and IE), and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a pharmaceutical composition comprising a compound of Formula I (such as Formulae IA, IB, IC, ID, and IE), and/or a stereoisomer, a stable isotope, or a pharmaceutically acceptable salt or solvate thereof disclosed herein and a pharmaceutically acceptable carrier.

Further disclosed herein is a method of treating a cancer in a patient, comprising administering to the patient in recognized need of such treatment, an effective amount of a pharmaceutical composition comprising a compound of Formula I (such as Formulae IA, IB, IC, ID, and IE), and/or a stereoisomer, a stable isotope, or a pharmaceutically acceptable salt or solvate thereof disclosed herein and a pharmaceutically acceptable carrier. In some embodiments, the cancer is colon cancer, gastric cancer, leukemia, lymphoma, melanoma, or pancreatic cancer.

Further disclosed herein is a method of treating an inflammatory disease in a patient, comprising administering to the patient in recognized need of such treatment, an effective amount of a pharmaceutical composition comprising a compound of Formula I (such Formulae IA, IB, IC, ID, and IE), and/or a stereoisomer, a stable isotope, or a pharmaceutically acceptable salt or solvate thereof disclosed herein and a pharmaceutically acceptable carrier. In some embodiments, the inflammatory disease is rheumatoid arthritis, psoriasis, or eczema.

Further disclosed herein is a use of a compound of Formula I (such as Formulae IA, IB, IC, ID, and IE), and/or a stereoisomer, a stable isotope, or a pharmaceutically acceptable salt or solvate thereof in preparation of a medication for treating a disease responsive to inhibition of RET, such as a cancer. In some embodiments, the cancer is lung cancers, thyroid cancers, pancreatic cancers, salivary gland cancers, spitz tumors, colorectal cancers, ovarian cancers, or myeloproliferative cancers.

The pharmaceutical composition comprising a compound of Formula I (such as Formulae IA, IB, IC, ID, and IE), and/or a stereoisomer, a stable isotope, or a pharmaceutically acceptable salt or solvate thereof, and a pharmaceutically acceptable carrier, can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The compound of Formula I (such as Formulae IA, IB, IC, ID, and IE), and/or a stereoisomer, a stable isotope, or a pharmaceutically acceptable salt or solvate thereof can be administered orally in solid dosage forms, such as capsules, tablets, troches, dragées, granules and powders, or in liquid dosage forms, such as elixirs, syrups, emulsions, dispersions, and suspensions. The compound of Formula I (such as Formulae IA, IB, IC, ID, and IE), and/or a stereoisomer, a stable isotope, or a pharmaceutically acceptable salt or solvate thereof can also be administered parenterally, in sterile liquid dosage forms, such as dispersions, suspensions or solutions. Other dosages forms that can also be used to administer the compound of Formula I (such as Formulae IA, IB, IC, ID, and IE), and/or a stereoisomer, a stable isotope, or a pharmaceutically acceptable salt or solvate thereof include ointment, cream, drops, transdermal patch or powder for topical administration, an ophthalmic solution or suspension formation, i.e., eye drops, for ocular administration, an aerosol spray or powder composition for inhalation or intranasal administration, or a cream, ointment, spray or suppository for rectal or vaginal administration.

Gelatin capsules containing the compound of Formula I (such as Formulae IA, IB, IC, ID, and IE), and/or a stereoisomer, a stable isotope, or a pharmaceutically acceptable salt or solvate thereof and at least one powdered carrier selected, for example, from lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound disclosed herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the pharmaceutical composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are disclosed in Remington's Pharmaceutical Sciences, A. Osol, a standard reference text in the art.

The compound of Formula I (such as Formulae IA, IB, IC, ID, and IE), and/or a stereoisomer, a stable isotope, or a pharmaceutically acceptable salt or solvate thereof can be examined for efficacy in treating cancer by in vivo assays. For example, the compound of Formula I (such as Formulae IA, IB, IC, ID, and IE), and/or a stereoisomer, a stable isotope, or a pharmaceutically acceptable salt or solvate thereof can be administered to an animal (e.g., a mouse model) having cancer and its therapeutic effects can be accessed. Positive results in one or more of such tests are sufficient to increase the scientific storehouse of knowledge and hence sufficient to demonstrate practical utility of the compounds and/or salts tested. Based on the results, an appropriate dosage range and administration route for animals, such as humans, can also be determined.

For administration by inhalation, the compound of Formula I (such as Formulae IA, IB, IC, ID, and IE), and/or a stereoisomer, a stable isotope, or a pharmaceutically acceptable salt or solvate thereof may be conveniently delivered in the form of an aerosol spray presentation from pressurized packs or nebulisers. The compound of Formula I (such as Formulae IA, IB, IC, ID, and IE), and/or a stereoisomer, a stable isotope, or a pharmaceutically acceptable salt or solvate thereof may also be delivered as powders, which may be formulated and the powder composition may be inhaled with the aid of an insufflation powder inhaler device. One exemplary delivery system for inhalation can be a metered dose inhalation (MDI) aerosol, which may be formulated as a suspension or solution of a compound of Formula I (such as Formulae IA, IB, IC, ID, and IE), and/or a stereoisomer, a stable isotope, or a pharmaceutically acceptable salt or solvate thereof in at least one suitable propellant, selected, for example, from fluorocarbons and hydrocarbons.

For ocular administration, an ophthalmic preparation may be formulated with an appropriate weight percentage of a solution or suspension of the compound of Formula I (such as Formulae IA, IB, IC, ID, and IE) and/or a stereoisomer, a stable isotope, or a pharmaceutically acceptable salt or solvate thereof in an appropriate ophthalmic vehicle, such that the compound of Formula I (such as Formulae IA, IB, IC, ID, and IE), and/or a stereoisomer, a stable isotope, or a pharmaceutically acceptable salt or solvate thereof is maintained in contact with the ocular surface for a sufficient time period to allow the compound to penetrate the corneal and internal regions of the eye.

Useful pharmaceutical dosage-forms for administration of the compound of Formula I (such as Formulae IA, IB, IC, ID, and IE), and/or a stereoisomer, a stable isotope, or a pharmaceutically acceptable salt or solvate thereof include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, the compound of Formula I (such as Formulae IA, IB, IC, ID, and IE), and/or a stereoisomer, a stable isotope, or a pharmaceutically acceptable salt or solvate thereof can be present in an amount of 1, 5, 10, 15, 20, 25, 50, 75, 80, 85, 90, 95, 100, 125, 150, 200, 250, 300, 400 and 500 mg in a capsule.

In some embodiments, a large number of unit capsules can be prepared by filling standard two-piece hard gelatin capsules each with, for example, 100 milligrams of the compound of Formula I (such as Formulae IA, IB, IC, ID, and IE), and/or a stereoisomer, a stable isotope, or a pharmaceutically acceptable salt or solvate thereof in powder, 150 milligrams of lactose, 50 milligrams of cellulose, and 6 milligrams magnesium stearate.

In some embodiments, a mixture of the compound of Formula I (such as Formulae IA, IB, IC, ID, and IE), and/or a stereoisomer, a stable isotope, or a pharmaceutically acceptable salt or solvate thereof and a digestible oil such as soybean oil, cottonseed oil or olive oil can be prepared and injected by means of a positive displacement pump into gelatin to form soft gelatin capsules containing 75 or 100 milligrams of the active ingredient. The capsules are washed and dried.

In some embodiments, the compound of Formula I (such as Formulae IA, IB, IC, ID, and IE), and/or a stereoisomer, a stable isotope, or a pharmaceutically acceptable salt or solvate thereof can be present in an amount of 1, 5, 10, 15, 20, 25, 50, 75, 80, 85, 90, 95, 100, 125, 150, 200, 250, 300, 400 and 500 mg in a tablet.

In some embodiments, a large number of tablets can be prepared by conventional procedures so that the dosage unit comprises, for example, 100 milligrams of the compound of Formula I (such as Formulae IA, IB, IC, ID, and IE), and/or a stereoisomer, a stable isotope, or a pharmaceutically acceptable salt or solvate thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may, for example, be applied to increase palatability or delay absorption.

In some embodiments, a parenteral composition suitable for administration by injection can be prepared by stirring 1.5% by weight of a compound of Formula I (such as Formulae IA, IB, IC, ID, and IE), and/or a stereoisomer, a stable isotope, or a pharmaceutically acceptable salt or solvate thereof in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound of Formula I (such as Formulae IA, IB, IC, ID, and IE), and/or a stereoisomer, a stable isotope, or a pharmaceutically acceptable salt or solvate thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound of Formula I (such as Formulae IA, IB, IC, ID, and IE), and/or a stereoisomer, a stable isotope, or a pharmaceutically acceptable salt or solvate thereof are administered stepwise or in conjunction with at least one other therapeutic agent. When drugs are administered in physical combination, the dosage form and administration route should be selected depending on the compatibility of the combined drugs. Thus, the term “co-administration” is understood to include the administration of at least two agents concomitantly or sequentially, or alternatively as a fixed dose combination of the at least two active components.

The compound of Formula I (such as Formulae IA, IB, IC, ID, and IE), and/or a stereoisomer, a stable isotope, or a pharmaceutically acceptable salt or solvate thereof can be administered as the sole active ingredient or in combination with at least one second active ingredient, selected, for example, from other active ingredients known to be useful for treating the target disease, such as cancers including, for example, colon cancer, gastric cancer, leukemia, lymphoma, melanoma, and pancreatis cancer in a patient.

As used herein, the term “optical isomer” or “stereoisomer” refers to any of the various stereo isomeric configurations which may exist for a given compound of the present disclosure and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. The present disclosure includes enantiomers, diastereomers or racemates of the compounds. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog lR-SJ system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and synthesis procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present disclosure includes all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration unless specified. If the compound contains a disubstituted cycloalkyl, the cycloalkyl substituent may have a cis- or trans-configuration, unless otherwise specified.

In many cases, the compounds of the present disclosure are capable of forming acid and/or base salts by virtue of the presence of amino and/or carboxyl groups or groups similar thereto. As used herein, the terms “salt” or “salts” refers to an acid addition or base addition salt of a compound of the disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable.

Pharmaceutically acceptable acid addition salts can be formed with inorganic acids and organic acids, e.g., acetate, adipate, aluminum, ascorbate, aspartate, benzoate, besylate, bromide/hydrobromide, bicarbonate/carbonate, bisulfate/sulfate, camphorsulfonate, caproate, chloride/hydrochloride, chloroprocaine, chlortheophyllonate, citrate, edetate, calcium edetate, ethandisulfonate, ethylsulfonate, ethylene diamine, fumarate, galactarate (mucate), gluceptate, gluconate, glucuronate, glutamate, glycolate, hexyl resorcinate, hippurate, hydroiodide/iodide, hydroxynapthoate (xinafoate), isethionate, lactate, lactobionate, laurylsulfate, lithium, malate, maleate, malonate, mandelate, mesylate, methylsulphate, naphthoate, napsylate, nicotinate, nitrate, octadecanoate, oleate, oxalate, palmitate, pamoate, pantothenate, phosphate/hydrogen phosphate/dihydrogen phosphate, polygalacturonate, procaine, propionate, salicylate, sebacate, stearate, subacetate, succinate, sulfate, sulfosalicylate, tannate, tartrate, bitartrate, tosylate, triphenylacetate, and trifluoroacetate salts. Lists of additional suitable salts can be found, e.g., in REMINGTON'S PHARMACEUTICAL SCIENCES, 20th ed., Mack Publishing Company, Easton, Pa., (1985); and in HANDBOOK OF PHARMACEUTICAL SALTS: PROPERTIES, SELECTION, AND USE, by Stahl and Wermuth (Wiley-VCH, Weinheim, Germany, 2002). Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic, sulfosalicylic acid, and the like.

Pharmaceutically acceptable base addition salts can be formed with inorganic or organic bases and can have inorganic or organic counterions.

Inorganic counterions for such base salts include, for example, ammonium salts and metals from columns I to XII of the periodic table. In certain embodiments, the counterion is selected from sodium, potassium, ammonium, alkylammonium having one to four C1-C4 alkyl groups, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Suitable organic amines include isopropylamine, benzathine, cholinate, diethanolamine, diethylamine, lysine, meglumine, piperazine and tromethamine.

The pharmaceutically acceptable salts of the present disclosure can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, tetrahydrofuran, toluene, chloroform, dichloromethane, methanol, ethanol, isopropanol, or acetonitrile is desirable, where practicable.

Any formula given herein is intended to represent unlabeled forms (i.e., compounds wherein all atoms are present at natural isotopic abundances and not isotopically enriched) as well as isotopically enriched or labeled forms of the compounds. Isotopically enriched or labeled compounds have structures depicted by the formulas given herein except that at least one atom of the compound is replaced by an atom of the same element but having an atomic mass or mass number different from the atomic mass or the atomic mass distribution that occurs naturally. Examples of isotopes that can be incorporated into enriched or labeled compounds of the disclosure include isotopes of hydrogen, carbon, nitrogen, oxygen, phosphorous, fluorine, and chlorine, such as ²H, ³H, ¹¹C, ¹³C, ⁴C, ⁵N, ¹⁸F, ³¹P, ³²P, ³⁵S, ³⁶Cl, and ¹²⁵I respectively. The present disclosure includes various isotopically labeled compounds as defined herein, for example those in which radioactive isotopes, such as 3H and ¹⁴C, or those in which non-radioactive isotopes, such as ²H and ¹³C, are present at levels significantly above the natural abundance for these isotopes. These isotopically labeled compounds are useful in metabolic studies (with ⁴C), reaction kinetic studies (with, for example ²H or 3H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of Formula I can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the Formula I if it is incorporated at substantially above the level of natural isotopic abundance. The present disclosure includes isotopically enriched versions of the compounds, e.g., deuterated versions as well as non-deuterated versions. Deuterated versions may be deuterated at a single site, or at multiple sites.

The degree of incorporation of such an isotope in an isotopically-enriched compound, particularly deuterium, may be defined by the isotopic enrichment factor. The term “isotopic enrichment factor” as used herein means the ratio between the isotopic abundance of a specified isotope in a sample, and the natural abundance of the isotope in a non-enriched sample. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the present disclosure include those wherein the solvent of crystallization may be isotopically substituted, e.g. D₂O, d₆-acetone, d₆-DMSO, as well as solvates with non-enriched solvents.

Compounds of the disclosure, e.g., compounds of Formula I (such as Formulae IA, IB, IC, ID, and IE), that contain groups capable of acting as donors and/or acceptors for hydrogen bonds, may be capable of forming co-crystals with suitable co-crystal formers. These co-crystals may be prepared from compounds of Formula I (such as Formulae IA, IB, IC, ID, and IE), by known co-crystal forming procedures. Such procedures include grinding, heating, co-subliming, co-melting, or contacting in solution compounds of Formula I (such as Formulae IA, IB, IC, ID, and IE), with the co-crystal former under crystallization conditions and isolating co-crystals thereby formed. Suitable co-crystal formers include those described in WO2004078163. Hence the present disclosure further provides co-crystals comprising a compound of Formula I (such as Formulae IA, IB, IC, ID, and IE).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, REMINGTON'S PHARMACEUTICAL SCIENCES, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “therapeutically effective amount” refers to the amount of the compound of the present disclosure that, when administered to a subject, is effective to (1) at least partially alleviate, inhibit, prevent and/or ameliorate a condition, or a disorder or a disease (i) mediated by a kinase such as RET or (ii) associated with activity of a kinase such as RET, or (iii) characterized by activity (normal or abnormal) of RET; or (2) reduce or inhibit the activity of RET or (3) reduce or inhibit the expression of RET.

In another non-limiting embodiment, the term “a therapeutically effective amount” refers to the amount of the compound of the present disclosure that, when administered to a cell, or a tissue, or a non-cellular biological material, or a medium, is effective to at least partially reduce or inhibit the activity of RET, or at least partially reduce or inhibit the expression of RET.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or inhibiting” refers to the reduction or suppression of a given condition, activity, effect, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “Treat”, “treating” or “treatment” refers to alleviating or ameliorating at least one physical parameter including those which may not be discernible by the patient. In yet another embodiment, “Treat”, “treating” or “treatment” refers to modulating the disease or disorder, either physically, (e.g., stabilization of a discernible symptom), physiologically, (e.g., stabilization of a physical parameter), or both. In yet another embodiment, “Treat”, “treating” or “treatment” refers to delaying the development or progression of the disease or disorder.

As used herein, a subject is “in need of” a treatment if such subject would be expected to benefit biologically, medically or in quality of life from such treatment.

As used herein, the term “a” “an” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosed otherwise claimed.

Any asymmetric atom (e.g., carbon or the like) of the compound(s) of the present disclosure can be present in racemic or enantiomerically enriched, for example, the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the (R)- or (S)-configuration; i.e., for optically active compounds, it is often, for example, to use one enantiomer to the substantial exclusion of the other enantiomer. Substituents at atoms with carbon-carbon double bonds may, where possible, be present in cis- (Z)- or trans- (E)-form, and both are included in the present disclosure unless otherwise indicated.

Accordingly, as used herein a compound of the present disclosure can be in the form of one of the possible isomers, rotamers, atropisomers, or as a mixture thereof, for example, as substantially pure geometric (cis or trans) isomers, diastereomers, optical isomers (antipodes), racemates or mixtures thereof. “Substantially pure” or “substantially free of other isomers” as used herein means the product contains less than 5%, and, such as, less than 2%, of other isomers relative to the amount of the preferred isomer, by weight.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present disclosure, including their salts, can also be obtained in the form of their hydrates, or include other solvents used for their crystallization. The compounds of the present disclosure may inherently or by design form solvates with pharmaceutically acceptable solvents (including water); therefore, it is intended that the present disclosure embraces both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Schemes 1-6 show general methods for preparing the compounds of the present disclosure as well as intermediates. The detailed description and syntheses are disclosed in the Examples below. Those skilled in the art will be able to find other synthetic methods or modify the methods described below using conventional chemistry for preparing suitable compounds encompassed by Formula I. So these methods are equally applicable to preparation of compounds with other embodiments. Although specific starting materials and reagents are depicted in the Schemes and discussed below, other starting materials and reagents can be easily substituted to provide a variety of compounds and/or reaction conditions.

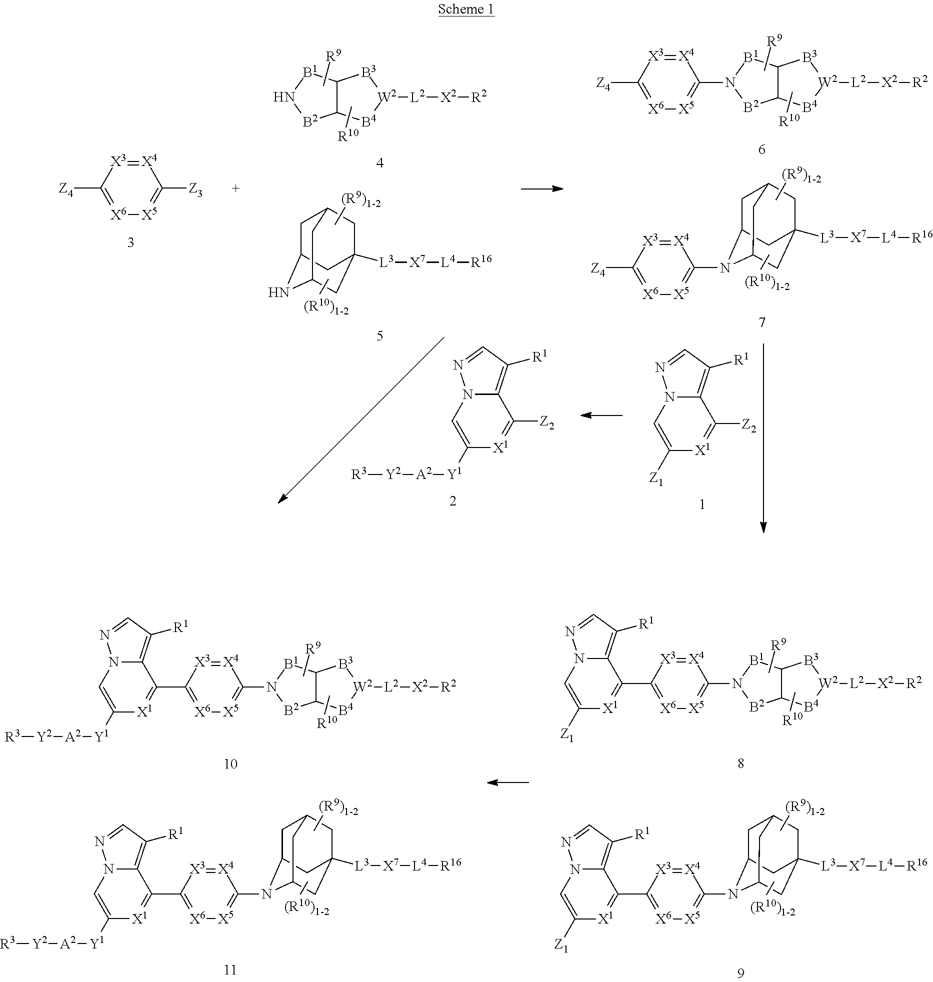

Compounds 10 and 11 of Formula I can be made by general synthetic method as illustrated in Scheme 1. Pyrazolo[1,5-a]pyridine 1 (Z₁ and Z₂ are independently Cl, Br, I, OTf, OH or OP, wherein P is a protecting group) can be converted to compound 2 wherein the side chain R³—Y²-A²-Y¹ is installed via many functional group transformations. For example, when Z₁ is Cl, Br, OTf, or I, it can undergo Suzuki reaction with arylboronic acid or heteroarylboronic acid (or its esters) using palladium catalyzed chemistry to give compound 2 wherein Y²-A²-Y¹ is a bond and R³ is aryl or heteroaryl. Similarly, compound 1 can react with alcohol under basic condition via nucleophilic displacement of Z₁ or under palladium catalyzed reaction conditions to produce compound 2 wherein R³—Y²-A²-Y¹ is R³O—. Compound 1 (when Z₁ is OH) can also react with alkyl halide or epoxide (such as 2,2-dimethyloxirane) catalyzed by a base such as K₂CO₃ to give compound 2 when R³—Y²-A²-Y¹ is R³O—, or (CH₃)₂C(OH)CH₂O—.

The reactive selectivity between Z₁ and Z₂ can be controlled by placing different groups at Z₁ and Z₂. For example, one can start with compound 1 wherein Z₁ is Br and Z₂ is Cl. Another method is to have Z₁ be halogen and Z₂ be OP (P is a protecting group); the latter can be deprotected and converted to triflate in the next reaction. Coupling of compound 3 (Z₃ and Z₄ are independently F, Cl, Br, I, or OTf) and amines 4 and 5 under Buchwald reaction conditions of palladium chemistry can give compounds 6 and 7. Compounds 4 and 5 can be made by many methods known to the skilled person or are commercially available. Compounds 6 and 7 can also be prepared by nucleophilic displacement of Z₃ of compound 3 by amines 4 and 5. Conversion of 6 and 7 to 8 and 9 can be accomplished by reacting with compound 1 using palladium catalyzed chemistry. Conversion of compounds 8 and 9 to compounds of formula 10 and 11, which are compounds of Formula I, using the same methods as described for compound 2. Alternatively, compounds 10 and 11 can be synthesized by coupling of compounds 6 and 7 with compound 2 using palladium catalyzed chemistry.

The Schemes 2-6 in some instances illustrate preparation of compounds 21, 25, 29, 31, 35, 36, 37, 42, 46 and 50, and intermediates 36B, 36C and 36D, but methods for preparing suitable compounds encompassed by Formula I are readily apparent to the skilled person in view of the many methods known for making the requisite intermediates, so these methods are equally applicable to preparation of compounds with other embodiments;
wherein R²¹ is —X⁹—R¹⁷, wherein X⁹ is a bond, —C(O)—, —SO₂—, —C(O)O—, —C(O)NR¹³—, —SO₂NR¹³—, or an optionally substituted group selected from C1-C6 alkyl, and wherein the optional substituents for R²¹ is 1-4 substituents independently selected from R⁴; R¹², R¹³ and R¹⁷ are as defined previously.

R²² is selected from H and C1-C4 alkyl optional substituted with 1-3 groups independently selected from R⁴;

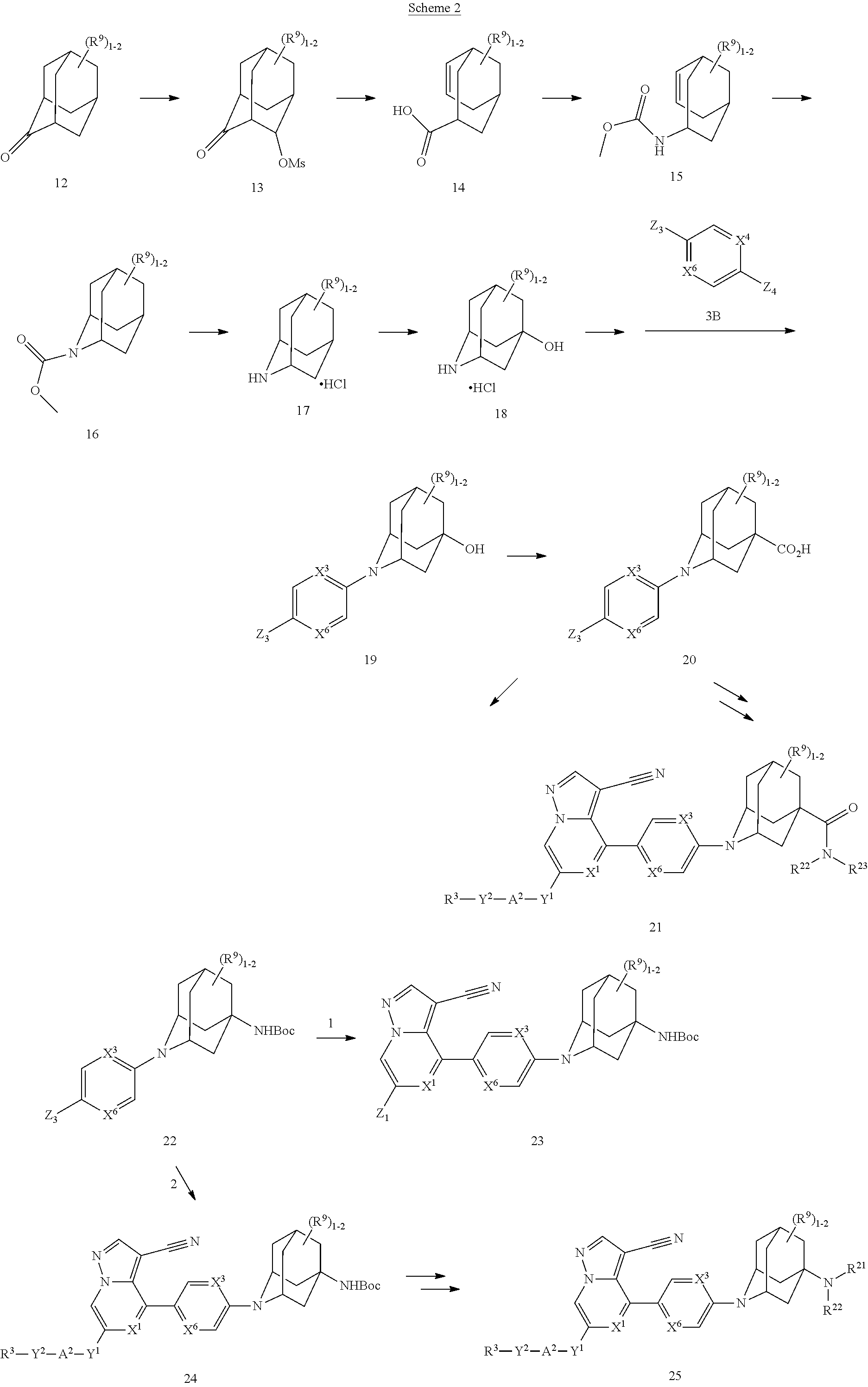

R²³ is selected from an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R²³ is 1-4 substituents independently selected from R⁴;
R²⁴ and R²⁵ are independently selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R²⁴ and R²⁵ are 1-4 substituents independently selected from R⁴; R²⁴ and R²⁵ can optionally be taken together to form a 3-6 membered ring that can optionally contains 1-2 heteroatoms selected from N, O and S and can optionally be substituted with 1-2 groups independently selected from R⁴.

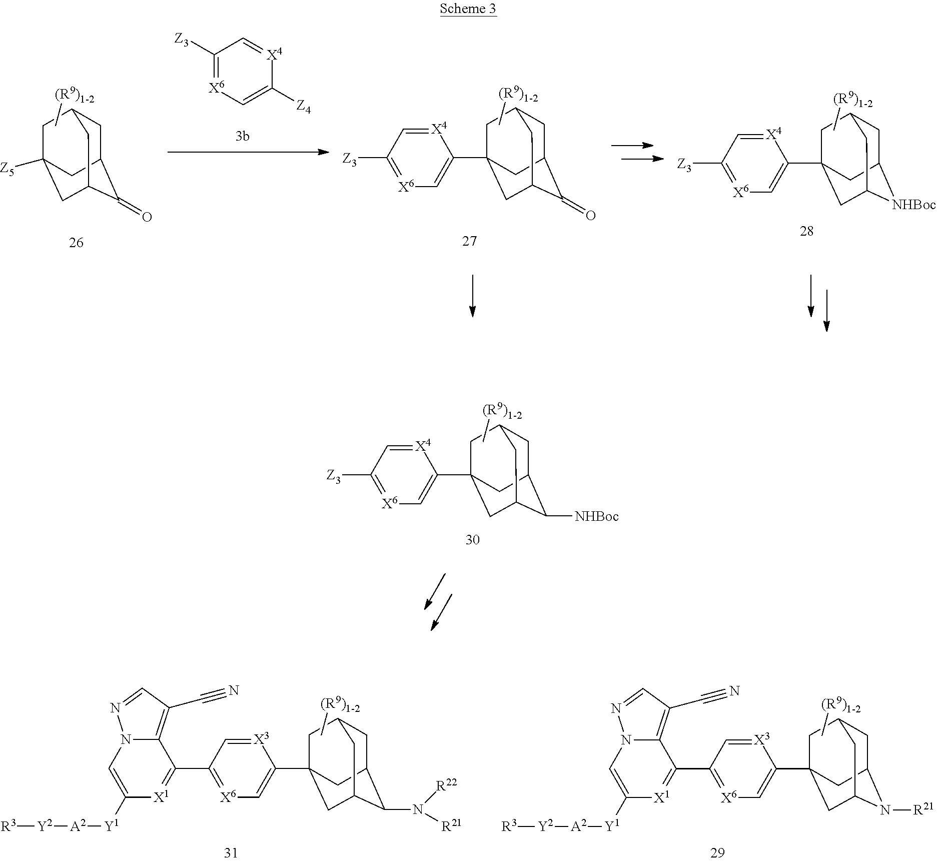

Compounds 21 and 25 can be made as shown in Scheme 2. The commercially available compound 12 reacts with sodium azide and methanesulfonic acid to form compound 13. Ring opening (to compound 14) under basic conditions and Curtius rearrangement can give compound 15. Ring closure catalyzed by triflic acid can give compound 16. Hydrolysis of 16 provides compound 17. Hydroxy insertion reaction of compound 17 forms compound 18. Coupling of compound 18 with 3B (Z₃ and Z₄ are independently F, Cl, Br, I, or OTf) under Buchwald reaction conditions of palladium chemistry or nucleophilic displacement of Z₄ of compound 3 forms compound 19. Koch reaction of 19 using sulfuric acid and oleum converts to carboxylic acid 20. Coupling reaction of compound 20 with the amines provides compound 21. Curtius rearrangement of compound 20 forms compound 22, which is converted to the final compound 25 using the similar methods as described for Scheme 1.

In Scheme 3, readily available compound 26 is treated with trifluoromethanesulfonic acid to convert to compound 27. Conversion of compound 27 to compound 28 is perfumed using the similar methods as described in Scheme 2. Reductive amination of ketone 27 and Boc protection provides compound 30, which is converted to compound 31 using the similar methods as described in Scheme 2.

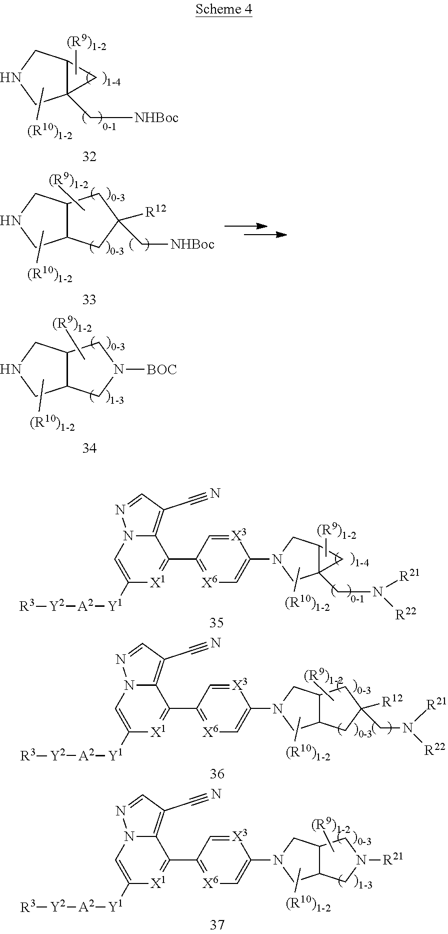

Scheme 4 illustrates the synthetic methods to produce compounds of formula 35, 36 and 37. Bicyclic amines 32, 33, and 34 can be made by many methods known to the skilled person. They can be converted to the final compounds of formula 35, 36 and 37 using the same methods as described in Scheme 1, which may require further modifications such as hydrogenation, deprotection, acylation or sulfonylation reactions by conventional methods leading to the desired substituents.

Scheme 5 illustrates the synthetic methods to produce intermediate 36B and compounds of formula 42. Commercially available compound 38, wherein P is a protecting group such as benzyl and CBS, reacts with a Grignard reagent, or an alkyllithium, at low temperature such as at −78° C. to form compound 39, which then reacts with trimethylsilanecarbonitrile in acetic acid and sulfuric acid to provide compound 40. Hydrolysis of 40 under acidic conditions or basic conditions, and subsequent protection with Boc group provides compound 36B, which can be converted to compound 42 using the same methods as described in Scheme 1.

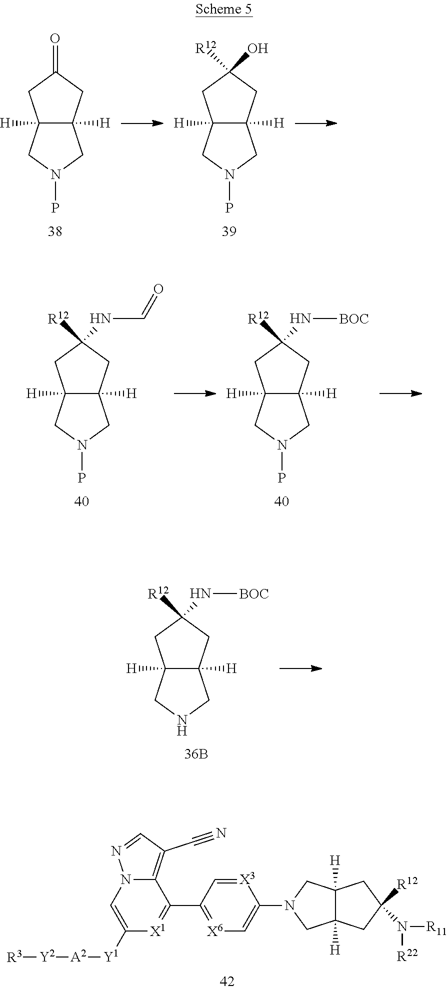

Scheme 6 illustrates the synthetic methods to intermediates 36C and 36D and the compounds of formula 46 and 50. Known compound 43, wherein P is a protecting group such as benzyl or CBS, can be reduced using a reducing agent, such as BH₃, or lithium aluminum hydride. Mitsunobu reaction of alcohol 44 with sodium azide or phthalimide yields the amine precursor or protected amine that can be easily reduced or hydrolyzed to give the amine, which is protected to provide compound 45. Removal of the protecting group of 45 under hydrogenolysis reaction conditions provides compound 36C. Compound 43 reacts with a Grignard reagent, or an alkyllithium, at low temperature such as at −78° C. to form compound 47, which then reacts with trimethylsilanecarbonitrile in acetic acid and sulfuric acid to provide compound 48. Hydrolysis of 48 under acidic conditions or basic conditions, and subsequent protection with Boc group provides compound 49. Removal of the protecting group of 49 under hydrogenolysis reaction conditions provides compound 36D. Compound 36C and 36D can be converted to compound 46 and 50 using the same methods as described in Scheme 1.

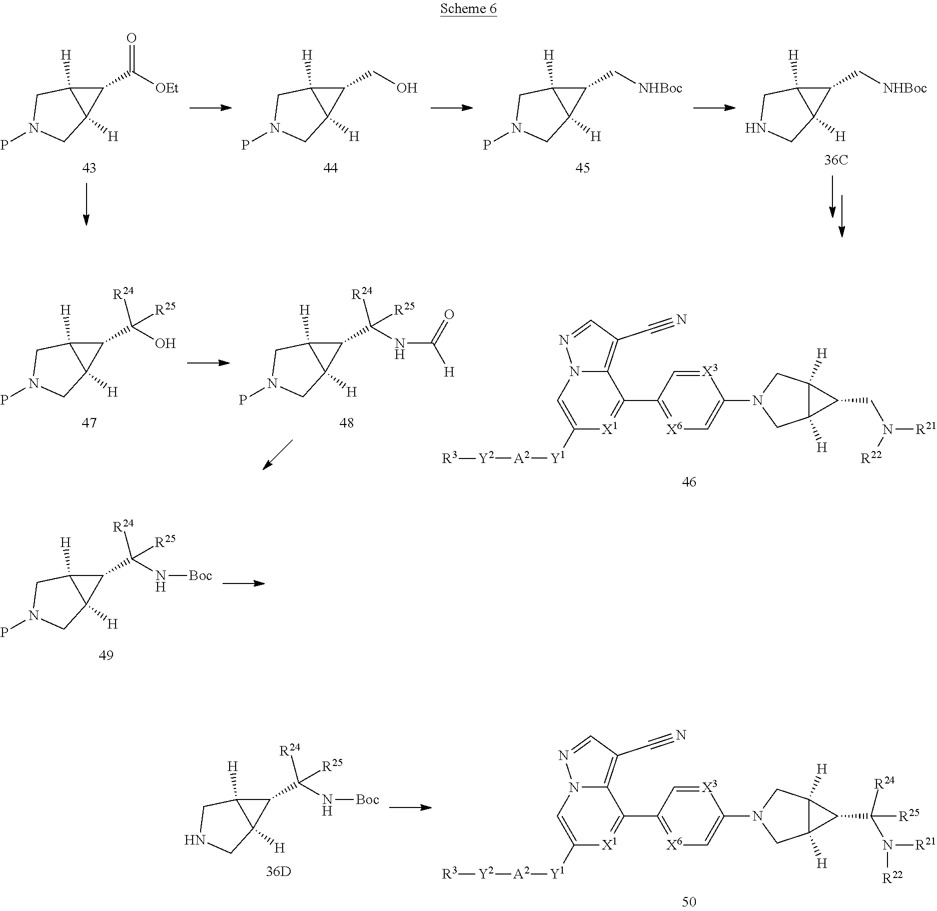

### EXAMPLES

The following examples illustrate certain embodiments of the present disclosure and how to make and use them. They are not intended to limit the scope of the invention. Those of skill in the art will readily recognize a variety of noncritical parameters and conditions which can be changed or modified to yield essentially the same results. The example compounds below were found to be inhibitors of RET according to one or more of the assays described herein.

In the following examples, the abbreviations below are used:
BINAP 2,2′-Bis(diphenylphosphino)-1,1′-binaphthyl
BOC tert-Butyloxycarbonyl
B₂(Pin)₂ Bis(pinacolato)diboron
BTEAC Benzyltriethylammonium chloride
CDI Carbonyldiimidazole
dba dibenzylideneacetone
DCE 1,2-Dichloroethene
DCM Dichloromethane
DHP Dihydropyran
DIAD Diisopropyl azodicarboxylate
DIPEA di-isopropylethylamine
DMA Dimethylacetamide
DMAP 4-Dimethylaminopyridine
DMF Dimethylformamide
DMSO Dimethylsulfoxide
dppf 1,1′Bis(diphenylphosphino)ferrocene
EDTA Ethylenediaminetetraacetic acid
EtOAc Ethyl acetate
EtOH Ethanol
HATU 1-[Bis(dimethylamino)methylene]-1H-1,2,3-triazolo[4,5-b]pyridinium 3-oxide hexafluorophosphate
KHMDS Potassium hexamethyldisilazane
LiHMDS Lithium hexamethyldisilazane
LG Leaving group
MeOH Methanol
MsCl Methanesulfonyl chloride
MTBE Methyl tert-butyl ether
Pd₂dba₃ Tris(dibenzylidenacetone)palladium
Pd(dppf)Cl₂ [1,1′Bis(diphenylphosphino)ferrocene]dichloropalladium(II)
PE Petroleum ether
PG Protecting group
PPTS Pyridinium p-toluenesulfonate
Prep-TLC Preparative Thin layer chromatography
PTSA p-toluenesulfonic acid
TBAF tetra-n-butylammonium fluoride
TBDMSCl t-Butyldimethylsilyl chloride
TEA Triethylamine
TES Triethylsilyl
TFA Trifluoacetic acid
Tf Triflyl
Tf₂O Trifluoromethanesulfonic anhydride
TLC Thin layer chromatography
THF Tetrahydrofuran
THP tetrahydropyran
TMS Trimethylsilyl
TosMIC Toluenesulfonylmethyl isocyanide
Xantphos 4,5-Bis(diphenylphosphino)-9,9-dimethylxanthene
XPhos 2-Dicyclohexylphosphino-2′,4′,6′-triisopropylbiphenyl

### Intermediate Synthesis

### Intermediate 1

### 3-cyano-6-(1-methyl-1H-pyrazol-4-yl)pyrazolo[1,5-a]pyridin-4-yl trifluoromethanesulfonate

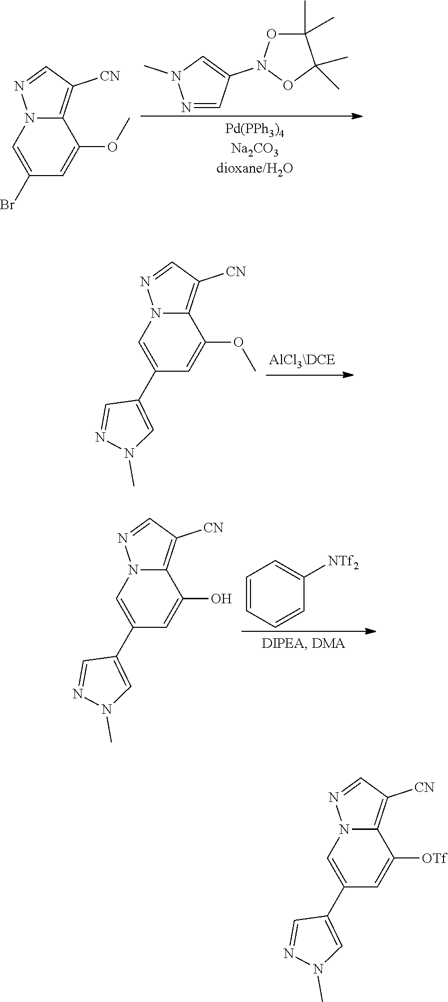

### Step 1. 4-methoxy-6-(1-methyl-1H-pyrazol-4-yl)pyrazolo[1,5-a]pyridine-3-carbonitrile

To a solution of the commercially available 6-bromo-4-methoxypyrazolo[1,5-a]pyridine-3-carbonitrile (63.5 g, 252 mmol) and 4,4,5,5-tetramethyl-2-(1-methyl-1H-pyrazol-4-yl)-1,3,2-dioxazolidine (62.9 g, 302.4 mmol) in dioxane/H₂O (850 mL/170 mL) was added Na₂CO₃(53.4 g, 50.4 mmol), followed by Pd(PPh₃)₄(5.8 g, 5.04 mmol). The reaction mixture was flushed with N₂, heated at 80° C. for 18h, cooled to rt, and vigorously stirred for 2 h. The suspension was filtered and the solid was washed with H₂O (2.3 L) and MTBE (3×300 mL), dried in vacuo overnight to yield the title compound, which was used in the next step without further purification (62 g, yield: 97%).

### Step 2. 4-hydroxy-6-(1-methyl-1H-pyrazol-4-yl)pyrazolo[1,5-a]pyridine-3-carbonitrile

To a suspension of AlCl₃ (197.5 g, 1.48 mol) in DCE (3 L) stirred at 50° C. for 1 h was added the product of Step 1 above (75 g, 296.3 mmol). The reaction mixture was stirred at 80° C. overnight, cooled to rt, diluted with DCE (1.5 L), and quenched with portions of H₂O (8×500 mL). The mixture was stirred at rt for 3 h. The resulting suspension was filtered off and the filter cake was dried in an oven at 40° C. under vacuum to give the title compound, which was used in the next step without further purification (65 g, yield: 92%).

### Step 3. 3-cyano-6-(1-methyl-1H-pyrazol-4-yl)pyrazolo[1,5-a]pyridin-4-yl trifluoromethanesulfonate

To a suspension of the product of Step 2 above (10 g, 41.8 mmol) in DMA (100 mL) was added DIPEA (10.8 g, 83.6 mmol), followed by 1,1,1-trifluoro-N-phenyl-N-((trifluoromethyl)sulfonyl)methanesulfonamide (16.4 g, 46 mmol). The resulting solution was stirred at rt for 2 h and then slowly poured into H₂O (300 mL). The resulting suspension was stirred for 2 h then filtered. The filter cake was rinsed with H₂O. The solid was dissolved in DCM (1.6 L), and filtered through celite. The filtrate was dried over anhydrous Na₂SO₄, filtered off, and concentrated in vacuo to give the title compound (15 g, yield: 96%).

### Intermediate 2

### 3-cyano-6-(1-methyl-1H-pyrazol-3-yl)pyrazolo[1,5-a]pyridin-4-yl trifluoromethanesulfonate

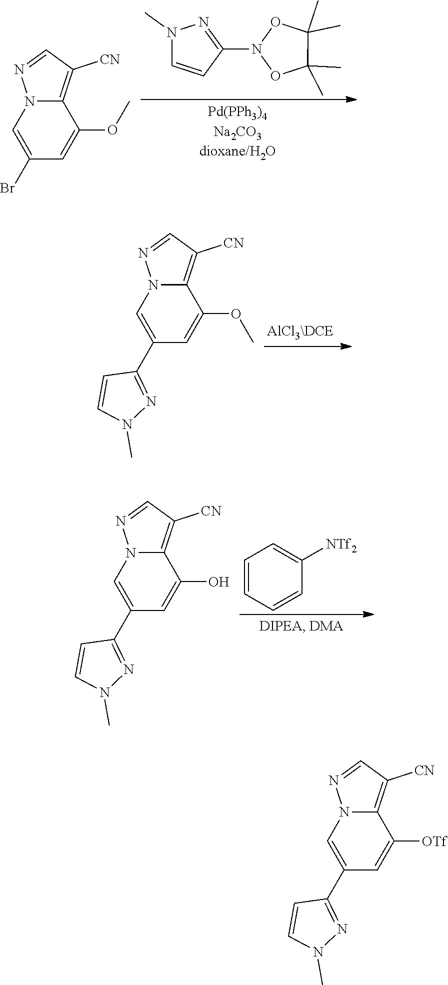

This intermediate was synthesized similarly by the procedure described in Intermediate 1 by using 4,4,5,5-tetramethyl-2-(1-methyl-1H-pyrazol-3-yl)-1,3,2-dioxazolidine in place of 4,4,5,5-tetramethyl-2-(1-methyl-1H-pyrazol-4-yl)-1,3,2-dioxazolidine.

### Intermediate 3

### 4-bromo-6-(2-hydroxy-2-methylpropoxy)pyrazolo[1,5-a]pyridine-3-carbonitrile

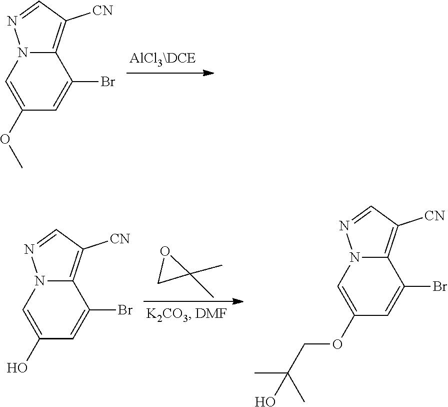

### Step 1. 4-bromo-6-hydroxypyrazolo[1,5-a]pyridine-3-carbonitrile

To a solution of commercially available 4-bromo-6-methoxypyrazolo[1,5-a]pyridine-3-carbonitrile (900 mg, 3.55 mmol) in DCE (40 mL) was added AlCl₃ (2.37 g, 17.78 mmol). The mixture was stirred at 80° C. overnight. After cooling to rt and diluted with THF, the mixture was treated with anhydrous Na₂SO₄ (7.5 g) and H₂O (9.5 g). The resulting suspension was stirred for 4h and filtered. The filter cake was rinsed with THF (50 mL) and the filtrate was concentrated to give the title compound (800 mg, yield: 95%).

### Step 2. 4-bromo-6-(2-hydroxy-2-methylpropoxy)pyrazolo[1,5-a]pyridine-3-carbonitrile

To a solution of the product of Step 1 above (800 mg, 3.36 mmol) and K₂CO₃ (1.39 g, 10.08 mmol) in DMF (5 mL) was added 2,2-dimethyloxirane (2.42 g, 33.6 mmol). The mixture was stirred at 60° C. for 12h and at 85° C. for another 12h. After cooling to rt, the mixture was diluted with H₂O (40 mL), and filtered off. The filtration was concentrated to give the title compound (835 mg, yield: 80%).

### Intermediate 4

### 4-bromo-6-ethoxypyrazolo[1,5-a]pyridine-3-carbonitrile

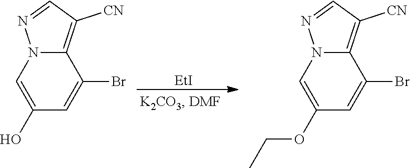

To a solution of the product of Step 1 of Intermediate 3 (2.3 g, 9.66 mmol) and K₂CO₃ (4.0 g, 29 mmol) in DMF (60 mL) was added ethyl iodide (2.26 g, 14.5 mmol). The mixture was stirred at 60° C. for 3h before cooling to rt, quenching with 28% ammonia/H₂O (1/1, 40 mL), and filtering off. The filtration was concentrated in vacuo to give the title compound (2.1 g, yield: 81%).

### Intermediate 5

### 4-(6-((3aR,6aS)-hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile hydrochloride

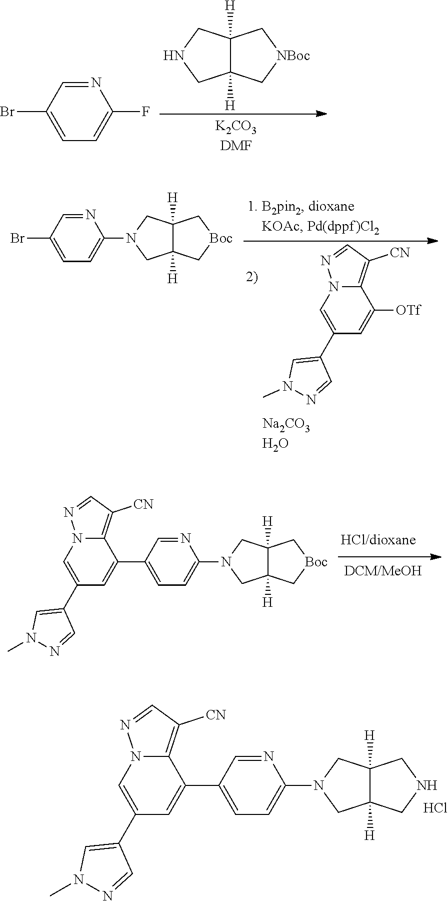

### Step 1. (3aR,6aS)-tert-butyl 5-(5-bromopyridin-2-yl)hexahydropyrrolo[3,4-c]pyrrole-2(1H)-carboxylate

To a solution of 5-bromo-2-fluoropyridine (2.42 g, 13.74 mmol) in DMF (30 mL) was added (3aR,6aS)-tert-butyl hexahydropyrrolo[3,4-c]pyrrole-2(1H)-carboxylate (3.5 g, 16.4 mmol) and K₂CO₃ (3.8 g, 27.48 mmol). The reaction mixture was stirred at 110° C. for 4 h. After cooling to rt, the mixture was concentrated in vacuo to remove the solvent. The residue was dissolved in EtOAc (200 mL), which was washed with H₂O (50 mL×2) and brine (50 mL), dried over anhydrous Na₂SO₄, filtered off and concentrated in vacuo. The residue was purified by flash column chromatography on silica gel (PE to PE/EtOAc=8/1) to give the title compound (4.15 g, yield: 88%).

### Step 2. (3aR,6aS)-tert-butyl 5-(5-(3-cyano-6-(1-methyl-1H-pyrazol-4-yl)pyrazolo[1,5-a]pyridin-4-yl)pyridin-2-yl)hexahydropyrrolo[3,4-c]pyrrole-2(1H)-carboxylate

To a solution of the product of Step 1 above (4 g, 11.68 mmol) in dioxane (50 mL) was added B₂Pin₂ (3.11 g, 12.27 mmol), Pd(dppf)Cl₂ DCM (477 mg, 0.58 mmol) and KOAc (2.3 g, 23.38 mmol). The reaction mixture was flushed with N₂ and stirred at 110° C. overnight. After cooling to rt, Intermediate 1 (4.33 g, 11.68 mmol), Na₂CO₃ (2.5 g, 23.36 mmol) and H₂O (10 mL) was added to the reaction mixture, which was flushed with N₂, stirred at 110° C. overnight. After cooling to rt, the mixture was filtered. The filtrate was diluted with DCM/MeOH (10/1, 200 mL), washed with H₂O (50 mL×2) and brine (50 mL), dried over anhydrous Na₂SO₄, filtered off, and concentrated in vacuo. The residue was purified by flash column chromatography on silica gel (DCM/MeOH=50/1 to 20/1) to give the title compound (800 mg, yield: 13%).

### Step 3. 4-(6-((3aR,6aS)-hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile hydrochloride

To a solution of the product of Step 2 above (800 mg, 1.567 mmol) in DCM/MeOH (16 mL/4 mL) was added HCl/dioxane (4 N, 8 mL, 32 mmol) at 0° C. The reaction solution was stirred at rt overnight and concentrated in vacuo to give the title compound (900 mg, yield: 100%).

### Intermediate 6

### 4-(6-((3aR,6aS)-hexahydropyrrolo[3,4-c]pyrrol-2(1H)-yl)pyridin-3-yl)-6-(1-methyl-1H-pyrazol-3-yl)pyrazolo[1,5-a]pyridine-3-carbonitrile hydrochloride

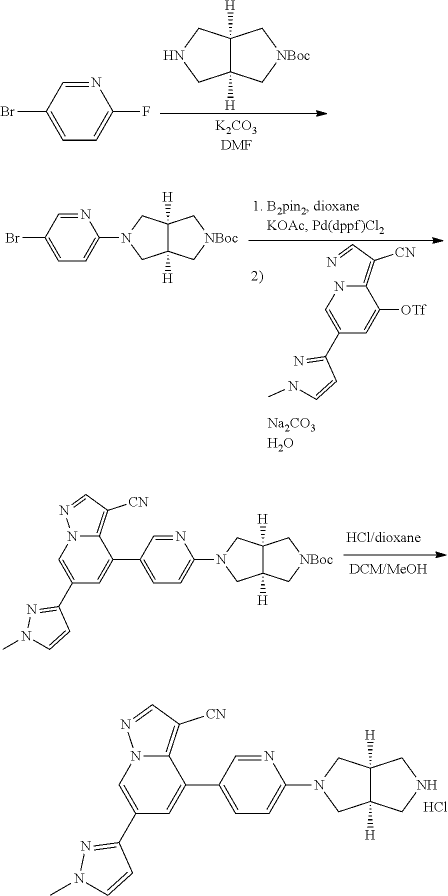

This intermediate was synthesized similarly by the procedure described in Intermediate 5 by using Intermediate 2 in place of Intermediate 1.

### Intermediate 7

### 4-(6-((1R,5S,6s)-6-amino-3-azabicyclo[3.1.0]hexan-3-yl)pyridin-3-yl)-6-(1-methyl-1H-pyrazol-4-yl)pyrazolo[1,5-a]pyridine-3-carbonitrile hydrochloride

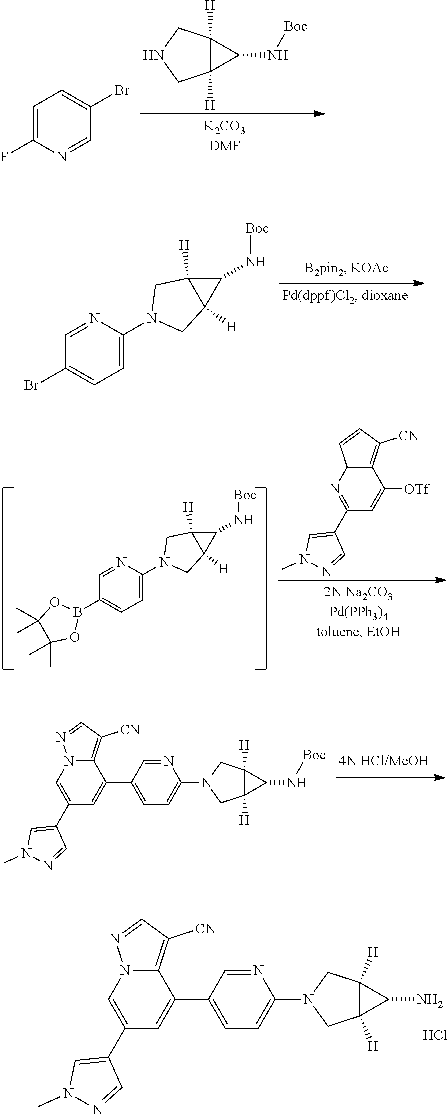

### Step 1. tert-butyl ((1R,5S,6s)-3-(5-bromopyridin-2-yl)-3-azabicyclo[3.1.0]hexan-6-yl)carbamate

To a solution of 5-bromo-2-fluoropyridine (924.6 mg, 5.25 mmol) in DMF (12 mL) was added tert-butyl (1R,5S,6s)-3-azabicyclo[3.1.0]hexan-6-ylcarbamate (1.25 g, 6.3 mmol) and K₂CO₃ (1.45 g, 10.5 mmol). The reaction mixture was stirred at 110° C. for 4 h before being concentrated in vacuo. The residue was dissolved in EtOAc (200 mL), which was washed with H₂O (50 mL×2) and brine (50 mL), dried over anhydrous Na₂SO₄, filtered off and concentrated in vacuo. The residue was purified by flash column chromatography on silica gel (PE/EtOAc=20/1˜4/1) to give the title compound (2.06 g, yield:100%).

### Step 2. tert-butyl ((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)carbamate

To a solution of the product of Step 1 above (1.76 g, 4.97 mmol) in dioxane (15 mL) was added B₂Pin₂ (1.33 g, 5.22 mmol), Pd(dppf)Cl₂ DCM (405 mg, 0.5 mmol), and KOAc (975.5 mg, 9.94 mmol). The reaction mixture was flushed with N₂ and stirred at 100° C. for 4 h. After cooling to rt, Intermediate 1 (1.43 g, 3.85 mmol), Pd(PPh₃)₄(222 mg, 0.2 mmol), aqueous Na₂CO₃ (2 N, 5.5 mL, 11 mmol), EtOH (11.5 mL), and toluene (12 mL) was added to the mixture. The resultant mixture was flushed with N₂, stirred at 100° C. overnight, and filtered off. The filtrate was diluted with DCM/MeOH (10/1, 200 mL), washed with H₂O (50 mL×2) and brine (50 mL), dried over anhydrous Na₂SO₄, filtered off and concentrated in vacuo. The residue was purified by flash column chromatography on silica gel (DCM/MeOH=50/1 to 10/1) to give the title compound (1.4 g, yield: 73%).

### Step 3. 4-(6-((1R,5S,6s)-6-amino-3-azabicyclo[3.1.0]hexan-3-yl)pyridin-3-yl)-6-(1-methyl-1H-pyrazol-4-yl)pyrazolo[1,5-a]pyridine-3-carbonitrile hydrochloride

To a solution of the product of Step 2 above (400 mg, 0.806 mmol) in MeOH (1 mL) was added HCl/MeOH (4 N, 4 mL, 16 mmol) at 0° C. The reaction solution was stirred at rt for 6 h before concentrating in vacuo to give the title compound (319 mg, yield: 100%).

### Intermediate 8

### 4-(6-((1R,5S,6r)-6-amino-3-azabicyclo[3.1.0]hexan-3-yl)pyridin-3-yl)-6-(1-methyl-1H-pyrazol-4-yl)pyrazolo[1,5-a]pyridine-3-carbonitrile hydrochloride

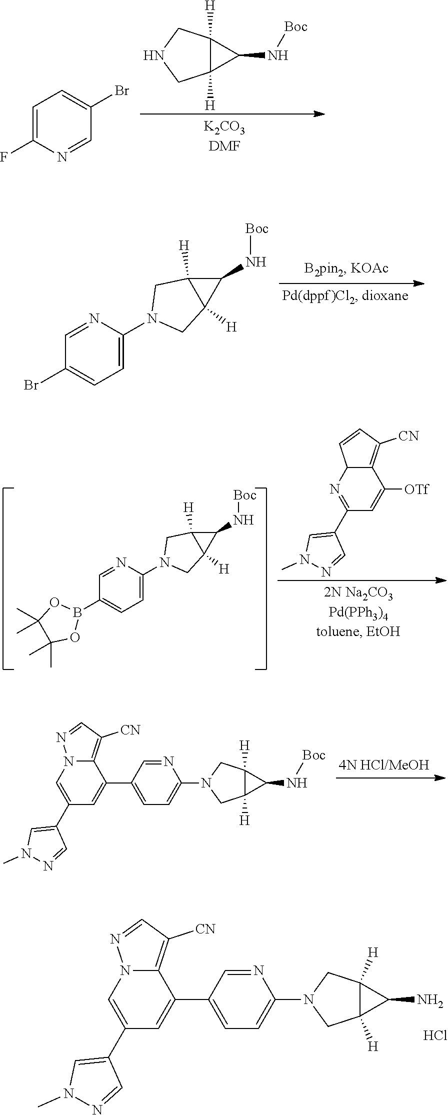

This intermediate was synthesized similarly by the procedure described in Intermediate 7 by using tert-butyl (1R,5S,6r)-3-azabicyclo[3.1.0]hexan-6-ylcarbamate in place of tert-butyl (1R,5S,6s)-3-azabicyclo[3.1.0]hexan-6-ylcarbamate.

### Intermediate 9

### (1R,5S,6s)-3-(4-bromophenyl)-3-azabicyclo[3.1.0]hexan-6-amine hydrochloride

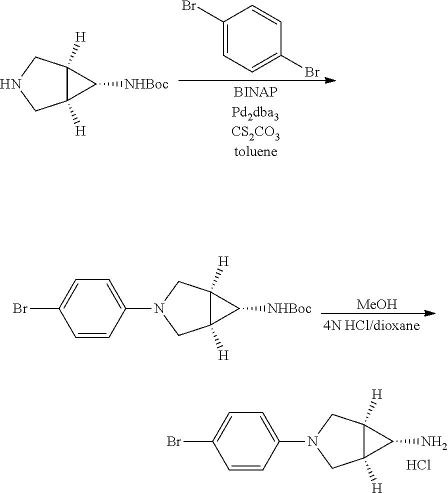 [SMILES: "MeOH" ; CC(C)(C)OC(=O)N[C@H]1[C@@H]2CNC[C@@H]21 ; Brc1ccc(Br)cc1 ; CC(C)(C)OC(=O)N[C@H]1[C@@H]2CN(c3ccc(Br)cc3)C[C@@H]21 ; N[C@@H]1[C@H]2CN(c3ccc(Br)cc3)C[C@@H]12 ; Cl]

### Step 1. tert-butyl ((1R,5S,6s)-3-(4-bromophenyl)-3-azabicyclo[3.1.0]hexan-6-yl)carbamate

A suspension of tert-butyl (1R,5S,6s)-3-azabicyclo[3.1.0]hexan-6-ylcarbamate (198 mg, 1.0 mmol), 1,4-dibromobenzene (261 mg, 1.1 mmol), Pd₂dba₃ (45.8 mg, 0.05 mmol), BINAP (77.8 mg, 0.125 mmol), and Cs₂CO₃ (512.3 mg, 1.6 mmol) in toluene (2 mL) was flushed with N₂ and stirred at 80° C. for 4 h. After cooling, the reaction mixture was diluted with EtOAc (100 mL), which was washed with H₂O (3 0 mL×2), brine (30 mL), dried over anhydrous Na₂SO₄, filtered off and concentrated. The residue was purified by flash column chromatography on silica gel (PE/EtOAc=8/1 to 4/1) to give the title compound (112 mg, yield: 32%).

### Step 2. (1R,5S,6s)-3-(4-bromophenyl)-3-azabicyclo[3.1.0]hexan-6-amine hydrochloride

To a solution of the product of Step 1 above (112 mg, 0.317 mmol) in MeOH (1.5 mL) was added 4N HCl/dioxane (1.5 mL) at 0° C. The reaction solution was stirred at rt for 2 h before concentrating in vacuo to give the crude title compound as a HCl salt (101 mg), which was used in the next step without any further purification.

### Intermediate 10 (1R,5S,6r)-3-(4-bromophenyl)-3-azabicyclo[3.1.0]hexan-6-amine hydrochloride

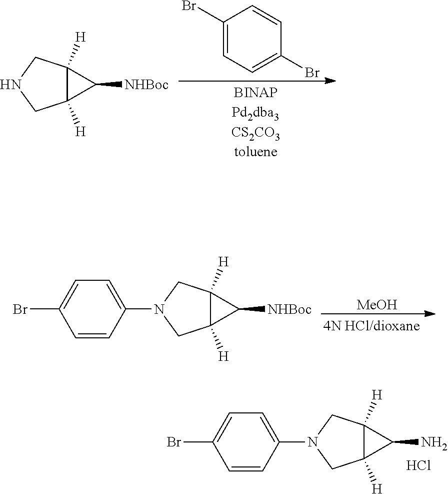 [SMILES: "MeOH" ; CC(C)(C)OC(=O)N[C@@H]1[C@@H]2CNC[C@@H]21 ; Brc1ccc(Br)cc1 ; CC(C)(C)OC(=O)N[C@@H]1[C@@H]2CN(c3ccc(Br)cc3)C[C@@H]21 ; N[C@H]1[C@H]2CN(c3ccc(Br)cc3)C[C@@H]12 ; Cl]

This intermediate was synthesized similarly by the procedure described in Intermediate 9 by using tert-butyl (1R,5S,6r)-3-azabicyclo[3.1.0]hexan-6-ylcarbamate in place of tert-butyl (1R,5S,6s)-3-azabicyclo[3.1.0]hexan-6-ylcarbamate.

### Intermediate 11

### 4-(6-((3aR,6aS)-hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile hydrochloride

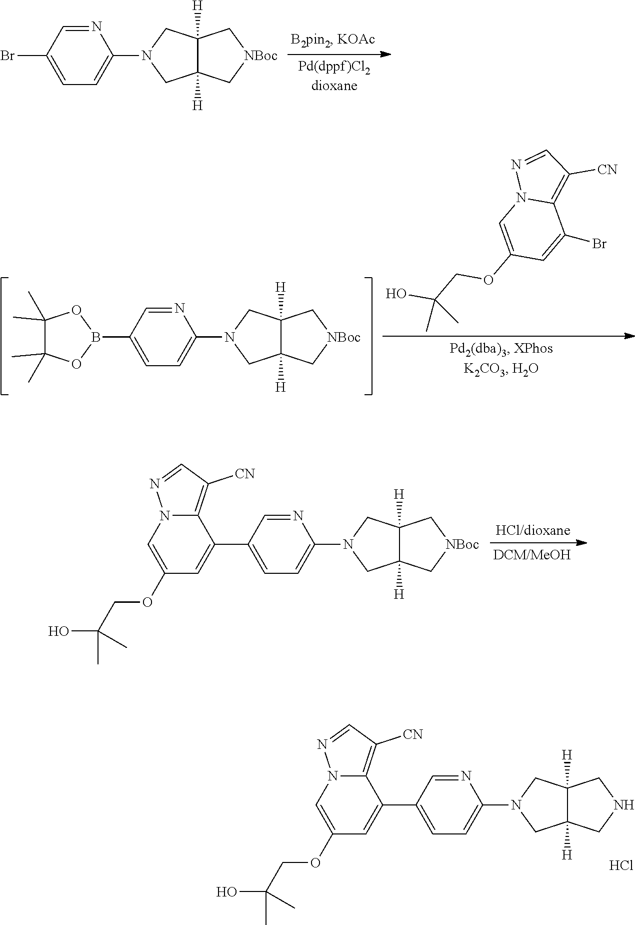

### Step 1. (3aR,6aS)-tert-butyl 5-(5-(3-cyano-6-(2-hydroxy-2-methylpropoxy)pyrazolo[1,5-a]pyridin-4-yl)pyridin-2-yl)hexahydropyrrolo[3,4-c]pyrrole-2(1H)-carboxylate

To a solution of the product of Step 1 of Intermediate 5 (100 mg, 0.27 mmol) in dioxane (1 mL) was added B₂Pin₂ (72 mg, 0.28 mmol), Pd(dppf)Cl₂ DCM (11 mg, 0.0135 mmol), and KOAc (53 mg, 0.54 mmol). The reaction mixture was flushed with N₂, stirred at 110° C. for 3 h. After cooling to rt, the mixture was treated with Intermediate 3 (90 mg, 0.29 mmol), K₂CO₃ (93 mg, 0.67 mmol), Pd₂dba₃ (10 mg, 0.011 mmol), XPhos (21 mg, 0.045 mmol), and H₂O (1 mL). The reaction mixture was flushed with N₂, stirred at 100° C. overnight. After cooling to rt, the mixture was diluted with DCM/MeOH (10/1, 200 mL), which was transferred to a separatory funnel, washed with H₂O (50 mL×2) and brine (50 mL), dried over anhydrous Na₂SO₄, filtered off and concentrated in vacuo. The residue was purified by flash column chromatography on silica gel (DCM/MeOH=50/1 to 10/1) to give the crude title compound, which was further purified via prep-TLC (DCM/MeOH=10/1) to give the title compound (100 mg, yield: 67%).

### Step 2. 4-(6-((3aR,6aS)-hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile hydrochloride

To a solution of the product of Step 1 above (100 mg, 0.19 mmol) in DCM/MeOH (4 mL/1 mL) was added HCl/dioxane (4 N, 2 mL, 8 mmol) at 0° C. The reaction solution was stirred at 40° C. for 1 h before concentrating in vacuo to give the title compound (86 mg, yield: 100%).

### Intermediate 12

### 4-(6-((1R,5S,6s)-6-amino-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

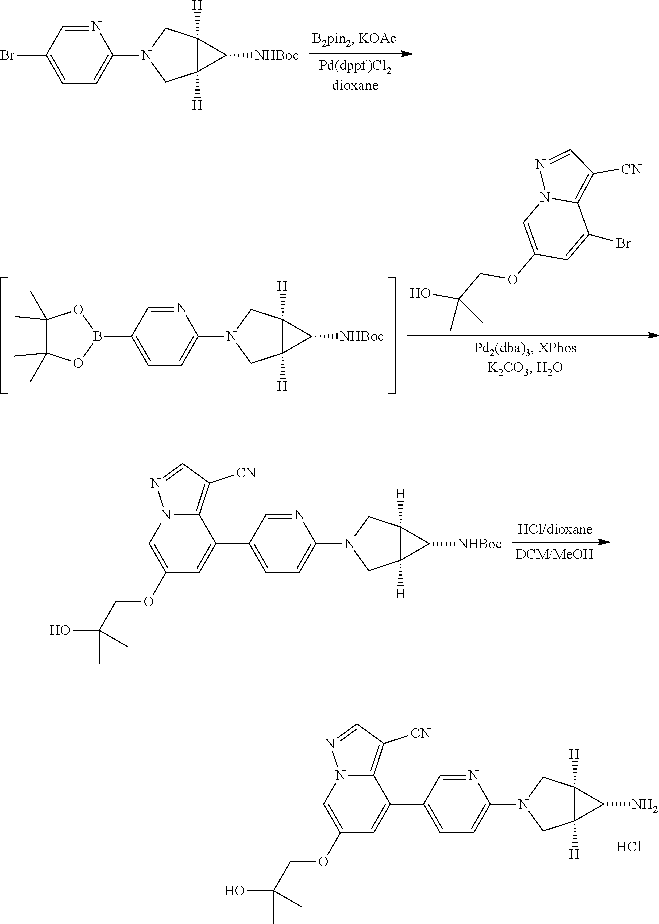

### Step 1. tert-butyl ((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)carbamate

To a solution of the product of step 1 of Intermediate 7 (400 mg, 1.13 mmol) in dioxane (4 mL) was added B₂Pin₂ (302 mg, 1.18 mmol), Pd(dppf)Cl₂ DCM (92 mg, 0.114 mmol), and KOAc (222 mg, 2.26 mmol) at rt. The reaction mixture was flushed with N₂, stirred at 100° C. for 4 h, and cooled to rt. To the mixture was added Intermediate 3 (319 mg, 1.03 mmol), K₂CO₃ (426 mg, 3.08 mmol), Pd₂dba₃ (47 mg, 0.051 mmol), XPhos (100 mg, 0.21 mmol), and H₂O (0.8 mL). The reaction mixture was stirred at 110° C. for 8 h before cooling to rt. The mixture was diluted with DCM/MeOH (10/1, 300 mL), washed with H₂O (50 mL×2), brine (50 mL), dried over anhydrous Na₂SO₄, filtered off and concentrated in vacuo. The residue was purified by flash column chromatography on silica gel (DCM/MeOH=100/1 to 10/1) to give the crude title compound, which was purified via the reverse phase flash column chromatography (MeOH/H₂O=10% to 90%) to give the title compound (100 mg, yield: 20%).

### Step 2. 4-(6-((1R,5S,6s)-6-amino-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

To a solution of the product of step 1 above (100 mg, 0.2 mmol) in DCM/MeOH (4 mL/1 mL) was added HCl/dioxane (4 N, 2 mL, 8 mmol) at 0° C. The reaction solution was stirred at 45° C. for 2 h before concentrated in vacuo to give the title compound (113 mg, yield: 100%).

### Intermediate 13

### 4-(6-((1R,5S,6s)-6-amino-3-azabicyclo[3.1.0]hexan-3-yl)pyridin-3-yl)-6-ethoxypyrazolo[1,5-a]pyridine-3-carbonitrile hydrochloride

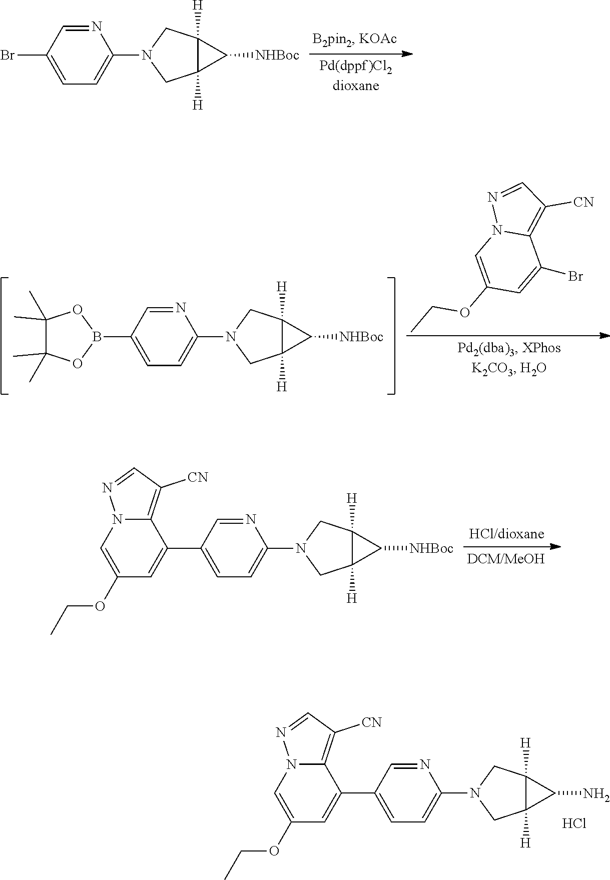

### Step 1. tert-butyl ((1R,5S,6s)-3-(5-(3-cyano-6-ethoxypyrazolo[1,5-a]pyridin-4-yl)pyridin-2-yl)-3-azabicyclo[3.1.0]hexan-6-yl)carbamate

To a solution of the product of Step 1 of Intermediate 7 (200 mg, 0.565 mmol) in dioxane (2 mL) was added B₂(Pin)₂ (151 mg, 0.593 mmol), Pd(dppf)Cl₂ DCM (46 mg, 0.057 mmol), and KOAc (111 mg, 1.13 mmol) at rt. The reaction mixture was flushed with N₂, stirred at 100° C. for 4 h before cooling to rt. To the mixture was added Intermediate 4 (136.8 mg, 0.514 mmol), K₂CO₃ (213 mg, 1.54 mmol), Pd₂dba₃ (23.5 mg, 0.026 mmol), XPhos (49 mg, 0.103 mmol), and H₂O (0.4 mL). The reaction mixture was stirred at 110° C. for 4 h. After cooling to rt, the mixture was diluted with DCM/MeOH (10/1, 200 mL), washed with H₂O (30 mL×2), brine (30 mL), dried over anhydrous Na₂SO₄, filtered off and concentrated in vacuo. The residue was purified by flash column chromatography on silica gel (DCM/MeOH=100/1 to 10/1) to give the title compound (200 mg, yield: 65%).

### Step 2. 4-(6-((1R,5S,6s)-6-amino-3-azabicyclo[3.1.0]hexan-3-yl)pyridin-3-yl)-6-ethoxypyrazolo[1,5-a]pyridine-3-carbonitrile hydrochloride

To a solution of the product of step 1 above (200 mg, 0.434 mmol) in DCM/MeOH (8 mL/2 mL) was added 4N HCl/dioxane (3 mL, 12 mmol) at 0° C. The reaction solution was stirred at 45° C. for 2h before being concentrated in vacuo to give the crude title compound (240 mg, crude, quantitatively), which was used directly without further purification.

### Intermediate 14

### 4-(6-((1R,5S,6r)-6-amino-3-azabicyclo[3.1.0]hexan-3-yl)pyridin-3-yl)-6-ethoxypyrazolo[1,5-a]pyridine-3-carbonitrile hydrochloride

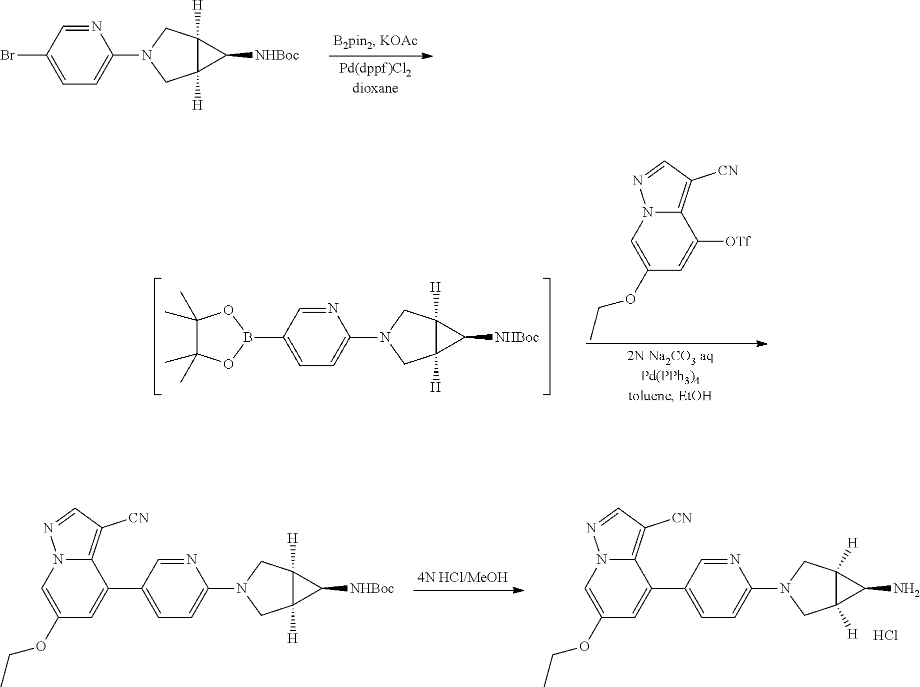

This intermediate was synthesized following the procedure used to make Intermediate 13 starting from the product of step 1 of Intermediate 8 in place of the product of Step 1 of Intermediate 7.

### Intermediate 15

### 4-(6-((1R,5S,6s)-6-amino-3-azabicyclo[3.1.0]hexan-3-yl)pyridin-3-yl)-6-methoxypyrazolo[1,5-a]pyridine-3-carbonitrile hydrochloride

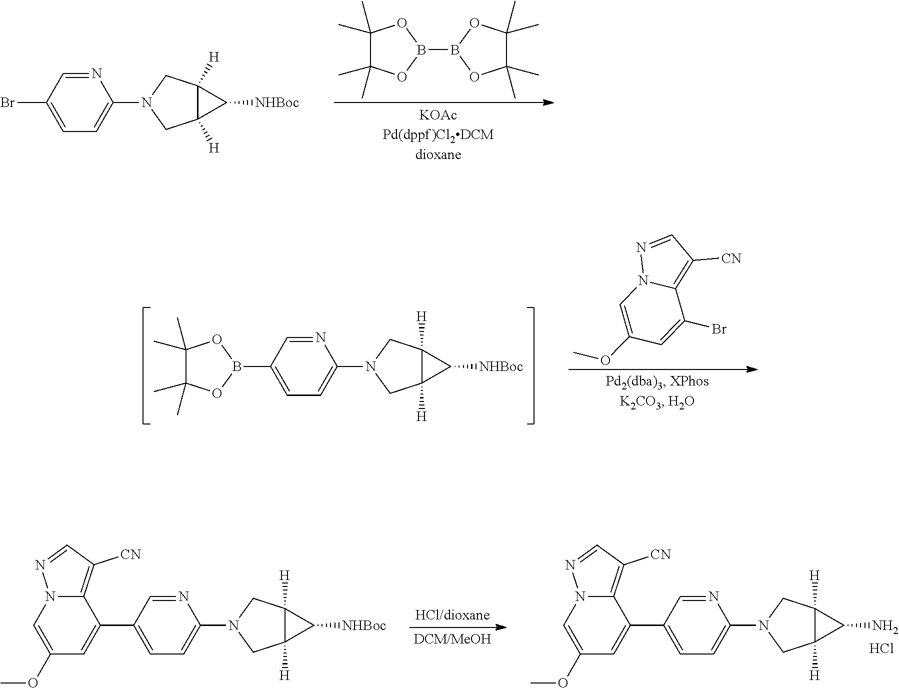

This intermediate was synthesized following the procedure used to make Intermediate 13 by using commercially available 4-bromo-6-methoxypyrazolo[1,5-a]pyridine-3-carbonitrile in place of Intermediate 4.

### Intermediate 16

### 4-(5-((1R,5S,6r)-6-amino-3-azabicyclo[3.1.0]hexan-3-yl)pyrazin-2-yl)-6-ethoxypyrazolo[1,5-a]pyridine-3-carbonitrile hydrochloride

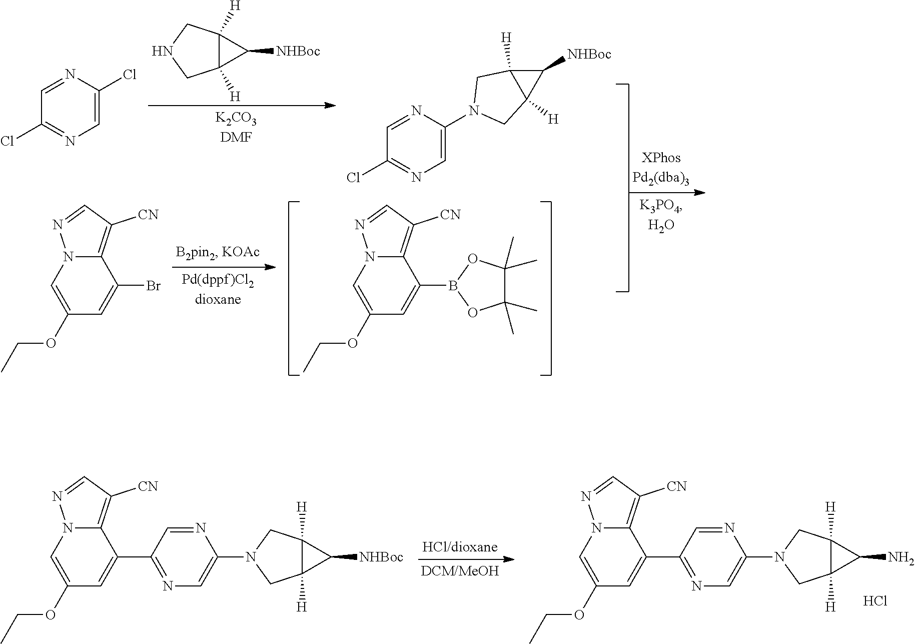

### Step 1. tert-butyl ((1R,5S,6r)-3-(5-chloropyrazin-2-yl)-3-azabicyclo[3.1.0]hexan-6-yl)carbamate

To a solution of 2,5-dichloropyrazine (63 mg, 0.42 mmol) in DMF (2 mL) was added tert-butyl (1R,5S,6r)-3-azabicyclo[3.1.0]hexan-6-ylcarbamate (100 mg, 0.5 mmol) and K₂CO₃ (116 mg, 0.84 mmol). The reaction mixture was stirred at 110° C. for 4 h before being cooled to rt. The reaction mixture was filtered off. The filtrate was concentrated in vacuo to remove solvent. The residue was extracted with DCM/MeOH (10/1, 100 mL), washed by H₂O (30 mL×2), brine (30 mL), dried over anhydrous Na₂SO₄, filtered off and concentrated in vacuo to give the title compound (132 mg, yield:100%).

### Step 2. tert-butyl ((1R,5S,6r)-3-(5-(3-cyano-6-ethoxypyrazolo[1,5-a]pyridin-4-yl)pyrazin-2-yl)-3-azabicyclo[3.1.0]hexan-6-yl)carbamate

To a solution of Intermediate 4 (98 mg, 0.368 mmol) in dioxane (1 mL) was added B₂Pin₂ (98 mg, 0.386 mmol), Pd(dppf)Cl₂ DCM (15 mg, 0.018 mmol), and KOAc (72 mg, 0.736 mmol) at rt. The reaction mixture was stirred at 100° C. under N₂ for 4 h before being cooled to rt. To the reaction mixture was added the product of step 1 above (117 mg, 0.368 mmol), K₃PO₄ (234 mg, 1.104 mmol), Pd₂dba₃ (17 mg, 0.018 mmol), XPhos (35 mg, 0.074 mmol), and H₂O (0.2 mL). The resultant mixture was flushed with N₂, stirred at 110° C. overnight. After cooling to rt, the mixture was diluted with DCM/MeOH (10/1, 100 mL), washed with H₂O (30 mL×2), brine (30 mL), dried over anhydrous Na₂SO₄, filtered off and concentrated in vacuo. The residue was purified by flash column chromatography on silica gel (DCM to DCM/MeOH=40/1) to give the title compound (149 mg, yield: 88%).

### Step 3. 4-(5-((1R,5S,6r)-6-amino-3-azabicyclo[3.1.0]hexan-3-yl)pyrazin-2-yl)-6-ethoxypyrazolo[1,5-a]pyridine-3-carbonitrile hydrochloride

To a solution of the product of step 2 above (225 mg, 0.173 mmol) in DCM/MeOH (4 mL/1 mL) was added 4N HCl/dioxane (1 mL, 4 mmol) at 0° C. The reaction mixture was stirred at rt overnight. The mixture was concentrated in vacuo to give the crude title compound (395 mg, crude), which was used directly to the next step.

### Intermediate 17

### 4-(5-((1R,5S,6s)-6-amino-3-azabicyclo[3.1.0]hexan-3-yl)pyrazin-2-yl)-6-ethoxypyrazolo[1,5-a]pyridine-3-carbonitrile hydrochloride

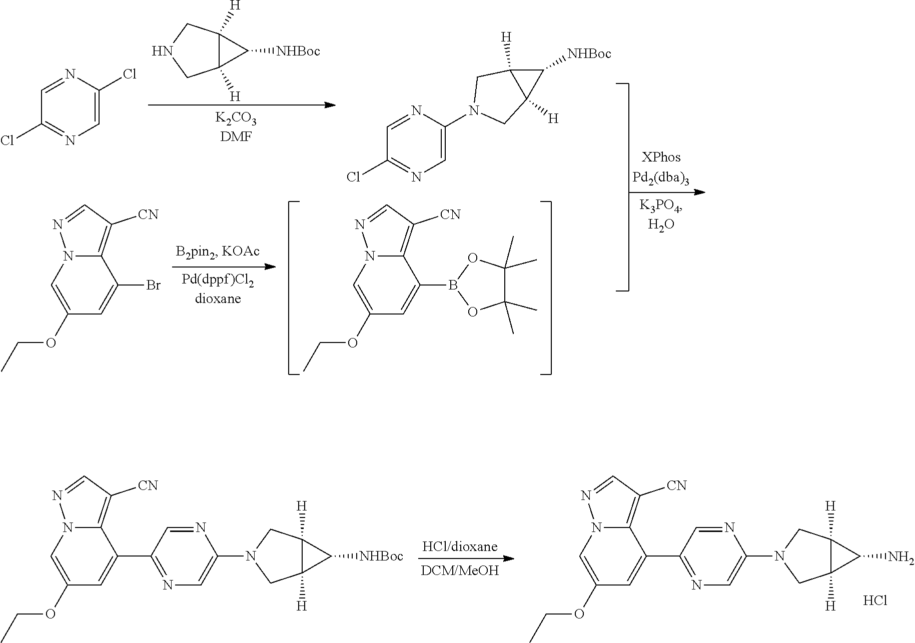

This intermediate was synthesized following the procedure used to make Intermediate 17 starting from tert-butyl (1R,5S,6s)-3-azabicyclo[3.1.0]hexan-6-ylcarbamate in place of tert-butyl (1R,5S,6r)-3-azabicyclo[3.1.0]hexan-6-ylcarbamate.

### Intermediate 18

### 4-(6-((1R,5S,6r)-6-amino-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

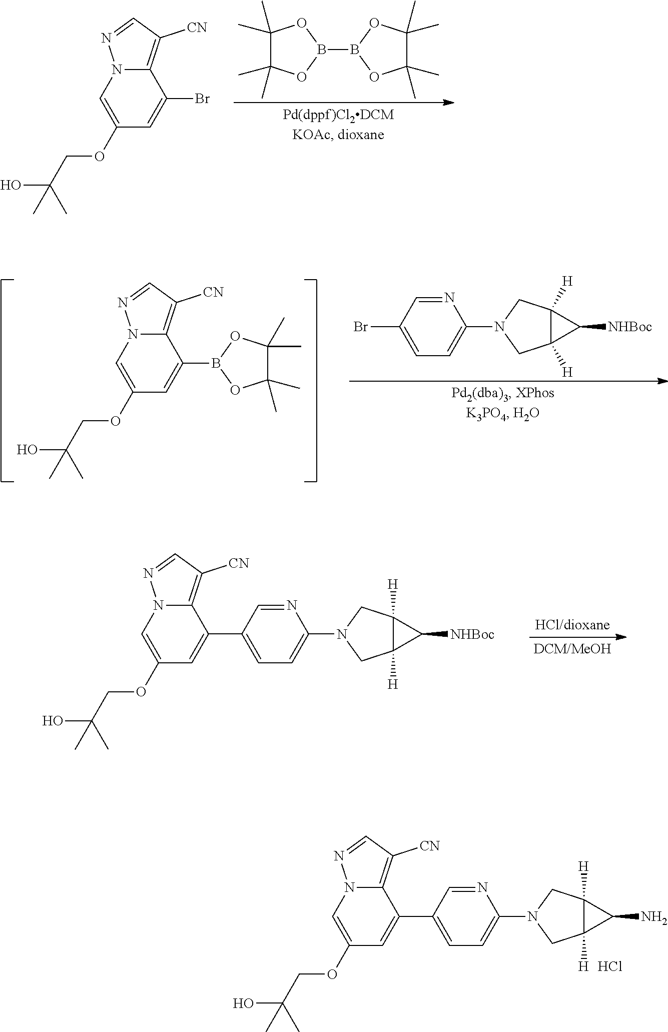

### Step 1. tert-butyl ((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)carbamate

To a solution of Intermediate 3 (102 mg, 0.33 mmol) in dioxane (1 mL) was added B₂Pin₂ (88 mg, 0.346 mmol), Pd(dppf)Cl₂ DCM (13.5 mg, 0.0165 mmol), and KOAc (65 mg, 0.66 mmol) at rt. The reaction mixture was stirred at 100° C. under N₂ for 4 h before being cooled to rt. To the reaction mixture was added the product of step 1 of Intermediate 8 (117 mg, 0.33 mmol), K₃PO₄ (210 mg, 0.99 mmol), Pd₂dba₃ (15 mg, 0.0165 mmol), XPhos (31.3 mg, 0.066 mmol), and H₂O (0.2 mL). The resultant mixture was flushed with N₂, stirred at 110° C. for 4h. After cooling to rt, the mixture was diluted with DCM/MeOH (10/1, 100 mL), washed with H₂O (30 mL×2), brine (30 mL), dried over anhydrous Na₂SO₄, and concentrated in vacuo. The residue was purified by reverse phase flash column chromatography (MeOH/H2O=5% to 95%) to give the title compound (60 mg, yield: 36%).

### Step 2. 4-(6-((1R,5S,6r)-6-amino-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

To a solution of the product of step 1 above (60 mg, 0.119 mmol) in DCM/MeOH (5 mL/1 mL) was added 4N HCl/dioxane (2 mL, 8 mmol) at 0° C. The reaction solution was stirred at rt for 2h before being concentrated in vacuo to give the crude title compound (48 mg, crude), which was used directly to the next step.

### Intermediate 19

### N-((3aR,5r,6aS)-2-(5-bromopyridin-2-yl)octahydrocyclopenta[c]pyrrol-5-yl)-3-chloropicolinamide

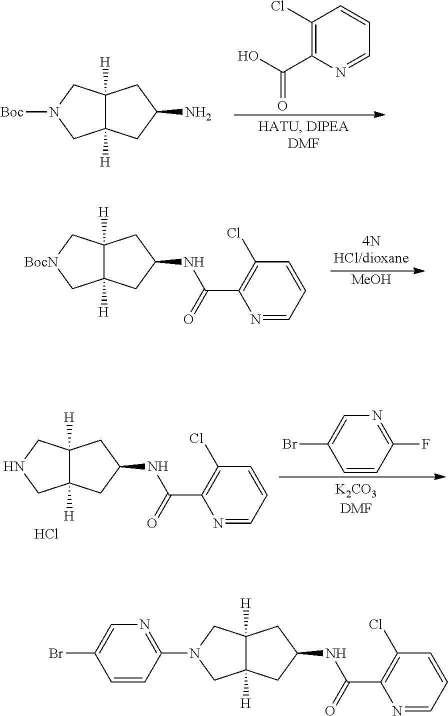

### Step 1. (3aR,5r,6aS)-tert-butyl 5-(3-chloropicolinamido)hexahydrocyclopenta[c]pyrrole-2(1H)-carboxylate

To a solution of (3aR,5r,6aS)-tert-butyl 5-aminohexahydrocyclopenta[c]pyrrole-2(1H)-carboxylate (400 mg, 1.767 mmol), 3-chloropicolinic acid (278 mg, 1.767 mmol) and HATU (1.008 g, 2.651 mmol) in DMF (3 mL) was added DIPEA (685 mg, 5.301 mg). The mixture was stirred at 50° C. for 1.5h. After cooling to rt, the mixture was directly purified by reverse phase flash column chromatography (MeOH/H₂O=5% to 95%) to give the title compound (129 mg, yield: 20%).

### Step 2. 3-chloro-N-((3aR,5r,6aS)-octahydrocyclopenta[c]pyrrol-5-yl)picolinamide hydrochloride

To a solution of the product of step 1 above (210 mg, 0.574 mmol) in MeOH (3 mL) was added 4N HCl/dioxane (3 mL, 12 mmol). The mixture was stirred at 50° C. for 4h and concentrated in vacuo to give the crude title compound (240 mg, crude yield: 138%.

### Step 3. N-((3aR,5r,6aS)-2-(5-bromopyridin-2-yl)octahydrocyclopenta[c]pyrrol-5-yl)-3-chloropicolinamide

A solution of 5-bromo-2-fluoropyridine (37 mg, 0.211 mmol), the product of step 2 above (73 mg, 0.232 mmol), and K₂CO₃ (87 mg, 0.633 mmol) in DMF (1.5 mL) was stirred at 110° C. for 1.5h. The mixture was cooled to rt, diluted with DCM/MeOH (10/1, 50 mL), washed with H₂O (20 mL×2), dried over anhydrous Na₂SO₄, filtered off, and concentrated in vacuo. The residue was purified by reverse phase flash column chromatography (MeOH/H₂O=5% to 95%) to give the title compound (72 mg, yield: 49%).

### Intermediate 20

### (3aR,5r,6aS)-octahydrocyclopenta[c]pyrrol-5-ol hydrochloride

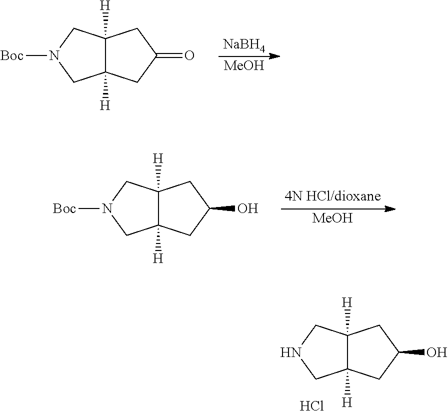

### Step 1. (3aR,5r,6aS)-tert-butyl 5-hydroxyhexahydrocyclopenta[c]pyrrole-2(1H)-carboxylate

To a solution of (3aR,6aS)-tert-butyl 5-oxohexahydrocyclopenta[c]pyrrole-2(1H)-carboxylate (2.0 g, 8.88 mmol) in MeOH (20 mL) cooled in an ice-H₂O bath was added NaBH₄ (504 mg, 13.32 mmol) portionwise while maintaining internal temperature <30° C. After the addition was completed, the mixture was stirred at rt for 0.5h. The reaction was quenched with acetone (2 mL), and concentrated. The residue was taken up in EtOAc (100 mL), which was washed with H₂O (40 mL×2), brine (40 mL), dried over anhydrous Na₂SO₄, filtered off and concentrated to give the crude title compound (2.8 g, crude yield: >100%).

### Step 2. (3aR,5r,6aS)-octahydrocyclopenta[c]pyrrol-5-ol hydrochloride

To a solution of crude product of step 1 above (2.8 g, ˜8.88 mmol) in MeOH (10 mL) was added 4N HCl/dioxane (10 mL, 40 mmol). The mixture was stirred at 30° C. for 2h and concentrated to give the crude title compound (1.56 g, crude yield: >100%).

### Intermediate 21

### 4-(5-((3aR,5s,6aS)-5-aminohexahydrocyclopenta[c]pyrrol-2(1H)-yl)pyrazin-2-yl)-6-ethoxypyrazolo[1,5-a]pyridine-3-carbonitrile

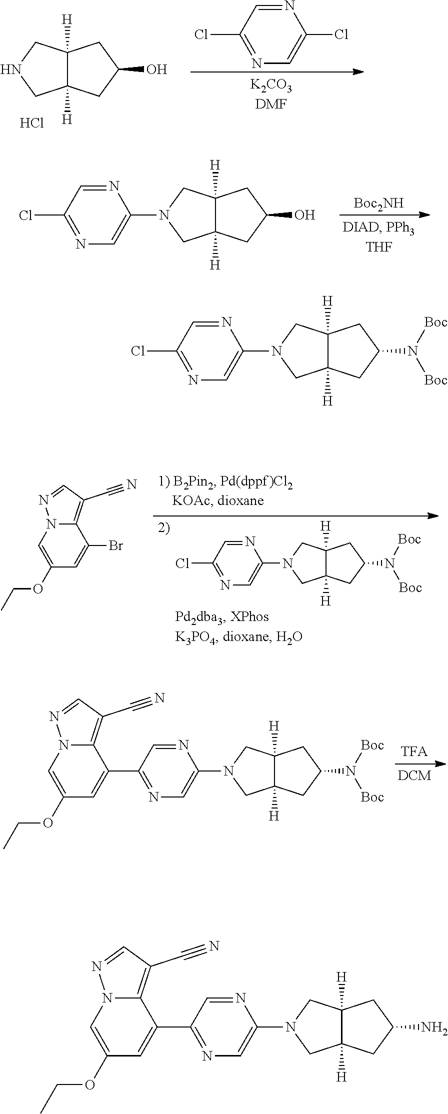

### Step 1. (3aR,5r,6aS)-2-(5-chloropyrazin-2-yl)octahydrocyclopenta[c]pyrrol-5-ol

A solution of Intermediate 20 (1.56 g, 9.53 mmol), 2,5-dichloropyrazine (1.29 g, 8.67 mmol), and K₂CO₃ (3.59 g, 26.01 mmol) in DMF (20 mL) was stirred at 105° C. overnight. The mixture was cooled to rt, diluted with H₂O (40 mL), and extracted with DCM/i-propanol (3/1, 100 ml×2). The combined organic layers were washed with H₂O (30 mL×2), brine (50 mL), dried over anhydrous Na₂SO₄, filtered off and concentrated to give the title compound (2.31 g, crude yield: >100%).

### Step 2. tert-Butyl N-[(tert-butoxy)carbonyl]-N-((3aR,5s,6aS)-2-(5-chloropyrazin-2-yl)octahydrocyclopenta[c]pyrrol-5-yl)carbamate

A solution of the product of step 1 above (1.0 g, 4.17 mmol), di-tert-butyl iminodicarboxylate (997 mg, 4.5 mmol), and PPh₃ (1.20 g, 4.5 mmol) in THF (15 mL) was cooled to 0° C. under N₂ atmosphere and DIAD (928 mg, 4.5 mmol) was added dropwise. After the addition was complete, the mixture was stirred at rt overnight, and diluted with EtOAc (100 mL). The mixture was washed with sat. aq. NaHCO₃ (25 mL), H₂O (25 mL), brine (50 mL), dried over anhydrous Na₂SO₄, filtered off and concentrated. The residue was purified by flash column chromatography on silica gel (PE/EtOAc=4/1) to give the title compound (1.19 g, yield: 63%).

### Step 3. tert-butyl N-((3aR,5s,6aS)-2-(5-(3-cyano-6-ethoxypyrazolo[1,5-a]pyridin-4-yl)pyrazin-2-yl)octahydrocyclopenta[c]pyrrol-5-yl)-N-[(tert-butoxy)carbonyl]-carbamate

A solution of Intermediate 4 (200 mg, 0.753 mmol), B₂Pin₂ (200 mg, 0.789 mmol), Pd(dppf)Cl₂ DCM (61 mg, 0.0752 mmol), and KOAc (148 mg, 1.504 mmol) in dioxane (2 mL) was stirred at 100° C. for 4h. The mixture was cooled to rt and the product of step 2 above (330 mg, 0.752 mmol), Pd₂dba₃ (34 mg, 0.0376 mmol), XPhos (72 mg, 0.1504 mmol), K₃PO₄ (475 mg, 2.256 mmol), and dioxane (4 mL) and H₂O (0.8 mL) was added. The reaction mixture was stirred at 110° C. for 4h. The mixture was filtered off, and the filtrate was diluted with DCM/MeOH (10/1, 100 mL), washed with H₂O (50 mL), brine (50 mL), dried over anhydrous Na₂SO₄, filtered off and concentrated in vacuo. The residue was purified by flash column chromatography on silica gel (DCM/MeOH=100/1 to 10/1) to give the title compound (308 mg, yield: 70%).

### Step 4. 4-(5-((3aR,5s,6aS)-5-aminohexahydrocyclopenta[c]pyrrol-2(1H)-yl)pyrazin-2-yl)-6-ethoxypyrazolo[1,5-a]pyridine-3-carbonitrile

To a solution of the product of step 3 above (248 mg, 0.421 mmol) in DCM (10 mL) was added TFA (1 mL) over an ice-H₂O bath. The mixture was stirred at rt overnight, diluted with H₂O (1 mL), adjusted to pH 8-9 with sat. aq. NaHCO₃, and extracted with DCM/MeOH (10/1, 100 mL). The organic layer was washed with brine (50 mL), dried over anhydrous Na₂SO₄, filtered off and concentrated to give the title compound (163 mg, yield: 99%).

### Intermediate 22

### 4-(6-((3aR,5s,6aS)-5-aminohexahydrocyclopenta[c]pyrrol-2(1H)-yl)pyridin-3-yl)-6-ethoxypyrazolo[1,5-a]pyridine-3-carbonitrile hydrochloride

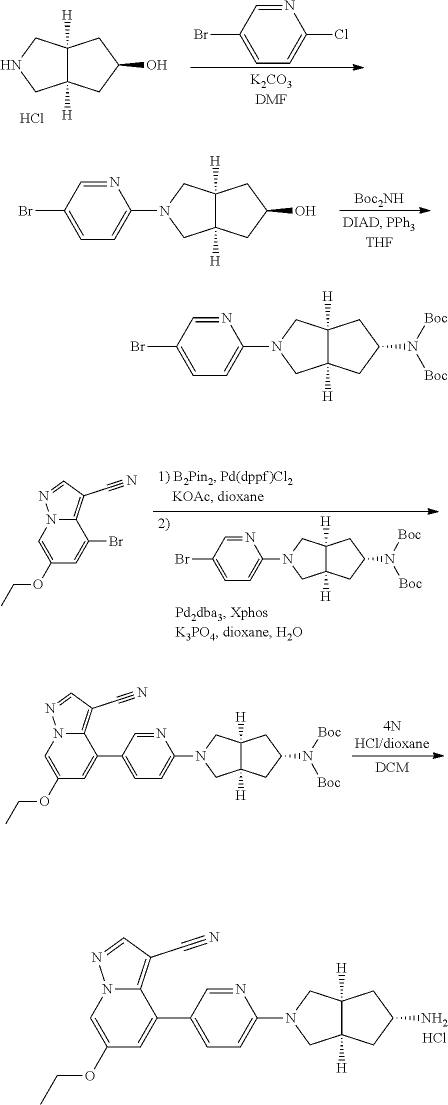

### Step 1. (3aR,5r,6aS)-2-(5-bromopyridin-2-yl)octahydrocyclopenta[c]pyrrol-5-ol

A solution of Intermediate 20 (794 mg, 4.85 mmol), 5-bromo-2-fluoropyridine (776 mg, 4.41 mmol), and K₂CO₃ (1.83 g, 13.23 mmol) in DMF (12 mL) was stirred at 110° C. overnight. After cooling to rt, the mixture was diluted with H₂O (40 mL), and extracted with DCM/MeOH (10/1, 60 ml×2). The combined organics were washed with H₂O (60 mL), brine (50 mL), dried over anhydrous Na₂SO₄, filtered and concentrated in vacuo to give the title compound (1.308 g, crude yield: >100%).

### Step 2. tert-butyl N-[(tert-butoxy)carbonyl]-N-((3aR,5s,6aS)-2-(5-bromopyridin-2-yl)octahydrocyclopenta[c]pyrrol-5-yl)carbamate

A solution of the product of step 1 above (1.208 g, 4.27 mmol), di-tert-butyl iminodicarboxylate (1.11 g, 5.12 mmol), and PPh₃ (1.34 g, 5.12 mmol) in THF (15 mL) was cooled to 0° C. under N₂ atmosphere and DIAD (1.04 g, 5.12 mmol) was added dropwise. After the addition was complete, the mixture was stirred at rt for 0.5h and concentrated. The residue was purified by flash column chromatography on silica gel (PE/EtOAc=10/1) to give the title compound (650 mg, yield: 32%).

### Step 3. tert-butyl N-[(tert-butoxy)carbonyl]-N-((3aR,5s,6aS)-2-(5-(3-cyano-6-ethoxypyrazolo[1,5-a]pyridin-4-yl)pyridin-2-yl)octahydrocyclopenta[c]pyrrol-5-yl)carbamate

A solution of Intermediate 4 (200 mg, 0.753 mmol), B₂Pin₂ (200 mg, 0.789 mmol), Pd(dppf)Cl₂ DCM (61 mg, 0.0752 mmol), and KOAc (148 mg, 1.504 mmol) in dioxane (2 mL) was stirred at 100° C. under N₂ for 4h. After cooling to rt, to the mixture added the product of step 2 above (360 mg, 0.752 mmol), Pd₂dba₃ (34 mg, 0.0376 mmol), XPhos (72 mg, 0.1504 mmol), K₃PO₄ (475 mg, 2.256 mmol), and dioxane (4 mL) and H₂O (0.8 mL). The reaction mixture was stirred at 110° C. under N₂ for 4h. The mixture was filtered off and the filtrate was diluted with EtOAc (100 mL), washed with H₂O (50 mL), brine (50 mL), dried over anhydrous Na₂SO₄, filtered off and concentrated in vacuo. The residue was purified by flash column chromatography on silica gel (DCM/MeOH=100/1 to 10/1) to give the title compound (420 mg, yield: 95%).

### Step 4. 4-(6-((3aR,5s,6aS)-5-aminohexahydrocyclopenta[c]pyrrol-2(1H)-yl)pyridin-3-yl)-6-ethoxypyrazolo[1,5-a]pyridine-3-carbonitrile hydrochloride

To a solution the product of step 3 above (400 mg, 0.679 mmol) in DCM (10 mL) was added 4N HCl/dioxane (8 mL) cooled in an ice-H₂O bath. The mixture was stirred at rt for 2h and concentrated in vacuo to give the crude title compound (417 mg, crude yield: >100%).

### Intermediate 23

### N-((3aR,5s,6aS)-2-(5-bromopyridin-2-yl)-5-methyloctahydrocyclopenta[c]pyrrol-5-yl)-3-chloropicolinamide

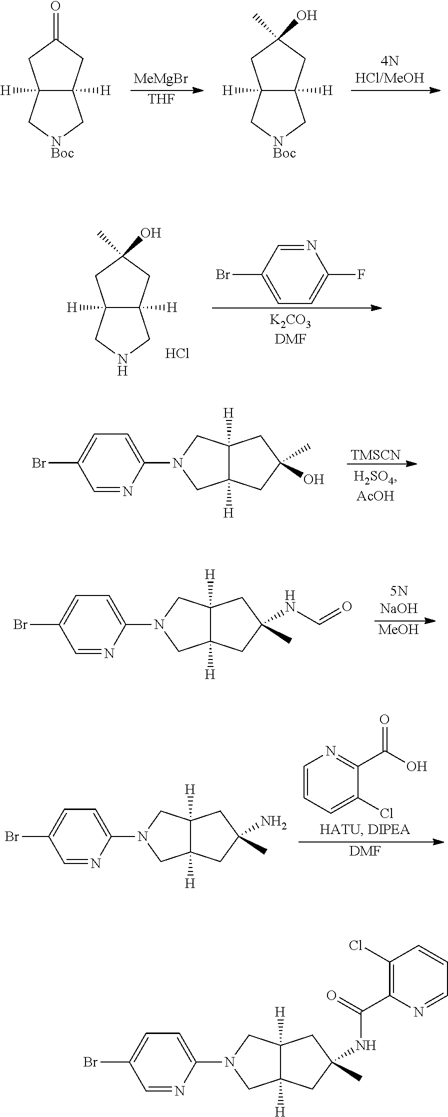

### Step 1. (3aR,5r,6aS)-tert-butyl 5-hydroxy-5-methylhexahydrocyclopenta[c]pyrrole-2(1H)-carboxylate

To a solution of (3aR,6aS)-tert-butyl 5-oxohexahydrocyclopenta[c]pyrrole-2(1H)-carboxylate (2.25 g, 10 mmol) in dry toluene (25 mL) was added methylmagnesium bromide (1.0 N, 25 mmol) at −30° C. The mixture was stirred at −30° C. for 2 h. The reaction was quenched by dropwise addition of MeOH (2 mL) and HCl (6 N, 10 mL) at −30° C. The mixture was diluted with EtOAc (100 mL), washed by H₂O (30×2 mL) and brine (30 mL), dried over anhydrous Na₂SO₄, filtered off and concentrated in vacuo. The residue was purified by flash column chromatography on silica gel (PE/EtOAc=4/1-1/1) to give the title compound (2.0 g, yield: 83%).

### Step 2. (3aR,5r,6aS)-5-methyloctahydrocyclopenta[c]pyrrol-5-ol hydrochloride

A solution of the product of step 1 above (1.5 g, 6.22 mmol) in HCl/MeOH (4 N, 10 mL) was stirred at 40° C. for 2 h. The reaction mixture was concentrated and dried in vacuo to give the crude title compound (quantitatively).

### Step 3. (3aR,5r,6aS)-2-(5-bromopyridin-2-yl)-5-methyloctahydrocyclopenta[c]pyrrol-5-ol

To a solution of the product of step 2 above (6.22 mmol) and K₂CO₃ (3.44 g, 24.9 mmol) in DMF (15 mL) was added 5-bromo-2-fluoropyridine (1.1 g, 6.22 mmol). The mixture was stirred at 110° C. for 2 h. After cooling to rt, the mixture was diluted with EtOAc (100 mL), washed by H₂O (30 mL×2) and brine (30 mL), dried over anhydrous Na₂SO₄, filtered off, and concentrated in vacuo. The residue was purified by flash column chromatography on silica gel (PE/EtOAc=3/1-1/1) to give the title compound (1.4 g, yield: 76%).

### Step 4. N-((3aR,5s,6aS)-2-(5-bromopyridin-2-yl)-5-methyloctahydrocyclopenta[c]pyrrol-5-yl)formamide

To a solution of the product of step 3 above (200 mg, 0.67 mmol) and trimethylsilanecarbonitrile (200 mg, 2.02 mmol) in HOAc (0.5 mL) was added conc. H₂SO₄ (0.4 mL) at 0° C. The mixture was stirred at rt for 2 h. The reaction was cooled in ice-H₂O bath, basified with aqueous NaOH (5 N) until pH 8-9. The mixture was extracted with DCM (30 mL×3). The combine organics were washed by H₂O (30 mL×2) and brine (30 mL), dried over anhydrous Na₂SO₄, filtered off, and concentrated. The residue was purified by Prep-TLC (PE/EtOAc=1/2) to give the title compound (200 mg, yield: 92%).

### Step 5. (3aR,5s,6aS)-2-(5-bromopyridin-2-yl)-5-methyloctahydrocyclopenta[c]pyrrol-5-amine

To a solution of the product of Step 4 above (200 mg, 0.62 mmol) in EtOH (3 mL) was added aqueous NaOH (5 N, 3 mL). The mixture was stirred at 80° C. for 2 h. After cooling to rt, the mixture was diluted with DCM/MeOH=10/1 (50 mL). The organic phase was collected, washed by brine (15 mL), dried over anhydrous Na₂SO₄, filtered off, and concentrated in vacuo to give the title compound (180 mg, yield: 98%).

### Step 6. N-((3aR,5s,6aS)-2-(5-bromopyridin-2-yl)-5-methyloctahydrocyclopenta[c]pyrrol-5-yl)-3-chloropicolinamide

To a solution of the product of step 5 above (160 mg, 0.54 mmol), 3-chloropicolinic acid (85 mg, 0.54 mmol), and HATU (308 mg, 0.81 mmol) in DMF (5 mL) was added DIPEA (209 mg, 1.62 mmol) at rt. The mixture was stirred at 40° C. for 2 h. After cooling to rt, the mixture was diluted with EtOAc (50 mL), washed by H₂O (15 mL×2) and brine (15 mL), dried over anhydrous Na₂SO₄, filtered off, and concentrated in vacuo. The residue was purified by Prep-TLC (PE/EtOAc=1/1) to give the title compound (190 mg, yield: 81%).

### Intermediate 24

### 4-(5-((3aR,5s,6aS)-5-amino-5-methylhexahydrocyclopenta[c]pyrrol-2(1H)-yl)pyrazin-2-yl)-6-ethoxypyrazolo[1,5-a]pyridine-3-carbonitrile

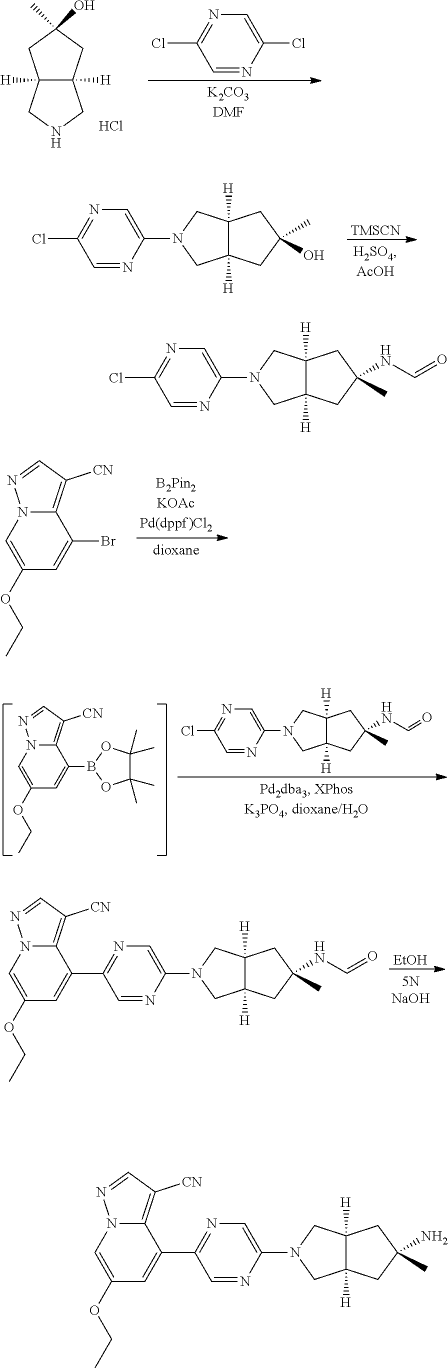

### Step 1. (3aR,5r,6aS)-2-(5-chloropyrazin-2-yl)-5-methyloctahydrocyclopenta[c]pyrrol-5-ol

To a solution of the product of Step 2 of Intermediate 23 (610 mg, 4.1 mmol) and K₂CO₃ (1.7 g, 12.3 mmol) in DMF (5 mL) was added 2,5-dichloropyrazine (0.8 g, 4.5 mmol). The mixture was stirred at 110° C. for 2 h. After cooling to rt, the mixture was diluted with EtOAc (100 mL), washed by H₂O (30 mL×2) and brine (30 mL), dried over anhydrous Na₂SO₄, filtered off, and concentrated in vacuo. The residue was purified by flash column chromatography on silica gel (PE/EtOAc=4/1 to 1/1) to give the title compound (630 mg, yield: 61%).

### Step 2. N-((3aR,5s,6aS)-2-(5-chloropyrazin-2-yl)-5-methyloctahydrocyclopenta[c]pyrrol-5-yl)formamide

To a solution of the product of step 1 above (200 mg, 0.79 mmol) and TMSCN (234 mg, 2.36 mmol) in HOAc (0.5 mL) was added concentrated H₂SO₄ (0.4 mL) at 0° C. The mixture was stirred at rt for 2 h. The reaction was quenched with ice, basified with aqueous NaOH (5 N) to pH 8-9, and extracted with DCM (50 mL×3). The combined organics were washed by H₂O (30 mL×2) and brine (30 mL), dried over anhydrous Na₂SO₄, filtered off, and concentrated in vacuo. The residue was purified by Prep-TLC (PE/EtOAc=1/1 to EtOAc) to give the title compound (215 mg, yield: 97%).

### Step 3. N-((3aR,5s,6aS)-2-(5-(3-cyano-6-ethoxypyrazolo[1,5-a]pyridin-4-yl)pyrazin-2-yl)-5-methyloctahydrocyclopenta[c]pyrrol-5-yl)formamide

A solution of Intermediate 4 (150 mg, 0.56 mmol), B₂Pin₂ (150 mg, 0.59 mmol), Pd(dppf)Cl₂ DCM (23 mg, 0.028 mmol), and KOAc (110 mg, 1.12 mmol) in dioxane (2 mL) was stirred at 105° C. for 2h under N₂. To the mixture after cooling to rt was added the product of step 2 above (215 mg, 0.765 mmol), Pd₂dba₃ (35 mg, 0.0382 mmol), XPhos (73 mg, 0.153 mmol), K₃PO₄ (487 mg, 2.295 mmol), and dioxane/H₂O (5/1 mL). The resultant mixture was flushed with N₂, stirred at 110° C. overnight. The mixture was diluted with DCM/MeOH (10/1, 100 mL), washed by H₂O (30 mL×2) and brine (30 mL), dried over anhydrous Na₂SO₄, filtered off, and concentrated in vacuo. The residue was purified by Prep-TLC (DCM/MeOH=30/1) to give the title compound (80 mg, yield: 33%).

### Step 4. 4-(5-((3aR,5s,6aS)-5-amino-5-methylhexahydrocyclopenta[c]pyrrol-2(1H)-yl)pyrazin-2-yl)-6-ethoxypyrazolo[1,5-a]pyridine-3-carbonitrile

To a solution of the product of step 3 above (70 mg, 0.16 mmol) in EtOH (5 mL) was added aqueous NaOH (5 N, 5 mL). The mixture was stirred at 80° C. for 2 h. After cooling to rt, the mixture was diluted with DCM/MeOH=10/1 (50 mL), washed by brine (30 mL), dried over anhydrous Na₂SO₄, filtered off, and concentrated in vacuo to give the compound (60 mg, yield: 92%).

### Intermediate 25

### 3-benzyl 6-ethyl (1R,5S,6r)-3-azabicyclo[3.1.0]hexane-3,6-dicarboxylate and 3-benzyl 6-ethyl (1R,5S,6s)-3-azabicyclo[3.1.0]hexane-3,6-dicarboxylate

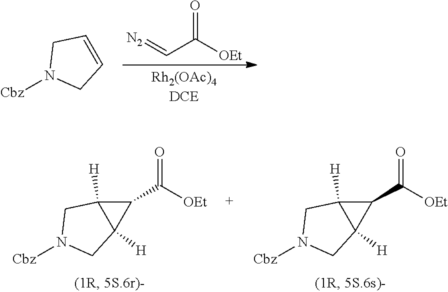

To a solution of benzyl 2,5-dihydro-1H-pyrrole-1-carboxylate (5.0 g, 24.6 mmol) and Rh₂(OAc)₄ (500 mg, 1.13 mmol) in DCE (50 mL) heated to 80° C. was added a solution of ethyl 2-diazoacetate (14 g, 123 mmol) in DCE (50 ml) was added dropwise over a period of 4h. After the addition is completed, the mixture was stirred at 80° C. overnight. After cooling, the mixture was concentrated in vacuo. The residue was purified by flash column chromatography on silica gel (PE/EtOAc=20/1 to 4/1) to give the exo-isomer (upper spot on TLC, 3.1 g, yield: 43%) and endo-isomer (lower spot on TLC, 1.6 g, yield: 22%).

### Intermediate 26

### 4-(6-((1R,5S,6s)-6-(aminomethyl)-3-azabicyclo[3.1.0]hexan-3-yl)pyridin-3-yl)-6-(1-methyl-1H-pyrazol-4-yl)pyrazolo[1,5-a]pyridine-3-carbonitrile TFA salt

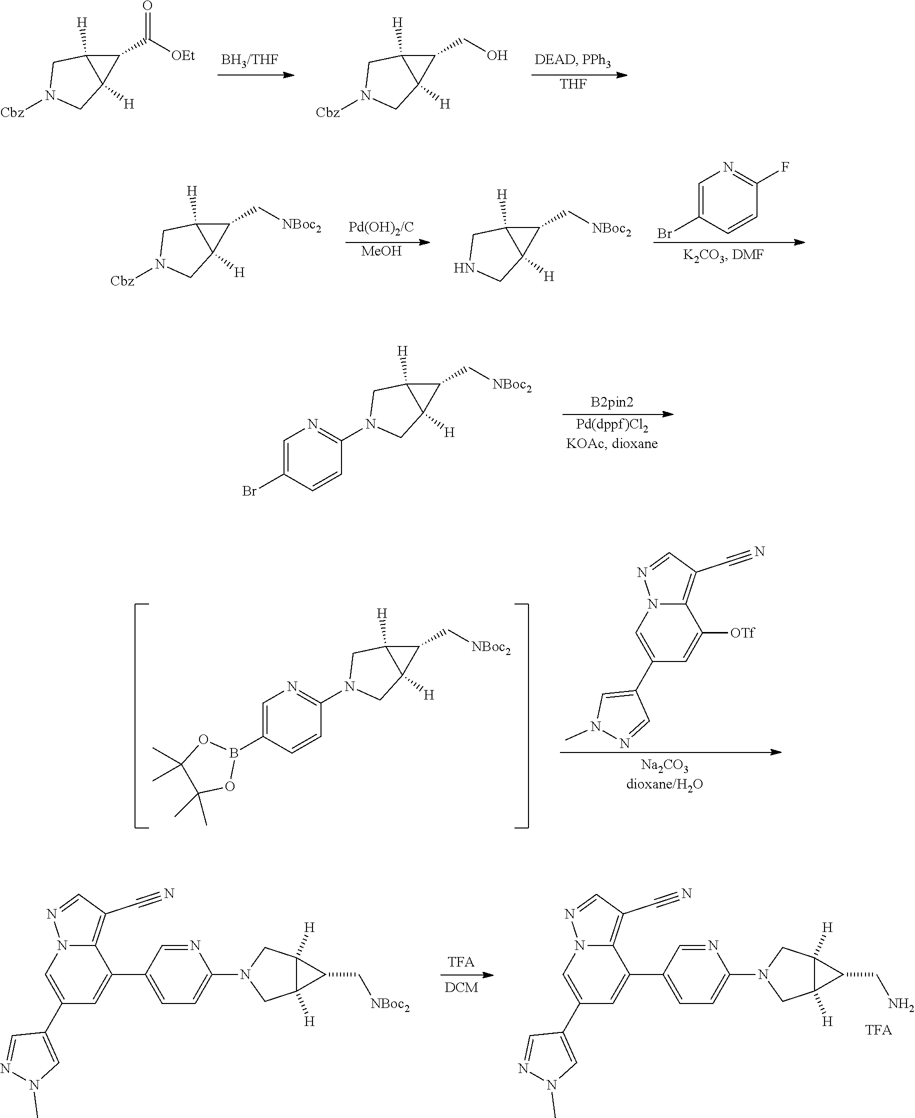

### Step 1. benzyl (1R,5S,6r)-6-(hydroxymethyl)-3-azabicyclo[3.1.0]hexane-3-carboxylate

To a solution of the (1R,5S,6r)-isomer of Intermediate 25 in THF (25 mL) was added dropwise BH₃/THF (1 N, 18 mL, 18 mmol) at 0° C. After the addition was completed, the mixture was heated to 70° C., stirred for 2h. The mixture was concentrated in vacuo and the residue was taken up in DCM (50 mL) and brine (30 mL) and the layers were separated. The aqueous layer was acidified to pH 5 with 1N HCl and extracted with DCM (50 mL×2). The combined organic layers were dried over anhydrous Na₂SO₄ and concentrated in vacuo. The residue was purified by flash column chromatography on silica gel (PE/EtOAc=1/1) to give the title compound (1.18 g, yield: 47%).

### Step 2. benzyl (1R,5S,6r)-6-((bis(tert-butoxycarbonyl)amino)methyl)-3-azabicyclo[3.1.0]hexane-3-carboxylate

To a solution of the product of step 1 above (1.13 g, 4.57 mmol), di-tert-butyl iminodicarboxylate (1.09 g, 5.03 mmol) and PPh₃ (1.56 g, 5.94 mmol) in THF (20 mL) was added dropwise DEAD (1.03 g, 5.94 mmol) at 0° C. under N₂. The mixture was allowed to warm to rt, heated to 50° C. and stirred overnight. The mixture was extracted with EtOAc (100 mL). The organic layer was washed with H₂O (30 mL) and brine (30 mL), dried over anhydrous Na₂SO₄, filtered off, and concentrated in vacuo. The residue was purified by flash column chromatography on silica gel (PE/EtOAc=12/1 to 8/1) to give the title compound (900 mg, 42%).

### Step 3. tert-butyl (((1R,5S,6r)-3-azabicyclo[3.1.0]hexan-6-yl)methyl)(tert-butoxycarbonyl)carbamate

To a solution of the product of step 2 above (900 mg, 2.02 mmol) in MeOH (15 mL) was added Pd(OH)₂/C (100 mg, 20% on carbon, ca. 50% H₂O). The mixture was stirred at rt for 1.5h over a hydrogen balloon. The mixture was filtered off and the filtrate was concentrated to give the title compound (616 mg, yield: 98%).

### Step 4. tert-butyl (((1R,5S,6r)-3-(5-bromopyridin-2-yl)-3-azabicyclo[3.1.0]hexan-6-yl)methyl)(tert-butoxycarbonyl)carbamate

A mixture of the product of step 3 above (560 mg, 1.79 mmol), 5-bromo-2-fluoropyridine (316 mg, 1.79 mmol) and K₂CO₃ (494 mg, 3.58 mmol) was stirred at 100° C. overnight. After cooling to rt, the mixture was concentrated in vacuo. The residue was purified by flash column chromatography on silica gel (PE/EtOAc=30/1 to 15/1) to give the title compound (550 mg, yield: 60%).

### Step 5. tert-butyl (tert-butoxycarbonyl)(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate

A solution of the product of Step 4 above (500 mg, 1.07 mmol), B₂Pin₂ (280 mg, 1.12 mmol), Pd(dppf)Cl₂ DCM (90 mg, 0.107 mmol), and KOAc (210 mg, 2.14 mmol) in dioxane (10 mL) was stirred at 100° C. for 3h. To the mixture after cooled to rt was added Intermediate 1 (400 mg, 1.07 mmol), Na₂CO₃ (230 mg, 2.14 mmol), Pd(dppf)Cl₂ DCM (90 mg, 0.107 mmol) and dioxane/H₂O (10 mL/2 mL). The reaction mixture was stirred at 110° C. for 5h. The mixture was filtered off and the filtrate was concentrated in vacuo. The residue was taken up in DCM/MeOH (10/1, 140 mL), washed with H₂O (30 mL) and brine (30 mL), dried over anhydrous Na₂SO₄, filtered off and concentrated in vacuo. The residue was purified by flash column chromatography on silica gel (PE/EtOAc=2/1, then DCM/EtOAc=2/1 to 1/1) to give the title compound (337 mg, yield: 47%).

### Step 6. 4-(6-((1R,5S,6s)-6-(aminomethyl)-3-azabicyclo[3.1.0]hexan-3-yl)pyridin-3-yl)-6-(1-methyl-1H-pyrazol-4-yl)pyrazolo[1,5-a]pyridine-3-carbonitrile TFA salt

To a solution of the product of step 5 above (270 mg, 0.44 mmol) in DCM (8 mL) was added TFA (2 mL) at 0° C. The mixture was stirred at rt for 2h and concentrated in vacuo to give the crude title compound (277 mg, yield: >100%), which was used in the next step without any further purification.

### Intermediate 27

### 4-(6-((1R,5S,6r)-6-(aminomethyl)-3-azabicyclo[3.1.0]hexan-3-yl)pyridin-3-yl)-6-(1-methyl-1H-pyrazol-4-yl)pyrazolo[1,5-a]pyridine-3-carbonitrile hydrochloride

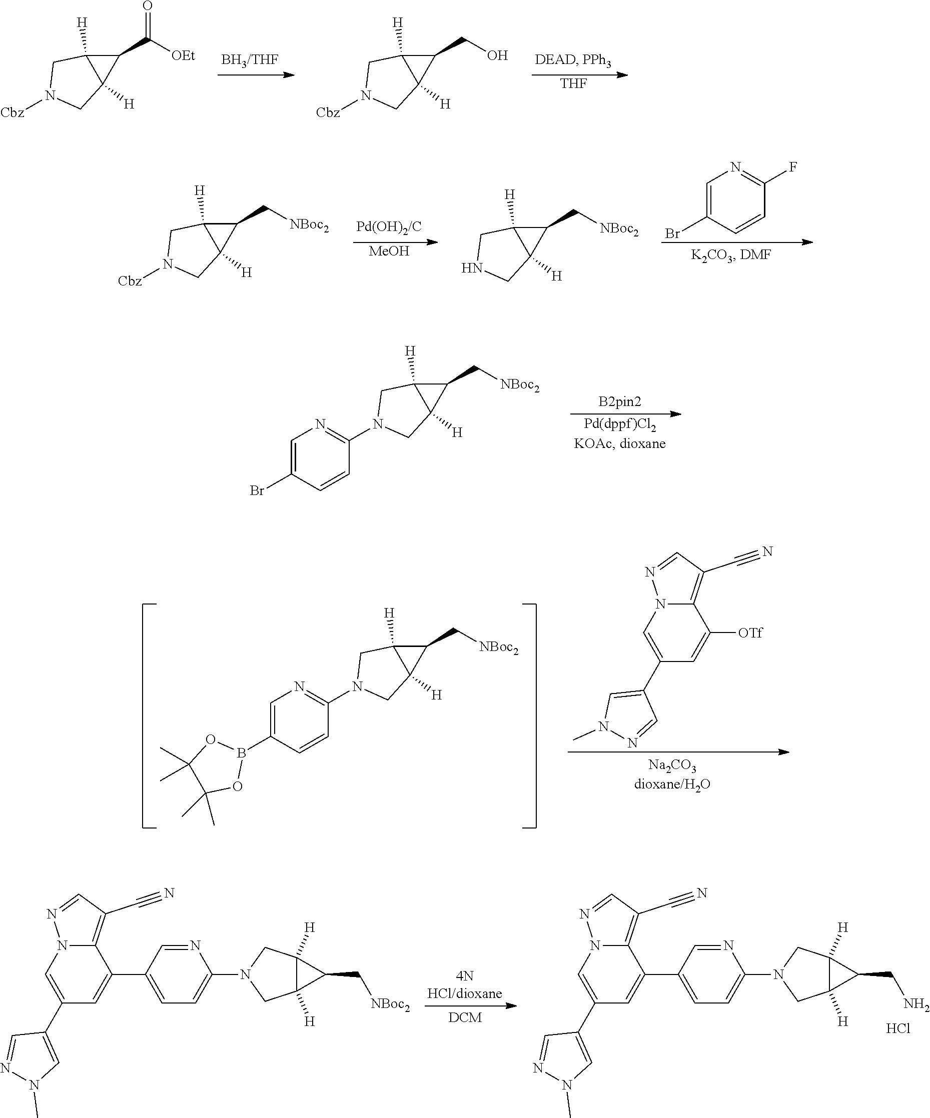

This intermediate was synthesized following the procedure used to make Intermediate 26 starting from the (1R,5S,6r)-isomer of Intermediate 25 in place of the exo isomer of Intermediate 25.

### Intermediate 28

### 4-(6-fluoropyridin-3-yl)-6-hydroxypyrazolo[1,5-a]pyridine-3-carbonitrile

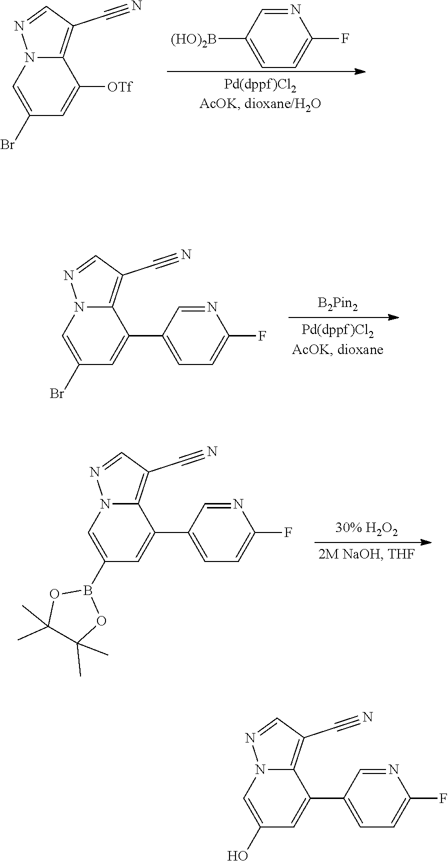 [SMILES: N#Cc1cnn2cc(Br)cc(OS(=O)(=O)C(F)(F)F)c12 ; OB(O)c1ccc(F)nc1 ; N#Cc1cnn2cc(Br)cc(-c3ccc(F)nc3)c12 ; CC1(C)OB(c2cc(-c3ccc(F)nc3)c3c(C#N)cnn3c2)OC1(C)C ; N#Cc1cnn2cc(O)cc(-c3ccc(F)nc3)c12]

### Step 1. 6-bromo-4-(6-fluoropyridin-3-yl)pyrazolo[1,5-a]pyridine-3-carbonitrile

A mixture of 6-bromo-3-cyanopyrazolo[1,5-a]pyridin-4-yl trifluoromethanesulfonate (4.4 g, 11.9 mmol), (6-fluoropyridin-3-yl)boronic acid (1.67 g, 11.9 mmol), Pd(dppf)Cl₂ DCM (195 mg, 0.238 mmol) and KOAc (2.33 g, 23.8 mmol) in dioxane/H₂O (50 mL/10 mL) was stirred at 25° C. overnight under N₂. The mixture was diluted with H₂O (100 mL). The precipitate formed was collected by filtration, rinsed with PE/EtOAc (2/1, 50 mL), and dried in vacuo to give the title compound (2.1 g, yield: 55%).

### Step 2. 4-(6-fluoropyridin-3-yl)-6-(4,4,5,5-tetramethyl-1,3,2-dioxaborolan-2-yl)pyrazolo[1,5-a]pyridine-3-carbonitrile

A mixture of the product of the Step 1 above (24 g, 76.68 mmol), B₂Pin₂ (20.18 g, 79.46 mmol), Pd(dppf)Cl₂ DCM (1.85 g, 2.27 mmol) and KOAc (14.85 g, 151.36 mmol) in dioxane (310 mL) was stirred at 70° C. overnight. The mixture was cooled to rt and concentrated in vacuo. The residue was taken up in DCM/MeOH (10/1, 1.0 L), washed with H₂O (300 mL×2) and brine (300 mL), dried over anhydrous Na₂SO₄, filtered off, and concentrated. The residue was purified by flash column chromatography on silica gel (PE/EtOAc=2/1 to 1/2) to give the crude compound, which was triturated with PE/EtOAc (2/1, 80 mL) and filtered to give the title compound (19.5 g, yield: 67%).

### Step 3. 4-(6-fluoropyridin-3-yl)-6-hydroxypyrazolo[1,5-a]pyridine-3-carbonitrile

To a solution of the product of Step 2 above (5.0 g, 13.72 mmol) in THF (75 mL) was added 2M NaOH (34.3 mL, 68.6 mmol). The mixture was cooled in ice-H₂O bath and 30% H₂O₂(8.48 mL, 82.37 mmol) was added dropwise. The mixture was stirred at rt for 3h, quenched by saturated aqueous NaHSO₃ (20 mL), acidified by 2M HCl to pH=5˜6, and extracted with DCM/IPA (3/1, 200 mL×2). The combined extracts were washed with H₂O (100 mL) and brine (100 mL), dried over anhydrous Na₂SO₄, filtered off, and concentrated. The residue was swirled with DCM/MeOH (10/1, 20 mL) and filtered to give the title compound (2.4 g, yield: 68%).

### Intermediate 29

### 4-(6-fluoropyridin-3-yl)-6-(2-hydroxy-2-methylpropoxy)pyrazolo[1,5-a]pyridine-3-carbonitrile

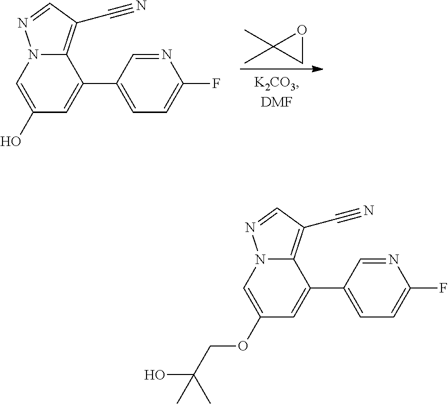

To Intermediate 28 (1.0 g, 3.93 mmol) and K₂CO₃ (1.63 g, 11.8 g) in DMF (6 mL) was added 2,2-dimethyloxirane (1.42 g, 19.69 mmol) at rt. The mixture was stirred at 80° C. under N₂ in a capped vial overnight. The mixture was cooled to rt, diluted with H₂O (30 mL), and extracted with EtOAc (10 mL×3). The combined organics were washed with H₂O (20 mL×4) and brine (20 mL), dried over anhydrous Na₂SO₄, filtered off, and concentrated. The residue was purified by flash column chromatography on silica gel (PE/EtOAc=10/1 to 1/1) to give the title compound (850 mg, yield: 66%).

### Intermediate 30

### 4-(6-fluoropyridin-3-yl)-6-(2-hydroxypropoxy)pyrazolo[1,5-a]pyridine-3-carbonitrile

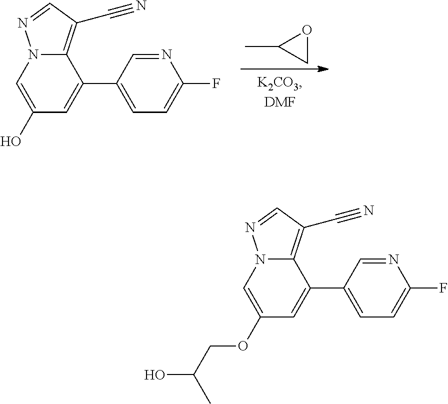

This intermediate was synthesized similarly by the procedure described in Intermediate 3 starting from Intermediate 28.

### Intermediate 31

### (3aR,4S,7R,7aS)-8-((6-methoxypyridin-3-yl)methyl)octahydro-1H-4,7-epiminoisoindole hydrochloride

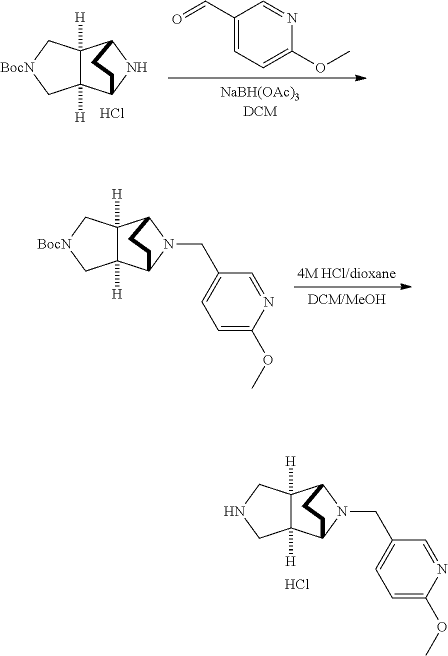

### Step 1. (3aR,4S,7R,7aS)-tert-butyl 8-((6-methoxypyridin-3-yl)methyl)hexahydro-1H-4,7-epiminoisoindole-2(3H)-carboxylate

To a solution of (3aR,4S,7R,7aS)-tert-butyl hexahydro-1H-4,7-epiminoisoindole-2(3H)-carboxylate hydrochloride (200 mg, 0.73 mmol) and 6-methoxynicotinaldehyde in (150 mg, 1.09 mmol) in DCM (5 mL) was added NaBH(OAc)₃ (309 mg, 1.46 mmol). The reaction mixture was stirred at rt for 6h, quenched with saturated aqueous NaHCO₃ (20 mL), and extracted with EtOAc (100 mL×2). The combined extracts were washed with H₂O (50 mL×2) and brine (50 mL), dried over anhydrous Na₂SO₄, filtered off, and concentrated in vacuo to give the title compound (234 mg, yield: 89%).

### Step 2. (3aR,4S,7R,7aS)-8-((6-methoxypyridin-3-yl)methyl)octahydro-1H-4,7-epiminoisoindole hydrochloride

To a solution of the product of Step 1 above (234 mg, 0.65 mmol) in DCM/MeOH (4/1, 5 mL) was added 4M HCl/dioxane (1 mL, 4.0 mmol). The mixture was stirred at rt for 4h, warmed to 50° C. and stirred for 2h, and concentrated in vacuo to give the title compound (quantitative).

### Intermediate 32

### (3aR,4S,7R,7aS)-2-(5-bromopyridin-2-yl)-8-((6-methoxypyridin-3-yl)methyl)octahydro-1H-4,7-epiminoisoindole

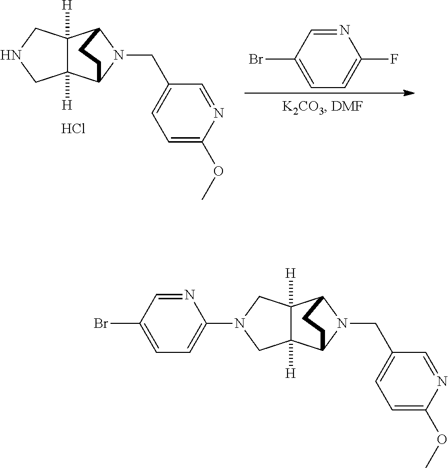

A mixture of 5-bromo-2-fluoropyridine (90 mg, 0.51 mmol), Intermediate 31 (150 mg, 0.51 mmol), and K₂CO₃ (140 mg, 1.0 mmol) in DMF (1 mL) was stirred at 110° C. under N₂ for 6h. The mixture was cooled to rt and purified by the reverse phase flash column chromatography on C18 (MeOH/H₂O) to give the title compound (25 mg, yield: 12%).

### Intermediate 33

### (6-methoxypyridin-3-yl)((3aR,4S,7R,7aS)-octahydro-1H-4,7-epiminoisoindol-8-yl)methanone hydrochloride

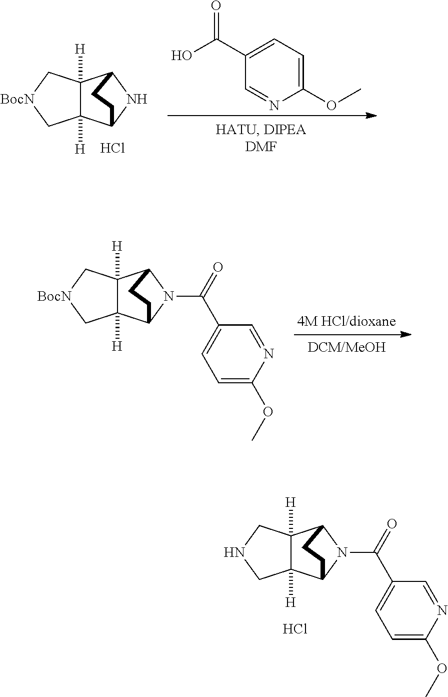

### Step 1. (3aR,4S,7R,7aS)-tert-butyl 8-(6-methoxynicotinoyl)hexahydro-1H-4,7-epiminoisoindole-2(3H)-carboxylate

To a solution of (3aR,4S,7R,7aS)-tert-butyl hexahydro-1H-4,7-epiminoisoindole-2(3H)-carboxylate hydrochloride (100 mg, 0.364 mmol), 6-methoxynicotinic acid (56 mg, 0.364 mmol), and HATU (207 mg, 0.546 mmol) in DMF (1 mL) was added DIPEA (236 mg, 1.82 mmol) at rt. The mixture was stirred at 50° C. for 3h, cooled to rt, and purified by reverse phase flash column chromatography on C18 (MeOH/H₂O) to give the title compound (120 mg, yield: 88%).

### Step 2. (6-methoxypyridin-3-yl)((3aR,4S,7R,7aS)-octahydro-1H-4,7-epiminoisoindol-8-yl)methanone hydrochloride

To a solution of the product of Step 1 above (120 mg, 032 mmol) in DCM/MeOH (4/1, 5 mL) was added 4M HCl/dioxane (1 mL) at rt. The mixture was stirred at rt for 4h and concentrated to give the title compound (quantitative).

### Intermediate 34

### 4-(6-((3aR,5s,6aS)-5-amino-5-methylhexahydrocyclopenta[c]pyrrol-2(1H)-yl)pyridin-3-yl)-6-(1-methyl-1H-pyrazol-3-yl)pyrazolo[1,5-a]pyridine-3-carbonitrile

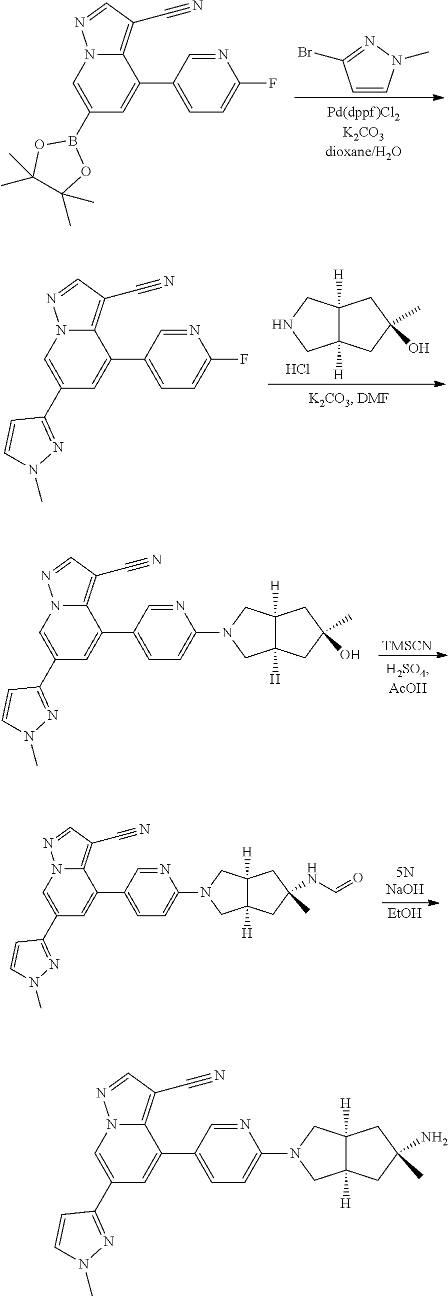

### Step 1. 4-(6-fluoropyridin-3-yl)-6-(1-methyl-1H-pyrazol-3-yl)pyrazolo[1,5-a]pyridine-3-carbonitrile

A mixture of 3-bromo-1-methyl-1H-pyrazole (1.33 g, 8.24 mmol), the product of Step 2 in Intermediate 28 (3.0 g, 8.24 mmol), Pd(dppf)Cl₂ DCM (340 mg, 0.412 mmol) and K₂CO₃ (2.3 g, 16.48 mmol) in dixoane/H₂O (40 mL/8 mL) was stirred at 80° C. overnight under N₂. The mixture was diluted with ice-H₂O (200 mL). The precipitate formed was collected by filtration, dried in vacuo, and purified by flash column chromatography on silica gel (DCM/MeOH=50/1 to 30/1) to give the title compound (2.0 g, yield: 76%).

### Step 2. 4-(6-((3aR,5r,6aS)-5-hydroxy-5-methylhexahydrocyclopenta[c]pyrrol-2(1H)-yl)pyridin-3-yl)-6-(1-methyl-1H-pyrazol-3-yl)pyrazolo[1,5-a]pyridine-3-carbonitrile

A mixture of the product of Step 1 above (2.0 g, 6.28 mmol), the product of Step 2 in Intermediate 23 (1.3 g, 7.54 mmol), and K₂CO₃ (2.6 g, 18.84 mmol) in DMF (40 mL) was stirred at 110° C. under N₂ for 4h. The mixture was cooled to rt, diluted with EtOAc (500 mL), washed with water (100 mL×3) and brine (100 mL), dried over anhydrous Na₂SO₄, filtered off, and concentrated. The residue was purified by flash column chromatography on silica gel (DCM/MeOH=60/1 to 30/1) to give the title compound (1.7 g, yield: 62%).

### Step 3. N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)formamide

To a solution of the product of Step 2 above (1.7 g, 3.87 mmol) and trimethylsilanecarbonitrile (1.2 g, 11.61 mmol) in HOAc (20 mL) was added concentrated H₂SO₄ (16 mL) dropwise at 0° C. The mixture was stirred at rt for 2 h, cooled in ice-H₂O bath, basified with aqueous NaOH (5 N) to pH=8-9, and extracted with DCM/MeOH (10/1, 200 mL×3). The combined organics were washed by H₂O (100 mL), dried over anhydrous Na₂SO₄, filtered off, and concentrated. The residue was purified by flash column chromatography on silica gel (DCM/MeOH=40/1 to 20/1) to give the title compound (1.8 g, yield: 100%).

### Step 4. 4-(6-((3aR,5s,6aS)-5-amino-5-methylhexahydrocyclopenta[c]pyrrol-2(1H)-yl)pyridin-3-yl)-6-(1-methyl-1H-pyrazol-3-yl)pyrazolo[1,5-a]pyridine-3-carbonitrile

To a solution of the product of Step 3 above (1.8 g, 3.86 mmol) in EtOH (25 mL) was added aqueous NaOH (5 N, 25 mL). The mixture was stirred at 80° C. for 3 h, cooled to rt, diluted with DCM/MeOH (10/1, 50 mL), washed with H₂O (50 mL×2) and brine (50 mL), dried over anhydrous Na₂SO₄, filtered off, and concentrated. The residue was swirled with PE/EtOAc (5/1, 50 mL), filtered and dried in vacuo to give the title compound (1.6 g, yield: 94%).

### Intermediate 35

### 4-(6-((3aR,5s,6aS)-5-amino-5-methylhexahydrocyclopenta[c]pyrrol-2(1H)-yl)pyridin-3-yl)-6-(1-methyl-1H-pyrazol-4-yl)pyrazolo[1,5-a]pyridine-3-carbonitrile

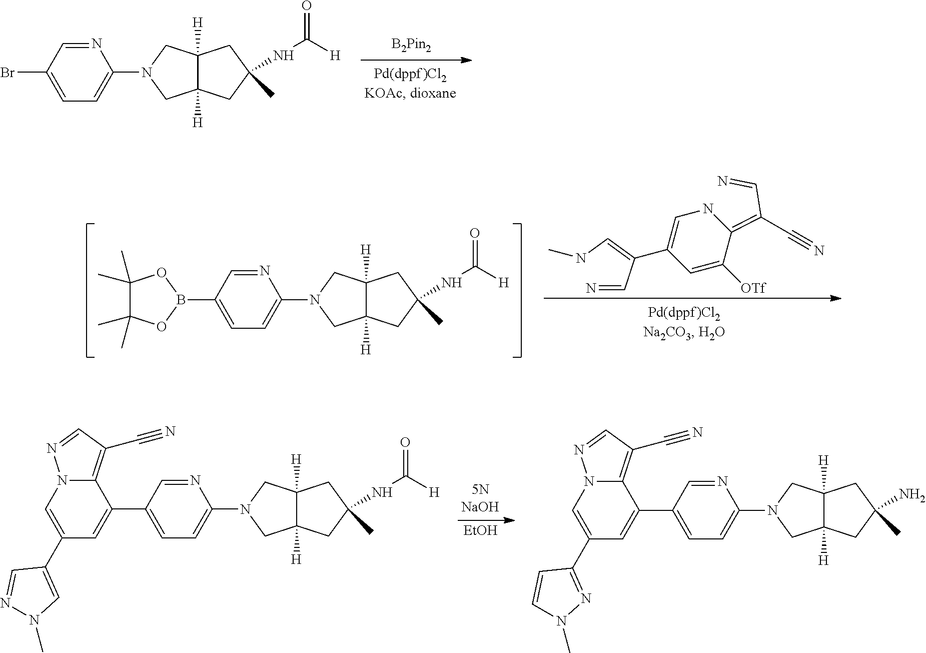

### Step 1. N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)formamide

A mixture of the product of Step 4 in Intermediate 23 (1.0 g, 3.10 mmol), B₂Pin₂ (825 mg, 3.25 mmol), Pd(dppf)Cl₂ DCM (126 mg, 0.155 mmol), and KOAc (608 mg, 6.2 mmol) in dioxane (10 mL) was stirred at 100° C. under N₂ for 4h. To the mixture cooled to rt was added Intermediate 1 (1.15 g, 3.1 mmol), Pd(dppf)Cl₂ DCM (126 mg, 0.155 mmol), Na₂CO₃ (657 mg, 6.20 mmol) and dioxane/H₂O (5 mL/3 mL). The mixture was stirred at 100° C. under N₂ overnight, cooled to rt, and filtered. The filtrate was diluted with DCM/MeOH (10/1, 200 mL), washed with H₂O (75 mL×2) and brine (75 mL), dried over anhydrous Na₂SO₄, filtered off, and concentrated. The residue was purified by flash column chromatography on silica gel (DCM/MeOH=100/1 to 30/1) to give the title compound (920 mg, yield: 64%)

### Step 2. 4-(6-((3aR,5s,6aS)-5-amino-5-methylhexahydrocyclopenta[c]pyrrol-2(1H)-yl)pyridin-3-yl)-6-(1-methyl-1H-pyrazol-4-yl)pyrazolo[1,5-a]pyridine-3-carbonitrile

To a solution of the product of Step 1 above (920 mg, 1.97 mmol) in EtOH (15 mL) was added aqueous NaOH (5 N, 15 mL). The mixture was stirred at 90° C. overnight, cooled to rt, diluted with DCM/MeOH (10/1, 150 mL), washed with H₂O (50 mL×2) and brine (50 mL), dried over anhydrous Na₂SO₄, filtered off, and concentrated to give the title compound (813 mg, yield: 94%).

### Intermediate 36

### (3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrole-5-carboxylic acid

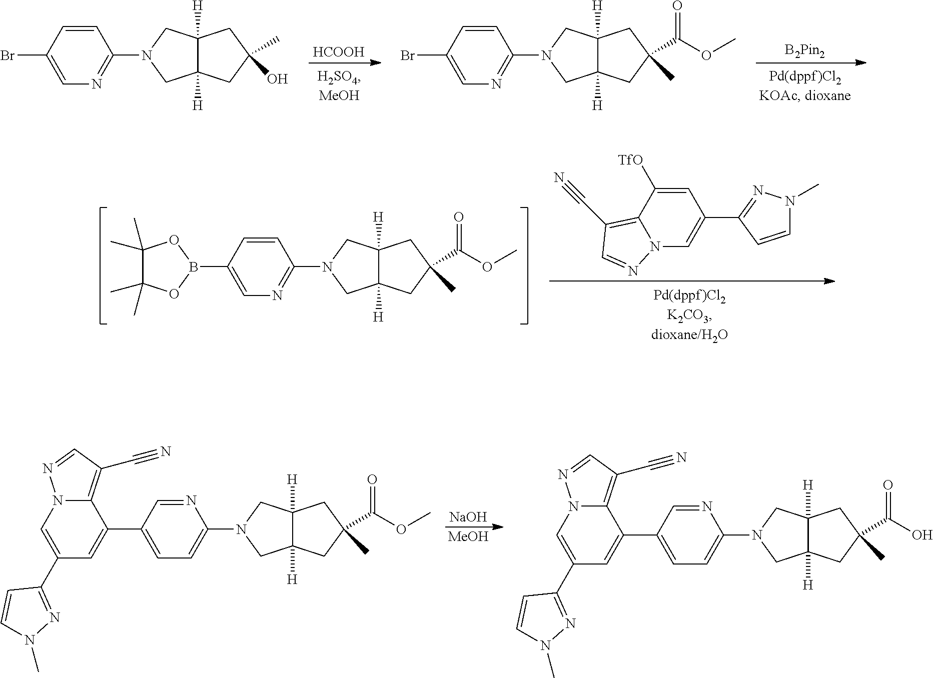

### Step 1. (3aR,5s,6aS)-methyl 2-(5-bromopyridin-2-yl)-5-methyloctahydrocyclopenta[c]pyrrole-5-carboxylate

A solution of the product of Step 3 in Intermediate 23 (149 mg, 0.5 mmol) in HCO₂H (2 mL) was added to concentrated H₂SO₄ (8 mL) slowly. Upon completion, HCOOH (2 mL) was added dropwise to the reaction mixture at 60° C. The mixture was stirred at 60° C. for 1 h, cooled to rt, treated with MeOH (15 mL), and stirred at rt overnight. The mixture was concentrated, treated with ice, neutralized to pH=9˜10 with solid NaOH, and extracted with DCM/MeOH (10/1, 50 mL×3). The combined organics were washed with brine, dried over anhydrous Na₂SO₄, filtered off, and concentrated. The residue was purified by prep-TLC (PE/EtOAc=3/1) to give the title compound (40 mg, yield: 12%).

### Step 2. (3aR,5s,6aS)-methyl 2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrole-5-carboxylate

A mixture of the product of Step 1 above (40 mg, 0.12 mmol), B₂Pin₂ (31 mg, 0.12 mmol), Pd(dppf)Cl₂ DCM (10 mg, 0.0012 mmol), and KOAc (24 mg, 0.24 mmol) in dioxane (2 mL) was stirred at 90° C. under N₂ for 4h. The mixture was cooled to rt and treated with Intermediate 2 (45 mg, 0.12 mmol), Pd(dppf)Cl₂ DCM (10 mg, 0.0012 mmol), K₂CO₃ (33 mg, 0.24 mmol) and dioxane/H₂O (5 mL/1 mL). The mixture was stirred at 100° C. under N₂ overnight, cooled to rt, diluted with DCM/MeOH (10/1, 100 mL), washed with H₂O (30 mL) and brine (30 mL), dried over anhydrous Na₂SO₄, filtered off, and concentrated. The residue was purified by prep-TLC (DCM/MeOH=25/1) to give the title compound (30 mg, yield: 52%)

### Step 3. (3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrole-5-carboxylic acid

To a solution of the product of Step 2 above (30 mg, 0.06 mmol) in MeOH (3 mL) was added 2M NaOH (3 mL) at rt. The mixture was stirred at 50° C. for 2 h, cooled to rt, acidified to pH=5˜6, and extracted with DCM/MeOH (10/1, 30 mL×3). The combined organics were washed with brine (30 mL), dried over anhydrous Na₂SO₄, and concentrated to give the title compound (30 mg, yield: 100%).

### Intermediate 37

### N-(((1R,5S,6s)-3-(5-bromopyridin-2-yl)-3-azabicyclo[3.1.0]hexan-6-yl)methyl)-3-chloropicolinamide

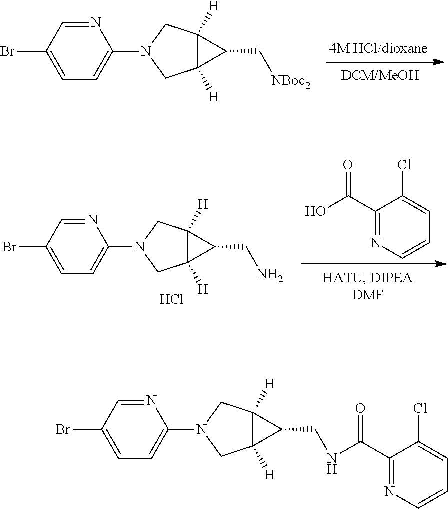

### Step 1. ((1R,5S,6s)-3-(5-bromopyridin-2-yl)-3-azabicyclo[3.1.0]hexan-6-yl)methanamine hydrochloride

To a solution of the product of Step 4 in Intermediate 26 (350 mg, 0.747 mmol) in DCM (4 mL) was added 4M HCl/dioxane (4 mL) at rt. The mixture was stirred at rt for 4h and concentrated to give the title compound (173 mg, yield: 76%).

### Step 2. N-(((1R,5S,6s)-3-(5-bromopyridin-2-yl)-3-azabicyclo[3.1.0]hexan-6-yl)methyl)-3-chloropicolinamide

To a solution of the product of Step 1 above (173 mg, 0.568 mmol), 3-chloropicolinic acid (98 mg, 0.625 mmol), and HATU (320 mg, 0.852 mmol) in DMF (2 mL) was added DIPEA (367 mg, 2.84 mmol) at rt. The mixture was stirred at 75° C. for 2h, cooled to rt and purified by reverse phase flash column chromatography on C18 (MeOH/H₂O) to give the title compound (89 mg, yield: 39%).

### Intermediate 38

### 4-(6-((1R,5S,6s)-6-(aminomethyl)-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

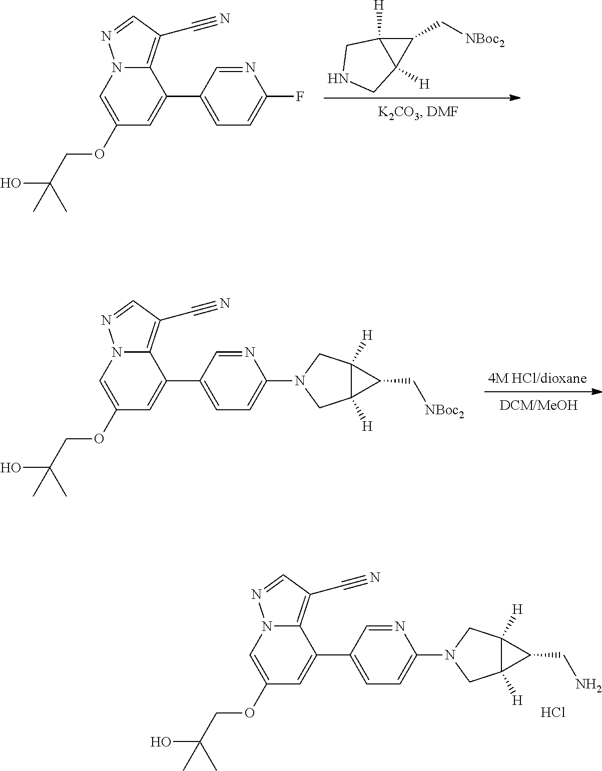

### Step 1. tert-butyl (tert-butoxycarbonyl)(((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)carbamate

A mixture of Intermediate 29 (190 mg, 0.58 mmol), the product of Step 3 in Intermediate 26 (200 mg, 0.64 mmol), and K₂CO₃ (160 mg, 1.16 mmol) in DMF (2 mL) was stirred at 110° C. under N₂ overnight. The mixture was cooled to rt, diluted with EtOAc (100 mL), washed with H₂O (30 mL×2) and brine (30 mL), dried over anhydrous Na₂SO₄, filtered off, and concentrated. The residue was purified by flash column chromatography on silica gel (DCM/MeOH=80/1 to 40/1) to give the title compound (110 mg, yield: 30%).

### Step 2. 4-(6-((1R,5S,6s)-6-(aminomethyl)-3-azabicyclo[3.1.0]hexan-3-yl)pyridin-3-yl)-6-(2-hydroxy-2-methylpropoxy)pyrazolo[1,5-a]pyridine-3-carbonitrile hydrochloride

To a solution of the product of Step 1 above (110 mg, 0.18 mmol) in DCM/MeOH (4/1, 5 mL) was added 4M HCl/dioxane (1 mL) at rt. The mixture was stirred at rt for 4h and concentrated to give the title compound (140 mg, quantitative).

### Intermediate 39

### 4-(6-((1R,5S,6s)-6-(aminomethyl)-3-azabicyclo[3.1.0]hexan-3-yl)pyridin-3-yl)-6-(2-hydroxypropoxy)pyrazolo[1,5-a]pyridine-3-carbonitrile hydrochloride

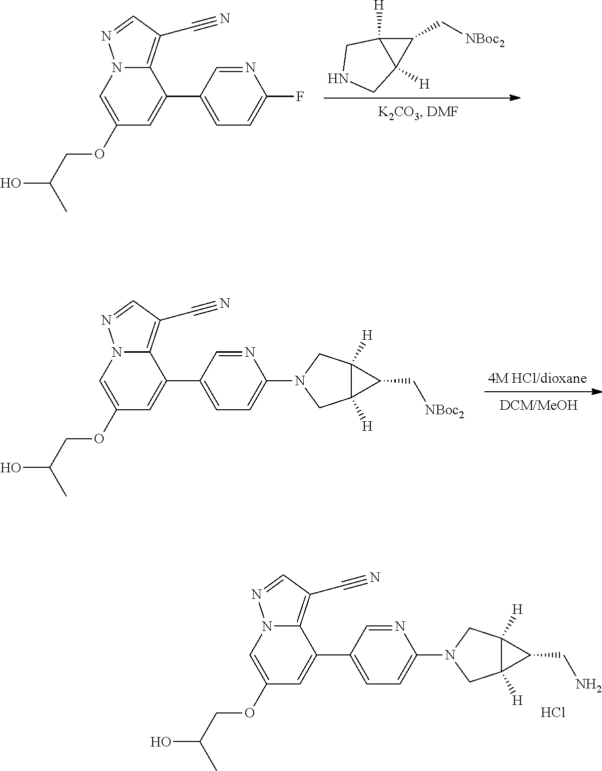

This intermediate was synthesized similarly by the procedure described in Intermediate 38 starting from by Intermediate 30.

### Intermediate 40

### N-((3aR,5s,6aS)-5-methyloctahydrocyclopenta[c]pyrrol-5-yl)formamide

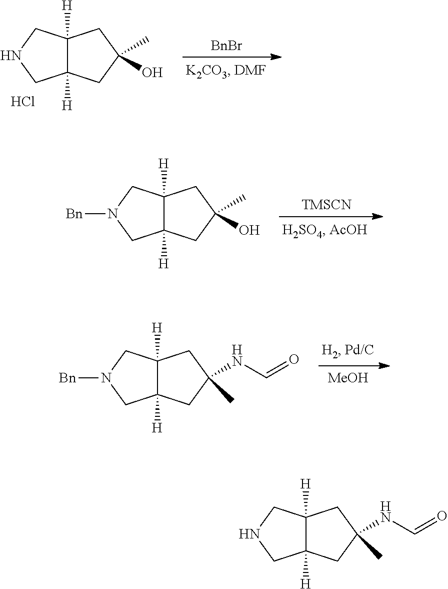

### Step 1. (3aR,5r,6aS)-2-benzyl-5-methyloctahydrocyclopenta[c]pyrrol-5-ol

A mixture of the product of Step 2 in Intermediate 23 (500 mg, 2.8 mmol), benzyl bromide (577 mg, 3.37 mmol), and K₂CO₃ (1.16 mg, 8.4 mmol) in DMF (5 mL) was stirred at 50° C. under N₂ overnight. The mixture was cooled to rt, diluted with EtOAc (150 mL), washed with H₂O (50 mL×2) and brine (50 mL), dried over anhydrous Na₂SO₄, filtered off, and concentrated. The residue was purified by flash column chromatography on silica gel (DCM/MeOH=80/1 to 60/1) to give the title compound (430 mg, yield: 66%).

### Step 2. N-((3aR,5s,6aS)-2-benzyl-5-methyloctahydrocyclopenta[c]pyrrol-5-yl)formamide

To a solution of the product of Step 1 above (300 mg, 1.30 mmol) and trimethylsilanecarbonitrile (387 mg, 3.9 mmol) in HOAc (1 mL) was added concentrated H₂SO₄ (0.8 mL) dropwise at 0° C. The mixture was stirred at rt overnight, basified with aqueous NaOH (5 N) to pH=8-9, and extracted with EtOAc (50 mL×3). The combined extracts were washed with brine (30 mL), dried over anhydrous Na₂SO₄, filtered off, and concentrated to give the title compound (439 mg, yield: 94%).

### Step 3. N-((3aR,5s,6aS)-5-methyloctahydrocyclopenta[c]pyrrol-5-yl)formamide

To a solution of the product of Step 2 above (439 mg, 1.7 mmol) in MeOH (10 mL) was added Pd/C (Palladium 10% on Carbon, ca. 50% water, 100 mg). The mixture was stirred at 80° C. under a hydrogen balloon for 3h, cooled to rt, filtered, and concentrated to give the title compound (290 mg, yield: 100%).

### Intermediate 41

### 4-(6-((3aR,5s,6aS)-5-amino-5-methylhexahydrocyclopenta[c]pyrrol-2(1H)-yl)pyridin-3-yl)-6-(2-hydroxy-2-methylpropoxy)pyrazolo[1,5-a]pyridine-3-carbonitrile

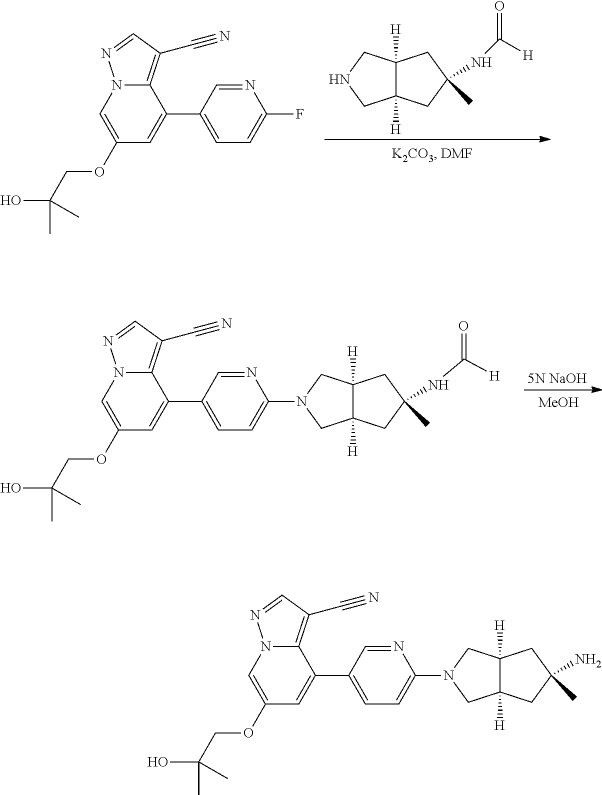

### Step 1. N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)formamide

A mixture of Intermediate 29 (506 mg, 1.55 mmol), Intermediate 40 (290 mg, 1.72 mmol), and K₂CO₃ (428 mg, 3.10 mmol) in DMF (10 mL) was stirred at 110° C. under N₂ overnight. The mixture was cooled to rt and diluted with H₂O (80 mL). The precipitate formed was collected by filtration, dissolved in EtOAc (150 mL), washed with H₂O (30 mL) and brine (30 mL), dried over anhydrous Na₂SO₄, filtered off, and concentrated. The residue was purified by flash column chromatography on silica gel (DCM/MeOH=40/1 to 15/1) to give the title compound (750 mg, yield: 100%).

### Step 2. 4-(6-((3aR,5s,6aS)-5-amino-5-methylhexahydrocyclopenta[c]pyrrol-2(1H)-yl)pyridin-3-yl)-6-(2-hydroxy-2-methylpropoxy)pyrazolo[1,5-a]pyridine-3-carbonitrile

To a solution of the product of Step 1 above (750 mg, 1.58 mmol) in EtOH (10 mL) was added aqueous NaOH (5 N, 10 mL). The mixture was stirred at 80° C. for 3 h, cooled to rt, diluted with DCM/MeOH (10/1, 100 mL), washed with H₂O (50 mL×2) and brine (50 mL), dried over anhydrous Na₂SO₄, filtered off, and concentrated. The residue was purified by reverse phase flash column chromatography on C18 (MeOH/H₂O) to give the title compound (180 mg, yield: 25%).

### Intermediate 42

### 4-(6-((1R,3 S,5s,7s)-5-amino-2-azaadamantan-2-yl)pyridin-3-yl)-6-(2-hydroxy-2-methylpropoxy)pyrazolo[1,5-a]pyridine-3-carbonitrile hydrochloride

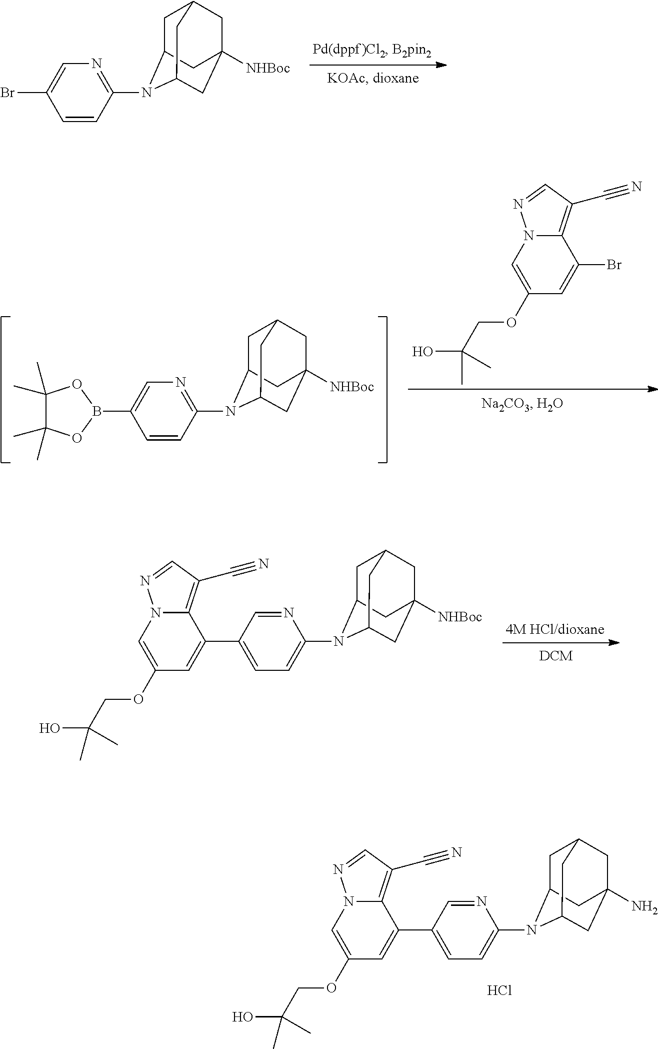

### Step 1. tert-butyl ((1R,3S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)carbamate

A mixture of the product of Step 13 in Example 89 (300 mg, 0.735 mmol), B₂Pin₂ (187 mg, 0.735 mmol), Pd(dppf)Cl₂ DCM (60 mg, 0.0735 mmol), and KOAc (144 mg, 1.47 mmol) in dioxane (3 mL) was stirred at 100° C. under N₂ for 3h. The mixture was cooled to rt and added Intermediate 3 (228 mg, 0.735 mmol), Pd(dppf)Cl₂ DCM (60 mg, 0.0735 mmol), Na₂CO₃ (156 mg, 1.47 mmol) and dioxane/H₂O (6 mL/1.6 mL). The mixture was stirred at 110° C. under N₂ for 6h, cooled to rt, filtered, and concentrated. The residue was taken up in EtOAc (150 mL), washed with H₂O (30 mL) and brine (30 mL), dried over anhydrous Na₂SO₄, filtered off, and concentrated. The residue was purified by flash column chromatography on silica gel (PE/EtOAc=2/1 to DCM/EtOAc=1/1) to give the title compound (260 mg, yield: 63%).

### Step 2. 4-(6-((1R,3S,5s,7s)-5-amino-2-azaadamantan-2-yl)pyridin-3-yl)-6-(2-hydroxy-2-methylpropoxy)pyrazolo[1,5-a]pyridine-3-carbonitrile hydrochloride

To a solution of the product of Step 2 above (255 mg, 0.449 mmol) in DCM (2 mL) was added 4M HCl/dioxane (4 mL) at rt. The mixture was stirred at rt for 3h and concentrated to give the title compound (260 mg, quantitative).

### Intermediate 43

### 4-(6-((1R,3 S,5s,7s)-5-amino-2-azaadamantan-2-yl)pyridin-3-yl)-6-ethoxypyrazolo[1,5-a]pyridine-3-carbonitrile hydrochloride

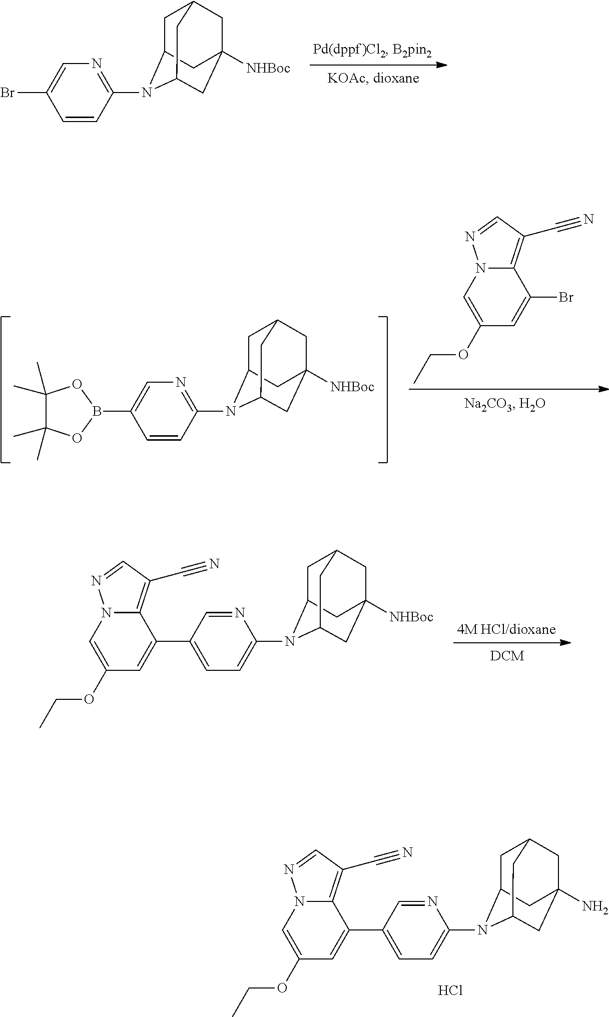

This intermediate was synthesized similarly by the procedure described in Intermediate 42 replacing Intermediate 3 with Intermediate 4.

### Intermediate 44

### 4-(5-((3aR,5s,6aS)-5-amino-5-methylhexahydrocyclopenta[c]pyrrol-2(1H)-yl)pyrazin-2-yl)-6-(1-methyl-1H-pyrazol-3-yl)pyrazolo[1,5-a]pyridine-3-carbonitrile

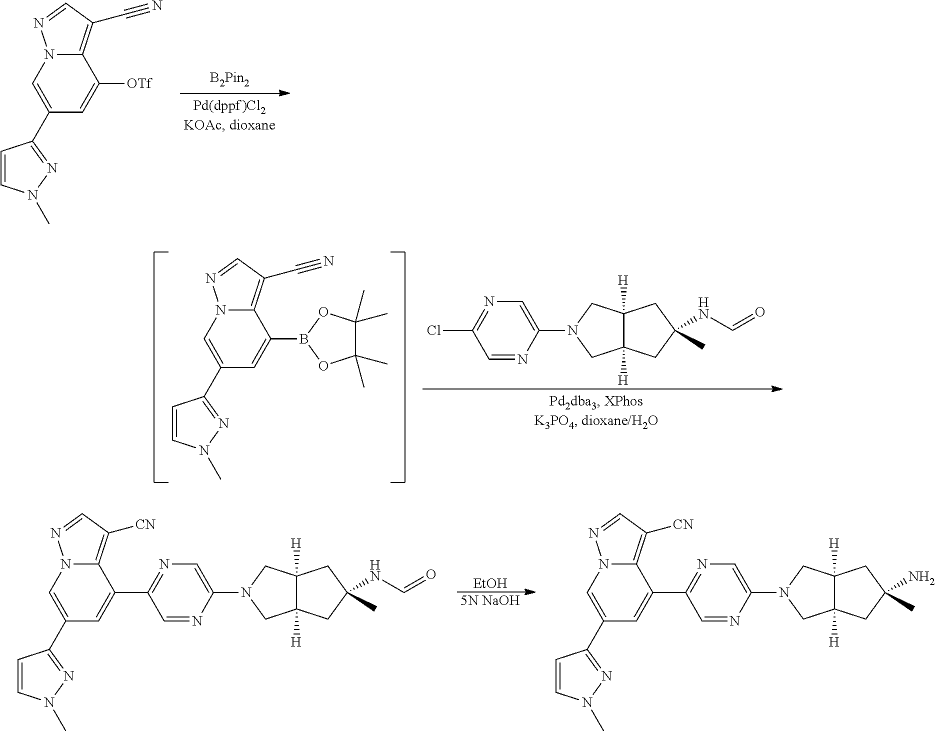

### Step 1. N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)formamide

A solution of Intermediate 2 (743 mg, 2.0 mmol), B₂Pin₂ (533 mg, 2.1 mmol), Pd(dppf)Cl₂ DCM (82 mg, 0.1), and KOAc (393 mg, 4.0 mmol) in dioxane (8 mL) was stirred at 90° C. for 24 under N₂. The mixture was cooled to rt and treated with the product of Step 2 in Intermediate 24 (533 mg, 1.9 mmol), Pd(dppf)Cl₂ DCM (82 mg, 0.1 mmol), K₂CO₃ (553 mg, 4.0 mmol) and dioxane/H₂O (15 mL/3 mL). The mixture was stirred at 100° C. under N₂ overnight, cooled to rt, filtered, and concentrated. The residue was taken up in EtOAc (200 mL), washed with H₂O (50 mL×2) and brine (50 mL), dried over anhydrous Na₂SO₄, filtered off, and concentrated. The residue was purified by flash column chromatography on silica gel (DCM/MeOH=50/1 to 20/1) to give the title compound (425 mg, yield: 48%).

### Step 2. 4-(5-((3aR,5s,6aS)-5-amino-5-methylhexahydrocyclopenta[c]pyrrol-2(1H)-yl)pyrazin-2-yl)-6-(1-methyl-1H-pyrazol-3-yl)pyrazolo[1,5-a]pyridine-3-carbonitrile

To a solution of the product of Step 1 above (425 mg, 0.91 mmol) in EtOH (10 mL) was added aqueous NaOH (5 N, 10 mL). The mixture was stirred at 80° C. overnight, cooled to rt, diluted with DCM/MeOH (10/1, 200 mL), washed with H₂O (50 mL×2) and brine (50 mL), dried over anhydrous Na₂SO₄, filtered off, and concentrated. The residue was triturated with MeOH (3 mL), filtered and dried in vacuo to give the title compound (280 mg, yield: 70%).

### Intermediate 45

### 4-(5-((3aR,5s,6aS)-5-amino-5-methylhexahydrocyclopenta[c]pyrrol-2(1H)-yl)pyrazin-2-yl)-6-(1-methyl-1H-pyrazol-4-yl)pyrazolo[1,5-a]pyridine-3-carbonitrile

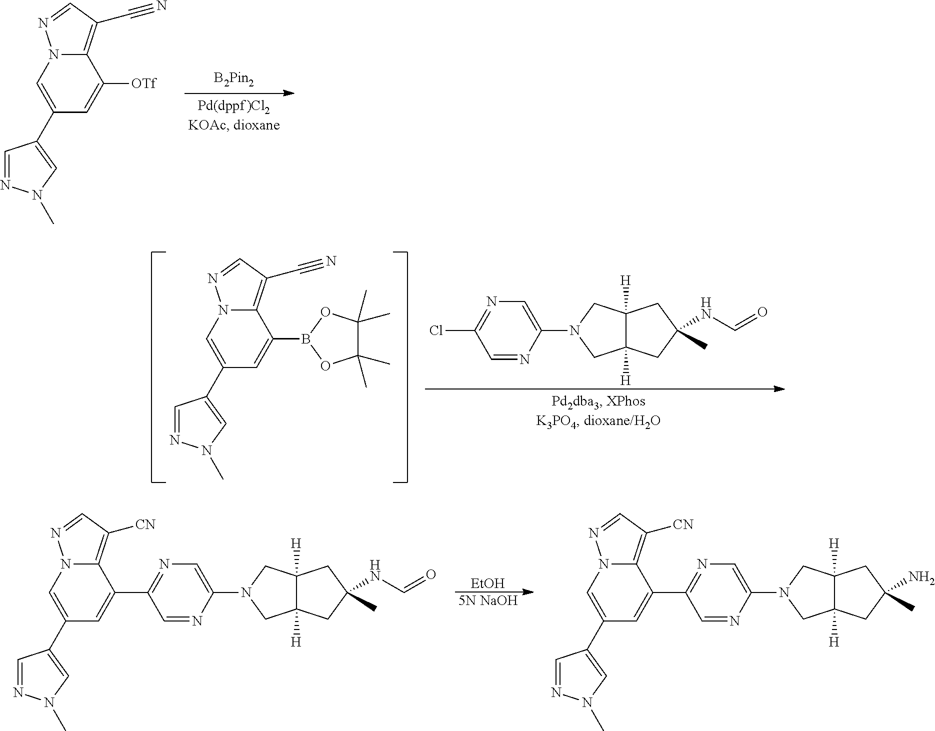

This intermediate was synthesized similarly by the procedure described in Intermediate 44 by replacing Intermediate 2 with Intermediate 1.

### Intermediate 46

### 4-(5-((3aR,5s,6aS)-5-amino-5-methylhexahydrocyclopenta[c]pyrrol-2(1H)-yl)pyrazin-2-yl)-6-(2-hydroxy-2-methylpropoxy)pyrazolo[1,5-a]pyridine-3-carbonitrile

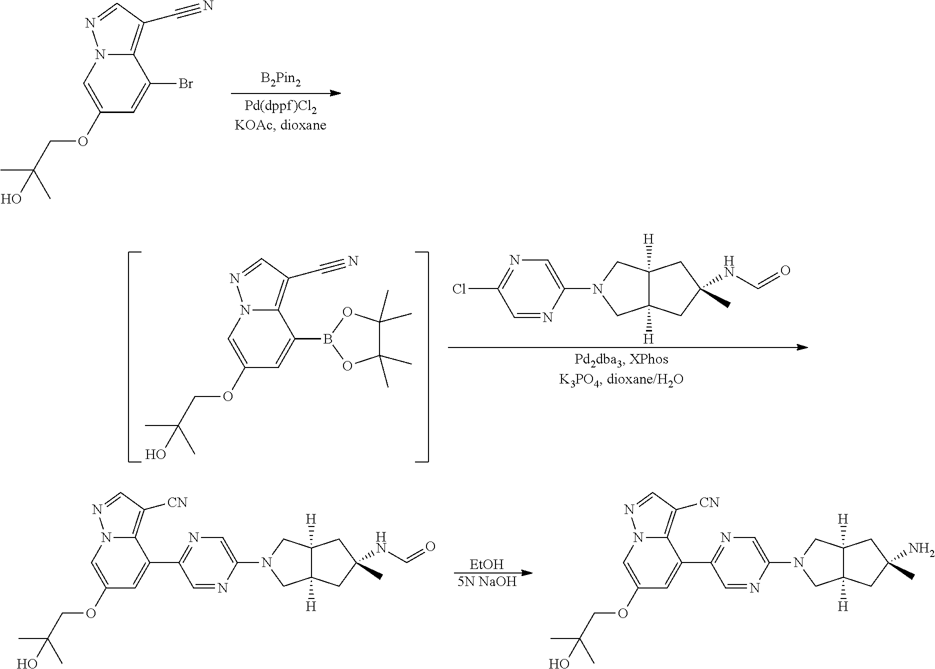

This intermediate was synthesized similarly by the procedure described in Intermediate 44 by replacing Intermediate 2 with Intermediate 3.

### Intermediate 47

### 4-(5-((1R,5S,6s)-6-amino-3-azabicyclo[3.1.0]hexan-3-yl)pyrazin-2-yl)-6-(1-methyl-1H-pyrazol-4-yl)pyrazolo[1,5-a]pyridine-3-carbonitrile hydrochloride

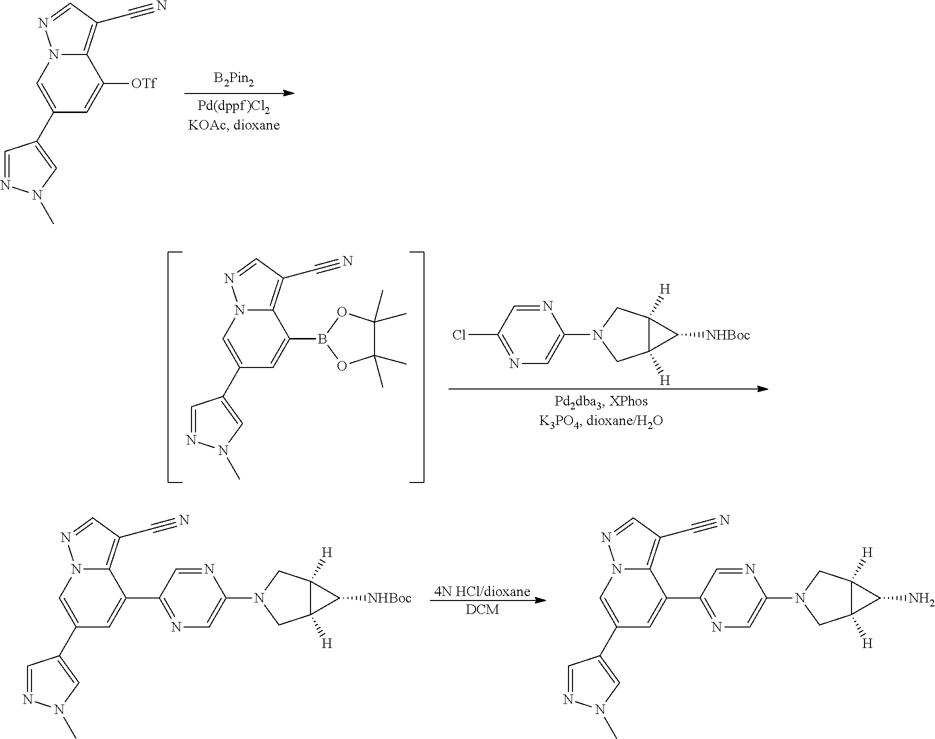

This intermediate was synthesized similarly by the procedure as described in Intermediate 17 by replacing Intermediate 4 with Intermediate 1.

### Intermediate 48

### tert-butyl N-(tert-butoxycarbonyl)-N-(((1R,5S,6r)-3-(5-chloropyrazin-2-yl)-3-azabicyclo[3.1.0]hexan-6-yl)methyl)carbamate

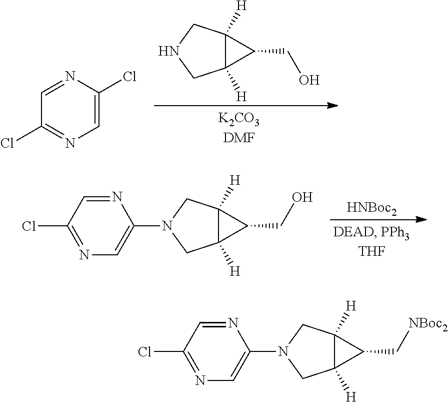

### Step 1. ((1R,5S,6r)-3-(5-chloropyrazin-2-yl)-3-azabicyclo[3.1.0]hexan-6-yl)methanol

A mixture of 2,5-dichloropyrazine (658 mg, 4.42 mmol), ((1R,5S,6r)-3-(5-chloropyrazin-2-yl)-3-azabicyclo[3.1.0]hexan-6-yl)methanol (500 mg, 4.42 mmol), and K₂CO₃ (1.22 g, 8.84 mmol) in DMF (10 mL) was stirred at 110° C. under N₂ for 6h. The mixture was cooled to rt, diluted with DCM/MeOH (10/1, 200 mL), washed with H₂O (30 mL×5) and brine (30 mL), dried over anhydrous Na₂SO₄, filtered off, and concentrated to give the title compound (880 mg, yield: 90%).

### Step 2. tert-butyl N-(tert-butoxycarbonyl)-N-((1R,5S,6r)-3-azabicyclo[3.1.0]hexan-6-ylmethyl)carbamate

To an ice-water cooled solution of the product of Step 1 above (480 mg, 2.13 mmol), Boc₂NH (508 mg, 2.34 mmol) and PPh3 (614 mg, 2.34 mmol) in THF (5 mL) was added DEAD (408 mg, 2.34 mmol) dropwise. The mixture was stirred at rt for 2h, concentrated, and purified by flash column chromatography on silica gel (PE/EtOAc=20/1 to 10/1) to give the title compound (650 mg, yield: 72%).

### Intermediate 49

### 4-(5-((1R,5S,6s)-6-(aminomethyl)-3-azabicyclo[3.1.0]hexan-3-yl)pyrazin-2-yl)-6-(2-hydroxy-2-methylpropoxy)pyrazolo[1,5-a]pyridine-3-carbonitrile trifluoroacetate

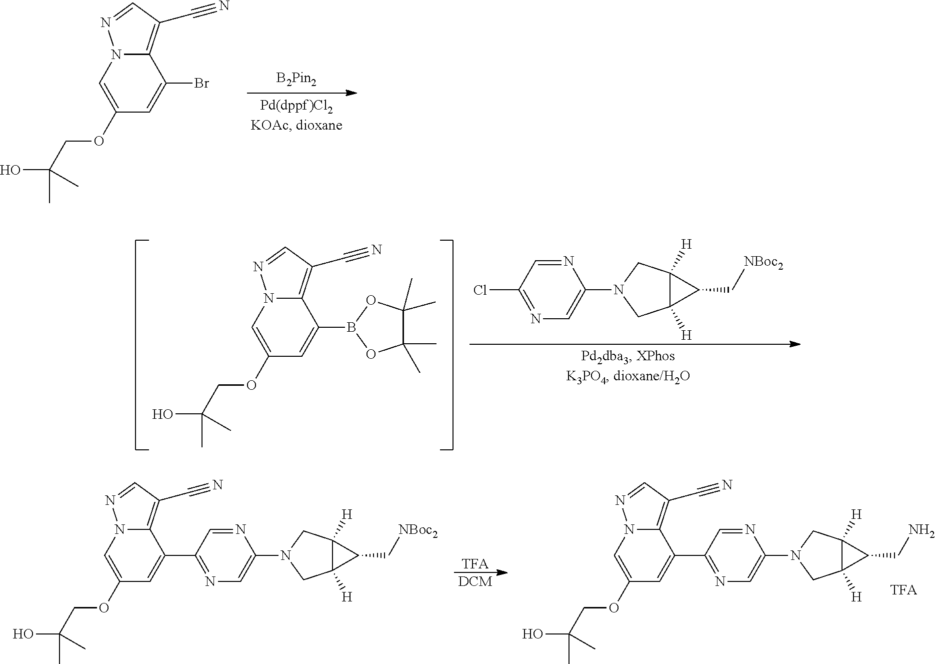

### Step 1. tert-butyl (tert-butoxycarbonyl)(((1R,5S,6r)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)carbamate

A mixture of Intermediate 3 (150 mg, 0.484 mmol), B₂Pin₂ (129 mg, 0.508 mmol), Pd(dppf)Cl₂ DCM (39 mg, 0.0484 mmol), and KOAc (95 mg, 0.968 mmol) in dioxane (1 mL) was stirred at 100° C. under N₂ for 4 h. The reaction mixture was cooled to rt and treated with Intermediate 48 (206 mg, 0.0.484 mmol), K₃PO₄ (308 mg, 1.452 mmol), Pd₂dba₃ (22 mg, 0.0242 mmol), XPhos (46 mg, 0.0968 mmol), and dioxane/H₂O (0.5 mL/1 mL). The resultant mixture was stirred at 110° C. under N₂, for 8h, cooled to rt, and filtered. The filtrate was diluted with DCM/MeOH (10/1, 80 mL), washed with H₂O (30 mL×2) and brine (30 mL), dried over anhydrous Na₂SO₄, filtered off and concentrated in vacuo. The residue was purified by flash column chromatography on silica gel (DCM/MeOH=100/1 to 50/1) to give the title compound (190 mg, yield: 64%).

### Step 2. 4-(5-((1R,5S,6s)-6-(aminomethyl)-3-azabicyclo[3.1.0]hexan-3-yl)pyrazin-2-yl)-6-(2-hydroxy-2-methylpropoxy)pyrazolo[1,5-a]pyridine-3-carbonitrile trifluoroacetate

To an ice-H₂O cooled solution of the product of Step 1 above (150 mg, 0.242 mmol) in DCM (9 mL) was added TFA (3 mL). The reaction mixture was stirred at rt for 1 h and concentrated in vacuo to give the crude title compound (171 mg, crude), which was used directly to the next step.

### Intermediate 50

### 4-(5-((1R,5S,6s)-6-(aminomethyl)-3-azabicyclo[3.1.0]hexan-3-yl)pyrazin-2-yl)-6-(1-methyl-1H-pyrazol-4-yl)pyrazolo[1,5-a]pyridine-3-carbonitrile trifluoroacetate

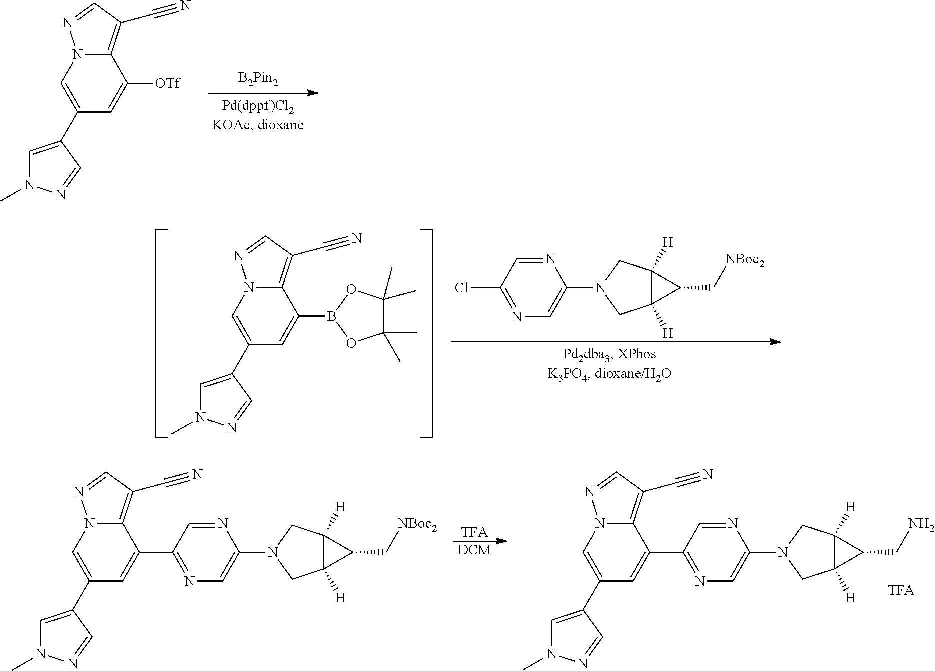

This intermediate was synthesized similarly by the procedure as described in Intermediate 49 by replacing Intermediate 3 with Intermediate 1.

### Intermediate 51 (3aR,6aS)-tert-butyl 5-(5-chloropyrazin-2-yl)hexahydropyrrolo[3,4-c]pyrrole-2(1H)-carboxylate

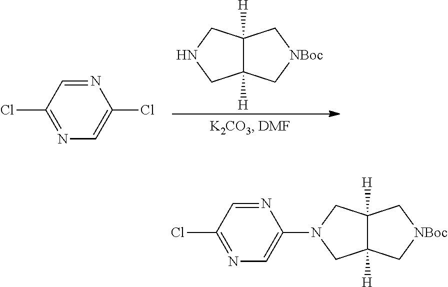 [SMILES: Clc1cnc(Cl)cn1 ; CC(C)(C)OC(=O)N1C[C@H]2CNC[C@H]2C1 ; CC(C)(C)OC(=O)N1C[C@@H]2CN(c3cnc(Cl)cn3)C[C@@H]2C1]

A mixture of 2,5-dichloropyrazine (500 mg, 3.36 mmol), (3aR,6aS)-tert-butyl hexahydropyrrolo[3,4-c]pyrrole-2(1H)-carboxylate (712 mg, 3.36 mmol), and K₂CO₃ (929 mg, 6.72 mmol) in DMF (10 mL) was stirred at 110° C. under N₂ overnight. The mixture was cooled to rt and concentrated. The residue was taken up in DCM/MeOH (10/1, 200 mL), washed with H₂O (50 mL×2) and brine (50 mL), dried over anhydrous Na₂SO₄, filtered off, and concentrated. The residue was purified by reverse phase flash column chromatography on C18 (MeOH/H₂O) to give the title compound (976 mg, yield: 89%).

### Intermediate 52

### 4-(5-((3aR,6aS)-hexahydropyrrolo[3,4-c]pyrrol-2(1H)-yl)pyrazin-2-yl)-6-(1-methyl-1H-pyrazol-4-yl)pyrazolo[1,5-a]pyridine-3-carbonitrile hydrochloride

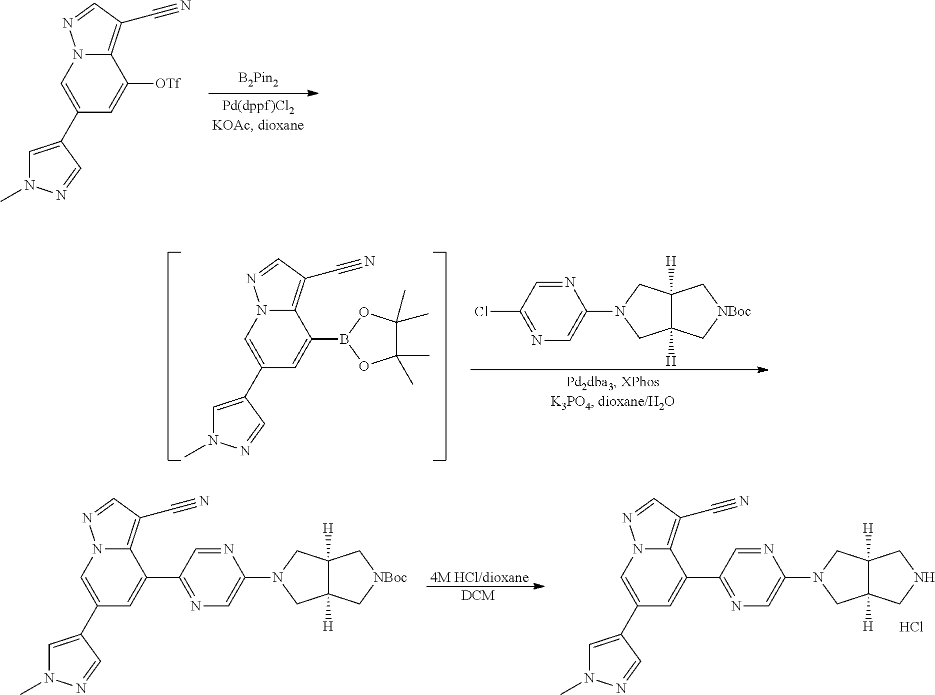

This intermediate was synthesized similarly by the procedure as described in Intermediate 49 by replacing Intermediate 3 with Intermediate 1 and by replacing Intermediate 48 with Intermediate 51.

### Intermediate 53

### 4-(5-((3aR,6aS)-hexahydropyrrolo[3,4-c]pyrrol-2(1H)-yl)pyrazin-2-yl)-6-(2-hydroxy-2-methylpropoxy)pyrazolo[1,5-a]pyridine-3-carbonitrile

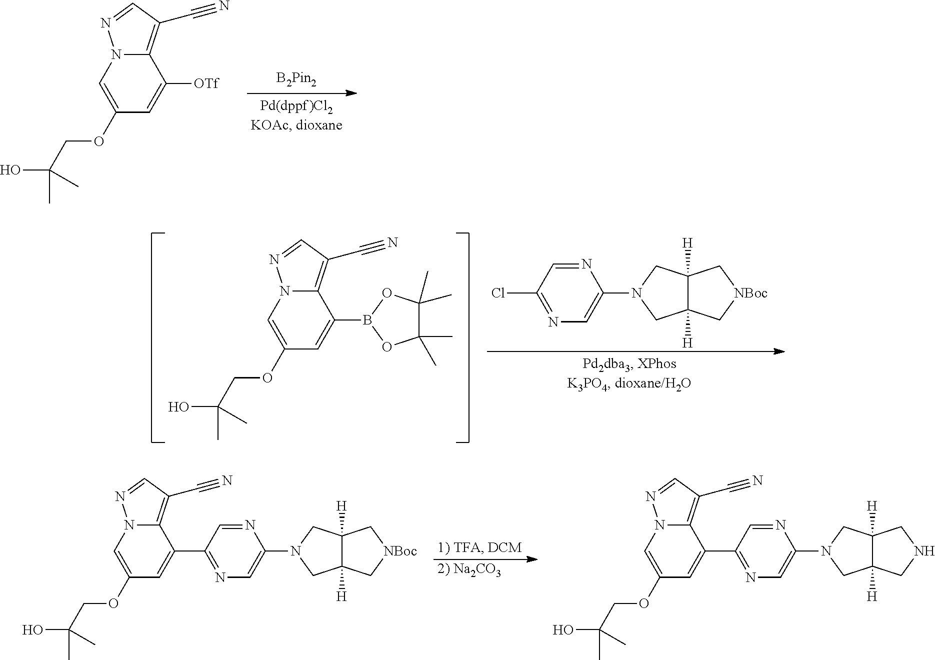

This intermediate was synthesized similarly by the procedure as described in Intermediate 48 by replacing Intermediate 48 with Intermediate 51.

### Intermediate 54

### N-((1R,3 S,5s,7s)-2-(5-chloropyrazin-2-yl)-2-azaadamantan-5-yl)formamide

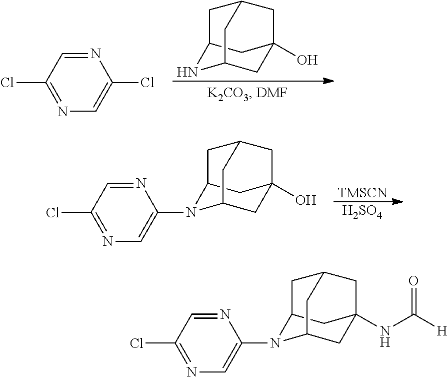

### Step 1. (1R,3 S,5s,7s)-2-(5-chloropyrazin-2-yl)-2-azaadamantan-5-ol

A mixture of 2,5-dichloropyrazine (3.6 g, 24 mmol), the product of Step 9 in Example 89 (5.0 g, 20 mmol), and K₂CO₃ (8.3 g, 60 mmol) in DMF (50 mL) was stirred at 130° C. under N₂ overnight. The mixture was cooled to rt and concentrated in vacuo. The residue was taken up in EtOAc (600 mL), washed with brine (100 mL), dried over anhydrous Na₂SO₄, filtered off, and concentrated. The residue was purified by flash column chromatography on silica gel (PE/EtOAc=2/1 to 1/1) to give the title compound (3.3 g, yield: 62%).

### Step 2. N-((1R,3S,5s,7s)-2-(5-chloropyrazin-2-yl)-2-azaadamantan-5-yl)formamide

To an ice-water cooled solution of the product of Step 1 above (2.2 g, 8.28 mmol) in concentrated H₂SO₄ (100 mL) was added TMSCN (8.2 g, 82.8 mmol) dropwise. The mixture was stirred at rt overnight, neutralized with saturated aqueous Na₂CO₃ to pH=8-9, and extracted with EtOAc (200 mL×3). The combined extracts were washed with brine (100 mL×2), dried over anhydrous Na₂SO₄, filtered off, and concentrated. The residue was purified by flash column chromatography on silica gel (PE/EtOAc=2/1 to EtOAc) to give the title compound (800 mg, yield: 33%).

### Intermediate 55

### (3aR,5r,6aS)-5-(pyridin-2-ylmethyl)octahydrocyclopenta[c]pyrrol-5-ol hydrochloride

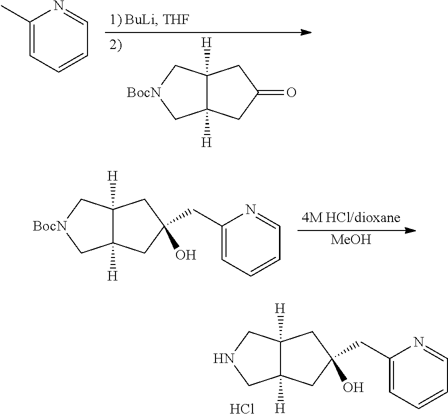

### Step 1. (3aR,5r,6aS)-tert-butyl 5-hydroxy-5-(pyridin-2-ylmethyl)hexahydrocyclopenta[c]pyrrole-2(1H)-carboxylate

A solution of 2-methylpyridine (2.25 g, 10 mmol) in dry THF (30 mL) was cooled to −50° C. and n-BuLi (2.5N in hexane, 4.2 mL) was added dropwise under N₂. The mixture was allowed to warm to rt and stirring was continued for 0.5h. The mixture was recooled to −78° C. and a solution of (3aR,6aS)-tert-butyl 5-oxohexahydrocyclopenta[c]pyrrole-2(1H)-carboxylate (930 mg, 10 mmol) in dry THF (5 mL) was added dropwise. The mixture was allowed to warm to rt, stirred overnight, cooled in ice-H₂O bath, quenched with saturated aqueous NH₄Cl (30 mL), and extracted with EtOAc (50 mL×2). The combined organics were washed with brine (30 mL), dried over anhydrous Na₂SO₄, filtered off, and concentrated. The residue was purified by flash column chromatography on silica gel (PE/EtOAc=3/1 to 2/1) to give title compound (1.0 g, yield: 31%).

### Step 2. (3aR,5r,6aS)-5-(pyridin-2-ylmethyl)octahydrocyclopenta[c]pyrrol-5-ol hydrochloride

To a solution of the product of Step 1 above (1.0 g, 3.1 mmol) in MeOH (5 mL) was added 4M HCl/dioxane (5 mL) at rt. The mixture was stirred at rt for 2h and concentrated to give the title compound (680 mg, quantitative).

### Intermediate 56

### N-((3aR,5r,6aS)-2-(5-chloropyrazin-2-yl)octahydrocyclopenta[c]pyrrol-5-yl)-6-methoxynicotinamide

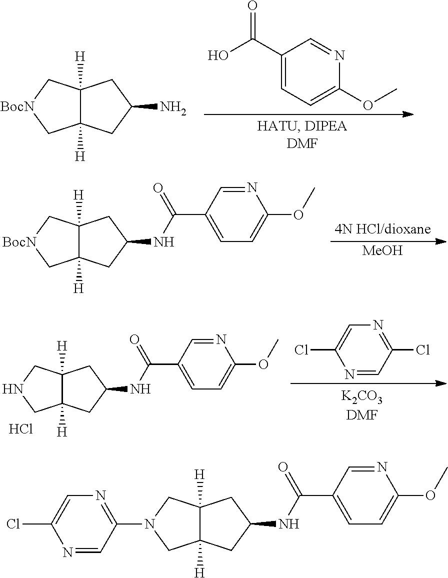

This intermediate was synthesized similarly by the procedure as described in Intermediate 19.

### Intermediate 57

### (1R,3S,5s,7s)-methyl 2-azaadamantane-5-carboxylate trifluoracetate

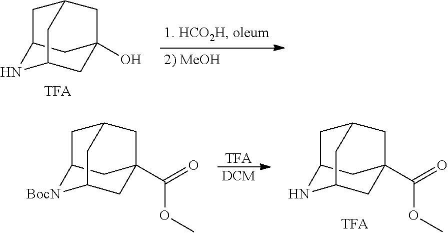

### Step 1. (1R,3S,5s,7s)-2-tert-butyl 5-methyl 2-azaadamantane-2,5-dicarboxylate

To 30% oleum (80 mL) was added dropwise the product of Step 9 in Example 89 (3.5 g, 13.1 mmol) in 98% formic acid (20 mL) at 60° C. Upon completion of this addition, 98% formic acid (20 mL) was added dropwise over a period of 30 minutes. The mixture was stirred at 100° C. for 3h. The mixture was cooled to rt and slowly poured into methanol (166 mL) cooled to 0° C. with vigorously stirring. The resulting mixture was stirred at 0˜rt overnight and concentrated in vacuo. The residue was poured into ice-H₂O (600 mL), basified with solid Na₂CO₃ to pH=10 and treated with THF (400 mL), TEA (2.65 g, 26.2 mmol), and Boc₂O (4.3 g, 19.65 mmol). The mixture was stirred at rt overnight and extracted with EtOAc (1L×2). The combined organics were dried over anhydrous Na₂SO₄, filtered off, and concentrated in vacuo. The residue was purified by flash column chromatography on silica gel (PE/EtA=10/1 to DCM/EtOAc=1/1) to give the title compound (2.2 g, yield: 57%).

### Step 2. (1R,3S,5s,7s)-methyl 2-azaadamantane-5-carboxylate trifluoracetate

To an ice-H₂O cooled solution of the product of Step 1 above (2.3 g, 7.78 mmol) in DCM (20 mL) was added TFA (5 mL). The reaction mixture was stirred at rt for 3h and concentrated in vacuo to give the crude title compound (2.4 g, 100%).

### Intermediate 58

### (1R,3S,5s,7s)-2-(5-chloropyrazin-2-yl)-N-(6-methoxypyridin-3-yl)-2-azaadamantane-5-carboxamide

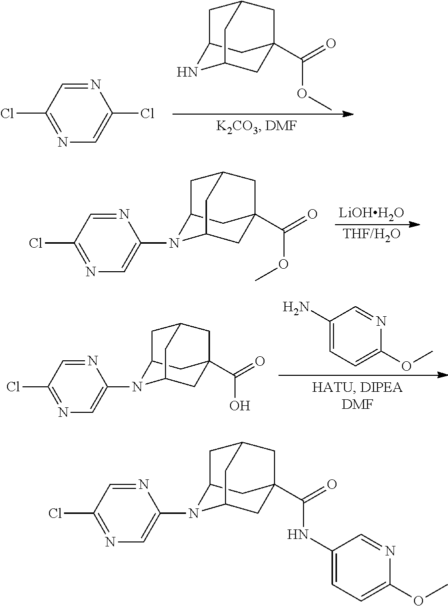

### Step 1. (1R,3 S,5s,7s)-methyl 2-(5-chloropyrazin-2-yl)-2-azaadamantane-5-carboxylate

A mixture of 2,5-dichloropyrazine (0.72 g, 4.85 mmol), Intermediate 57 (1.0 g, 3.24 mmol), and K₂CO₃ (1.79 g, 12.96 mmol) in DMF (10 mL) was stirred at 130° C. under N₂ overnight. The mixture was cooled to rt and concentrated. The residue was taken up in EtOAc (200 mL), washed with H₂O (50 mL) and brine (30 mL), dried over anhydrous Na₂SO₄, filtered off, and concentrated. The residue was purified by flash column chromatography on silica gel (PE/EtOAc=10/1) to give the title compound (689 mg, yield: 69%).

### Step 2. (1R,3S,5s,7s)-2-(5-chloropyrazin-2-yl)-2-azaadamantane-5-carboxylic acid

To a solution of the product of Step 1 above (689 mg, 2.24 mmol) in THF/H₂O (6 mL/6 mL) was added LiOH H₂O (282 mg, 6.72 mmol) at rt. The mixture was stirred at rt overnight, acidified to pH=5 with 1N HCl, and extracted with EtOAc (10 mL). The organic phase was washed with brine (10 mL), dried over anhydrous Na₂SO₄, and concentrated to give the title compound (640 mg, yield: 97%).

### Step 3. (1R,3S,5s,7s)-2-(5-chloropyrazin-2-yl)-N-(6-methoxypyridin-3-yl)-2-azaadamantane-5-carboxamide

To a solution of the product of Step 1 above (300 mg, 2.02 mmol), 6-methoxypyridin-3-amine (190 mg, 2.54 mmol), and HATU (585 mg, 2.54 mmol) in DMF (4 mL) was added DIPEA (395 mg, 3.06 mmol) at rt. The mixture was stirred at rt overnight and purified by reverse phase flash column chromatography on C18 (MeOH/H₂O) to give the title compound (360 mg, yield: 88%).

### Intermediate 59

### (3aR,5r,6aS)-2-(5-bromopyridin-2-yl)-5-methyloctahydrocyclopenta[c]pyrrole-5-carboxylic acid

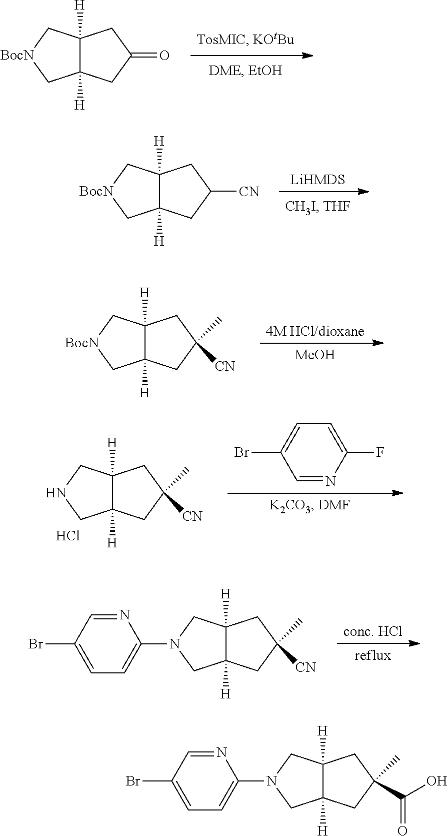

### Step 1. (3aR,6aS)-tert-butyl 5-cyanohexahydrocyclopenta[c]pyrrole-2(1H)-carboxylate

To an ice-water cooled solution of (3aR,6aS)-tert-butyl 5-oxohexahydrocyclopenta[c]pyrrole-2(1H)-carboxylate (10 g, 44 mmol) in DME (150 mL) and EtOH (4.4 mL) were added TosMIC (9 g, 46 mmol) and t-BuOK (9.8 g, 88 mmol) sequentially. After addition, the mixture was allowed to warm up to rt and stirred for 3 h. The reaction mixture was filtered off and the filtrate was concentrated. The residue was purified via flash column chromatography on silica gel (PE/EtOAc=6/1˜4/1) to give the title compound (4.3 g, yield: 41%).

### Step 2. (3aR,5r,6aS)-tert-butyl 5-cyano-5-methylhexahydrocyclopenta[c]pyrrole-2(1H)-carboxylate

To an ice-water cooled solution of the product of Step 1 above (4.3 g, 18.2 mmol) in THF (80 mL) was added L¹HMDS (1N in THF, 72.8 mL, 72.8 mmol) slowly. The mixture was stirred at that temperature for 0.5 h and Mel (10.3 g, 72.8 mmol) was added. After addition, the mixture was allowed to warm up to rt, stirred for 3 h, diluted with EtOAc (200 mL), washed with H₂O (30 mL×2) and brine (30 mL), dried over anhydrous Na₂SO₄, filtered off, and concentrated. The residue was purified via flash column chromatography on silica gel (PE/EtOAc=6/1 to 4/1) to give the title compound (1.9 g, yield:41%).

### Step 3. (3aR,5r,6aS)-5-methyloctahydrocyclopenta[c]pyrrole-5-carbonitrile hydrochloride

To a solution of the product of Step 2 above (1.9 g, 7.6 mmol) in MeOH (5 mL) was added HCl/dioxane (4 N, 5 mL, 20 mmol) at RT. The reaction mixture was stirred at rt overnight and concentrated to give the crude title compound (quantitative), which was used in the next step directly without further purification.

### Step 4. (3aR,5r,6aS)-2-(5-bromopyridin-2-yl)-5-methyloctahydrocyclopenta[c]pyrrole-5-carbonitrile

To a solution of the product of Step 3 above (crude, 7.6 mmol), followed by K₂CO₃ (4.2 g, 30.4 mmol). The reaction mixture was stirred for 3 h at 110° C., cooled to rt, diluted with EtOAc (150 mL), washed with H₂O (30 mL×2) and brine (30 mL), dried over anhydrous Na₂SO₄, filtered off, and concentrated. The residue was purified via flash column chromatography on silica gel (PE/EtOAc=6/1 to 4/1) to give the title compound (1.2 g, yield:52%).

### Step 5. (3aR,5r,6aS)-2-(5-bromopyridin-2-yl)-5-methyloctahydrocyclopenta[c]pyrrole-5-carboxylic acid

A solution of the product of Step 4 above (700 mg, 2.29 mmol) in concentrated HCl (12 N, 20 mL) was stirred at 110° C. overnight, cooled to 50° C. and concentrated. The residue was diluted with cold H₂O (0° C., 50 mL) and adjusted pH to 6˜7 with solid Na₂CO₃. The resulting suspension was filtered. The cake was rinsed with H₂O (10 mL) and dried in vacuo to give the title compound (720 mg, yield:97%).

### Intermediate 60

### tert-butyl ((3aR,5r,6aS)-2-(5-bromopyridin-2-yl)-5-methyloctahydrocyclopenta[c]pyrrol-5-yl)carbamate

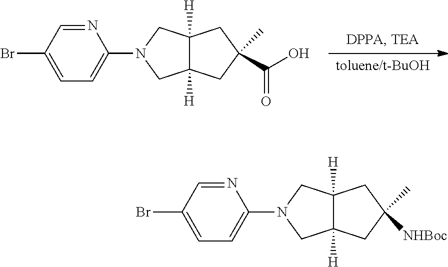 [SMILES: C[C@]1(C(=O)O)C[C@H]2CN(c3ccc(Br)cn3)C[C@H]2C1 ; "DPPA, TEA" ; "toluene/t-BuOH" ; CC(C)(C)OC(=O)N[C@@]1(C)C[C@H]2CN(c3ccc(Br)cn3)C[C@H]2C1]

A solution of Intermediate 59 (400 mg, 1.23 mmol), DPPA (506 mg, 1.84 mmol) and TEA (502 mg, 4.96 mmol) in toluene/t-BuOH (8 mL/8 mL) was stirred at 120° C. overnight. The mixture was cooled to rt and concentrated in vacuo. The residue was taken up in EtOAc (150 mL), washed with H₂O (50 mL×2) and brine (50 mL), dried over anhydrous Na₂SO₄, filtered off, and concentrated. The residue was purified by flash column chromatography on silica gel (PE/EtOAc=6/1) to give the title compound (490 mg, yield: 81%).

### Intermediate 61

### 4-(6-((3aR,5r,6aS)-5-amino-5-methylhexahydrocyclopenta[c]pyrrol-2(1H)-yl)pyridin-3-yl)-6-(2-hydroxy-2-methylpropoxy)pyrazolo[1,5-a]pyridine-3-carbonitrile hydrochloride

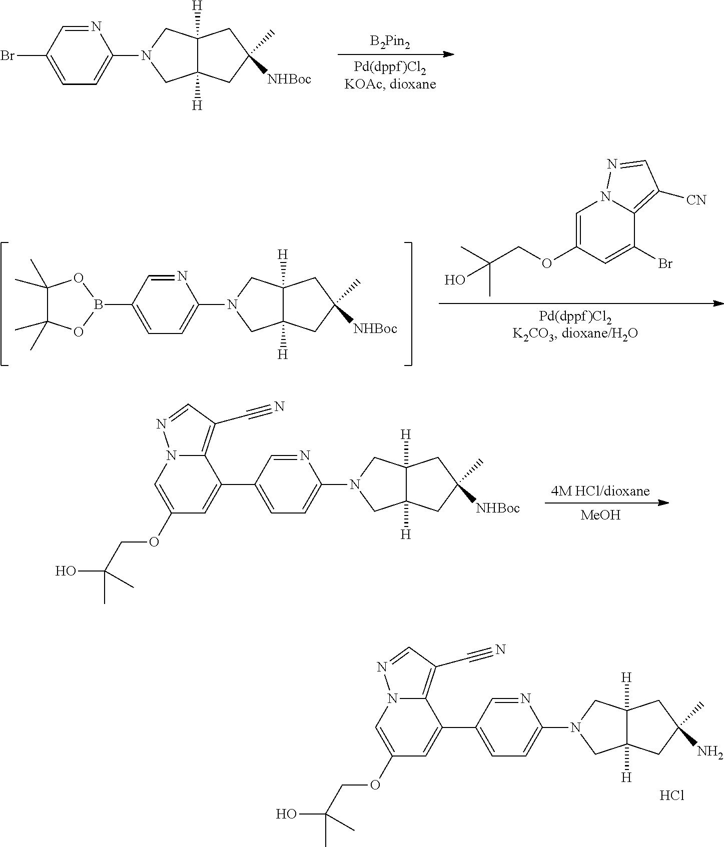

### Step 1. tert-butyl ((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)carbamate

A mixture of Intermediate 60 (200 mg, 0.51 mmol), B₂Pin₂ (135 mg, 0.53 mmol), AcOK (100 mg, 1.02 mmol) and Pd(dppf)Cl₂.DCM (41 mg, 0.05 mmol) in dioxane (5 mL) was stirred at 100° C. for 3h under N₂. The mixture was cooled to rt and to which Intermediate 3 (142 mg, 0.46 mmol), K₂CO₃ (141 mg, 1.02 mmol), Pd(dppf)Cl₂DCM (41 mg, 0.05 mmol), and dioxane/H₂O (10 mL/2 mL) were added. The reaction mixture was stirred at 110° C. overnight under N₂, cooled to rt, diluted with DCM/MeOH (10/1, 100 mL), washed with H₂O (30 mL×2) and brine (30 mL), dried over anhydrous Na₂SO₄, filtered off, and concentrated. The residue was purified by flash column chromatography on silica gel (DCM/MeOH=50/1) to give the crude product, which was further purified by perp-TLC (DCM/MeOH=30/1) to give the title compound (150 mg, yield: 60%).

### Step 2. 4-(6-((3aR,5r,6aS)-5-amino-5-methylhexahydrocyclopenta[c]pyrrol-2(1H)-yl)pyridin-3-yl)-6-(2-hydroxy-2-methylpropoxy)pyrazolo[1,5-a]pyridine-3-carbonitrile hydrochloride

To a solution of the product of Step 1 above (150 mg, 0.274 mmol) in MeOH (3 mL) was added 4M HCl/dioxane (5 mL) at rt. The mixture was stirred at rt for 4 h and concentrated to give the title compound (150 mg, crude, quantitative).

### Intermediate 62

### 4-(6-((3aR,5r,6aS)-5-amino-5-methylhexahydrocyclopenta[c]pyrrol-2(1H)-yl)pyridin-3-yl)-6-(1-methyl-1H-pyrazol-4-yl)pyrazolo[1,5-a]pyridine-3-carbonitrile hydrochloride

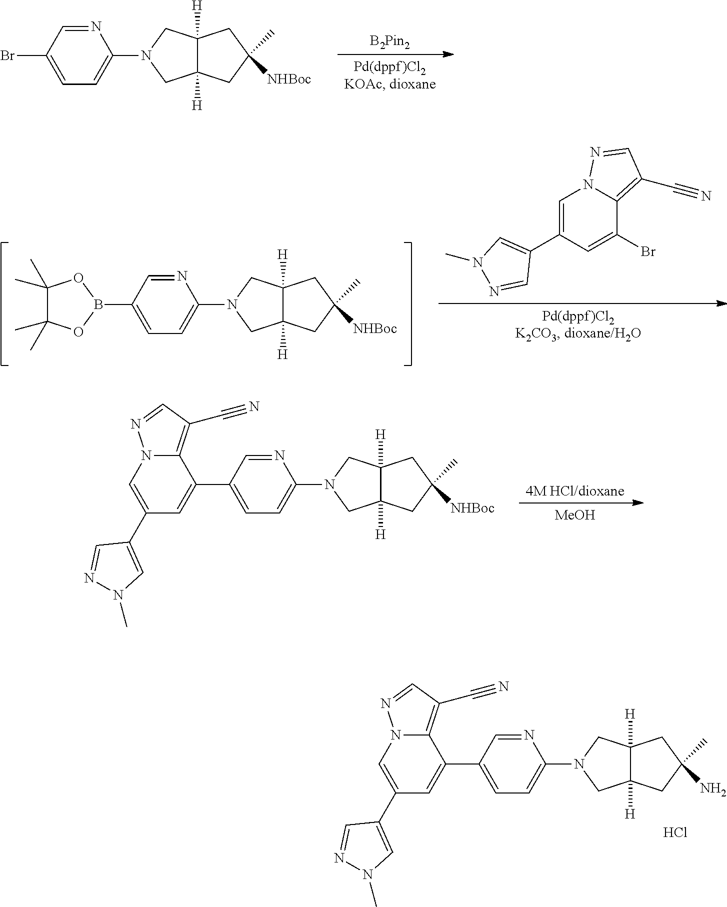

This intermediate was synthesized via similar procedure as described Example 11.

### Intermediate 63

### (1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexane-6-carboxylic acid

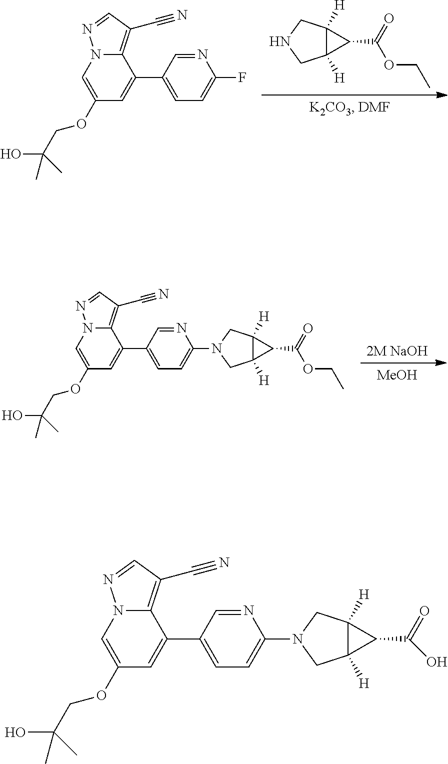

### Step 1. (1R,5S,6r)-ethyl 3-(5-(3-cyano-6-(2-hydroxy-2-methylpropoxy)pyrazolo[1,5-a]pyridin-4-yl)pyridin-2-yl)-3-azabicyclo[3.1.0]hexane-6-carboxylate

A mixture of Intermediate 29 (150 mg, 0.46 mmol), (1R,5S,6r)-ethyl 3-azabicyclo[3.1.0]hexane-6-carboxylate (85.6 mg, 0.55 mmol), and K₂CO₃ (127 mg, 2.0 mmol) in DMF (5 mL) was stirred at 110° C. under N₂ overnight. The mixture was cooled to rt, diluted with DCM/MeOH (10/1, 30 mL), washed with H₂O (10 mL×2) and brine (10 mL), dried over anhydrous Na₂SO₄, filtered off, and concentrated. The residue was purified by prep-TLC (DCM/MeOH=15/1) to give the title compound (130 mg, yield: 61%).

### Step 2. (1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexane-6-carboxylic acid

To a solution of the product of Step 1 above (130 mg, 0.282 mmol) in MeOH (6 mL) was added 2M NaOH (0.5 mL, 1.0 mmol) at rt. The mixture was stirred at 50° C. for 2 h, concentrated to remove most MeOH, diluted with H₂O (10 mL), acidified to pH=3 with 2M HCl, and extracted with DCM (20 mL×2). The combined organics were washed with brine (20 m), dried over anhydrous Na₂SO₄, filtered off and concentrated to give the title compound (100 mg, yield: 82%).

### Intermediate 64

### N-(2-((1R,5S,6r)-3-azabicyclo[3.1.0]hexan-6-yl)propan-2-yl)formamide hydrochloride

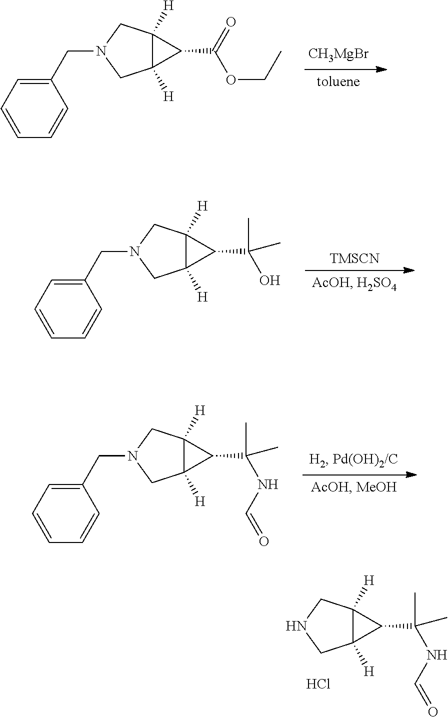

### Step 1. 2-((1R,5S,6r)-3-benzyl-3-azabicyclo[3.1.0]hexan-6-yl)propan-2-ol

To a solution of (1R,5S,6r)-ethyl 3-benzyl-3-azabicyclo[3.1.0]hexane-6-carboxylate (600 mg, 2.45 mmol) in toluene (15 mL) was added MeMgBr (3N in MeTHF, 4.1 mL, 12.25 mmol) dropwise at rt. The reaction mixture was stirred at rt overnight, quenched with saturated aqueous NH₄Cl (100 mL), and extracted with EtOAc (100 mL×2). The combined organics were washed with brine (100 mL×2), dried over anhydrous Na₂SO₄, filtered off, and concentrated in vacuo to give the title compound (550 mg, yield: 97%)

### Step 2. N-(2-((1R,5S,6r)-3-benzyl-3-azabicyclo[3.1.0]hexan-6-yl)propan-2-yl)formamide

To an ice-water cooled solution of the product of Step 1 above (250 mg, 1.08 mmol) in AcOH (2.5 mL) was added TMSCN (322 mg, 3.24 mmol), and then H₂SO₄ (concd, 2.5 mL) was added dropwise at 0° C. while stirring. After addition was complete, the reaction mixture was allowed to warm up to rt and stirred for 3 h. Then reaction mixture was cooled with an ice-H₂O bath, basified with saturated aqueous Na₂CO₃ to pH=9˜10, and extracted with EtOAc (100 mL×2). The combined organics were dried over anhydrous Na₂SO₄, filtered off, and purified via flash column chromatography on silica gel (PE/EtOAc=1/1 to DCM/MeOH/NH₃.H₂O=10/1/0.1) to give the title compound (253 mg, yield: 91%).

### Step 3. N-(2-((1R,5S,6r)-3-azabicyclo[3.1.0]hexan-6-yl)propan-2-yl)formamide hydrochloride

To a solution of the product of Step 2 above (304 mg, 1.18 mmol) in MeOH (10 mL) was added AcOH (1 mL), followed by the addition of Pd(OH)₂/C (30 mg). The reaction mixture was stirred under H₂ balloon at 60° C. overnight. The reaction suspension was filtered through a pad of Celite and the pad was washed with MeOH (10 mL). HCl (2N aq., 2 mL) was added to the filtrate and stirred for 30 min. The above solution was concentrated in vacuo to give the crude title compound (256 mg, which was used in the next step without any further purification.

### Intermediate 65

### 4-(6-((1R,5S,6r)-6-(2-aminopropan-2-yl)-3-azabicyclo[3.1.0]hexan-3-yl)pyridin-3-yl)-6-(2-hydroxy-2-methylpropoxy)pyrazolo[1,5-a]pyridine-3-carbonitrile

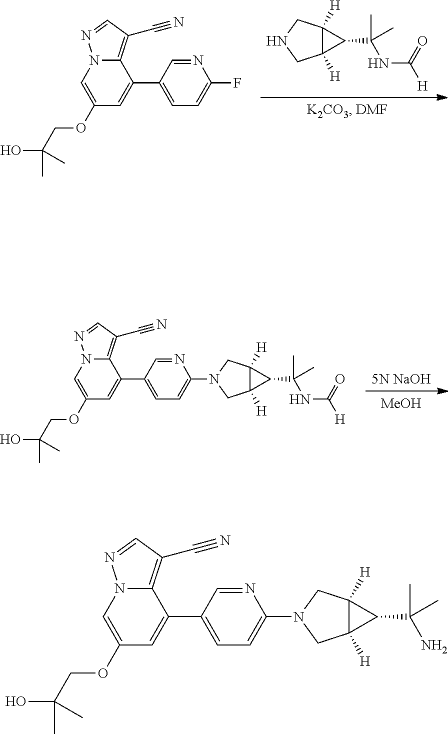

This intermediate was synthesized via similar procedure as described in Intermediate 38.

### Example 1

### 4-(6-((3aR,6aS)-5-(6-methoxynicotinoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile

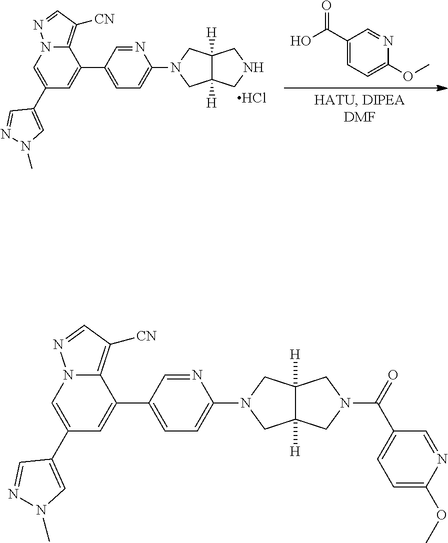

To a solution of Intermediate 5 (crude, 0.190 mmol), 6-methoxynicotinic acid (33.6 mg, 0.219 mmol), and HATU (125 mg, 0.328 mmol) in DMF (5 mL) was added DIPA (170 mg, 1.3 mmol) at rt. The reaction solution was stirred at rt overnight. The mixture was diluted with DCM/MeOH (10/1, 50 mL), washed with H₂ (20 mL×3) and brine (20 mL), dried over anhydrous Na₂SO₄, filtered offm and concentrated in vacuo. The residue was purified by reverse phase flash column chromatography (MeOH:H₂O=4000 to 950%) to give the title compound (40 mg, yield: 33%). ESI-MS (m/z): 546.3 [M+1]⁺. ¹H NMR (400 MHz, DMSO-d₆) δ 9.21 (d, J=1.1 Hz, 1H), 8.64 (s, 1H), 8.41 (d, J=2.1 Hz, 1H), 8.38 (s, 1H), 8.36 (d, J=2.3 Hz, 1H), 8.11 (s, 1H), 7.91 (dd, J=8.6, 2.4 Hz, 1H), 7.80 (dd, J=8.7, 2.4 Hz, 1H), 7.74 (d, J=1.1 Hz, 1H), 6.86 (d, J=8.6 Hz, 1H), 6.59 (d, J=8.8 Hz, 1H), 3.89 (s, 3H), 3.88 (s, 3H), 3.86-3.79 (m, 2H), 3.78-3.61 (m, 2H), 3.56-33.43 (m, 4H), 3.15-3.03 (m, 2H).

Table 1 lists examples that were prepared according to the procedures as described in Example 1 by using the corresponding intermediates and reagents under appropriate conditions that could be accomplished by the skilled persons.

TABLE 1

| Ex. | Structure | Chemical | Mass | |
|---|---|---|---|---|
| # | (Synthetic Method) | Name | m/z | 1H NMR |
| 2 | 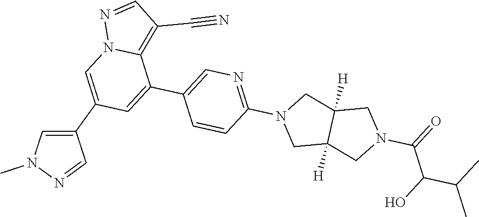 [SMILES: CC(C)C(O)C(=O)N1C[C@@H]2CN(c3ccc(-c4cc(-c5cnn(C)c5)cn5ncc(C#N)c45)cn3)C[C@@H]2C1] | 4-(6-((3aR,6aS)- 5-(2-hydroxy-3- methylbutanoyl) hexahydropyrrolo [3,4-c]pyrrol- 2(1H)-pyridin- 3-yl)-6-(1-methyl- 1H-pyrazol-4-yl) pyrazolo[1,5-a] pyridine-3- carbonitrile | 511.4 | ¹H NMR (400 MHz, DMSO- d₆) δ 9.21 (d, J = 1.1 Hz, 1H), 8.63 (s, 1H), 8.38 (s, 1H), 8.36 (d, J = 2.2 Hz, 1H), 8.11 (s, 1H), 7.80 (dd, J = 8.7, 2.3 Hz, 1H), 7.74 (s, 1H), 6.59 (d, J = 8.8 Hz, 1H), 4.67 (d, J = 7.1 Hz, 1H), 3.88 (s, 3H), 3.82-3.53 (m, 4H), 3.49- 3.31 (m, 4H), 3.15-3.06 (m, 1H), 3.05-2.96 (m, 1H), 1.95-1.82 (m, 1H), 0.89- 0.80 (m, 6H). |
| 3 | 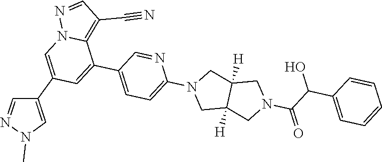 [SMILES: Cn1cc(-c2cc(-c3ccc(N4C[C@H]5CN(C(=O)C(O)c6ccccc6)C[C@H]5C4)nc3)c3c(C#N)cnn3c2)cn1] | 4-(6-((3aR,6aS)- 5-(2-hydroxy-2- phenylacetyl) hexahydropyrrolo [3,4-c] pyrrol-2(1H)-yl) pyridin-3-yl)- 6-(1-methyl- 1H-pyrazol- 4-yl)pyrazolo[1,5- | 545.3 | ¹H NMR (400 MHz, CD₃OD) δ 8.84 (s, 1H), 8.33 (s, 1H), 8.25 (dd, J = 15.5, 2.1 Hz, 1H), 8.04 (d, J = 2.2 Hz, 1H), 7.89 (d, J = 2.2 Hz, 1H), 7.79- 7.71 (m, 1H), 7.58-7.53 (m, 1H), 7.43-7.37 (m, 2H), 7.38-7.32 (m, 3H), 7.32- 7.27 (m, 1H), 5.23 (d, J = 6.0 Hz, 1H), 3.96 (s, 3H), 3.76 |
| | | a]pyridine-3- | | (m, 2H), 3.70-3.65 (m, 1H), |
| | | carbonitrile | | 3.57-3.53 (m, 1H), 3.52- |
| | | | | 3.36 (m, 2H), 3.15-2.97 (m, |
| | | | | 4H). |
| 4 | 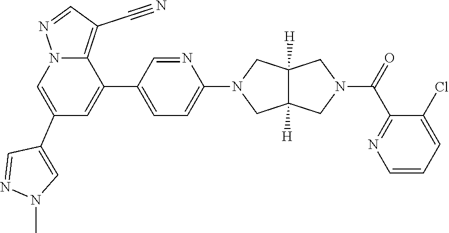 [SMILES: Cn1cc(-c2cc(-c3ccc(N4C[C@H]5CN(C(=O)c6ncccc6Cl)C[C@H]5C4)nc3)c3c(C#N)cnn3c2)cn1] | 4-(6-((3aR,6aS)- 5-(3- chloropicolinoyl) hexahydropyrrolo [3,4-c]pyrrol- 2(1H)-yl) pyridin-3-yl)-6- (1-methyl- 1H-pyrazol-4- yl)pyrazolo[1,5- a]pyridine-3- carbonitrile | 550.4 | ¹H NMR (400 MHz, DMSO- d₆) δ 9.20 (s, 1H), 8.62 (s, 1H), 8.55 (dd, J = 4.7, 1.2 Hz, 1H), 8.37 (s, 1H), 8.34 (d, J = 2.3 Hz, 1H), 8.10 (s, 1H), 8.05 (dd, J = 8.2, 1.1 Hz, 1H), 7.79 (dd, J = 8.7, 2.4 Hz, 1H), 7.73 (d, J = 1.1 Hz, 1H), 7.51 (dd, J = 8.2, 4.7 Hz, 1H), 6.58 (d, J = 8.7 Hz, 1H), 3.86 (s, 3H), 3.84- 3.71 (m, 2H), 3.66 (m, 1H), 3.54 (m, 1H), 3.51-3.39 (m, 2H), 3.37-3.31 (m, 1H), 3.13 (m, 1H), 3.10-3.01 (m, 2H). |
| 5 | 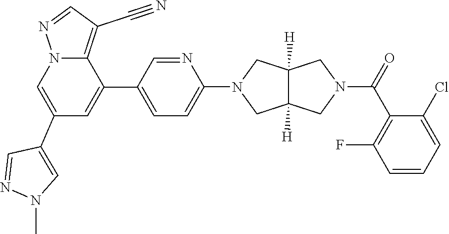 [SMILES: Cn1cc(-c2cc(-c3ccc(N4C[C@H]5CN(C(=O)c6c(F)cccc6Cl)C[C@H]5C4)nc3)c3c(C#N)cnn3c2)cn1] | 4-(6-((3aR,6aS)- 5-(2-chloro-6- fluorobenzoyl) hexahydropyrrolo [3,4-c]pyrrol- 2(1H)-yl) pyridin-3-yl)- 6-(1-methyl- 1H-pyrazol-4- yl)pyrazolo[1,5- a]pyridine-3- carbonitrile | 567.2 | ¹H NMR (400 MHz, DMSO- d₆) δ 9.20 (s, 1H), 8.62 (s, 1H), 8.37 (s, 1H), 8.34 (d, J = 1.8 Hz, 1H), 8.10 (s, 1H), 7.82-7.76 (m, 1H), 7.73 (d, J = 1.1 Hz, 1H), 7.50 (dd, J = 14.5, 8.0 Hz, 1H), 7.45-7.37 (m, 1H), 7.36-7.29 (m, 1H), 6.57 (d, J = 8.7 Hz, 1H), 3.86 (s, 3H), 3.84-3.71 (m, 2H), 3.68 (m, 1H), 3.58-3.47 (m, 2H), 3.42 (m, 1H), 3.38- 3.32 (m, 1H), 3.18-3.02 (m, 3H). |
| 6 | 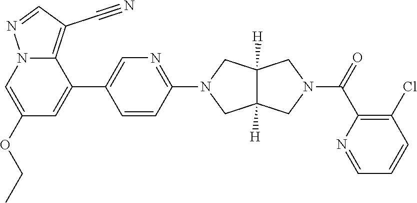 [SMILES: CCOc1cc(-c2ccc(N3C[C@H]4CN(C(=O)c5ncccc5Cl)C[C@H]4C3)nc2)c2c(C#N)cnn2c1] | 4-(6-((3aR,6aS)- 5-(3- chloropicolinoyl) hexahydropyrrolo [3,4-c]pyrrol- 2(1H)-yl) pyridin-3-yl)-6- ethoxypyrazolo [1,5-a]pyridine-3- carbonitrile | 514.0 | ¹H NMR (400 MHz, DMSO- d₆) δ 8.62 (d, J = 2.0 Hz, 1H), 8.58-8.53 (m, 2H), 8.29 (d, J = 2.3 Hz, 1H), 8.09-8.02 (m, 1H), 7.74 (dd, J = 8.7, 2.4 Hz, 1H), 7.50 (dd, J = 8.3, 4.7 Hz, 1H), 7.22 (d, J = 2.0 Hz, 1H), 6.55 (d, J = 8.7 Hz, 1H), 4.13 (q, J = 6.9 Hz, 2H), 3.85-3.62 (m, 2H), 3.58- 3.32 (m, 4H), 3.06 (m, 4H), 1.36 (t, J = 6.9 Hz, 3H). |
| 7 | 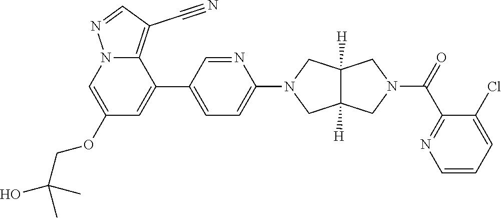 [SMILES: CC(C)(O)COc1cc(-c2ccc(N3C[C@H]4CN(C(=O)c5ncccc5Cl)C[C@H]4C3)nc2)c2c(C#N)cnn2c1] | 4-(6-((3aR,6aS)- 5-(3- chloropicolinoyl) hexahydropyrrolo [3,4-c]pyrrol- 2(1H)-yl) pyridin-3-yl)-6- (2-hydroxy-2- methylpropoxy) pyrazolo[1,5-a] pyridine-3- carbonitrile | 558.2 | ¹H NMR (400 MHz, DMSO- d₆) δ 8.64 (d, J = 1.9 Hz, 1H), 8.55 (d, J = 2.4 Hz, 2H), 8.30 (d, J = 2.2 Hz, 1H), 8.05 (d, J = 8.2 Hz, 1H), 7.74 (dd, J = 8.7, 2.4 Hz, 1H), 7.50 (dd, J = 8.2, 4.7 Hz, 1H), 7.24 (d, J = 1.9 Hz, 1H), 6.56 (d, J = 8.8 Hz, 1H), 4.69 (s, 1H), 3.85 (s, 2H), 3.80-3.73 (m, 1H), 3.65 (m, 1H), 3.57- 3.40 (m, 4H), 3.10 (m, 4H), 1.21 (s, 6H). |
| 8 | 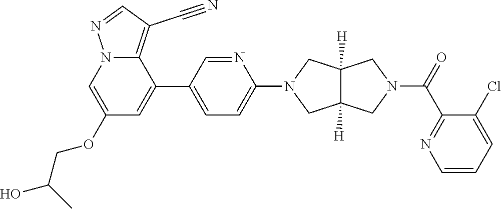 [SMILES: CC(O)COc1cc(-c2ccc(N3C[C@H]4CN(C(=O)c5ncccc5Cl)C[C@H]4C3)nc2)c2c(C#N)cnn2c1] | 4-(6-((3aR,6aS)- 5-(3- chloropicolinoyl) hexahydropyrrolo [3,4-c]pyrrol- 2(1H)-yl)pyridin- 3-yl)-6-(2- hydroxypropoxy) pyrazolo[1,5-a] pyridine-3- carbonitrile | 544.3 | ¹H NMR (400 MHz, CD₃OD) δ 8.54 (d, J = Hz, 1H), 8.43 (s, 1H), 8.32 (s, 1H), 8.25 (s, 1H), 8.00 (d, J = 7.9 Hz, 1H), 7.76 (d, J = 7.0 Hz, 1H), 7.50 (dd, J = 8.1, 4.7 Hz, 1H), 7.27 (s, 1H), 6.66 (d, J = 8.7 Hz, 1H), 4.15 (s, 1H), 4.02 (m, 1H), 3.94 (m, 2H), 3.88-3.81 (m, 1H), 3.74 (m, 2H), 3.66-3.53 (m, 2H), 3.46 (m, 1H), 3.22 (m, 3H), 1.29 (d, J = 6.3 Hz, 3H). |
| 9 | 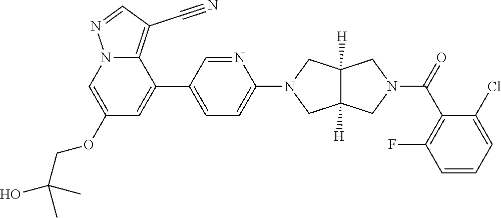 [SMILES: CC(C)(O)COc1cc(-c2ccc(N3C[C@H]4CN(C(=O)c5c(F)cccc5Cl)C[C@H]4C3)nc2)c2c(C#N)cnn2c1] | 4-(6-((3aR,6aS)- 5-(2-chloro-6- fluorobenzoyl) hexahydropyrrolo [3,4-c]pyrrol- 2(1H)-yl) pyridin-3-yl)-6- (2-hydroxy-2- methylpropoxy) pyrazolo[1,5-a] pyridine-3- carbonitrile | 575.4 | ¹H NMR (400 MHz, DMSO- d6) δ 8.64 (s, 1H), 8.55 (s, 1H), 8.30 (s, 1H), 7.74 (d, J = 8.6 Hz, 1H), 7.54-7.46 (m, 1H), 7.44-7.29 (m, 2H), 7.24 (s, 1H), 6.55 (d, J = 8.5 Hz, 1H), 4.69 (s, 1H), 3.85 (s, 2H), 3.82-3.63 (m, 3H), 3.52 (m, 2H), 3.41 (m, 2H), 3.14 (m, 1H), 3.07 (m, 2H), 1.21 (s, 6H). |

### Example 10

### 4-(6-((3aR,6aS)-5-isobutyrylhexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile

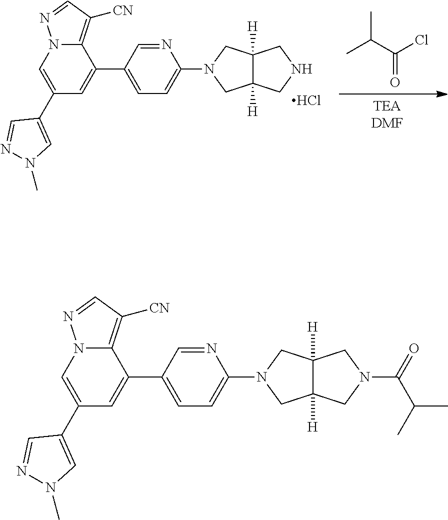

To a solution of Intermediate 5 (35 mg, 0.085 mmol) in DMF (2 mL) was added TEA (0.5 mL) at 0° C., followed by the addition of isobutyryl chloride (10 mg, 0.094 mmol) dropwise. The reaction solution was stirred at rt overnight. After concentrating in vacuo, the residue was diluted with DCM/MeOH (10/1, 100 mL), washed with H₂O (30 mL×2) and brine (30 mL), dried over anhydrous Na₂SO₄, filtered off, and concentrated in vacuo. The residue was purified by prep-TLC (DCM/MeOH=20/1) to give the title compound (14 mg, yield: 31%). ESI-MS (m/z): 481.2 [M+1]⁺. ¹H NMR (400 MHz, DMSO-d₆) δ 9.20 (d, J=1.0 Hz, 1H), 8.62 (s, 1H), 8.37 (s, 1H), 8.34 (d, J=2.3 Hz, 1H), 8.10 (s, 1H), 7.78 (dd, J=8.7, 2.4 Hz, 1H), 7.72 (d, J=1.1 Hz, 1H), 6.58 (d, J=8.7 Hz, 1H), 3.86 (s, 3H), 3.80 (m, 10H), 2.71-2.59 (m, 1H), 0.98 (t, J=6.5 Hz, 6H).

### Example 11

### 4-(6-((3aR,6aS)-5-(2-chloro-6-fluorophenylsulfonyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile

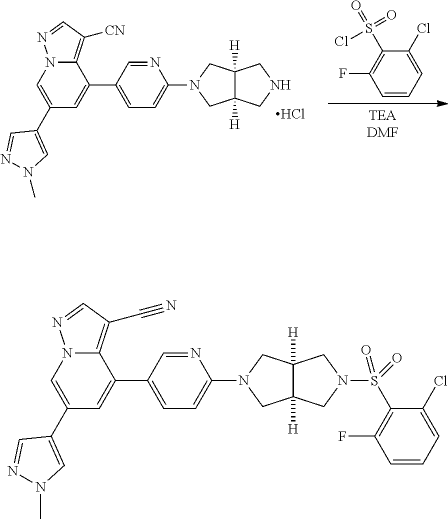

To a solution of Intermediate 5 (75 mg, 0.183 mmol) in DMF (1 mL) was added TEA (55.5 mg, 0.548 mmol), followed by the dropwise addition of 2-chloro-6-fluorobenzene-1-sulfonyl chloride (41.8 mg, 0.183 mmol) at −20° C. The reaction solution was stirred at rt overnight. The precipitate formed was collected by filtration, washed with H₂O, and dried in vacuo to give the title compound (40 mg, yield: 36%). ESI-MS (m/z): 603.4 [M+1]⁺. ¹H NMR (400 MHz, DMSO-d₆) δ 9.20 (s, 1H), 8.62 (s, 1H), 8.37 (s, 1H), 8.34 (d, J=2.1 Hz, 1H), 8.10 (s, 1H), 7.78 (dd, J=8.7, 2.3 Hz, 1H), 7.73 (s, 1H), 7.69-7.62 (m, 1H), 7.52 (d, J=8.0 Hz, 1H), 7.48-7.39 (m, 1H), 6.56 (d, J=8.7 Hz, 1H), 3.86 (s, 3H), 3.72-3.58 (m, 4H), 3.39-3.32 (m, 4H), 3.10 (m, 2H).

### Example 12

### 4-(6-((3aR,6aS)-5-((6-methoxypyridin-3-yl)methyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile

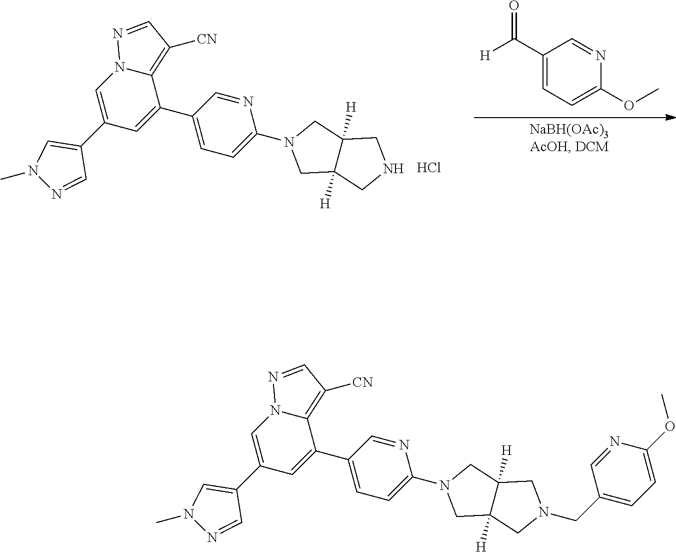

To a solution of Intermediate 5 (50 mg, 0.12 mmol) in DCM (1 mL) was added 6-methoxynicotinaldehyde (16.7 mg, 0.12 mmol) and a drop of AcOH at rt. The reaction mixture was stirred at rt for 30 min before adding NaBH(OAc)₃ (51.6 mg, 0.24 mmol). The reaction mixture was stirred at rt overnight. After concentrating in vacuo, the residue was dissolved in DCM/MeOH (10/1, 100 mL), washed with H₂O (30 mL×2) and brine (30 mL), dried over anhydrous Na₂SO₄, filtered off, and concentrated in vacuo. The residue was purified by prep-TLC (DCM/MeOH=10/1) to give the title compound (7 mg, yield: 10%). ESI-MS (m/z): 532.2 [M+1]⁺. ¹H NMR (400 MHz, DMSO-d₆) δ 9.21 (s, 1H), 8.63 (s, 1H), 8.37 (s, 1H), 8.34 (s, 1H), 8.10 (s, 1H), 8.04 (s, 1H), 7.78 (d, J=8.6 Hz, 1H), 7.74 (s, 1H), 7.61 (s, 1H), 6.76 (d, J=8.4 Hz, 1H), 6.62 (d, J=8.5 Hz, 1H), 3.86 (s, 3H), 3.81 (s, 3H), 3.62 (m, 2H), 3.51 (m, 2H), 3.35 (m, 2H), 3.29-3.24 (m, 2H), 2.92 (m, 2H), 2.61 (m, 2H).

Table 2 lists examples that were prepared according to the procedures as described in Examples 10-12 by using the corresponding intermediates and reagents under appropriate conditions that could be accomplished by the skilled persons.

TABLE 2

| Ex. | Structure | Chemical | Mass | |
|---|---|---|---|---|
| # | (Synthetic Method) | Name | m/z | 1H NMR |
| 13 | 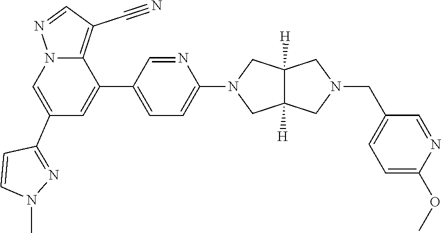 [SMILES: COc1ccc(CN2C[C@@H]3CN(c4ccc(-c5cc(-c6ccn(C)n6)cn6ncc(C#N)c56)cn4)C[C@@H]3C2)cn1] | 4-(6-((3aR,6aS)-5-((6- methoxypyridin-3- yl)methyl) hexahydropyrrolo [3,4-c]pyrrol-2(1H)- yl)pyridin-3-yl)-6-(1- methyl-1H-pyrazol-3- yl)pyrazolo[1,5- a]pyridine-3- carbonitrile | 532.3 | ¹H NMR (400 MHz, DMSO-d₆) δ 9.25 (s, 1H), 8.67 (s, 1H), 8.35 (s, 1H), 8.05 (s, 1H), 7.85 (s, 1H), 7.80 (d, J = 2.1 Hz, 2H), 7.63 (s, 1H), 6.97 (d, J = 2.2 Hz, 1H), 6.78 (s, 1H), 6.64 (s, 1H), 3.90 (s, 3H), 3.82 (s, 3H), 3.57 (m, 8H), 2.94 (s, 2H), 2.60 (s, 2H). |
| 14 | 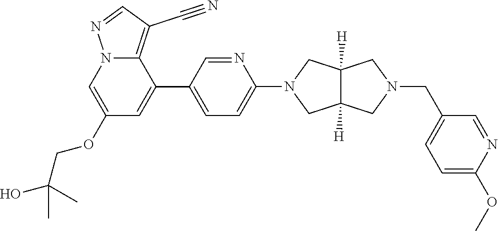 [SMILES: COc1ccc(CN2C[C@@H]3CN(c4ccc(-c5cc(OCC(C)(C)O)cn6ncc(C#N)c56)cn4)C[C@@H]3C2)cn1] | 6-(2-hydroxy-2- methylpropoxy)-4-(6- ((3aR,6aS)-5-((6- methoxypyridin-3- yl)methyl) hexahydropyrrolo [3,4-c]pyrrol-2(1H)- yl)pyridin-3- yl)pyrazolo[1,5- a]pyridine-3- carbonitrile | 540.3 | ¹H NMR (400 MHz, DMSO-d₆) δ 8.64 (d, J = 1.8 Hz, 1H), 8.55 (s, 1H), 8.29 (d, J = 2.2 Hz, 1H), 8.03 (s, 1H), 7.72 (dd, J = 8.7, 2.3 Hz, 1H), 7.64- 7.57 (m, 1H), 7.25 (d, J = 1.8 Hz, 1H), 6.74 (d, J = 8.5 Hz, 1H), 6.59 (d, J = 8.7 Hz, 1H), 4.67 (s, 1H), 3.85 (s, 2H), 3.81 (s, 3H), 3.67- 3.58 (m, 2H), 3.50 (s, 2H), 3.33 (s, 2H), 2.91 (s, 2H), 2.59 (d, J = 5.9 Hz, 2H), 2.44 (s, 2H), 1.21 (s, 6H). |

### Example 15

### N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-6-methoxynicotinamide

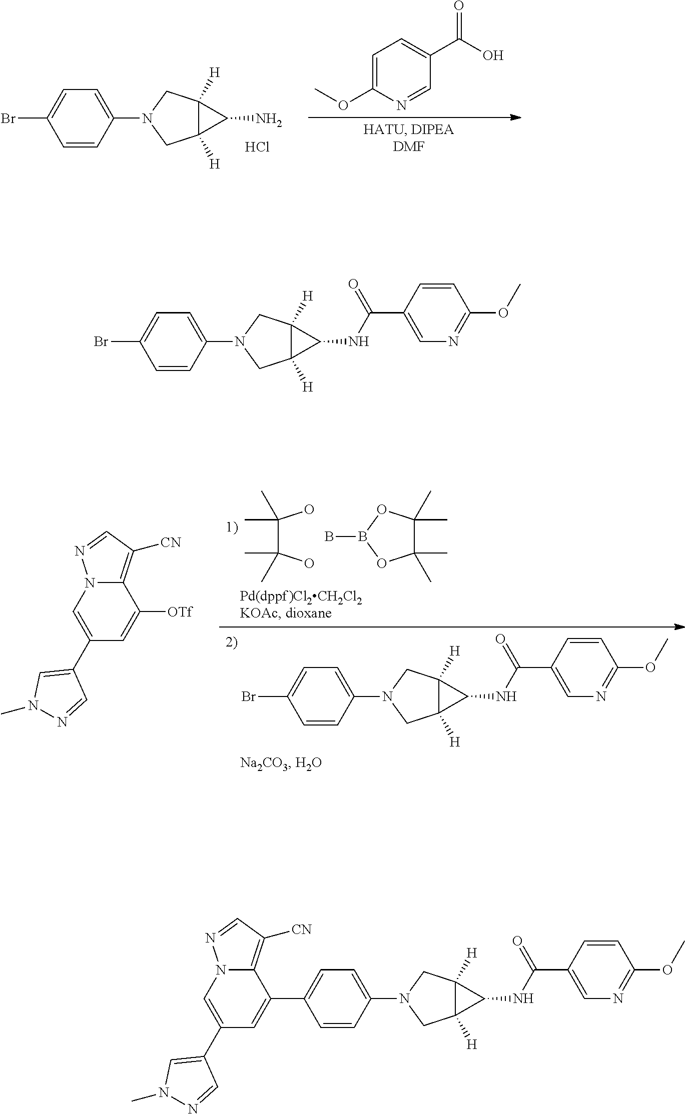

### Step 1. N-((1R,5S,6s)-3-(4-bromophenyl)-3-azabicyclo[3.1.0]hexan-6-yl)-6-methoxynicotinamide

To a solution of Intermediate 9 (crude 101 mg, 0.317 mmol), 6-methoxynicotinic acid (47.1 mg, 0.399 mmol), and HATU (227.5 mg, 0.598 mmol) in DMF (2 mL) was added DIPEA (257.7 mg, 1.994 mmol) at rt. The reaction solution was stirred at rt overnight. After concentrating in vacuo, the residue was dissolved in DCM/MeOH (10/1, 100 mL), which was washed with H₂O (30 mL×2) and brine (30 mL), dried over anhydrous Na₂SO₄, filtered off, and concentrated in vacuo. The residue was purified by prep-TLC (DCM:MeOH=10/1) to give the title compound (100 mg, yield: 65%).

### Step 2. N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-6-methoxynicotinamide

To a suspension of Intermediate 1 (96 mg, 0.258 mmol) and B₂Pin₂ (69 mg, 0.271 mmol) in dry dioxane (0.5 mL) were added dry potassium acetate (50.6 mg, 0.516 mmol) and Pd(dppf)Cl₂ DCM (10.6 mg, 0.0129 mmol). The mixture was flushed with N₂, stirred at 80° C. for 3 h. To the mixture after cooling to rt was added the product of Step 1 above (95 mg, 0.258 mmol), sodium carbonate (54.7 mg, 0.516 mmol) and H₂O (0.1 mL). The reaction mixture was flushed with N₂, stirred at 100° C. overnight. After cooling to rt, the mixture was diluted with DCM/MeOH (10/1, 100 mL). The organic phase was separated, washed with H₂O (30 mL×2), dried over anhydrous Na₂SO₄, filtered off, and concentrated in vacuo. The residue obtained was purified by prep-TLC (DCM:MeOH=10/1) to give the title compound (18 mg, yield: 14%). ESI-MS (m/z): 531.2 [M+1]⁺. ¹H NMR (400 MHz, DMSO-d₆) δ 9.17 (s, 1H), 8.64 (d, J=12.5 Hz, 3H), 8.39 (s, 1H), 8.12 (d, J=6.6 Hz, 2H), 7.67 (s, 1H), 7.47 (d, J=7.9 Hz, 2H), 6.90 (d, J=8.5 Hz, 1H), 6.73 (d, J=8.3 Hz, 2H), 3.91 (s, 3H), 3.88 (s, 3H), 3.74-3.67 (m, 2H), 3.38-3.37 (m, 2H), 2.72-2.67 (m, 1H), 2.04-1.99 (m, 2H).

Table 3 lists examples that were prepared according to the procedures as described in Example 15 by using the corresponding intermediates and reagents under appropriate conditions that could be accomplished by the skilled persons.

TABLE 3

| Ex. | Structure | Chemical | Mass | |
|---|---|---|---|---|
| # | (Synthetic Method) | Name | m/z | 1H NMR |
| 16 | 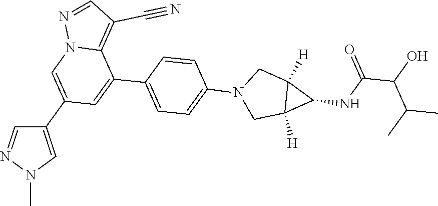 [SMILES: CC(C)C(O)C(=O)N[C@H]1[C@@H]2CN(c3ccc(-c4cc(-c5cnn(C)c5)cn5ncc(C#N)c45)cc3)C[C@@H]21] | N-((1R,5S,6s)-3-(4-(3- cyano-6-(1-methyl-1H- pyrazol-4- yl)pyrazolo[1,5- a]pyridin-4-yl)phenyl)- 3-azabicyclo [3.1.0]hexan- 6-yl)-2-hydroxy-3- methylbutanamide | 496.6 | ¹H NMR (400 MHz, CD₃OD) δ 8.88 (d, J = 1.3 Hz, 1H), 8.37 (s, 1H), 8.13 (s, 1H), 7.96 (s, 1H), 7.58 (d, J = 1.3 Hz, 1H), 7.43 (d, J = 8.6 Hz, 2H), 6.71 (d, J = 8.7 Hz, 2H), 3.95 (s, 3H), 3.82 (d, J = 3.8 Hz, 1H), 3.76 (dd, J = 9.4, 2.6 Hz, 2H), 3.36 (d, J = 3.6 Hz, 1H), 3.34 (d, J = 3.7 Hz, 1H), 2.57-2.53 (m, 1H), 2.12-2.02 (m, 1H), 1.96-1.92 (m, 2H), 1.00 (d, J = 6.9 Hz, 3H), 0.86 (d, J = |
| | | | | 6.8 Hz, 3H). |
| 17 | 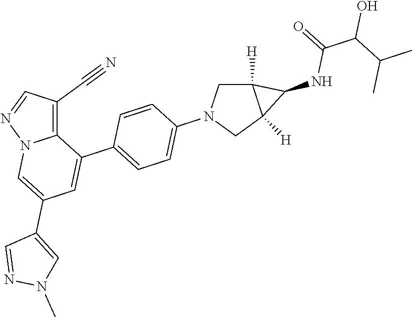 [SMILES: CC(C)C(O)C(=O)N[C@@H]1[C@@H]2CN(c3ccc(-c4cc(-c5cnn(C)c5)cn5ncc(C#N)c45)cc3)C[C@@H]21] | N-((1R,5S,6r)-3-(4-(3- cyano-6-(1-methyl-1H- pyrazol-4- yl)pyrazolo[1,5- a]pyridin-4-yl)phenyl)- 3-azabicyclo [3.1.0]hexan- 6-yl)-2-hydroxy-3- methylbutanamide | 496.6 | ¹H NMR (400 MHz, DMSO- d₆) δ 9.16 (s, 1H), 8.62 (s, 1H), 8.38 (s, 1H), 8.10 (s, 1H), 7.89 (s, 1H), 7.66 (s, 1H), 7.45 (d, J = 8.5 Hz, 2H), 6.69 (d, J = 8.6 Hz, 2H), 5.30 (d, J = 5.5 Hz, 1H), 3.92 (s, 2H), 3.87 (s, 3H), 3.67-3.61 (m, 3H), 2.58- 2.53 (m, 1H), 1.98-1.92 (m, 2H), 1.92-1.86 (m, 1H), 0.89 (d, J = 6.9 Hz, 3H), 0.77 (d, J = 6.8 Hz, 3H). |

### Example 18

### (R)-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-hydroxy-2-phenylacetamide

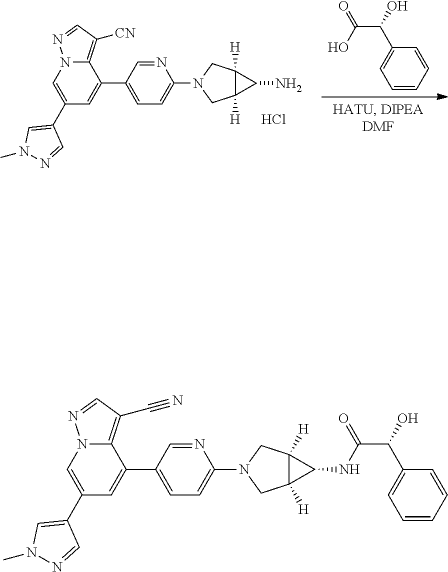

To a solution of Intermediate 7 (50 mg, 0.126 mmol), (R)-2-hydroxy-2-phenylacetic acid (19.2 mg, 0.126 mmol), and HATU (72 mg, 0.189 mmol) in DMF (1 mL) was added DIPEA (81 mg, 0.63 mmol) at rt. The reaction solution was stirred at rt overnight, which was diluted with DCM/MeOH (10/1, 50 mL), washed with H₂O (20 mL×3) and brine (20 mL), dried over anhydrous Na₂SO₄, filtered off, and concentrated in vacuo. The residue obtained was purified by prep-TLC (DCM:MeOH=10/1) to give the title compound (19 mg, yield: 29%). ESI-MS (m/z): 531.2 [M+1]⁺. ¹H NMR (400 MHz, DMSO-d₆) δ 9.19 (s, 1H), 8.62 (s, 1H), 8.37 (s, 1H), 8.32 (d, J=2.3 Hz, 1H), 8.21 (d, J=4.4 Hz, 1H), 8.09 (s, 1H), 7.76 (dd, J=8.7, 2.4 Hz, 1H), 7.72 (s, 1H), 7.40 (d, J=7.3 Hz, 2H), 7.31 (t, J=7.3 Hz, 2H), 7.26 (d, J=7.2 Hz, 1H), 6.58 (d, J=8.9 Hz, 1H), 6.13 (d, J=4.6 Hz, 1H), 4.88 (d, J=4.6 Hz, 1H), 3.86 (s, 3H), 3.74 (in), 3.46 (m, 2H), 2.45-2.43 (m, 1H), 1.92 (in, 2H).

Table 4 lists examples that were prepared according to the procedures as described in Example 18 by using the corresponding intermediates and reagents under appropriate conditions that could be accomplished by the skilled persons.

TABLE 4

| Ex. | Structure | Chemical | Mass | |
|---|---|---|---|---|
| # | (Synthetic Method) | Name | m/z | 1H NMR |
| 19 | 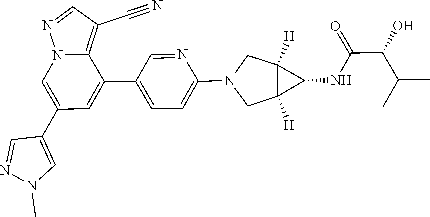 [SMILES: CC(C)[C@@H](O)C(=O)N[C@H]1[C@@H]2CN(c3ccc(-c4cc(-c5cnn(C)c5)cn5ncc(C#N)c45)cn3)C[C@@H]21] | (R)-N- ((1R,5S,6s)-3- (5-(3-cyano-6-(1- methyl-1H-pyrazol- 4-yl)pyrazolo[1,5- a]pyridin-4- yl)pyridin-2-yl)-3- azabicyclo[3.1.0] hexan-6-yl)- 2-hydroxy-3- methylbutanamide | 497.4 | ¹H NMR (400 MHz, DMSO-d₆) δ 9.20 (d, J = 1.2 Hz, 1H), 8.62 (s, 1H), 8.37 (s, 1H), 8.33 (d, J = 2.3 Hz, 1H), 8.10 (s, 1H), 7.89 (d, J = 4.2 Hz, 1H), 7.77 (dd, J = 8.7, 2.4 Hz, 1H), 7.73 (d, J = 1.2 Hz, 1H), 6.60 (d, J = 8.8 Hz, 1H), 5.30 (d, J = 5.5 Hz, 1H), 3.86 (s, 3H), 3.77 (d, J = 10.5 Hz, 2H), 3.47 (s, 2H), 2.43 (s, 2H), 1.94-1.88 (m, 3H), 0.87 (d, J = 6.9 Hz, 3H), 0.75 (d, J = 6.8 Hz, 3H). |
| 20 | 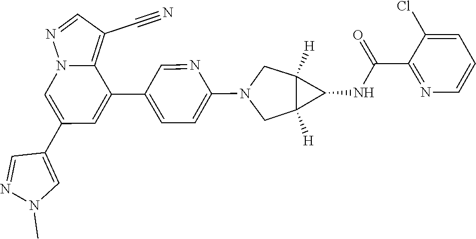 [SMILES: Cn1cc(-c2cc(-c3ccc(N4C[C@@H]5[C@H](C4)[C@H]5NC(=O)c4ncccc4Cl)nc3)c3c(C#N)cnn3c2)cn1] | 3-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6- yl)picolinamide | 536.2 | ¹H NMR (400 MHz, DMSO-d₆) δ 9.21 (s, 1H), 8.84 (d, J = 4.1 Hz, 1H), 8.63 (s, 1H), 8.54 (d, J = 4.6 Hz, 1H), 8.38 (s, 1H), 8.35 (s, 1H), 8.11 (s, 1H), 8.02 (d, J = 8.1 Hz, 1H), 7.79 (dd, J = 8.7, 2.2 Hz, 1H), 7.75 (s, 1H), 7.53 (dd, J = 8.1, 4.6 Hz, 1H), 6.63 (d, J = 8.7 Hz, 1H), 3.86 (s, 3H), 3.83 (m, 2H), 3.51 (d, J = 9.8 Hz, 2H), 2.64 (s, 1H), 1.98 (s, 2H). |
| 21 | 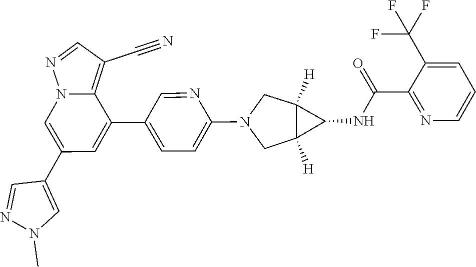 [SMILES: Cn1cc(-c2cc(-c3ccc(N4C[C@@H]5[C@H](C4)[C@H]5NC(=O)c4ncccc4C(F)(F)F)nc3)c3c(C#N)cnn3c2)cn1] | N-((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)-3- (trifluoromethyl) picolinamide | 570.2 | ¹H NMR (400 MHz, DMSO-d₆) δ 9.21 (s, 1H), 8.91 (d, J = 3.9 Hz, 1H), 8.84 (d, J = 4.7 Hz, 1H), 8.63 (s, 1H), 8.38 (s, 1H), 8.35 (d, J = 2.2 Hz, 1H), 8.30 (d, J = 1.5 Hz, 1H), 8.11 (s, 1H), 7.79 (dd, J = 8.7, 2.4 Hz, 1H), 7.76-7.71 (m, 2H), 6.63 (d, J = 8.9 Hz, 1H), 3.86 (s, 3H), 3.83 (s, 2H), 3.52 (d, J = 8.8 Hz, 2H), 2.61 (s, 1H), 1.96 (s, 2H). |
| 22 | 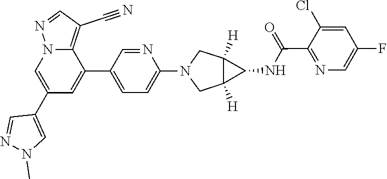 [SMILES: Cn1cc(-c2cc(-c3ccc(N4C[C@@H]5[C@H](C4)[C@H]5NC(=O)c4ncc(F)cc4Cl)nc3)c3c(C#N)cnn3c2)cn1] | 3-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)-5- fluoropicolinamide | 554.2 | ¹H NMR (400 MHz, DMSO-d₆) δ 9.21 (s, 1H), 8.86 (d, J = 4.0 Hz, 1H), 8.65-8.60 (m, 2H), 8.38 (s, 1H), 8.35 (d, J = 2.3 Hz, 1H), 8.20 (dd, J = 8.8, 2.3 Hz, 1H), 8.11 (s, 1H), 7.79 (dd, J = 8.6, 2.4 Hz, 1H), 7.74 (s, 1H), 6.63 (d, J = 8.8 Hz, 1H), 3.86 (s, 3H), 3.84 (d, J = 11.1 Hz, 2H), 3.51 (d, J = 9.5 Hz, 2H), 2.64 (s, 1H), 1.98 (s, 2H). |
| 23 | 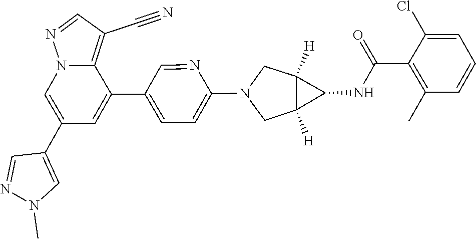 [SMILES: Cc1cccc(Cl)c1C(=O)N[C@H]1[C@@H]2CN(c3ccc(-c4cc(-c5cnn(C)c5)cn5ncc(C#N)c45)cn3)C[C@@H]21] | 2-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)-6- methylbenzamide | 549.4 | 1H NMR (400 MHz, DMSO- d₆) δ 9.21 (d, J = 1.1 Hz, 1H), 8.68 (d, J = 3.7 Hz, 1H), 8.63 (s, 1H), 8.38 (s, 1H), 8.35 (d, J = 2.2 Hz, 1H), 8.10 (s, 1H), 7.79 (dd, J = 8.7, 2.4 Hz, 1H), 7.74 (d, J = 1.2 Hz, 1H), 7.29 (d, J = 4.6 Hz, 2H), 7.21 (dd, J = 8.8, 4.2 Hz, 1H), 6.62 (d, J = 8.8 Hz, 1H), 3.86 (s, 3H), 3.83 (d, J = 10.8 Hz, 2H), 3.51 (d, J = 9.8 Hz, 2H), 2.61-2.57 (m, 1H), 2.24 (s, 3H), 1.93 (s, 2H). |
| 24 | 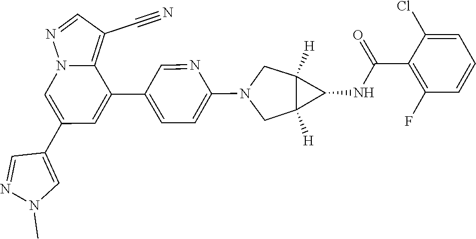 [SMILES: Cn1cc(-c2cc(-c3ccc(N4C[C@@H]5[C@H](C4)[C@H]5NC(=O)c4c(F)cccc4Cl)nc3)c3c(C#N)cnn3c2)cn1] | 2-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)-6- fluorobenzamide | 553.2 | 1H NMR (400 MHz, DMSO- d₆) δ 9.20 (d, J = 2.1 Hz, 1H), 8.91 (d, J = 4.0 Hz, 1H), 8.63 (s, 1H), 8.38 (s, 1H), 8.35 (d, J = 2.2 Hz, 1H), 8.10 (s, 1H), 7.79 (dd, J = 8.7, 2.4 Hz, 1H), 7.74 (d, J = 1.2 Hz, 1H), 7.51- 7.44 (m, 1H), 7.37 (d, J = 8.0 Hz, 1H), 7.30 (t, J = 8.6 Hz, 1H), 6.62 (d, J = 8.7 Hz, 1H), 3.86 (s, 3H), 3.84 (d, J = 10.9 Hz, 2H), 3.51 (d, J = 9.6 Hz, 2H), 2.66-2.60 (m, 1H), 1.92 (s, 2H). |
| 25 | 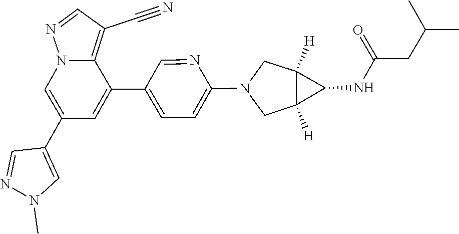 [SMILES: CC(C)CC(=O)N[C@H]1[C@@H]2CN(c3ccc(-c4cc(-c5cnn(C)c5)cn5ncc(C#N)c45)cn3)C[C@@H]21] | N-((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)-3- methylbutanamide | 481.2 | 1H NMR (400 MHz, DMSO- d₆) δ 9.20 (s, 1H), 8.62 (s, 1H), 8.37 (s, 1H), 8.33 (d, J = 2.2 Hz, 1H), 8.10 (s, 1H), 7.98 (d, J = 3.6 Hz, 1H), 7.77 (dd, J = 8.7, 2.4 Hz, 1H), 7.73 (s, 1H), 6.59 (d, J = 8.8 Hz, 1H), 3.86 (s, 3H), 3.76 (d, J = 10.6 Hz, 2H), 3.46 (d, J = 9.9 Hz, 2H), 2.38 (s, 1H), 1.99-1.93 (m, 1H), 1.90 (d, J = 5.9 Hz, 2H), 1.79 (s, 2H), 0.85 (d, J = 6.2 Hz, 6H). |
| 26 | 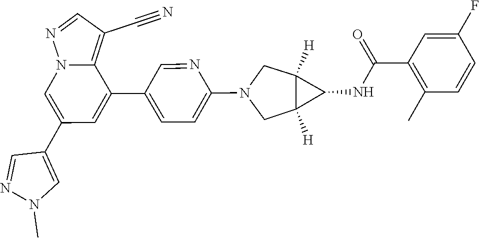 [SMILES: Cc1ccc(F)cc1C(=O)N[C@H]1[C@@H]2CN(c3ccc(-c4cc(-c5cnn(C)c5)cn5ncc(C#N)c45)cn3)C[C@@H]21] | N-((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)- 5-fluoro-2- methylbenzamide | 533.3 | ¹H NMR (400 MHz, DMSO-d₆) δ 9.21 (s, 1H), 8.63 (s, 1H), 8.51 (s, 1H), 8.36 (d, J = 11.7 Hz, 2H), 8.10 (s, 1H), 7.79 (d, J = 8.3 Hz, 1H), 7.74 (s, 1H), 7.26 (s, 1H), 7.15 (d, J = 7.8 Hz, 2H), 6.62 (d, J = 8.1 Hz, 1H), 3.86 (s, 3H), 3.83 (d, J = 11.0 Hz, 2H), 3.51 (d, J = 10.0 Hz, 2H), 2.61 (s, 1H), 2.29 (s, 3H), 1.96 (s, 2H). |
| 27 | 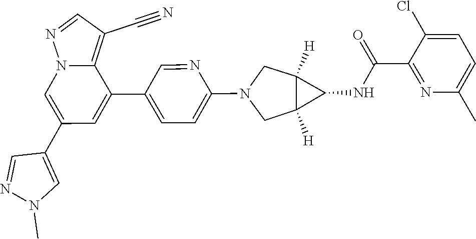 [SMILES: Cc1ccc(Cl)c(C(=O)N[C@H]2[C@@H]3CN(c4ccc(-c5cc(-c6cnn(C)c6)cn6ncc(C#N)c56)cn4)C[C@@H]32)n1] | 3-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)-6- methylpicolinamide | 550.4 | 1H NMR (400 MHz, DMSO- d₆) δ 9.20 (d, J = 1.1 Hz, 1H), 8.71 (d, J = 4.1 Hz, 1H), 8.63 (s, 1H), 8.42-8.34 (m, 2H), 8.10 (s, 1H), 7.86-7.77 (m, 2H), 7.74 (d, J = 1.1 Hz, 1H), 7.54 (d, J = 8.1 Hz, 1H), 6.63 (d, J = 8.7 Hz, 1H), 3.86 (s, 3H), 3.84 (d, J = 10.7 Hz, 2H), 3.51 (d, J = 9.8 Hz, 2H), 2.65- 2.60 (m, 1H), 2.44 (s, 3H), 2.03 (s, 2H). |
| 28 | 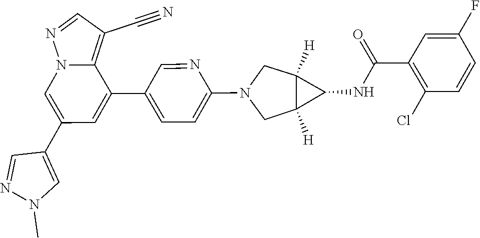 [SMILES: Cn1cc(-c2cc(-c3ccc(N4C[C@@H]5[C@H](C4)[C@H]5NC(=O)c4cc(F)ccc4Cl)nc3)c3c(C#N)cnn3c2)cn1] | 2-chloro-N- ((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)-5- fluorobenzamide | 553.3 | ¹H NMR (400 MHz, DMSO-d₆) δ 9.20 (s, 1H), 8.71 (d, J = 4.0 Hz, 1H), 8.63 (s, 1H), 8.37 (s, 1H), 8.34 (d, J = 2.3 Hz, 1H), 8.10 (s, 1H), 7.79 (dd, J = 8.7, 2.4 Hz, 1H), 7.74 (d, J = 1.0 Hz, 1H), 7.54 (dd, J = 8.8, 4.9 Hz, 1H), 7.40-7.28 (m, 2H), 6.62 (d, J = 8.8 Hz, 1H), 3.86 (s, 3H), 3.83 (d, J = 10.7 Hz, 2H), 3.51 (d, J = 9.7 Hz, 2H), 2.64-2.58 (m, 1H), 1.96 (s, 2H). |
| 29 | 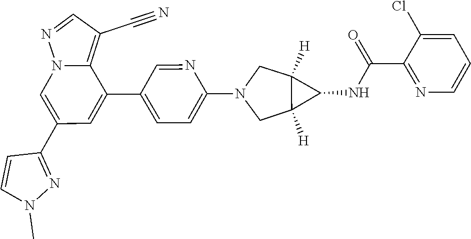 [SMILES: Cn1ccc(-c2cc(-c3ccc(N4C[C@@H]5[C@H](C4)[C@H]5NC(=O)c4ncccc4Cl)nc3)c3c(C#N)cnn3c2)n1] | 3-chloro-N- ((1R,5S,6s)-3-(5-(3- cyano-6-(1-methyl- 1H-pyrazol-3- yl)pyrazolo[1,5- a]pyridin-4- yl)pyridin-2-yl)-3- azabicyclo[3.1.0] hexan-6-yl) picolinamide | 536.3 | ¹H NMR (400 MHz, DMSO-d₆) δ 9.26 (s, 1H), 8.86 (s, 1H), 8.68 (s, 1H), 8.54 (s, 1H), 8.34 (s, 1H), 8.02 (s, 1H), 7.85 (s, 1H), 7.80 (s, 2H), 7.53 (s, 1H), 6.98 (s, 1H), 6.64 (s, 1H), 3.89 (s, 3H), 3.83 (d, J = 9.5 Hz, 2H), 3.51 (d, J = 8.8 Hz, 2H), 2.65 (s, 1H), 1.98 (s, 2H). |
| 30 | 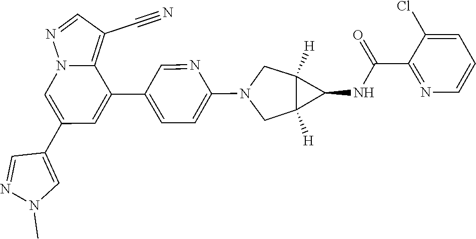 [SMILES: Cn1cc(-c2cc(-c3ccc(N4C[C@@H]5[C@H](C4)[C@@H]5NC(=O)c4ncccc4Cl)nc3)c3c(C#N)cnn3c2)cn1] | 3-chloro-N- ((1R,5S,6r)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl) picolinamide | 536.3 | ¹H NMR (400 MHz, DMSO-d₆) δ 9.18 (s, 1H), 8.61 (s, 1H), 8.47 (s, 1H), 8.42-8.34 (m, 2H), 8.30 (s, 1H), 8.10 (s, 1H), 7.88 (d, J = 7.9 Hz, 1H), 7.74 (d, J = 8.7 Hz, 1H), 7.68 (s, 1H), 7.41 (dd, J = 8.0, 4.6 Hz, 1H), 6.47 (d, J = 8.7 Hz, 1H), 3.86 (s, 3H), 3.68 (m, 4H), 2.96 (s, 1H), 2.10 (d, J = 5.2 Hz, 2H). |

### Example 31

### N-((1R,5 S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)isobutyramide

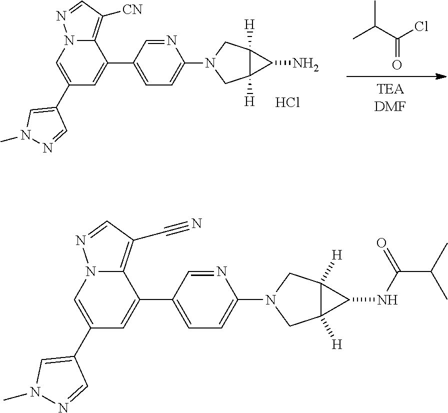

To a solution of Intermediate 7 (50 mg, 0.126 mmol) in DMF (2 mL) was added TEA (0.5 mL) at 0 TC, followed by the addition of isobutyryl chloride (14.8 mg, 0.138 mmol) dropwisely. The reaction solution was stirred at rt overnight. After concentrating in vacuo, the residue was diluted with DCM/MeOH (10/1, 100 mL), washed with H₂O (30 mL×2) and brine (30 mL), dried over anhydrous Na₂SO₄, filtered off, and concentrated in vacuo. The residue was purified by prep-TLC (DCM/MeOH=10/1) to give the title compound (25 mg, yield: 43%). ESI-MS (m/z): 467.3 [M+1]⁺. ¹H NMR (400 MHz, DMSO-d₆) δ 9.20 (s, 1H), 8.62 (s, 1H), 8.37 (s, 1H), 8.33 (s, 1H), 8.10 (s, 1H), 7.92 (d, J=3.7 Hz, 1H), 7.77 (d, J=10.7 Hz, 1H), 7.73 (s, 1H), 6.59 (d, J=8.7 Hz, 1H), 3.86 (s, 3H), 3.75 (d, J=10.6 Hz, 2H), 3.47 (d, J=9.0 Hz, 2H), 2.40 (s, 1H), 2.31-2.24 (m, 1H), 1.80 (s, 2H), 0.98 (d, J=6.8 Hz, 6H).

### Example 32

### 2-amino-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-phenylacetamide

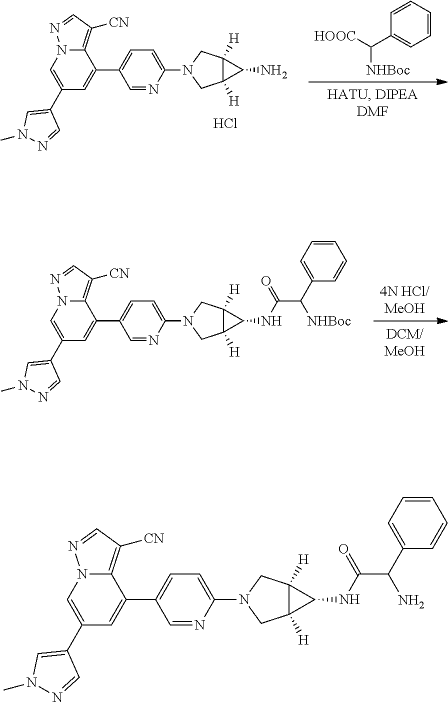

### Step 1. tert-butyl (2-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)amino)-2-oxo-1-phenylethyl)carbamate

This compound was synthesized by following the procedure used to Example 18 starting from 2-(tert-butoxycarbonylamino)-2-phenylacetic acid in place of (R)-2-hydroxy-2-phenylacetic acid.

### Step 2. 2-amino-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-phenylacetamide

To a solution of the product of step 1 above (89 mg, 0.141 mmol) in DCM/MeOH (4/1, 10 mL) was added HCl/dioxane (4 N, 4 mL, 4 mmol) at 0° C. The reaction solution was stirred at rt for 4h before neutralizing with saturated aqueous Na₂CO₃ to pH 7-8. The mixture was extracted with DCM/MeOH (10/1, 100 mL×2). The combined organics were dried over and concentrated in vacuo. The residue was purified by prep-TLC (DCM/MeOH=10/1) to give the title compound (43 mg, yield: 61%). ESI-MS (m/z): 530.2 [M+1]⁺. ¹H NMR (400 MHz, DMSO-d₆) δ 9.19 (d, J=1.2 Hz, 1H), 8.62 (s, 1H), 8.36 (s, 1H), 8.32 (d, J=2.3 Hz, 1H), 8.26 (s, 1H), 8.09 (s, 1H), 7.76 (dd, J=8.7, 2.4 Hz, 1H), 7.72 (d, J=1.3 Hz, 1H), 7.37 (d, J=7.3 Hz, 2H), 7.30 (t, J=7.4 Hz, 2H), 7.23 (t, J=7.2 Hz, 1H), 6.58 (d, J=8.8 Hz, 1H), 4.31 (s, 1H), 3.86 (s, 3H), 3.75 (dd, J=10.6, 6.9 Hz, 2H), 3.46 (dd, J=9.8, 5.2 Hz, 2H), 2.43 (s, 1H), 1.83 (s, 2H).

### Example 33

### 4-(6-((1R,5S,6s)-6-(((6-methoxypyridin-3-yl)methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile

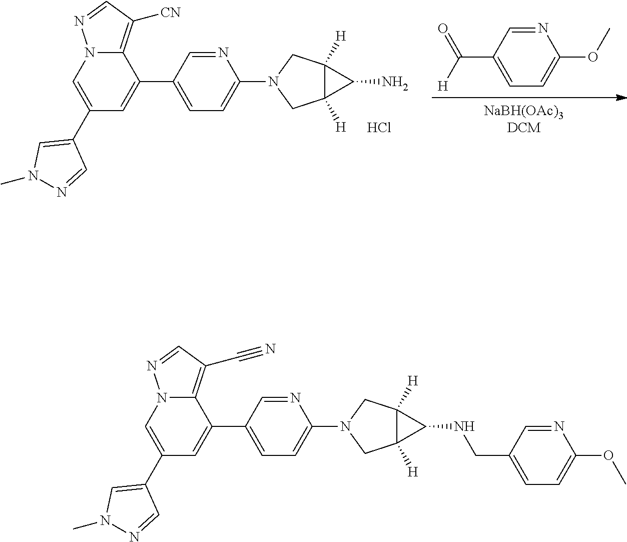

To a solution of Intermediate 7 (50 mg, 0.126 mmol) in DCM (1 mL) was added 6-methoxynicotinaldehyde (17.3 mg, 0.126 mmol). The mixture was stirred at rt for 30 min before adding NaBH(OAc)₃ (53.3 mg, 0.252 mmol). The mixture was stirred at rt overnight, which was basified with saturated aqueous NaHCO₃ to pH 8-9. The mixture was extracted with DCM/MeOH (10/1, 100 mL). The organic layer was washed with H₂O (30 mL×2) and brine (30 mL), dried over anhydrous Na₂SO₄, filtered off, and concentrated in vacuo. The residue was purified by prep-TLC (DCM/MeOH=10/1) to give the title compound (23 mg, yield: 34%). ESI-MS (m/z): 518.4 [M+1]⁺. ¹H NMR (400 MHz, DMSO-d₆) δ 9.19 (s, 1H), 8.62 (s, 1H), 8.36 (s, 1H), 8.30 (d, J=2.2 Hz, 1H), 8.09 (s, 1H), 8.07 (s, 1H), 7.74 (dd, J=8.7, 2.3 Hz, 1H), 7.71 (s, 1H), 7.68-7.62 (m, 1H), 6.75 (d, J=8.4 Hz, 1H), 6.51 (d, J=8.7 Hz, 1H), 3.86 (s, 3H), 3.80 (s, 3H), 3.66 (s, 2H), 3.60 (d, J=10.6 Hz, 2H), 3.41 (d, J=9.5 Hz, 2H), 1.80 (s, 1H), 1.67 (s, 2H).

### Example 34

### 4-(6-((1R,5S,6s)-6-(((6-methoxypyridin-3-yl)methyl)(methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile

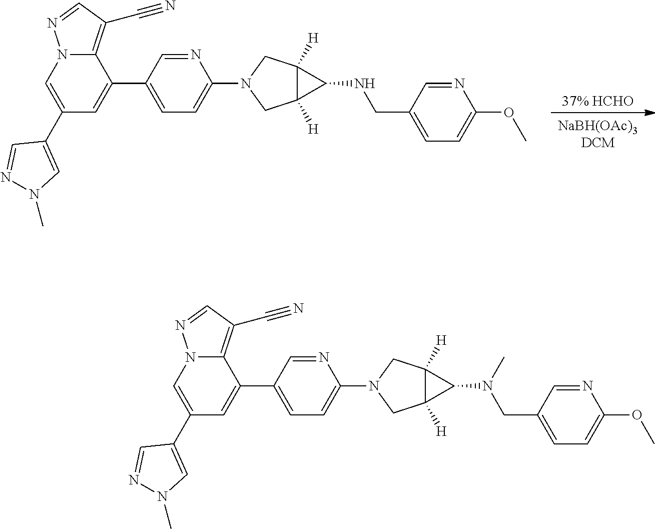 [SMILES: COc1ccc(CN[C@H]2[C@@H]3CN(c4ccc(-c5cc(-c6cnn(C)c6)cn6ncc(C#N)c56)cn4)C[C@@H]32)cn1 ; "37% HCHO" ; "NaBH(OAc)3DCM" ; COc1ccc(CN(C)[C@H]2[C@@H]3CN(c4ccc(-c5cc(-c6cnn(C)c6)cn6ncc(C#N)c56)cn4)C[C@@H]32)cn1]

To a solution of Example 33 (115 mg, 0.222 mmol) in DCM/MeOH (10/1, 4 mL) was added 37% formaldehyde (89 mg, 1.11 mmol). The mixture was stirred at rt for 30 min before adding NaBH(OAc)₃ (94 mg, 0.444 mmol) and AcOH (13 mg, 0.222 mmol). The mixture was stirred at rt overnight. The mixture was diluted with DCM/MeOH (10/1, 100 mL), which was washed with saturated aqueous NaHCO₃ (20 mL) and brine (20 mL), dried over anhydrous Na₂SO₄, filtered off, and concentrated in vacuo. The residue was purified by prep-TLC (DCM/MeOH=20/1) to give the title compound (23 mg, yield: 20%). ESI-MS (m/z): 532.1 [M+1]⁺. ¹H NMR (400 MHz, CDCl₃) δ 8.60 (d, J=1.3 Hz, 1H), 8.25-8.16 (m, 2H), 7.98 (d, J=1.9 Hz, 1H), 7.74 (s, 1H), 7.70 (s, 1H), 7.64 (dd, J=8.7, 2.4 Hz, 1H), 7.50 (dd, J=8.4, 2.1 Hz, 1H), 7.34 (d, J=1.3 Hz, 1H), 6.69 (d, J=8.4 Hz, 1H), 6.41 (d, J=8.8 Hz, 1H), 3.92 (s, 3H), 3.87 (s, 3H), 3.64 (d, J=10.3 Hz, 2H), 3.57 (s, 2H), 3.46 (d, J=10.1 Hz, 2H), 2.27 (s, 3H), 1.73 (s, 2H), 1.57 (s, 1H).

### Example 35

### 2-chloro-N-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzenesulfonamide

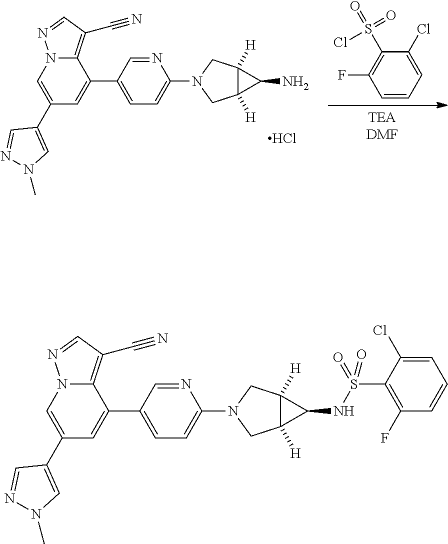

This compound was synthesized by following the procedure used to make Example 11 starting from Intermediate 8 in place of Intermediate 5 (ESI-MS (m/z): 589.3 [M+1]⁺. ¹H NMR (400 MHz, DMSO-d₆) δ 9.19 (s, 1H), 8.63 (s, 1H), 8.37 (d, J=13.9 Hz, 2H), 8.30 (s, 1H), 8.11 (s, 1H), 7.74 (d, J=7.9 Hz, 2H), 7.60 (d, J=5.6 Hz, 1H), 7.44 (d, J=7.9 Hz, 1H), 7.37 (t, J=9.5 Hz, 1H), 6.39 (d, J=8.7 Hz, 1H), 3.86 (s, 3H), 3.70 (d, J=10.2 Hz, 2H), 3.58 (d, J=9.6 Hz, 2H), 3.30 (s, 1H), 1.97 (s, 2H).

### Example 36

### 1-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-phenylurea

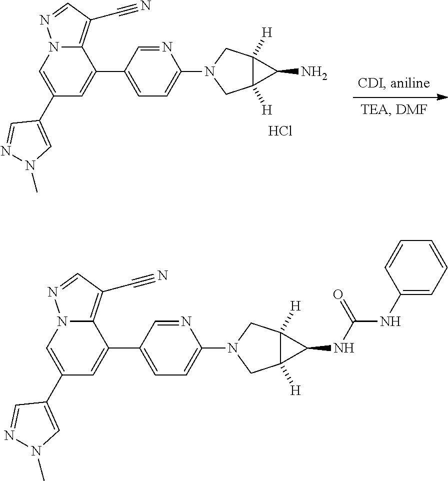 [SMILES: Cn1cc(-c2cc(-c3ccc(N4C[C@@H]5[C@H](N)[C@@H]5C4)nc3)c3c(C#N)cnn3c2)cn1 ; Cl ; "CDI, aniline" ; "TEA, DMF" ; Cn1cc(-c2cc(-c3ccc(N4C[C@@H]5[C@H](C4)[C@@H]5NC(=O)Nc4ccccc4)nc3)c3c(C#N)cnn3c2)cn1]

To a solution of Intermediate 8 (100 mg, 0.17 mmol) in DMF (2 mL) was added TEA (0.5 mL), followed by the addition of CDI (86 mg, 0.53 mmol). The mixture was stirred at rt for 10 min before adding aniline (32 mg, 0.34 mmol). The reaction mixture was stirred at 50° C. for 4h. The reaction mixture was diluted with EtOAc (100 mL), washed with H₂O (30 mL×2) and brine (30 mL), dried over anhydrous Na₂SO₄, filtered off, and concentrated in vacuo. The residue was purified by prep-TLC to give the title compound (25 mg, yield: 28%). ESI-MS (m/z): 516.4 [M+1]⁺. ¹H NMR (400 MHz, DMSO-d₆) δ 9.19 (s, 1H), 8.63 (s, 1H), 8.49 (s, 1H), 8.40-8.30 (m, 2H), 8.09 (s, 1H), 7.81-7.74 (m, 1H), 7.70 (s, 1H), 7.34 (d, J=8.0 Hz, 2H), 7.18 (t, J=7.8 Hz, 2H), 6.86 (t, J=7.4 Hz, 1H), 6.54 (d, J=8.8 Hz, 1H), 6.11 (s, 1H), 3.85 (s, 3H), 3.68 (d, J=10.7 Hz, 2H), 3.47 (d, J=11.1 Hz, 2H), 2.93-2.83 (m, 1H), 2.01 (d, J=6.2 Hz, 2H).

### Example 37

### 3-chloro-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)picolinamide

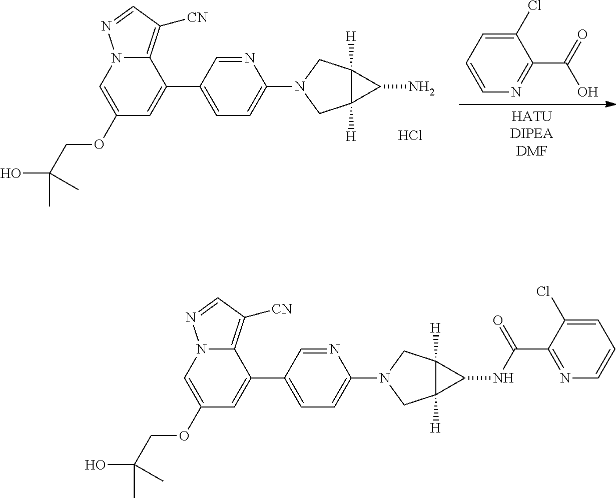

To a solution of Intermediate 12 (50 mg, crude, 0.1 mmol) in DMF (1 mL) was added 3-chloropicolinic acid (19.5 mg, 0.124 mmol), HATU (71 mg, 0.186 mmol) and DIPEA (80 mg, 0.62 mmol) sequentially at rt. The reaction mixture was stirred at 45° C. overnight. After cooling to rt, the mixture was directly purified via reverse phase flash column chromatography (H₂O/MeOH=9/1 to MeOH) to give the title compound (21 mg, yield: 31%). ESI-MS (m/z): 544.3 [M+1]⁺. ¹H NMR (400 MHz, CD₃OD) δ 8.52 (d, J=4.6 Hz, 1H), 8.44 (d, J=1.9 Hz, 1H), 8.33 (s, 1H), 8.25 (d, J=2.2 Hz, 1H), 7.97 (d, J=8.2 Hz, 1H), 7.76 (dd, J=8.8, 2.3 Hz, 1H), 7.50 (dd, J=8.2, 4.7 Hz, 14), 7.29 (d, J=1.9 Hz, 16), 6.67 (d, J=8.8 Hz, 1H), 3.96 (d, J=10.5 Hz, 2H), 3.91 (s, 2H), 3.61 (d, J=11.3 Hz, 2H), 2.69 (s, 1H), 2.09 (s, 2H), 1.35 (s, 6H).

Table 5 lists examples that were prepared according to the procedures as described in Example 31-37 by using the corresponding intermediates and reagents under appropriate conditions that could be accomplished by the skilled persons.

TABLE 5

| Ex. | Structure | Chemical | Mass | |
|---|---|---|---|---|
| # | (Synthetic Method) | Name | m/z | 1H NMR |
| 38 | 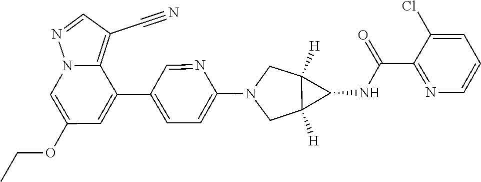 [SMILES: CCOc1cc(-c2ccc(N3C[C@@H]4[C@H](C3)[C@H]4NC(=O)c3ncccc3Cl)nc2)c2c(C#N)cnn2c1] | 3-chloro-N- ((1R,5S,6s)-3- (5-(3-cyano-6- ethoxypyrazolo [1,5-a]pyridin-4- yl)pyridin-2-yl)-3- azabicyclo[3.1.0] hexan-6-yl) picolinamide | 500.2 | ¹H NMR (400 MHz, CD₃OD) δ 8.51 (d, J = 4.6 Hz, 1H), 8.36 (d, J = 2.0 Hz, 1H), 8.30 (s, 1H), 8.24 (s, 1H), 7.95 (d, J = 7.1 Hz, 1H), 7.74 (d, J = 8.7 Hz, 1H), 7.49 (dd, J = 7.9, 4.3 Hz, 1H), 7.19 (d, J = 2.0 Hz, 1H), 6.65 (d, J = 8.6 Hz, 1H), 4.14 (q, J = 7.0 Hz, 2H), 3.96 (m, 2H), 3.60 (m, 2H), 2.69 (s, 1H), 2.09 (m, 2H), 1.46 (t, J = 7.0 Hz, 3H). |
| 39 | 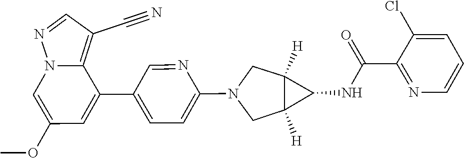 [SMILES: COc1cc(-c2ccc(N3C[C@@H]4[C@H](C3)[C@H]4NC(=O)c3ncccc3Cl)nc2)c2c(C#N)cnn2c1] | 3-chloro-N- ((1R,5S,6s)-3- (5-(3-cyano-6- methoxypyrazolo [1,5-a]pyridin-4- yl)pyridin-2-yl)-3- azabicyclo[3.1.0] hexan-6-yl) picolinamide | 486.2 | ¹H NMR (400 MHz, CD₃OD) δ 8.50 (d, J = 3.5 Hz, 1H), 8.37 (d, J = 2.0 Hz, 1H), 8.29 (s, 1H), 8.23 (s, 1H), 7.94 (d, J = 6.9 Hz, 1H), 7.73 (d, J = 11.3 Hz, 1H), 7.49 (dd, J = 8.2, 4.7 Hz, 1H), 7.19 (d, J = 2.0 Hz, 1H), 6.64 (d, J = 8.7 Hz, 1H), 3.96 (m, 2H), 3.92 (s, 3H), 3.62 (m, 2H), 2.69 (s, 1H), 2.08 (s, 2H). |
| 40 | 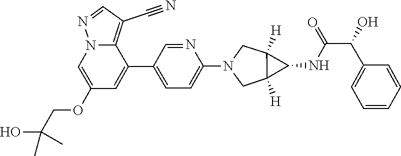 [SMILES: CC(C)(O)COc1cc(-c2ccc(N3C[C@@H]4[C@H](C3)[C@H]4NC(=O)[C@H](O)c3ccccc3)nc2)c2c(C#N)cnn2c1] | (R)-N-((1R,5S,6s)- 3-(5-(3-cyano-6- (2-hydroxy-2- methylpropoxy) pyrazolo[1,5-a] pyridin-4- yl)pyridin-2-yl)-3- azabicyclo[3.1.0] hexan-6-yl)-2- hydroxy-2- | 539.3 | ¹H NMR (400 MHz, CD₃OD) δ 8.42 (d, J = 2.0 Hz, 1H), 8.31 (s, 1H), 8.22 (d, J = 2.2 Hz, 1H), 7.73 (dd, 8.8, 2.4 Hz, 1H), 7.45 (d, J = 7.0 Hz, 2H), 7.37- 7.26 (m, 4H), 6.62 (d, J = 8.8 Hz, 1H), 5.00 (s, 1H), 3.90 (s, 2H), 3.89-3.85 (m, 2H), 3.61- 3.50 (m, 2H), 2.48 (s, 1H), 1.97 (m, 2H), 1.34 (s, 6H). |
| | | phenylacetamide | | |
| 41 | 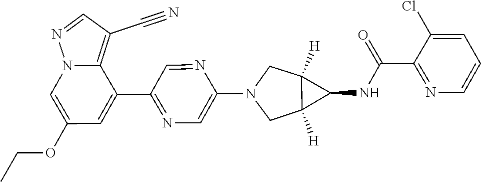 [SMILES: CCOc1cc(-c2cnc(N3C[C@@H]4[C@H](C3)[C@@H]4NC(=O)c3ncccc3Cl)cn2)c2c(C#N)cnn2c1] | 3-chloro-N- ((1R,5S,6r)-3- (5-(3-cyano-6- ethoxypyrazolo [1,5-a]pyridin-4- yl)pyrazin-2-yl)-3- azabicyclo[3.1.0] hexan-6-yl) picolinamide | 501.2 | ¹H NMR (400 MHz, DMSO-d₆) δ 8.63 (d, J = 2.0 Hz, 1H), 8.55 (s, 1H), 8.53 (d, J = 1.1 Hz, 1H), 8.50 (d, J = 2.0 Hz, 1H), 8.36- 8.32 (m, 1H), 7.93 (d, J = 1.1 Hz, 1H), 7.86 (dd, J = 8.2, 1.1 Hz, 1H), 7.50 (d, J = 2.0 Hz, 1H), 7.38 (dd, J = 8.2, 4.7 Hz, 1H), 4.15 (q, J = 7.0 Hz, 2H), 3.75 (m, 4H), 2.94 (m, 1H), 2.13 (m, 2H), 1.37 (t, J = 6.9 Hz, 3H). |
| 42 | 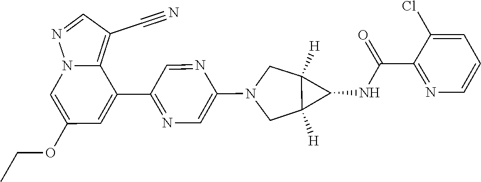 [SMILES: CCOc1cc(-c2cnc(N3C[C@@H]4[C@H](C3)[C@H]4NC(=O)c3ncccc3Cl)cn2)c2c(C#N)cnn2c1] | 3-chloro-N- ((1R,5S,6s)-3- (5-(3-cyano-6- ethoxypyrazolo [1,5-a]pyridin-4- yl)pyrazin-2-yl)-3- azabicyclo[3.1.0] hexan-6-yl) picolinamide | 501.2 | ¹H NMR (400 MHz, DMSO-d₆) δ 8.85 (d, J = 4.1 Hz, 1H), 8.65 (d, J = 2.0 Hz, 1H), 8.56 (s, 1H), 8.54 (d, J = 6.2 Hz, 2H), 8.09 (s, 1H), 8.02 (d, J = 8.3 Hz, 1H), 7.53 (dd, J = 7.4, 5.4 Hz, 2H), 4.15 (q, J = 6.9 Hz, 2H), 3.90 (d, J = 10.9 Hz, 2H), 3.60 (d, J = 9.0 Hz, 2H), 2.65 (s, 1H), 2.02 (s, 2H), 1.37 (t, J = 6.9 Hz, 3H). |
| 43 | 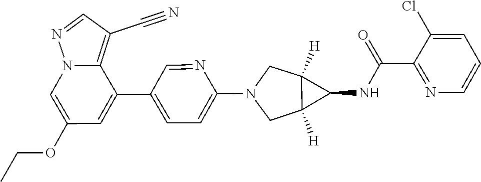 [SMILES: CCOc1cc(-c2ccc(N3C[C@@H]4[C@H](C3)[C@@H]4NC(=O)c3ncccc3Cl)nc2)c2c(C#N)cnn2c1] | 3-chloro-N- ((1R,5S,6r)-3- (5-(3-cyano-6- ethoxypyrazolo [1,5-a]pyridin-4- yl)pyridin-2-yl)-3- azabicyclo[3.1.0] hexan-6-yl) picolinamide | 500.2 | ¹H NMR (400 MHz, DMSO-d₆) δ 8.61 (d, J = 1.8 Hz, 1H), 8.54 (s, 1H), 8.46 (s, 1H), 8.38 (d, J = 3.7 Hz, 1H), 8.24 (d, J = 2.3 Hz, 1H), 7.87 (d, J = 7.2 Hz, 1H), 7.69 (dd, J = 8.7, 2.4 Hz, 1H), 7.41 (dd, J = 8.2, 4.7 Hz, 1H), 7.16 (d, J = 1.9 Hz, 1H), 6.45 (d, J = 8.8 Hz, 1H), 4.13 (q, J = 6.9 Hz, 2H), 3.65 (q, J = 11.1 Hz, 4H), 2.95 (d, J = 2.7 Hz, 1H), |
| | | | | 2.09 (d, J = 6.4 Hz, 2H), 1.36 (t, |
| | | | | J = 6.9 Hz, 3H). |
| 44 | 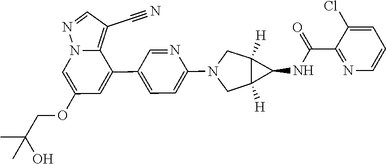 [SMILES: CC(C)(O)COc1cc(-c2ccc(N3C[C@@H]4[C@H](C3)[C@@H]4NC(=O)c3ncccc3Cl)nc2)c2c(C#N)cnn2c1] | 3-chloro-N- ((1R,5S,6r)-3- (5-(3-cyano-6- (2-hydroxy-2- methylpropoxy) pyrazolo[1,5-a] pyridin-4-yl) pyridin-2-yl)-3- azabicyclo[3.1.0] hexan-6-yl) | 544.2 | ¹H NMR (400 MHz, DMSO-d₆) δ 8.62 (d, J = 1.8 Hz, 1H), 8.54 (s, 1H), 8.46 (d, J = 2.2 Hz, 1H), 8.39 (d, J = 4.5 Hz, 1H), 8.25 (d, J = 2.3 Hz, 1H), 7.87 (d, J = 8.2 Hz, 1H), 7.69 (dd, J = 8.7, 2.4 Hz, 1H), 7.41 (dd, J = 8.2, 4.7 Hz, 1H), 7.18 (d, J = 1.9 Hz, 1H), 6.45 (d, J = 8.8 Hz, 1H), 4.68 (s, 1H), 3.85 (s, 2H), 3.66 |
| | | picolinamide | | (q, J = 10.9 Hz, 4H), 2.95 (d, |
| | | | | J = 2.7 Hz, 1H), 2.09 (d, J = 6.3 |
| | | | | Hz, 2H), 1.22 (s, 6H). |
| 45 | 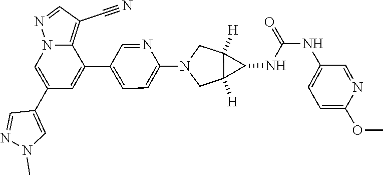 [SMILES: COc1ccc(NC(=O)N[C@H]2[C@@H]3CN(c4ccc(-c5cc(-c6cnn(C)c6)cn6ncc(C#N)c56)cn4)C[C@@H]32)cn1] | 1-((1R,5S,6s)- 3-(5-(3-cyano- 6-(1-methyl- 1H-pyrazol-4- yl)pyrazolo[1,5- a]pyridin-4- yl)pyridin-2-yl)-3- azabicyclo[3.1.0] hexan-6-yl)-3-(6- methoxypyridin-3- yl)urea | 547.4 | ¹H NMR (400 MHz, DMSO-d₆) δ 9.20 (s, 1H), 8.62 (s, 1H), 8.37 (s, 1H), 8.33 (d, J = 2.9 Hz, 2H), 8.14 (d, J = 2.8 Hz, 1H), 8.10 (s, 1H), 7.81-7.70 (m, 3H), 6.71 (d, J = 8.9 Hz, 1H), 6.59 (d, J = 8.8 Hz, 1H), 6.55 (s, 1H), 3.86 (s, 3H), 3.81 (m, 2H), 3.77 (s, 3H), 3.48 (m, 2H), 2.37 (s, 1H), 1.87 (s, 2H). |
| 46 | 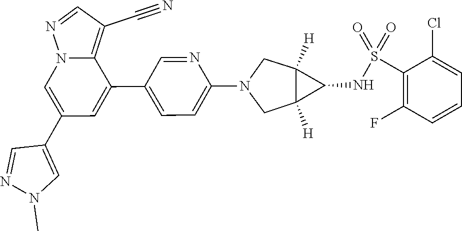 [SMILES: Cn1cc(-c2cc(-c3ccc(N4C[C@@H]5[C@H](C4)[C@H]5NS(=O)(=O)c4c(F)cccc4Cl)nc3)c3c(C#N)cnn3c2)cn1] | 2-chloro-N- ((1R,5S,6s)- 3-(5-(3-cyano- 6-(1-methyl- 1H-pyrazol-4- yl)pyrazolo[1,5- a]pyridin-4- yl)pyridin-2-yl)-3- azabicyclo[3.1.0] hexan-6-yl)-6- fluorobenzene- sulfonamide | 589.4 | ¹H NMR (400 MHz, DMSO-d₆) δ 9.19 (s, 1H), 8.63 (d, J = 17.4 Hz, 2H), 8.32 (d, J = 24.2 Hz, 2H), 8.09 (s, 1H), 7.79-7.62 (m, 3H), 7.52 (d, J = 8.0 Hz, 1H), 7.46 (d, J = 9.9 Hz, 1H), 6.54 (d, J = 8.7 Hz, 1H), 3.85 (s, 3H), 3.61 (d, J = 10.7 Hz, 2H), 3.40 (d, J = 10.4 Hz, 2H), 2.08 (s, 1H), 1.90 (s, 2H). |
| 47 | 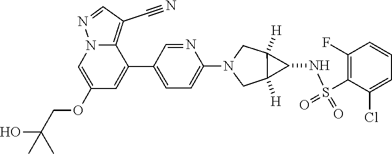 [SMILES: CC(C)(O)COc1cc(-c2ccc(N3C[C@@H]4[C@H](C3)[C@H]4NS(=O)(=O)c3c(F)cccc3Cl)nc2)c2c(C#N)cnn2c1] | 2-chloro-N- ((1R,5S,6s)- 3-(5-(3-cyano-6- (2-hydroxy-2- methylpropoxy) pyrazolo[1,5-a] pyridin-4- yl)pyridin-2-yl)-3- azabicyclo[3.1.0] hexan-6-yl)-6- | 597.3 | ¹H NMR (400 MHz, CDCl₃) δ 8.26 (s, 1H), 8.16 (d, J = 15.8 Hz, 2H), 7.66 (d, J = 8.2 Hz, 1H), 7.48 (d, J = 6.0 Hz, 1H), 7.38 (d, J = 7.9 Hz, 1H), 7.20 (t, J = 9.4 Hz, 1H), 7.09 (s, 1H), 6.42 (d, J = 8.6 Hz, 1H), 5.67 (s, 1H), 3.84 (s, 2H), 3.74 (m, 2H), 3.56 (m, 2H), 2.95 (s, 1H), 2.88 (s, 1H), 2.23 (s, 2H), 1.38 (s, |
| | | fluorobenzene- | | 6H) |
| | | sulfonamide | | |

### Example 48

### 3-chloro-N-((3aR,5r,6aS)-2-(5-(3-cyano-6-ethoxypyrazolo[1,5-a]pyridin-4-yl)pyridin-2-yl)octahydrocyclopenta[c]pyrrol-5-yl)picolinamide

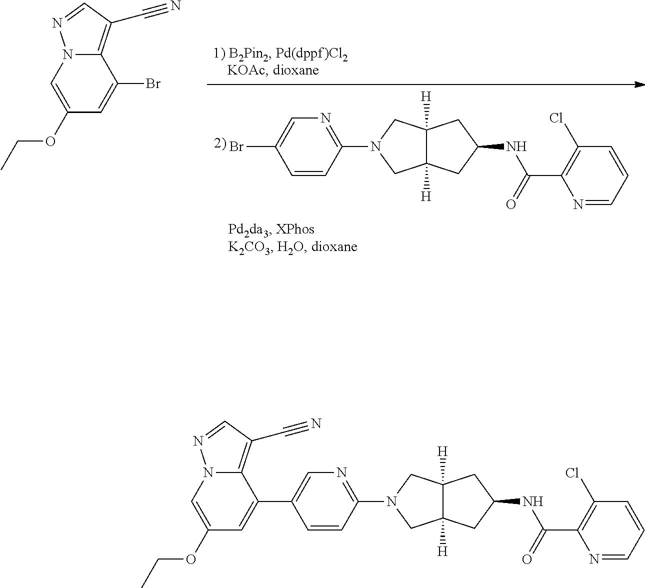

A solution of Intermediate 4 (68 mg, 0.256 mmol), B₂Pin₂ (68 mg, 0.269 mmol), Pd(dppf)Cl₂ DCM (21 mg, 0.0256 mmol), and KOAc (50 mg, 0.512 mmol) was charged with N₂(g) and stirred at 100° C. for 2.5h. To the mixture cooled to rt was added Intermediate 19 (72 mg, 0.171 mmol), Pd₂dba₃ (8 mg, 0.00855 mmol), XPhos (16 mg, 0.0342 mmol), K₂CO₃ (71 mg, 0.513 mmol), and H₂O (1 mL) and dioxane (3 mL) were added successively. The resultant reaction mixture was stirred at 110° C. overnight under N₂. After cooling to rt, the mixture was diluted with DCM/MeOH (10/1, 50 mL), washed with brine (20 mL), dried over anhydrous Na₂SO₄, filtered off, and concentrated in vacuo. The residue was purified by reverse phase flash column chromatography (H₂O/MeOH=20:80 to 80:20) to give the title compound (26 mg, yield: 30%). ESI-MS (m/z): 528.3 [M+1]⁺. ¹H NMR (400 MHz, DMSO-d₆) δ 8.68 (d, J=7.9 Hz, 1H), 8.62 (d, J=1.9 Hz, 1H), 8.55 (s, 1H), 8.48 (d, J=4.6 Hz, 1H), 8.28 (d, J=2.3 Hz, 1H), 8.02-7.93 (m, 1H), 7.72 (dd, J=8.7, 2.4 Hz, 1H), 7.48 (dd, J=8.2, 4.7 Hz, 1H), 7.21 (d, J=1.9 Hz, 1H), 6.59 (d, J=8.8 Hz, 1H), 4.41-4.27 (m, 1H), 4.13 (q, J=6.9 Hz, 2H), 3.61-3.43 (m, 4H), 2.85-2.71 (m, 2H), 2.36-2.25 (m, 2H), 1.55-1.43 (m, 2H), 1.36 (t, J=7.0 Hz, 3H).

### Example 49

### 3-chloro-N-((3aR,5s,6aS)-2-(5-(3-cyano-6-ethoxypyrazolo[1,5-a]pyridin-4-yl)pyridin-2-yl)octahydrocyclopenta[c]pyrrol-5-yl)picolinamide

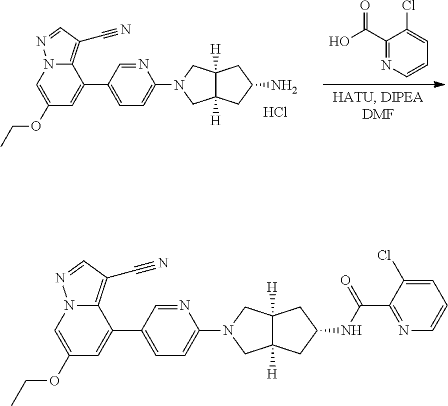 [SMILES: CCOc1cc(-c2ccc(N3C[C@H]4C[C@H](N)C[C@H]4C3)nc2)c2c(C#N)cnn2c1 ; Cl ; O=C(O)c1ncccc1Cl ; "HATU, DIPEADMF" ; CCOc1cc(-c2ccc(N3C[C@H]4C[C@H](NC(=O)c5ncccc5Cl)C[C@H]4C3)nc2)c2c(C#N)cnn2c1]

To a solution of Intermediate 22 (100 mg, 0.235 mmol) in DMF (1 mL) were added 3-chloropicolinic acid (41 mg, 0.259 mmol), HATU (134 mg, 0.353 mmol), and DIPEA (152 mg, 1.175 mmol). The mixture was stirred at 60° C. for 2h. After cooling to rt, the mixture was directly purified by reverse phase flash column chromatography (H₂O/MeOH=80:20 to 40:60) to give the crude compound, which was further purified by prep-TLC (DCM/acetone=2/1) to give the title compound (47 mg, yield: 38%). ESI-MS (m/z): 528.2 [M+1]⁺. ¹H NMR (400 MHz, DMSO-d₆) δ 8.67-8.60 (m, 2H), 8.55 (s, 1H), 8.52 (dd, J=4.6, 1.1 Hz, 1H), 8.29 (d, J=2.3 Hz, 1H), 8.00 (dd, J=8.2, 1.1 Hz, 1H), 7.73 (dd, J=8.7, 2.4 Hz, 1H), 7.50 (dd, J=8.2, 4.7 Hz, 1H), 7.23 (d, J=2.0 Hz, 1H), 6.60 (d, J=8.8 Hz, 1H), 4.46-4.34 (m, 1H), 4.14 (q, J=6.9 Hz, 2H), 3.62 (dd, J=10.7, 7.7 Hz, 2H), 3.34 (dd, J=10.9, 2.8 Hz, 2H), 3.03-2.85 (m, 2H), 1.92-1.85 (m, 4H), 1.36 (t, J=6.9 Hz, 3H).

### Example 50

### 3-chloro-N-((3aR,5s,6aS)-2-(5-(3-cyano-6-ethoxypyrazolo[1,5-a]pyridin-4-yl)pyrazin-2-yl)octahydrocyclopenta[c]pyrrol-5-yl)picolinamide

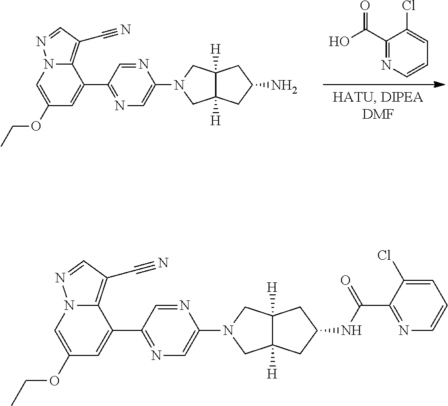 [SMILES: CCOc1cc(-c2cnc(N3C[C@H]4C[C@H](N)C[C@H]4C3)cn2)c2c(C#N)cnn2c1 ; O=C(O)c1ncccc1Cl ; "HATU, DIPEADMF" ; CCOc1cc(-c2cnc(N3C[C@H]4C[C@H](NC(=O)c5ncccc5Cl)C[C@H]4C3)cn2)c2c(C#N)cnn2c1]

To a solution of Intermediate 21 (50 mg, 0.128 mmol) in DMF (0.8 mL) was added 3-chloropicolinic acid (23 mg, 0.141 mmol), HATU (72 mg, 0.192 mmol), and DIPEA (66 mg, 0.512 mmol). The mixture was stirred at 60° C. for 2h. After cooling to rt, the mixture was directly purified by reverse phase flash column chromatography (H₂O/MeOH=80:20 to 40:60) to give the title compound (12 mg, yield: 19%). ESI-MS (m/z): 529.3 [M+1]⁺. ¹H NMR (400 MHz, DMSO-d₆) δ 8.65 (d, J=7.8 Hz, 2H), 8.60-8.44 (m, 3H), 8.09 (s, 1H), 8.04-7.92 (m, 1H), 7.57-7.45 (m, 2H), 4.49-4.32 (m, 1H), 4.15 (q, J=6.9 Hz, 2H), 3.70 (dd, J=10.9, 7.8 Hz, 2H), 3.43 (dd, J=11.2, 2.9 Hz, 2H), 2.96 (s, 2H), 1.94-1.84 (m, 4H), 1.37 (t, J=6.9 Hz, 3H).

### Example 51

### 3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)picolinamide

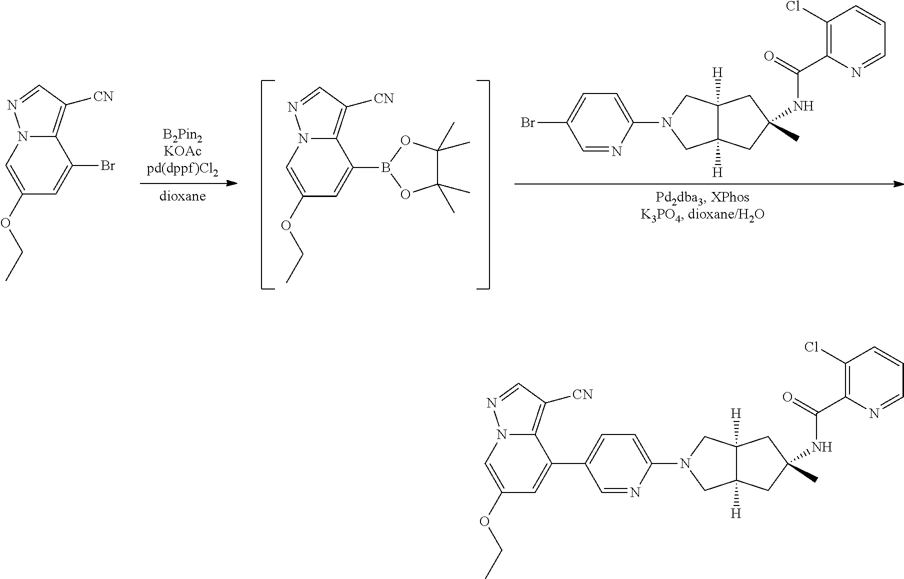

A solution of Intermediate 4 (80 mg, 0.3 mmol), B₂Pin₂ (80 mg, 0.32 mmol), Pd(dppf)Cl₂ DCM (12 mg, 0.02 mmol), and KOAc (59 mg, 0.6 mmol) in dioxane (2 mL) was stirred at 100° C. for 4 h under N₂. To the mixture cooled to rt was added Intermediate 23 (131 mg, 0.3 mmol), Pd₂dba₃ (14 mg, 0.015 mmol), XPhos (29 mg, 0.06 mmol), K₃PO₄ (191 mg, 0.9 mmol), and dioxane/H₂O (5/1 mL). The resultant mixture was flushed with N₂, stirred at 110° C. overnight. After cooling to rt, the mixture was diluted with DCM/MeOH=10/1 (100 mL), washed by H₂O (30 mL×2) and brine (30 mL), dried over anhydrous Na₂SO₄, filtered off, and concentrated in vacuo. The residue was purified by Prep-TLC (DCM/EtOAc=1/1) to give the title compound (23 mg, yield: 14%). ESI-MS (m/z): 542.0 [M+1]⁺. ¹H NMR (400 MHz, CDCl₃) δ 8.45 (d, J=3.7 Hz, 1H), 8.31 (d, J=2.2 Hz, 1H), 8.18 (s, 1H), 8.09 (d, J=1.8 Hz, 1H), 7.82 (d, J=8.0 Hz, 1H), 7.76 (s, 1H), 7.69 (dd, J=8.7, 2.3 Hz, 1H), 7.36 (dd, J=8.1, 4.5 Hz, 1H), 7.07 (d, J=1.8 Hz, 1H), 6.54 (d, J=8.8 Hz, 1H), 4.08 (q, J=6.9 Hz, 2H), 3.67-3.57 (m, 2H), 3.53 (m, 2H), 3.07 (m, 2H), 2.73 (m, 2H), 1.70-1.62 (m, 5H), 1.49 (t, J=6.9 Hz, 3H).

Table 6 lists examples that were prepared according to the procedures as described in Examples 48-51 by using the corresponding intermediates and reagents under appropriate conditions that could be accomplished by the skilled persons.

TABLE 6

| Ex. | Structure | Chemical | Mass | |
|---|---|---|---|---|
| # | (Synthetic Method) | Name | m/z | 1H NMR |
| 52 | 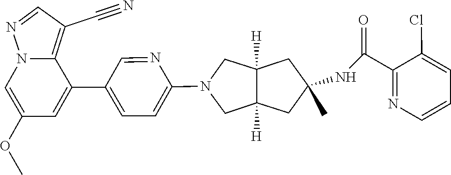 [SMILES: COc1cc(-c2ccc(N3C[C@@H]4C[C@@](C)(NC(=O)c5ncccc5Cl)C[C@@H]4C3)nc2)c2c(C#N)cnn2c1] | 3-chloro-N- ((3aR,5s,6aS)-2- (5-(3-cyano-6- methoxypyrazolo [1,5-a]pyridin-4- yl)pyridin-2-yl)-5- methyloctahydro- cyclopenta[c] pyrrol-5-yl) picolinamide | 528.1 | ¹H NMR (400 MHz, CDCl₃) δ 8.47-8.38 (m, 1H), 8.34-8.24 (m, 1H), 8.19 (d, J = 2.8 Hz, 1H), 8.10 (d, J = 2.1 Hz, 1H), 7.82 (dd, J = 8.1, 1.2 Hz, 1H), 7.74 (m, 1H), 7.71-7.63 (m, 1H), 7.36 (dd, J = 8.1, 4.5 Hz, 1H), 7.06 (dd, 9.2, 2.0 Hz, 1H), 6.54 (d, J = 8.8 Hz, 1H), 3.90 (s, 3H), 3.61 (m, 2H), 3.55-3.48 (m, 2H), 3.11-3.01 (m, 2H), |
| | | | | 2.72 (m, 2H), 1.69-1.59 (m, |
| | | | | 2H), 1.63 (s, 1H). |
| 53 | 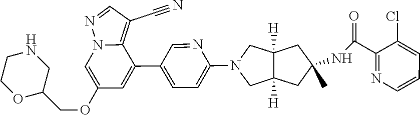 [SMILES: C[C@@]1(NC(=O)c2ncccc2Cl)C[C@H]2CN(c3ccc(-c4cc(OCC5CNCCO5)cn5ncc(C#N)c45)cn3)C[C@H]2C1] | 3-chloro-N- ((3aR,5s,6aS)-2- (5-(3-cyano-6- morpholin-2- ylmethoxy) pyrazolo[1,5-a] pyridin-4-yl) pyridin-2-yl)-5- | 613.3 | ¹H NMR (400 MHz, CD₃OD) δ 8.49 (d, J = 4.7 Hz, 1H), 8.42 (d, J = 12.6 Hz, 1H), 8.32 (s, 1H), 8.23 (s, 1H), 7.95 (d, J = 8.2 Hz, 1H), 7.72 (t, J = 9.3 Hz, 1H), 7.48 (dd, J = 8.2, 4.7 Hz, 1H), 7.22 (d, J = 7.0 Hz, 1H), 6.68 (t, J = 9.0 Hz, 1H), 4.08 (d, J = 4.5 |
| | | methyloctahydro- | | Hz, 2H), 3.90 (m, 2H), 3.76- |
| | | cyclopenta[c] | | 3.44 (m, 5H), 3.08 (m, 2H), 3.00 |
| | | pyrrol-5-yl) | | (m, 2H), 2.88-2.79 (m, 2H), |
| | | picolinamide | | 2.75 (m, 2H), 1.63-1.55 (m, |
| | | | | 2H), 1.62 (s, 3H). |
| 54 | 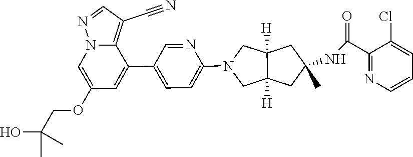 [SMILES: CC(C)(O)COc1cc(-c2ccc(N3C[C@@H]4C[C@@](C)(NC(=O)c5ncccc5Cl)C[C@@H]4C3)nc2)c2c(C#N)cnn2c1] | 3-chloro-N- ((3aR,5s,6aS)-2- (5-(3-cyano-6-(2- hydroxy-2- methylpropoxy) pyrazolo[1,5-a] pyridin-4-yl) pyridin-2-yl)-5- methyloctahydro- cyclopenta[c] | 585.9 | ¹H NMR (400 MHz, CD₃OD) δ 8.50 (d, J = 4.6 Hz, 1H), 8.44 (s, 2H), 8.32 (d, J = 8.7 Hz, 1H), 8.24 (s, 1H), 7.95 (t, J = 8.9 Hz, 1H), 7.77 (d, J = 7.2 Hz, 1H), 7.48 (dt, J = 10.0, 5.0 Hz, 1H), 7.28 (d, J = 11.4 Hz, 1H), 6.72 (t, J = 7.2 Hz, 1H), 3.91 (s, 2H), 3.70-3.50 (m, 4H), 3.10 (m, 2H), 2.72 (m, 2H), 1.68-1.54 |
| | | pyrrol-5- | | (m, 2H), 1.63 (s, 3H), 1.35 (s, |
| | | yl)picolinamide | | 6H). |
| 55 | 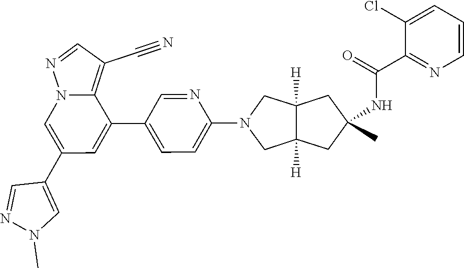 [SMILES: Cn1cc(-c2cc(-c3ccc(N4C[C@@H]5C[C@@](C)(NC(=O)c6ncccc6Cl)C[C@@H]5C4)nc3)c3c(C#N)cnn3c2)cn1] | 3-chloro-N- ((3aR,5s,6aS)-2- (5-(3-cyano-6-(1- methyl-1H- pyrazol-4-yl) pyrazolo[1,5- a]pyridin-4- yl)pyridin-2-yl)-5- methyloctahydro- cyclopenta[c] pyrrol-5- yl)picolinamide | 578.3 | ¹H NMR (400 MHz, CD₃OD) δ 8.95 (s, 1H), 8.50 (d, J = 4.3 Hz, 1H), 8.39 (m, 1H), 8.28 (m, 1H), 8.15 (m, 1H), 7.98 (m, 2H), 7.79 (d, J = 8.7 Hz, 1H), 7.65 (m, 1H), 7.48 (dd, J = 8.2, 4.7 Hz, 1H), 6.72 (d, J = 8.7 Hz, 1H), 3.95 (s, 3H), 3.77-3.44 (m, 4H), 3.10 (m, 2H), 2.82-2.66 (m, 2H), 1.68-1.53 (m, 2H), 1.63 (s, 3H). |
| 56 | 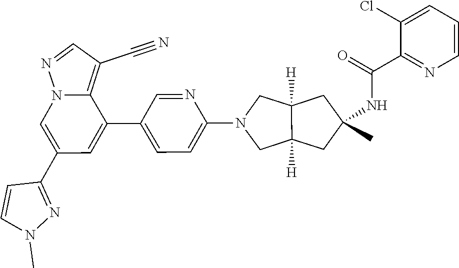 [SMILES: Cn1ccc(-c2cc(-c3ccc(N4C[C@@H]5C[C@@](C)(NC(=O)c6ncccc6Cl)C[C@@H]5C4)nc3)c3c(C#N)cnn3c2)n1] | 3-chloro-N- ((3aR,5s,6aS)-2- (5-(3-cyano-6-(1- methyl-1H- pyrazol-3-yl) pyrazolo[1,5- a]pyridin-4- yl)pyridin-2-yl)-5- methyloctahydro- cyclopenta[c] pyrrol-5- yl)picolinamide | 578.4 | ¹H NMR (400 MHz, CD₃OD) δ 9.07 (s, 1H), 8.46 (m, 2H), 8.28 (m, 1H), 8.00-7.87 (m, 1H), 7.90-7.76 (m, 2H), 7.68 (s, 1H), 7.45 (m, 1H), 6.80 (s, 1H), 6.71 m, 1H), 3.97 (s, 3H), 3.72-3.49 (m, 4H), 3.05 (m, 2H), 2.75 (m, 2H), 1.67-1.51 (m, 2H), 1.63 (s, 3H). |
| 57 | 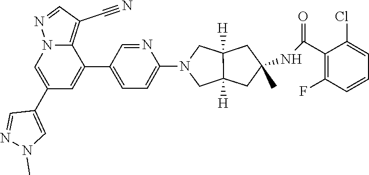 [SMILES: Cn1cc(-c2cc(-c3ccc(N4C[C@@H]5C[C@@](C)(NC(=O)c6c(F)cccc6Cl)C[C@@H]5C4)nc3)c3c(C#N)cnn3c2)cn1] | 2-chloro-N- ((3aR,5s,6aS)-2- (5-(3-cyano-6-(1- methyl-1H- pyrazol-4-yl) pyrazolo[1,5- a]pyridin-4- yl)pyridin-2-yl)-5- methyloctahydro- cyclopenta[c] pyrrol-5-yl)-6- | 595.4 | ¹H NMR (400 MHz, DMSO-d₆) δ 9.20 (s, 1H), 8.65 (d, J = 19.9 Hz, 1H), 8.48-8.23 (m, 3H), 8.10 (s, 1H), 7.86-7.66 (m, 2H), 7.43 (dt, J = 14.9, 7.5 Hz, 1H), 7.36- 7.16 (m, 2H), 6.64 (t, J = 10.3 Hz, 1H), 3.86 (s, 3H), 3.60-3.44 (m, 4H), 2.90 (m, 2H), 2.64 (m, 1.6H, rotamer), 2.21-2.09 (m, 0.2H, rotamer), 2.02-1.92 (m, 0.2H, rotamer), 1.49 (s, 3H), |
| | | fluorobenzamide | | 1.43-1.33 (m, 2H). |
| 58 | 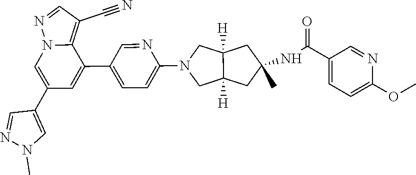 [SMILES: COc1ccc(C(=O)N[C@]2(C)C[C@H]3CN(c4ccc(-c5cc(-c6cnn(C)c6)cn6ncc(C#N)c56)cn4)C[C@H]3C2)cn1] | N-((3aR,5s,6aS)- 2-(5-(3-cyano- 6-(1-methyl- 1H-pyrazol-4- yl)pyrazolo[1,5- a]pyridin-4-yl) pyridin-2-yl)-5- methyloctahydro- cyclopenta[c] pyrrol-5-yl)-6- methoxy- | 574.5 | ¹H NMR (400 MHz, DMSO-d₆) δ 9.20 (s, 1H), 8.62 (s, 2H), 8.36 (d, J = 11.3 Hz, 2H), 8.09 (d, J = 8.8 Hz, 2H), 7.87 (s, 1H), 7.78 (d, J = 8.2 Hz, 1H), 7.72 (d, J = 11.5 Hz, 1H), 6.85 (d, J = 8.6 Hz, 1H), 6.62 (m, 1H), 3.88 (s, 3H), 3.86 (s, 3H), 3.46 (m, 4H), 2.94 (m, 2H), 2.72-2.15 (m, 2H), 1.48 (m, 5H). |
| | | nicotinamide | | |

### Example 59

### 2-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-fluorobenzenesulfonamide

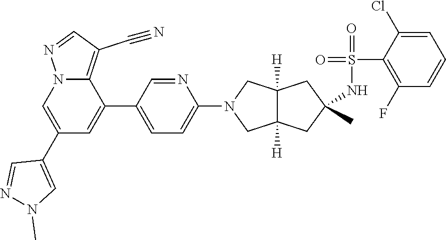 [SMILES: Cn1cc(-c2cc(-c3ccc(N4C[C@@H]5C[C@@](C)(NS(=O)(=O)c6c(F)cccc6Cl)C[C@@H]5C4)nc3)c3c(C#N)cnn3c2)cn1]

The compound was prepared according to the similar procedure of Example 35. ESI-MS (m/z): 631.6 [M+1]⁺. ¹H NMR (400 MHz, DMSO-d₆) δ 9.19 (s, 1H), 8.62 (d, J=6.9 Hz, 1H), 8.36 (s, 1H), 8.31 (s, 1H), 8.09 (s, 1H), 8.04 (s, 1H), 7.78-7.69 (m, 2H), 7.63 (d, J=5.5 Hz, 1H), 7.50 (d, J=8.0 Hz, 1H), 7.44 (d, J=8.5 Hz, 1H), 6.60 (d, J=8.4 Hz, 1H), 3.86 (s, 3H), 3.37 (m, 4H), 2.80-1.73 (m, 4H), 1.31-1.28 (m, 5H).

### Example 60

### 3-chloro-N-((3aR,5s,6aS)-2-(5-(3-cyano-6-ethoxypyrazolo[1,5-a]pyridin-4-yl)pyrazin-2-yl)-5-methyloctahydrocyclopenta[c]pyrrol-5-yl)picolinamide

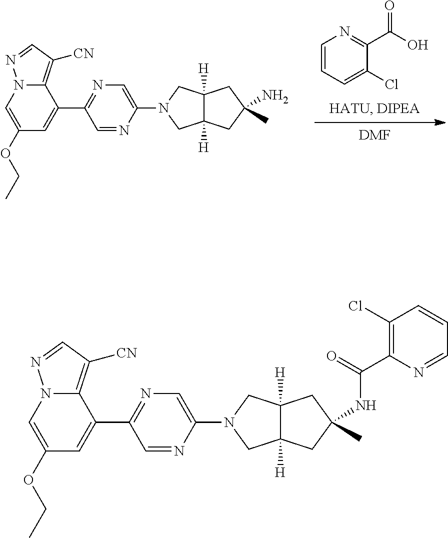

To a solution of Intermediate 24 (60 mg, 0.15 mmol), 3-chloropicolinic acid (28 mg, 0.18 mmol), HATU (85 mg, 0.22 mmol) in DMF (5 mL) was added DIPEA (58 mg, 0.45 mmol). The mixture was stirred at 40° C. for 2 h. After cooling to rt, the mixture was diluted with EtOAc (50 mL), washed by H₂O (15 mL×2) and brine (15 mL), dried over anhydrous Na₂SO₄, filtered off, and concentrated in vacuo. The residue was purified by Prep-TLC (DCMMeOH=25/1) to give the title compound (57 mg, yield: 70%). ESI-MS (m/z): 543.2 [M+1]⁺. ¹H NMR (400 MHz, CDCN₃) δ 8.46-8.43 (N, 1H), 8.41 (s, 1H), 8.22 (d, J=2.9 Hz, 1H), 8.14-8.06 (m, 2H), 7.81 (dd, J=10.4, 9.2 Hz, 2H), 7.37 (dd, J=8.1, 4.5 Hz, 1H), 7.29 (t, J=3.6 Hz, 1H), 4.09 (q, J=6.9 Hz, 2H), 3.69 (dd, J=10.9, 7.6 Hz, 2H), 3.57 (m, 2H), 3.14-3.06 (m, 2H), 2.75 (m, 2H), 1.69-1.62 (m, 5H), 1.49 (t, J=6.9 Hz, 3H).

Table 7 lists examples that were prepared according to the procedures as described in Examples 59-60 by using the corresponding intermediates and reagents under appropriate conditions that could be accomplished by the skilled persons.

TABLE 7

| Ex. | Structure | Chemical | Mass | |
|---|---|---|---|---|
| # | (Synthetic Method) | Name | m/z | 1H NMR |
| 61 | 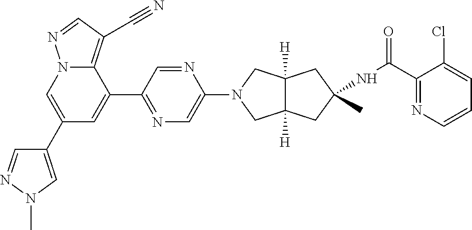 [SMILES: Cn1cc(-c2cc(-c3cnc(N4C[C@@H]5C[C@@](C)(NC(=O)c6ncccc6Cl)C[C@@H]5C4)cn3)c3c(C#N)cnn3c2)cn1] | 3-chloro-N- ((3aR,5s,6aS)-2-(5-(3- cyano-6-(1-methyl-1H- pyrazol-3- yl)pyrazolo[1,5- a]pyridin-4-yl)pyrazin- 2-yl)-5- methyloctahydrocyclo penta[c]pyrrol-5- yl)picolinainide | 579.4 | ¹H NMR (400 MHz, DMSO- d₆) δ 9.23 (s, 1H), 8.63 (s, 1H), 8.61 (d, J = 1.2 Hz, 1H), 8.51 (dd, J = 4.7, 1.2 Hz, 1H), 8.39 (s, 1H), 8.29 (s, 1H), 8.13 (d, J = 2.2 Hz, 2H), 8.02-7.97 (m, 2H), 7.49 (dd, J = 8.2, 4.7 Hz, 1H), 3.88 (s, 3H), 3.57 (m, 7.9 Hz, 4H), 2.98 (m, 2H), 2.65 (m, 2H), 1.51 (s, 3H), 1.44 (m, 2H). |
| 62 | 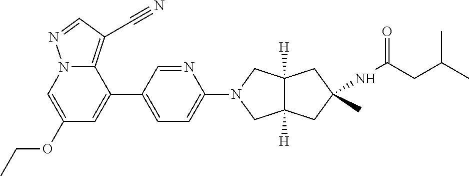 [SMILES: CCOc1cc(-c2ccc(N3C[C@@H]4C[C@@](C)(NC(=O)CC(C)C)C[C@@H]4C3)nc2)c2c(C#N)cnn2c1] | N-((3aR,5s,6aS)-2-(5- (3-cyano-6- ethoxypyrazolo[1,5- a]pyridin-4-yl)pyridin- 2-yl)-5- methyloctahydrocyclo penta[c]pyrrol-5-yl)-3- methylbutanamide | 487.2 | ¹H NMR (400 MHz, CD₃OD) δ 8.21 (m, 3H), 7.68 (dt, J = 6.8, 3.4 Hz, 1H), 7.12 (d, J = 2.0 Hz, 1H), 6.59 (t, J = 8.4 Hz, 1H), 4.09 (t, J = 7.0 Hz, 2H), 3.56 (m, 2H), 3.45 (m, 2H), 2.96 (m, 2H), 2.56 (m, 2H), 2.05-1.95 (m, 3H), (dd, J = 13.6, 7.1 Hz, 1H), 1.46 (m, 5H), 0.93 (d, J = 6.2 Hz, 6H). |
| 63 | 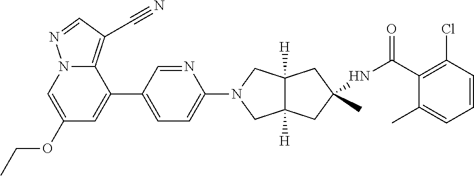 [SMILES: CCOc1cc(-c2ccc(N3C[C@@H]4C[C@@](C)(NC(=O)c5c(C)cccc5Cl)C[C@@H]4C3)nc2)c2c(C#N)cnn2c1] | 2-chloro-N- ((3aR,5s,6aS)-2-(5-(3- cyano-6- ethoxypyrazolo[1,5- a]pyridin-4-yl)pyridin- 2-yl)-5- methyloctahydrocyclo penta[c]pyrrol-5-yl)-6- methylbenzainide | 555.3 | ¹H NMR (400 MHz, CD₃OD) δ 8.23-8.16 (m, 3H), 7.69 (dd, J = 8.8, 2.4 Hz, 1H), 7.22- 7.14 (m, 2H), 7.10 (m, 2H), 6.60 (t, J = 9.1 Hz, 1H), 4.09 (q, J = 7.0 Hz, 2H), 3.58 (m, 2H), 3.48 (m, 2H), 3.05 (m, 2H), 2.72 (m, 2H), 2.35 (s, 3H), 1.61 (s, 3H), 1.56-1.49 (m, 2H), 1.45 (t, J = 6.9 Hz, 3H). |
| 64 | 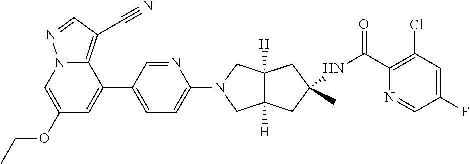 [SMILES: CCOc1cc(-c2ccc(N3C[C@@H]4C[C@@](C)(NC(=O)c5ncc(F)cc5Cl)C[C@@H]4C3)nc2)c2c(C#N)cnn2c1] | 3-chloro-N- ((3aR,5s,6aS)-2-(5-(3- cyano-6- ethoxypyrazolo[1,5- a]pyridin-4-yl)pyridin- 2-yl)-5- methyloctahydrocyclo penta[c]pyrrol-5-yl)-5- fluoropicolinamide | 560.2 | ¹H NMR (400 MHz, CD₃OD) δ 8.36 (m, 1H), 8.24-8.15 (m, 3H), 7.72-7.64 (m, 2H), 7.13-7.05 (m, 1H), 6.60 (d, J = 8.8 Hz, 1H), 4.10 (q, J = 6.9 Hz, 2H), 3.58 (m, 2H), 3.49 (m, 2H), 3.04 (m, 2H), 2.70 (m, 2H), 1.68-1.54 (m, 2H), 1.60 (s, 3H), 1.46 (t, J = 7.0 Hz, 3H). |
| 65 | 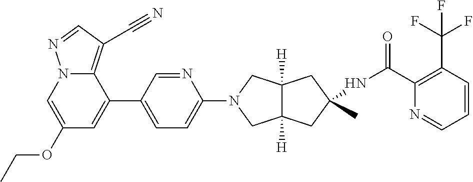 [SMILES: CCOc1cc(-c2ccc(N3C[C@@H]4C[C@@](C)(NC(=O)c5ncccc5C(F)(F)F)C[C@@H]4C3)nc2)c2c(C#N)cnn2c1] | N-((3aR,5s,6aS)-2-(5- (3-cyano-6- ethoxypyrazolo[1,5- a]pyridin-4-yl)pyridin- 2-yl)-5- methyloctahydrocyclo penta[c]pyrrol-5-yl)-3- (trifluoromethyl) picolinamide | 576.5 | ¹H NMR (400 MHz, CD₃OD) δ 8.74 (d, J = 4.1 Hz, 1H), 8.21 (m, 3H), 8.15 (d, J = 8.8, 1.2 Hz, 1H), 7.72-7.64 (m, 1H), 7.60 (dd, J = 8.0, 5.1 Hz, 1H), 7.14-7.07 (m, 1H), 6.60 (d, J = 8.7 Hz, 1H), 4.14- 4.04 (q, J = 6.8 Hz, 3H), 3.59 (m, 3H), 3.49 (m, 2H), 3.04 (m, 2H), 2.70 (m, 2H), 1.62- 1.53 (m, 2H), 1.06 (s, 1H). 1.46 (t, J = 7.0 Hz, 3H). |
| 66 | 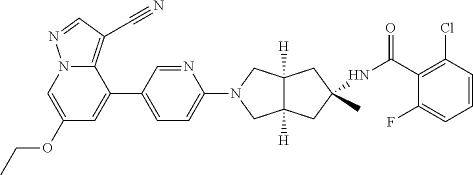 [SMILES: CCOc1cc(-c2ccc(N3C[C@@H]4C[C@@](C)(NC(=O)c5c(F)cccc5Cl)C[C@@H]4C3)nc2)c2c(C#N)cnn2c1] | 2-chloro-N- ((3aR,5s,6aS)-2-(5-(3- cyano-6- ethoxypyrazolo[1,5- a]pyridin-4-yl)pyridin- 2-yl)-5- methyloctaliydrocyclo penta[c]pyrrol-5-yl)-6- fluorobenzamide | 559.3 | ¹H NMR (400 MHz, CD₃OD) δ 8.20 (m, 3H), 7.68 (m, 1H), 7.35-7.24 (m, 1H), 7.19 (m, 1H), 7.11 m, 1H), 7.09-6.96 (m, 1H), 6.58 (m, 1H), 4.14- 4.03 (q, J = 6.9 Hz, 2H), 3.57 (m, 4H), 3.00 (m, 2H), 2.70 (m, 1H), 1.59 (s, 3H), 1.56- 1.49 (m, 2H), 1.46 (t, J = 6.9 Hz, 3H). |
| 67 | 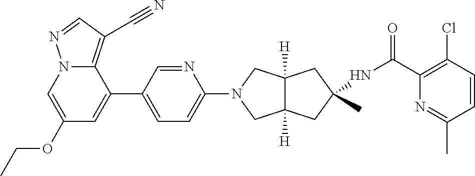 [SMILES: CCOc1cc(-c2ccc(N3C[C@@H]4C[C@@](C)(NC(=O)c5nc(C)ccc5Cl)C[C@@H]4C3)nc2)c2c(C#N)cnn2c1] | 3-chloro-N- ((3aR,5s,6aS)-2-(5-(3- cyano-6- ethoxypyrazolo[1,5- a]pyridin-4-yl)pyridin- 2-yl)-5- methyloctahydrocyclo penta[c]pyrrol-5-yl)-6- methylpicolinaniide | 556.3 | ¹H NMR (400 MHz, CD₃OD) δ 8.24-8.14 (m, 3H), 7.68 (dd, J = 8.8, 2.4 Hz, 1H), 7.60 (d, J = 8.1 Hz, 1H), 7.35 (d, J = 8.1 Hz, 1H), 7.10 (dd, J = 8.9, 2.0 Hz, 1H), 6.59 (d, J = 8.8 Hz, 1H), 4.09 (q, J = 6.9 Hz, 2H), 3.60 (m, 2H), 3.49 (dd, J = 10.6, 2.1 Hz, 2H), 3.09-2.99 (m, 2H), 2.69 (dd, J = 13.9, 7.5 Hz, 2H), 2.62 (s, |
| | | | | 3H), 1.67-1.54 (m, 2H), 1.58 |
| | | | | (s, 3H), 1.46 (t, J = 6.9 Hz, |
| | | | | 3H). |
| 68 | 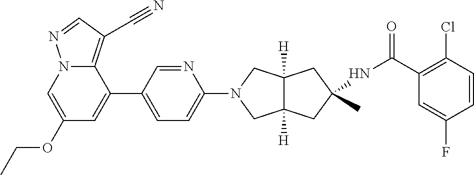 [SMILES: CCOc1cc(-c2ccc(N3C[C@@H]4C[C@@](C)(NC(=O)c5cc(F)ccc5Cl)C[C@@H]4C3)nc2)c2c(C#N)cnn2c1] | 2-chloro-N- ((3aR,5s,6aS)-2-(5-(3- cyano-6- ethoxypyrazolo[1,5- a]pyridin-4-yl)pyridin- 2-yl)-5- methyloctahydrocyclo penta[c]pyrrol-5-yl)-5- fluorobenzamide | 559.3 | ¹H NMR (400 MHz, CD₃OD) δ 8.30 (m, 1H), 8.26 (m, 1H), 8.22 (m, 1H), 7.71 (ddd, J- 7.7, 5.2, 2.5 Hz, 1H), 7.40 (m, 1H), 7.18-7.03 (m, 3H), 6.65 (m, 1H), 4.12 (q, J = 6.9 Hz, 2H), 3.70-3.44 (m, 4H), 3.05 (m, 2H), 2.70 (m, 1H), 2.32- 2.09 (m, 1H), 1.62-1.51 (m, 3H), 1.59 (s, 3H), 1.46 (t, J = 7.0 Hz, 3H). |
| 69 | 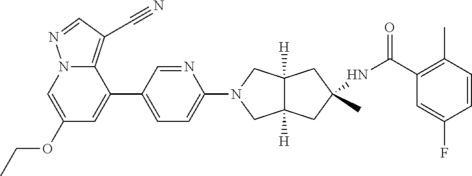 [SMILES: CCOc1cc(-c2ccc(N3C[C@@H]4C[C@@](C)(NC(=O)c5cc(F)ccc5C)C[C@@H]4C3)nc2)c2c(C#N)cnn2c1] | N-((3aR,5s,6aS)-2-(5- (3-cyano-6- ethoxypyrazolo[1,5- a]pyridin-4-yl)pyridin- 2-yl)-5- methyloctahydrocyclo penta[c]pyrrol-5-yl)-5- fluoro-2- methylbenzamide | 539.4 | ¹H NMR (400 MHz, CD₃OD) δ 8.29-8.14 (m, 3H), 7.68 (m, 1H), 7.21-7.07 (m, 2H), 7.02-6.88 (m, 2H), 6.61 (m, 1H), 4.14-4.05 (q, J = 6.9 Hz, 2H), 3.67-3.43 (m, 4H), 3.09-2.90 (m, 2H), 2.70 (m, 1.2H), 2.35, 2.30 (s, 3H, rotamer), 2.32-2.28 (m, 0.4H, rotamer), 2.09-1.95 (m, 0.4H, rotamer), 1.61- |
| | | | | 1.49 (m, 2H), 1.59, 1.50 (s, |
| | | | | 3H, rotamer), 1.46 (t, J = |
| | | | | 6.9 Hz, 3H). |
| 70 | 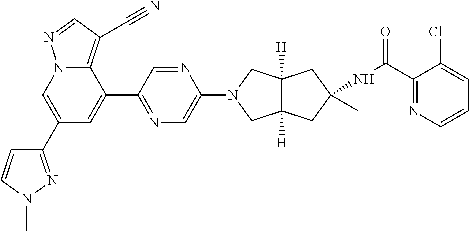 [SMILES: Cn1ccc(-c2cc(-c3cnc(N4C[C@@H]5C[C@@](C)(NC(=O)c6ncccc6Cl)C[C@@H]5C4)cn3)c3c(C#N)cnn3c2)n1] | 3-chloro-N- ((3aR,5s,6aS)-2-(5-(3- cyano-6-(1-methyl-1H- pyrazol-3- yl)pyrazolo[1,5- a]pyridin-4-yl)pyrazin- 2-yl)-5- methyloctahydrocyclo penta[c]pyrrol-5- yl)picolinamide | 579.4 | ¹H NMR (400 MHz, cdcl₃) δ 8.92 (s, 1H), 8.50 (s, 1H), 8.45 (s, 1H), 8.31 (s, 1H), 8.11 (s, 1H), 8.01 (s, 1H), 7.86-7.72 (m, 2H), 7.45 (s, 1H), 7.40- 7.31 (m, 1H), 6.59 (s, 1H), 3.98 (s, 3H), 3.69 (d, J = 5.9 Hz, 2H), 3.59 (m, 2H), 3.12 (m, 2H), 2.76 (m, 2H), 1.61 (m, 5H). |

### Example 71

### tert-butyl (((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate

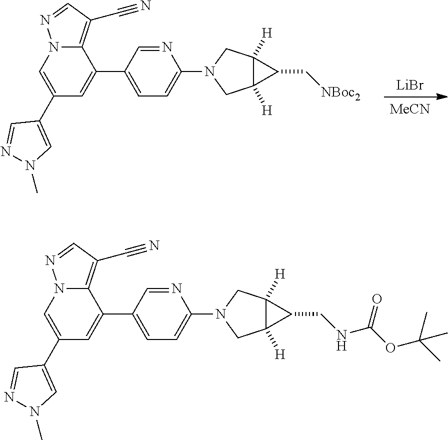

To a solution of the product of step 5 of Intermediate 26 (40 mg, 0.065 mmol) in MeCN (2 mL) was added LiBr (9 mg, 0.098 mmol). The mixture was stirred at 80° C. for 6h. The mixture was concentrated in vacuo. The residue was taken up in EtOAc (50 mL), washed with H₂O (10 mL) and brine (10 mL), dried over anhydrous Na₂SO₄, filtered off, and concentrated in vacuo. The residue was triturated with MeCN (2 mL), filtered, washed with MeCN (2 mL) and dried in vacuo to give the title compound (25 mg, yield: 60%). ESI-MS (m/z): 511.4 [M+1]⁺. ¹H NMR (400 MHz, DMSO-d₆) δ 9.19 (s, 1H), 8.62 (s, 1H), 8.37 (s, 1H), 8.30 (d, J=2.3 Hz, 1H), 8.09 (s, 1H), 7.78-7.69 (m, 2H), 6.92 (s, 1H), 6.55 (d, J=8.8 Hz, 1H), 3.86 (s, 3H), 3.68 (d, J=10.4 Hz, 2H), 3.40 (d, J=9.7 Hz, 2H), 2.90 (t, J=6.0 Hz, 2H), 1.61 (s, 2H), 1.37 (s, 9H), 0.81-0.72 (m, 1H).

### Example 72

### tert-butyl (((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate

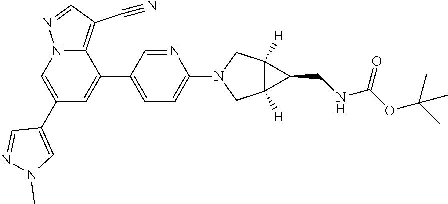 [SMILES: Cn1cc(-c2cc(-c3ccc(N4C[C@@H]5[C@H](CNC(=O)OC(C)(C)C)[C@@H]5C4)nc3)c3c(C#N)cnn3c2)cn1]

This compound was synthesized by following the procedure used to make Example 71 starting from the product of Step 5 of Intermediate 27. ESI-MS (m/z): 511.4 [M+1]⁺. ¹H NMR (400 MHz, DMSO-d₆) δ 9.19 (s, 1H), 8.62 (s, 1H), 8.37 (s, 1H), 8.32 (d, J=2.3 Hz, 1H), 8.10 (s, 1H), 7.82-7.73 (m, 2H), 7.02-6.85 (m, 1H), 6.51 (d, J=8.8 Hz, 1H), 3.86 (s, 3H), 3.58 (dd, J=21.8, 10.3 Hz, 4H), 2.83 (t, J=6.2 Hz, 2H), 1.89-1.73 (m, 2H), 1.35 (s, 9H), 0.85-0.80 (m, 1H).

### Example 73

### 3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)picolinamide

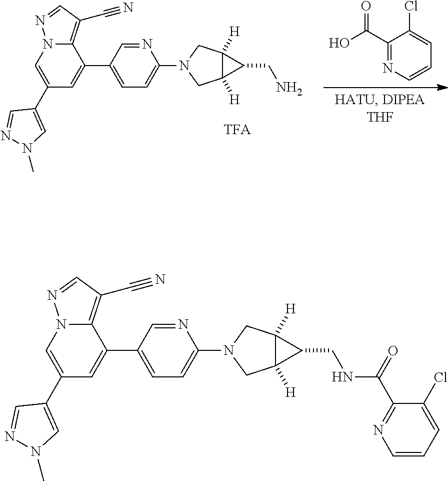 [SMILES: Cn1cc(-c2cc(-c3ccc(N4C[C@@H]5[C@@H](CN)[C@@H]5C4)nc3)c3c(C#N)cnn3c2)cn1 ; O=C(O)c1ncccc1Cl ; "HATU, DIPEATHF" ; Cn1cc(-c2cc(-c3ccc(N4C[C@@H]5[C@@H](CNC(=O)c6ncccc6Cl)[C@@H]5C4)nc3)c3c(C#N)cnn3c2)cn1]

To a solution of Intermediate 26 (60 mg, 0.15 mmol), 3-chloropicolinic acid (23 mg, 0.148 mmol), HATU (56 mg, 0.148 mmol) in THF (2 mL) was added DIPEA (64 mg, 0.495 mmol). The mixture was stirred at 60° C. for 6 h. The mixture was concentrated in vacuo and the residue was purified by reverse phase flash column chromatography (MeOH/H₂O=5% to 95%) to give the title compound (15 mg, yield: 27%). ESI-MS (m/z): 550.4 [M+1]⁺. ¹H NMR (400 MHz, CDCl₃) δ 8.65 (d, J=1.3 Hz, 1H), 8.50 (dd, J=4.5, 1.3 Hz, 1H), 8.29 (d, J=2.4 Hz, 1H), 8.26 (s, 111), 7.85 (dd, J=8.2, 1.3 Hz, 111), 7.80 (s, 111), 7.76-7.69 (m, 2H), 7.44-7.36 (m, 2H), 6.52 (d, J=8.8 Hz, 1H), 3.99 (s, 3H), 3.84 (d, J=10.2 Hz, 2H), 3.59 (d, J=10.0 Hz, 2H), 3.50-3.38 (m, 2H), 1.80 (s, 2H), 1.14-1.03 (m, 1H).

### Example 74

### 2-chloro-N-(((1R,5S,6r)-3-(5-(3-cyano-6-(1-methyl-H-pyrazol-4-yl)pyrazolo[1,5-a]pyridin-4-yl)pyridin-2-yl)-3-azabicyclo[3.1.0]hexan-6-yl)methyl)-6-fluorobenzenesulfonamide

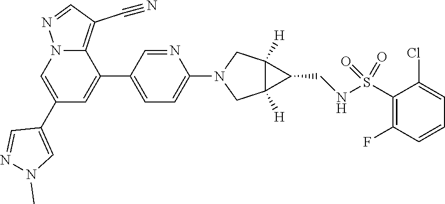 [SMILES: Cn1cc(-c2cc(-c3ccc(N4C[C@@H]5[C@@H](CNS(=O)(=O)c6c(F)cccc6Cl)[C@@H]5C4)nc3)c3c(C#N)cnn3c2)cn1]

The compound was prepared according to the similar procedure of Example 35 starting from Intermediate 26. ESI-MS (m/z): 603.4 [M+1]. H NMR (400 MHz, CDCl₃+CD₃OD) δ 8.61 (s, 1H), 8.21 (s, 2H), 7.74 (d, J=8.1 Hz, 2H), 7.66 (d, J=8.3 Hz, 1), 7.46-7.29 (m, 3H), 7.12 (t, J=9.3 Hz, 1H), 6.41 (d, J=8.8 Hz, 1H), 3.93 (s, 3H), 3.49 (dd, J=50.8, 9.7 Hz, 4H), 3.04 (d, J=6.8 Hz, 2H), 1.58 (m, 2H), 0.79 (in, 1H).

Table 8 lists examples that were prepared according to the procedures as described in Examples 71-74 by using the corresponding intermediates and reagents under appropriate conditions that could be accomplished by the skilled persons.

TABLE 8

| Ex. | Structure | | Mass | |
|---|---|---|---|---|
| # | (Synthetic Method) | Chemical Name | m/z | 1H NMR |
| 75 | 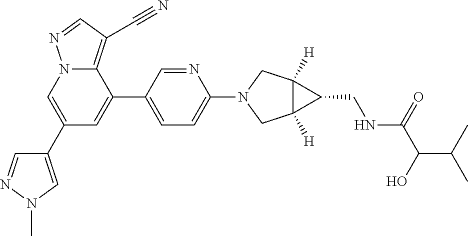 [SMILES: CC(C)C(O)C(=O)NC[C@@H]1[C@H]2CN(c3ccc(-c4cc(-c5cnn(C)c5)cn5ncc(C#N)c45)cn3)C[C@@H]12] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 2-hydroxy-3- methylbutanamide | 511.4 | 1H NMR (400 MHz, CDCl3) δ 8.61 (s, 1H), 8.28-8.19 (m, 2H), 7.76 (s, 1H), 7.70 (s, 1H), 7.66 (dd, J = 8.7, 2.3 Hz, 1H), 7.36 (s, 1H), 7.05 (s, 1H), 6.45 (d, J = 8.8 Hz, 1H), 3.95 (s, 3H), 3.89 (d, J = 3.0 Hz, 1H), 3.73 (d, J = 10.2 Hz, 2H), 3.50 (d, J = 9.5 Hz, 2H), 3.22 (t, J = 6.4 Hz, 2H), 2.16- 2.09 (m, 1H), 1.67 (s, 2H), 0.99 (d, J = 6.9 Hz, 3H), 0.96-0.88 (m, 1H), 0.83 (d, J = 6.8 Hz, 3H). |
| 76 | 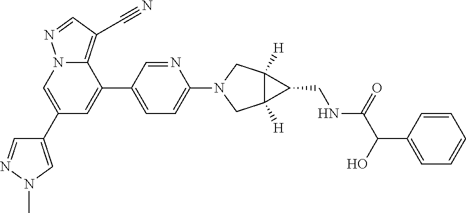 [SMILES: Cn1cc(-c2cc(-c3ccc(N4C[C@@H]5[C@@H](CNC(=O)C(O)c6ccccc6)[C@@H]5C4)nc3)c3c(C#N)cnn3c2)cn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 2-hydroxy-2- phenylacetamide | 545.5 | ¹H NMR (400 MHz, CDCl₃) δ 8.60 (s, 1H), 8.20 (s, 2H), 7.72 (d, J = 12.2 Hz, 2H), 7.63 (dd, J = 8.6, 1.7 Hz, 1H), 7.42-7.33 (m, 3H), 7.30 (t, J = 7.2 Hz, 2H), 7.24 (s, 1H), 6.42 (d, J = 8.8 Hz, 1H), 5.00 (s, 1H), 3.91 (s, 3H), 3.68 (d, J = 10.2 Hz, 2H), 3.45 (d, J = 9.6 Hz, 2H), 3.18 (dd, J = 13.2, 6.8 Hz, 2H), 1.61 (s, 2H), 0.88 (s, 1H). |
| 77 | 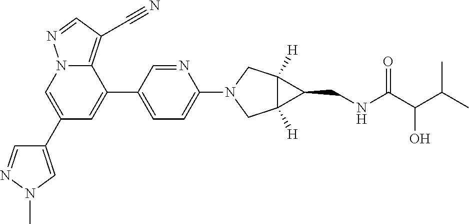 [SMILES: CC(C)C(O)C(=O)NC[C@H]1[C@H]2CN(c3ccc(-c4cc(-c5cnn(C)c5)cn5ncc(C#N)c45)cn3)C[C@@H]12] | N-(((1R,5S,6r)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 2-hydroxy-3- methylbutanamide | 511.4 | ¹H NMR (400 MHz, DMSO-d₆) δ 9.19 (d, J = 1.3 Hz, 1H), 8.62 (s, 1H), 8.37 (s, 1H), 8.32 (d, J = 2.3 Hz, 1H), 8.10 (s, 1H), 7.82-7.73 (m, 3H), 6.51 (d, J = 8.8 Hz, 1H), 5.27 (d, J = 5.7 Hz, 1H), 3.86 (s, 3H), 3.71-3.62 (m, 3H), 3.61- 3.53 (m, 2H), 3.01 (t, J = 6.4 Hz, 2H), 1.98-1.90 (m, 1H), 1.88- 1.78 (m, 2H), 0.88 (d, J = 6.9 Hz, 3H), 0.82-0.80 (m, 1H), 0.74 (d, J = 6.8 Hz, 3H). |
| 78 | 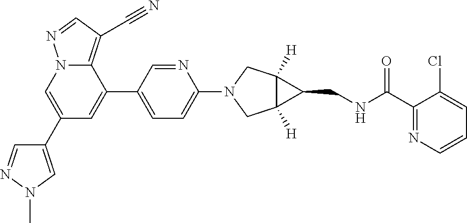 [SMILES: Cn1cc(-c2cc(-c3ccc(N4C[C@@H]5[C@H](CNC(=O)c6ncccc6Cl)[C@@H]5C4)nc3)c3c(C#N)cnn3c2)cn1] | 3-chloro-N- (((1R,5S,6r)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6- yl)methyl) picolinamide | 550.5 | ¹H NMR (400 MHz, DMSO-d₆) δ 9.20 (d, J = 1.3 Hz, 1H), 8.75 (t, J = 5.4 Hz, 1H), 8.62 (s, 1H), 8.53 (dd, J = 4.6, 1.3 Hz, 1H), 8.38 (s, 1H), 8.34 (d, J = 2.3 Hz, 1H), 8.10 (s, 1H), 8.00 (dd, J = 8.2, 1.3 Hz, 1H), 7.84-7.75 (m, 2H), 7.51 (dd, J = 8.2, 4.7 Hz, 1H), 6.54 (d, J = 8.8 Hz, 1H), 3.85 (s, 3H), 3.66 (dd, J = 36.1, 9.9 Hz, 4H), 3.16 (dd, J = 11.8, 5.8 Hz, 2H), 1.89 (dd. J = 7.8, 1.9 Hz, 2H), 0.86-0.78 (m, 1H). |
| 79 | 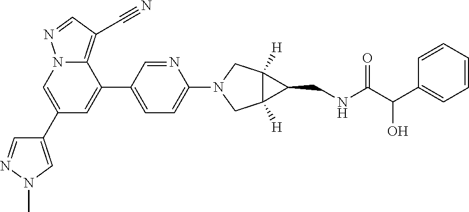 [SMILES: Cn1cc(-c2cc(-c3ccc(N4C[C@@H]5[C@H](CNC(=O)C(O)c6ccccc6)[C@@H]5C4)nc3)c3c(C#N)cnn3c2)cn1] | N-(((1R,5S,6r)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 2-hydroxy-2- phenylacetamide | 545.4 | ¹H NMR (400 MHz, DMSO-d₆) δ 9.19 (d, J = 1.2 Hz, 1H), 8.62 (s, 1H), 8.37 (s, 1H), 8.30 (d, J = 2.3 Hz, 1H), 8.14-8.07 (m, 2H), 7.79-7.71 (m, 2H), 7.43-7.35 (m, 2H), 7.30 (t, J = 7.3 Hz, 2H), 7.26-7.20 (m, 1H), 6.49 (d, J = 8.8 Hz, 1H), 6.10 (d, J = 4.8 Hz, 1H), 4.88 (d, J = 4.8 Hz, 1H), 3.85 (s, 3H), 3.69-3.52 (m, 4H), 3.03-2.93 (m, 2H), 1.91-1.78 (m, 2H), 0.87-0.77 (m, 1H). |
| 80 | 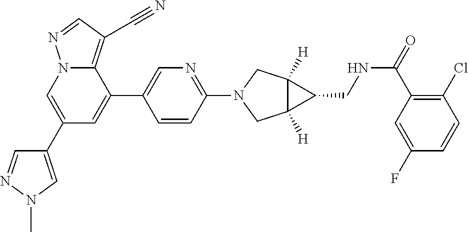 [SMILES: Cn1cc(-c2cc(-c3ccc(N4C[C@@H]5[C@@H](CNC(=O)c6cc(F)ccc6Cl)[C@@H]5C4)nc3)c3c(C#N)cnn3c2)cn1] | 2-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)methyl)- 5-fluorobenzamide | 567.3 | ¹H NMR (400 MHz, DMSO-d₆) δ 9.19 (d, J = 1.1 Hz, 1H), 8.68- 8.59 (m, 2H), 8.37 (s, 1H), 8.32 (d, J = 2.3 Hz, 1H), 8.10 (s, 1H), 7.75 (dd, J = 8.7, 2.4 Hz, 1H), 7.72 (d, J = 1.1 Hz, 1H), 7.54 (dd, J = 8.7, 4.9 Hz, 1H), 7.32 (m, 2H), 6.57 (d, J = 8.8 Hz, 1H), 3.86 (s, 3H), 3.73 (d, J = 10.4 Hz, 2H), 3.43 (d, J = 9.6 Hz, 2H), 3.22 (m, 2H), 1.73 (s, 2H), 0.90- 0.87 (m, 1H). |
| 81 | 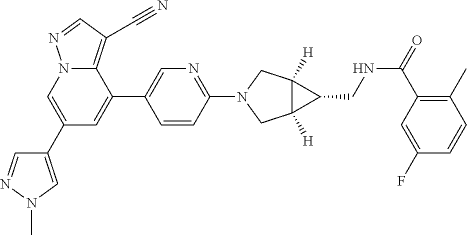 [SMILES: Cc1ccc(F)cc1C(=O)NC[C@@H]1[C@H]2CN(c3ccc(-c4cc(-c5cnn(C)c5)cn5ncc(C#N)c45)cn3)C[C@@H]12] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 5-fluoro-2- methylbenzamide | 547.4 | ¹H NMR (400 MHz, DMSO-d₆) δ 9.19 (d, J = 1.1 Hz, 1H), 8.68- 8.59 (m, 2H), 8.37 (s, 1H), 8.32 (d, J = 2.3 Hz, 1H), 8.10 (s, 1H), 7.75 (dd, J = 8.7, 2.4 Hz, 1H), 7.72 (d, J = 1.1 Hz, 1H), 7.54 (dd, J = 8.7, 4.9 Hz, 1H), 7.32 (m, 2H), 6.57 (d, J = 8.8 Hz, 1H), 3.86 (s, 3H), 3.73 (d, J = 10.4 Hz, 2H), 3.43 (d, J = 9.6 Hz, 2H), 3.22 (m, 2H), 1.73 (s, 2H), 0.90- 0.87 (m, 1H). |
| 82 | 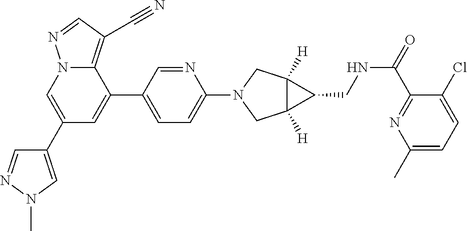 [SMILES: Cc1ccc(Cl)c(C(=O)NC[C@@H]2[C@H]3CN(c4ccc(-c5cc(-c6cnn(C)c6)cn6ncc(C#N)c56)cn4)C[C@@H]23)n1] | 3-chloro-N- (((1R,5S,6s)-3-(5-(3- cyano-6-(1-methyl- 1H-pyrazol-4- yl)pyrazolo[1.5- a]pyridin-4- yl)pyridin-2-yl)-3- azabicyclo[3.1.0] hexan-6-yl)methyl)- 6- methylpicolinamide | 564.2 | ¹H NMR (400 MHz, DMSO-d₆) δ 9.20 (s, 1H), 8.69 (s, 1H), 8.62 (s, 1H), 8.37 (s, 1H), 8.31 (s, 1H), 8.10 (s, 1H), 7.80 (d, J = 8.1 Hz, 1H), 7.77-7.70 (m, 2H), 7.54 (d, J = 8.2 Hz, 1H), 6.56 (d, J = 8.9 Hz, 1H), 3.86 (s, 3H), 3.72 (d, J = 10.5 Hz, 2H), 3.42 (d, J = 9.5 Hz, 2H), 3.24 (m, 2H), 2.45 (s, 3H), 1.73 (s, 2H). 0.94 (s, 1H). |
| 83 | 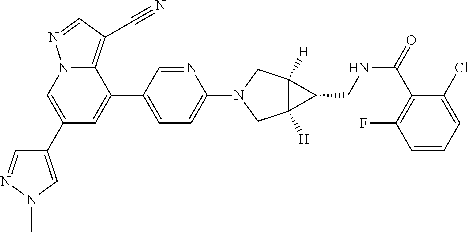 [SMILES: Cn1cc(-c2cc(-c3ccc(N4C[C@@H]5[C@@H](CNC(=O)c6c(F)cccc6Cl)[C@@H]5C4)nc3)c3c(C#N)cnn3c2)cn1] | 2-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)methyl)- 6- fluorobenzamide | 567.9 | ¹H NMR (400 MHz, DMSO-d₆) δ 9.20 (s, 1H), 8.88-8.78 (m, 2H), 8.62 (s, 1H), 8.37 (s, 1H), 8.30 (m, 2H), 8.10 (s, 1H), 7.75 (d, J = 9.3 Hz, 1H), 7.72 (d, J = 8.3 Hz, 2H), 6.57 (d, J = 8.7 Hz, 1H), 3.86 (s, 3H), 3.71 (d, J = 10.5 Hz, 2H), 3.43 (d, J = 9.6 Hz, 2H), 2.46-2.41 (m, 2H), 1.73 (s, 2H), 0.90 (s, 1H). |
| 84 | 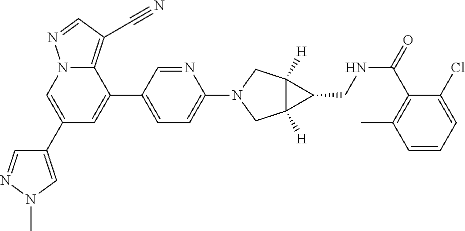 [SMILES: Cc1cccc(Cl)c1C(=O)NC[C@@H]1[C@H]2CN(c3ccc(-c4cc(-c5cnn(C)c5)cn5ncc(C#N)c45)cn3)C[C@@H]12] | 2-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)methyl)- 6- methylbenzamide | 563.3 | ¹H NMR (400 MHz, DMSO-d₆) δ 9.19 (d, J = 1.2 Hz, 1H), 8.85 (d, J = 4.5 Hz, 1H). 8.80 (t, J = 5.6 Hz, 1H), 8.62 (s, 1H), 8.36 (s, 1H), 8.30 (dd, J = 9.2, 5.2 Hz, 2H), 8.09 (s, 1H), 7.74 (ddd, J = 13.5, 8.5, 4.8 Hz, 3H), 6.57 (d, J = 8.8 Hz, 1H), 3.86 (s, 3H), 3.71 (d, J = 10.4 Hz, 2H), 3.43 (d, J = 9.7 Hz, 2H), 3.25 (t, J = 6.3 Hz, 2H), 1.73 (s, 2H), 0.95-0.87 (in. 1H). |
| 85 | 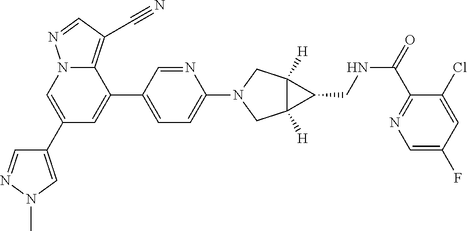 [SMILES: Cn1cc(-c2cc(-c3ccc(N4C[C@@H]5[C@@H](CNC(=O)c6ncc(F)cc6Cl)[C@@H]5C4)nc3)c3c(C#N)cnn3c2)cn1] | 3-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan-6-yl)methyl)- 5- fluoropicolinamide | 568.2 | ¹H NMR (400 MHz, DMSO-d₆) δ 9.19 (s, 1H), 8.76 (t, J = 5.4 Hz, 1H), 8.65-8.58 (m, 2H), 8.36 (s, 1H), 8.31 (d, J = 2.0 Hz, 1H), 8.19 (dd, J = 8.7, 2.3 Hz, 1H), 8.09 (s, 1H), 7.79-7.69 (m, 2H), 6.57 (d, J = 8.7 Hz, 1H), 3.86 (s, 3H), 3.72 (d, J = 10.4 Hz, 2H), 3.43 (d. J = 9.7 Hz, 2H), 3.27- 3.22 (m, 2H), 1.73 (s, 2H), 0.91 (s, 1H). |
| 86 | 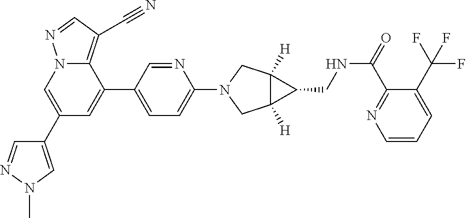 [SMILES: Cn1cc(-c2cc(-c3ccc(N4C[C@@H]5[C@@H](CNC(=O)c6ncccc6C(F)(F)F)[C@@H]5C4)nc3)c3c(C#N)cnn3c2)cn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 3- (trifluoromethyl) picolinamide | 584.3 | ¹H NMR (400 MHz, DMSO-d₆) δ 9.19 (d, J = 1.3 Hz, 1H), 8.85 (d, J = 4.4 Hz, 1H), 8.81 (t, J = 5.6 Hz, 1H), 8.62 (s, 1H), 8.36 (s, 1H), 8.30 (dd, J = 8.5, 5.2 Hz, 2H), 8.09 (s, 1H), 7.74 (m, 3H), 6.57 (d, J = 8.8 Hz, 1H), 3.86 (s, 3H), 3.71 (d, J = 10.5 Hz, 2H), 3.43 (d, J = 9.8 Hz, 2H), 3.25 (t, J = 6.3 Hz, 2H), 1.73 (s, 2H), 0.90 (m, 1H). |
| 87 | 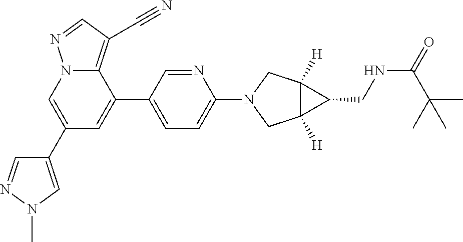 [SMILES: Cn1cc(-c2cc(-c3ccc(N4C[C@@H]5[C@@H](CNC(=O)C(C)(C)C)[C@@H]5C4)nc3)c3c(C#N)cnn3c2)cn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6- yl)methyl)pivalamid | 495.5 | ¹H NMR (400 MHz, DMSO-d₆) δ 9.19 (s, 1H), 8.61 (s, 1H), 8.37 (s, 1H), 8.30 (s, 1H), 8.09 (s, 1H), 7.73 (m, 2H), 7.55 (s, 1H), 6.55 (d, J = 8.7 Hz, 1H), 3.86 (s, 3H), 3.68 (d, J = 10.4 Hz, 2H), 3.39 (d, J = 9.4 Hz, 2H), 3.04 (t, J = 5.7 Hz, 2H), 1.62 (s, 2H), 1.08 (s, 9H), 0.80 (s, 1H). |
| 88 | 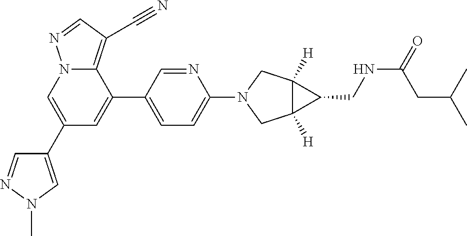 [SMILES: CC(C)CC(=O)NC[C@@H]1[C@H]2CN(c3ccc(-c4cc(-c5cnn(C)c5)cn5ncc(C#N)c45)cn3)C[C@@H]12] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan-6-yl)methyl)- 3- methylbutanamide | 495.5 | ¹H NMR (400 MHz, DMSO-d₆) δ 9.19 (s, 1H), 8.62 (s, 1H), 8.37 (s, 1H), 8.31 (d, J = 2.2 Hz, 1H), 8.09 (s, 1H), 7.90 (t, J = 5.4 Hz, 1H), 7.77-7.71 (m, 2H), 6.55 (d, J = 8.7 Hz, 1H), 3.86 (s, 3H), 3.69 (d, J = 10.4 Hz, 2H), 3.41 (d, J = 9.2 Hz, 2H), 3.02 (t, J = 6.1 Hz, 2H), 2.02-1.87 (m, 3H), 1.63 (s, 2H), 0.86 (d, J = 6.0 Hz, 6H), 0.81-0.74 (m, 1H). |

### Example 89

### 3-chloro-N-((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)picolinamide

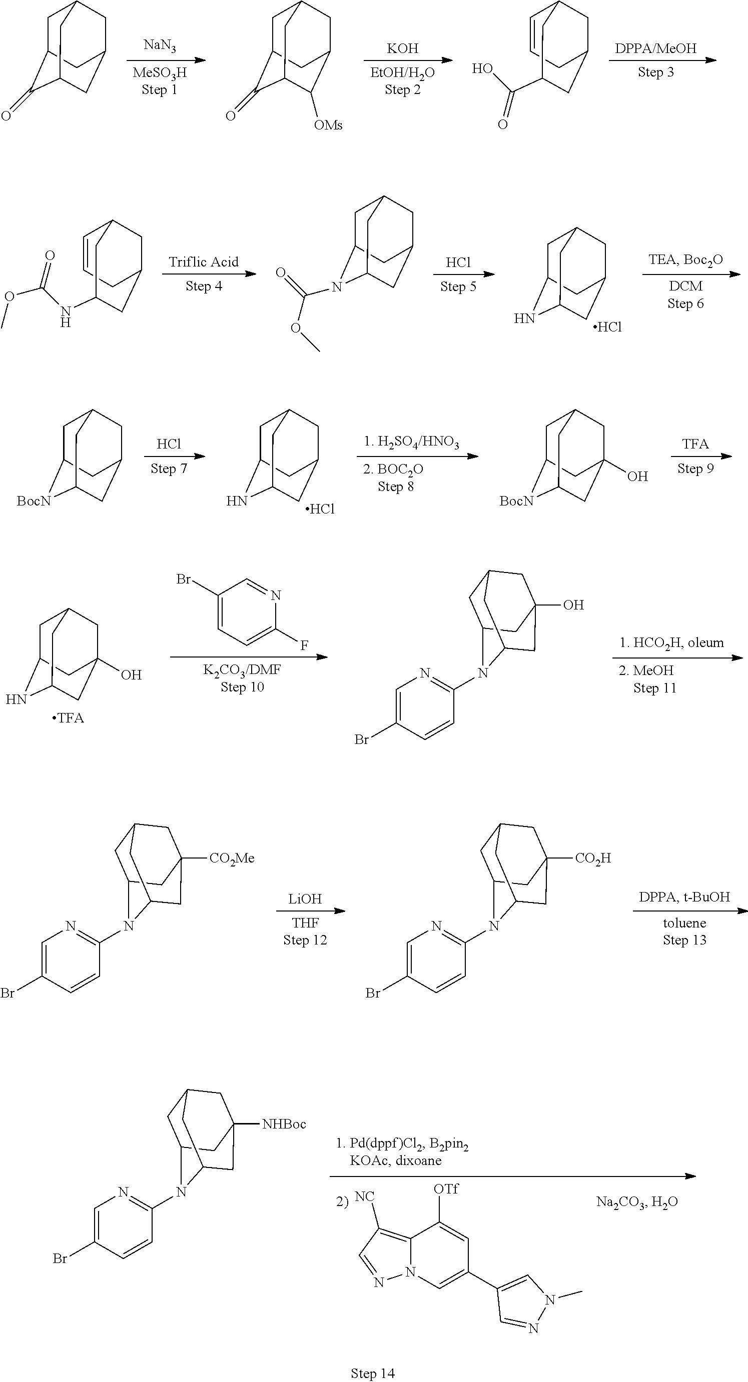

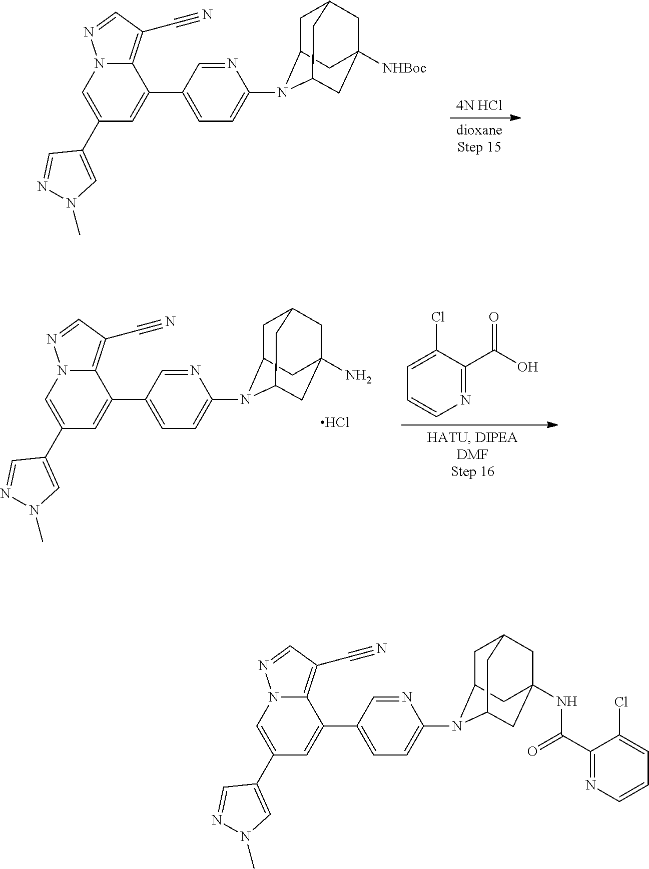

### Step 1. (1R,2S,3R,5S,7S)-4-oxoadamantan-2-yl methanesulfonate

To a solution of (1r,3r,5r,7r)-adamantan-2-one (50 g, 333 mmol) in MeSO₃H (416 g, 4329 mmol) was added portionwise NaN₃ (23 g, 351 mmol) over a period of 2 hours at 0° C. The reaction was stirred at rt for 3 days. The mixture was quenched with ice-water (2 L), and extracted with DCM/isopropanol (3/1, 2×3 L). The combined organic layers were washed with brine (1.5 L), dried over anhydrous Na₂SO₄, filtered off, and concentrated in vacuo to give the title compound (62 g, 62% yield).

### Step 2. bicyclo[3.3.1]non-6-ene-3-carboxylic acid

To a solution of the product of step 1 above (62 g, 254 mmol) in EtOH (600 mL) and water (600 mL) was added KOH (43 g, 762 mmol). The mixture was heated to 110° C. overnight. After cooling to rt, the mixture was acidified with 1N HCl to pH 2. After removing the most ethanol in vacuo, the mixture was extracted with EtOAc (2×2 L). The combined organic layers were washed with brine (500 mL), dried over anhydrous Na₂SO₄, filtered off, and concentrated in vacuo to give the title compound (42 g, 99% yield).

### Step 3. methyl bicyclo[3.3.1]non-6-en-3-ylcarbamate

To a solution of the product of step 2 above (42 g, 253 mmol) in toluene (400 mL) were added DPPA (76.5 g, 278 mmol) and TEA (38.3 g, 380 mmol). The mixture was stirred at 90° C. for 2 h under nitrogen atmosphere. After cooled to 0° C., to the mixture was added methanol (400 mL). The resulting mixture was heated to 100° C. overnight. The mixture was concentrated in vacuo and the residue was taken in EtOAc (2 L), washed with 1N HCl (500 mL), saturated aqueous NaHCO₃ (500 mL) and brine (500 mL), dried over anhydrous Na₂SO₄, filtered off, and concentrated in vacuo to give the title compound (20 g, 41% yield).

### Step 4. (1r,3r,5r,7r)-methyl 2-azaadamantane-2-carboxylate

To a solution of the product of Step 3 above (20 g, 102.5 mmol) in DCM (200 mL) was added triflic acid (77 g, 512 mmol) at 0° C. The mixture was stirred at rt overnight, quenched with ice-water (300 mL), extracted with DCM (2×500 mL). The combined organic layers were washed with saturated aqueous NaHCO₃ (200 mL) and brine (200 mL), dried over anhydrous Na₂SO₄, filtered off, and concentrated in vacuo to give the title compound (20 g, 100% yield).

### Step 5. (1r,3r,5r,7r)-2-azaadamantane hydrochloride

The product of step 4 above (20 g, 102.5 mmol) was added to 4N HCl/dioxane (200 mL) and concentrated hydrochloric acid (200 mL) at 0° C. The mixture was stirred at 90° C. overnight and concentrated in vacuo to give the title compound (18 g, 100% yield).

### Step 6. (1r,3r,5r,7r)-tert-butyl 2-azaadamantane-2-carboxylate

To a solution of the product of step 5 above (18 g, 103 mmol) in DCM (200 mL) was added TEA (31 g, 309 mmol) and Boc₂O (29 g, 134 mmol) at 0° C. The mixture was stirred at 0˜rt overnight. The mixture was diluted with DCM (300 mL), which was washed with water (100 mL) and brine (100 mL), dried over anhydrous Na₂SO₄, filtered off, and concentrated in vacuo. The residue was purified by flash column chromatography on silica gel (PE:EtOAc=50:1 to 20:1) to give the title compound (10 g, 41% yield).

### Step 7. (1r,3r,5r,7r)-2-azaadamantane hydrochloride

The product of step 6 above (10 g, 102.5 mmol) was added to 4N HCl/dioxane (100 mL) at 0° C. The mixture was stirred at rt for 2h. The mixture was concentrated in vacuo and the residue was triturated with hexane:ether (1:1, 50 mL×2) to give the title compound (4.8 g, 65% yield). LC-MS (m/z): 138.1

### Step 8. (1R,3S,5s,7s)-tert-butyl 5-hydroxy-2-azaadamantane-2-carboxylate

The product of step 7 above (4.3 g, 24.7 mmol) was added to concentrated nitric acid (43 mL) and H₂SO₄ (7.2 mL) at 0° C. The mixture was stirred at 80° C. overnight. After cooling to rt, the mixture was quenched with ice-water (200 mL), and basified with solid Na₂CO₃. The aqueous layer was washed with DCM. The aqueous layer was diluted with THF (200 mL), cooled to 0° C., and treated with TEA (5 g, 49.4 mmol) and Boc₂O (7 g, 32.1 mmol). The resulting mixture was stirred at 0˜rt overnight and extracted with EtOAc (300 mL×2). The combined organic layers were washed with water (100 mL) and brine (100 mL), dried over anhydrous Na₂SO₄, filtered off, and concentrated in vacuo. The residue was purified by flash column chromatography on silica gel (PE:EtOAc=8:1 to 2:1) to give the title compound (2.47 g, 40% yield) as a colorless oil. ¹H NMR (400 MHz, CDCl₃) δ 4.46 (s, 2H), 2.29 (s, 1H), 1.79 (s, 2H), 1.73 (t, J=14.2 Hz, 4H), 1.67 (s, 1H), 1.64 (s, 1H), 1.61 (s, 2H), 1.53 (d, J=12.2 Hz, 2H), 1.48-1.40 (m, 9H).

### Step 9. (1R,3S,5s,7s)-2-azaadamantan-5-ol TFA salt

To a solution of the product of step 8 above (2.47 g, 9.76 mmol) in DCM (30 mL) was added TFA (6 mL) at 0° C. The reaction was stirred at 0° C.-rt for 4 h. The mixture was concentrated in vacuo and the residue was triturated with hexane:ether (1:1, 20 mL×2) to give the title compound (2.5 g, 100% yield).

### Step 10. (5s,7s)-2-(5-bromopyridin-2-yl)-2-azaadamantan-5-ol

To a solution of the product of step 9 above (1.75 g, 7 mmol) in DMF (20 mL) were added K₂CO₃ (2.9 g, 21 mmol) and 5-bromo-2-fluoropyridine (1.48 g, 8.4 mmol) successively. The reaction was stirred at 100° C. overnight. After cooling to rt, the mixture was diluted with EtOAc (200 mL), washed with water (50 mL×3) and brine (50 mL), dried over anhydrous Na₂SO₄, filtered off, and concentrated in vacuo. The residue was purified by flash column chromatography on silica gel (PE:EtOAc=10:1 to 2:1) to give the title compound (914 mg, 37% yield).

### Step 11. (5s,7s)-methyl 2-(5-bromopyridin-2-yl)-2-azaadamantane-5-carboxylate

To 15% oleum (16 mL) was added dropwise the product of step 10 above (914 mg, 2.97 mmol) in 98% formic acid (4.55 mL) at 60° C. Upon completion of this addition, 98% formic acid (4.55 mL) was added dropwise over a period of 10 minutes. The mixture was stirred at 100° C. for 1 h. The mixture was slowly poured into methanol (38 mL) cooled to 0° C. with vigorously stirring. The resulting mixture was stirred at 0˜rt overnight. The mixture was concentrated in vacuo. The residue was poured into ice-water (100 mL), basified with solid Na₂CO₃, and extracted with DCM:MeOH (10:1, 50 mL×3). The organic layer was dried over anhydrous Na₂SO₄, filtered off, and concentrated in vacuo. The residue was purified by flash column chromatography on silica gel (PE:EtOAc=6:1 to 1:1) to give the title compound (846 mg, 81% yield).

### Step 12. (5s,7s)-2-(5-bromopyridin-2-yl)-2-azaadamantane-5-carboxylic acid

To a solution of the product of step 11 above (846 mg, 2.41 mmol) in THF (9 mL) and water (6 mL) was added LiOH H₂O (304 mg, 7.23 mmol). The reaction was stirred at 45° C. overnight and acidified with concentrated hydrochloric acid to pH 5 at 0° C. The mixture was extracted with EtOAc (100 mL) and DCM: isopropanol (3:1, 100 mL). The combined organic layers were washed with water (20 mL) and brine (20 mL), dried over anhydrous Na₂SO₄, filtered off, and concentrated in vacuo to give the title compound (800 mg, 99% yield).

### Step 13. tert-butyl ((5s,7s)-2-(5-bromopyridin-2-yl)-2-azaadamantan-5-yl)carbamate

To a solution of the product of step 12 above (600 mg, 1.78 mmol) in toluene (6 mL) and t-BuOH (6 mL) were added DPPA (734 mg, 2.67 mmol) and TEA (360 mg, 3.56 mmol). The mixture was stirred at 100° C. overnight under nitrogen atmosphere. The mixture was concentrated in vacuo. The residue was washed with water (30 mL) and brine (30 mL), dried over anhydrous Na₂SO₄, filtered off, and concentrated in vacuo. The residue was purified by flash column chromatography on silica gel (PE:EtOAc=30:1 to 15:1) to give the title compound (360 mg, 50% yield).

### Step 14. tert-butyl ((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)carbamate

To a solution of the product of Step 13 above (150 mg, 0.368 mmol) in dioxane (1.5 mL) was added B2pin2 (93 mg, 0.368 mmol), KOAc (72 mg, 0.736 mmol) and Pd(dppf)Cl₂ DCM (30 mg, 0.0368 mmol). The mixture was stirred at 100° C. for 3 h under nitrogen atmosphere.

To the mixture after cooling to rt was added Intermediate 1 (137 mg, 0.368 mmol), Na₂CO₃ (78 mg, 0.736 mmol), Pd(dppf)Cl₂ DCM (30 mg, 0.0368 mmol), dioxane (1.5 mL) and water (0.3 mL). The reaction mixture was stirred at 110° C. for 5 h under nitrogen atmosphere. The mixture was diluted with EtOAc (100 mL), which was washed with water (20 mL) and brine (20 mL), dried over anhydrous Na₂SO₄, filtered off, and concentrated in vacuo. The residue was purified by flash column chromatography on silica gel (DCM:EtOAc=2:1 to 1:1) to give the title compound (180 mg, 89% yield).

### Step 15. 4-(6-((1R,3S,5s,7s)-5-amino-2-azaadamantan-2-yl)pyridin-3-yl)-6-(1-methyl-1H-pyrazol-4-yl)pyrazolo[1,5-a]pyridine-3-carbonitrile hydrochloride

To a solution of the product of step 14 above (180 mg, 0.327 mmol) in THF (2 mL) was added 4N HCl/dioxane (4 mL) at 0° C. The mixture was stirred at rt for 2h before being concentrated to give the title compound (200 mg, 100% yield).

### Step 16. 3-chloro-N-((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)picolinamide

To a solution of the product of step 15 above (80 mg, 0.143 mmol) in DMF (1 mL) was added 3-chloropicolinic acid (34 mg, 0.214 mmol), HATU (82 mg, 0.214 mmol) and DIPEA (111 mg, 0.858 mmol). The mixture was stirred at 50° C. overnight. The mixture was filtered off and the filtrate was directly purified by reverse phase flash column chromatography (MeOH/H₂O) to give the title compound (20 mg, 24% yield). ESI-MS (m/z): 590.2 [M+1]⁺.

¹H NMR (400 MHz, CDCl₃) δ 8.61 (s, 1H), 8.38 (d, J=3.2 Hz, 1H), 8.29 (s, 1H), 8.22 (s, 1H), 7.76 (s, 2H), 7.74-7.64 (m, 3H), 7.39 (s, 1H), 7.32 (dd, J=7.9, 4.4 Hz, 1H), 6.77 (d, J=8.9 Hz, 1H), 3.94 (s, 3H), 3.09 (d, J=6.8 Hz, 2H), 2.34 (s, 3H), 2.28 (d, J=11.8 Hz, 2H), 2.17 (d, J=11.2 Hz, 2H), 1.91 (d, J=12.3 Hz, 2H), 1.78 (d, J=12.3 Hz, 2H).

### Example 90

### (1R,3S,5s,7s)-N-(3-chloropyridin-2-yl)-2-(5-(3-cyano-6-(1-methyl-1H-pyrazol-4-yl)pyrazolo[1,5-a]pyridin-4-yl)pyridin-2-yl)-2-azaadamantane-5-carboxamide

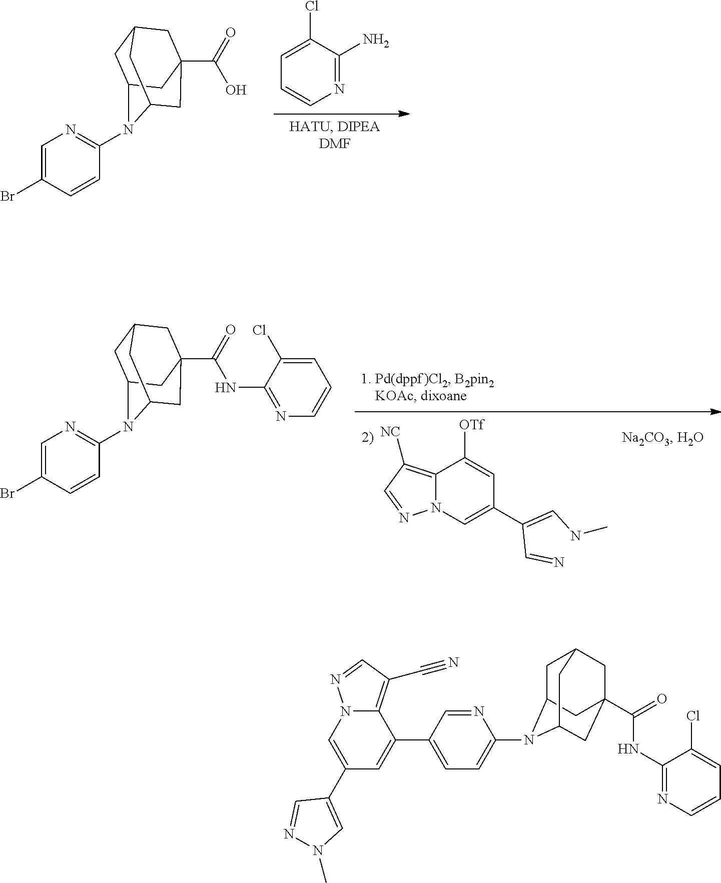

### Step 1. (5s,7s)-2-(5-bromopyridin-2-yl)-N-(3-chloropyridin-2-yl)-2-azaadamantane-5-carboxamide

To a solution of the product of step 12 of Example 89 (200 mg, 0.593 mmol) in DMF (3 mL) was added 3-chloropyridin-2-amine (114 mg, 0.890 mmol), HATU (338 mg, 0.890 mmol) and DIPEA (229 mg, 1.779 mmol). The mixture was stirred at 50° C. overnight. After cooling to rt, the mixture was filtered off and the filtrate was purified by reverse phase flash column chromatography (MeOH/H₂O) to give the title compound (86 mg, 32% yield).

### Step 2. (1R,3S,5s,7s)-N-(3-chloropyridin-2-yl)-2-(5-(3-cyano-6-(1-methyl-1H-pyrazol-4-yl)pyrazolo[1,5-a]pyridin-4-yl)pyridin-2-yl)-2-azaadamantane-5-carboxamide

To a solution of the product of Step 1 above (43 mg, 0.096 mmol) in dioxane (1 mL) was added B₂Pin₂ (24 mg, 0.096 mmol), KOAc (19 mg, 0.192 mmol) and Pd(dppf)Cl₂ DCM (8 mg, 0.0096 mmol). The mixture was stirred at 100° C. for 8 h under nitrogen atmosphere. To the mixture cooled to rt was added Intermediate 1 (36 mg, 0.096 mmol), Na₂CO₃ (20 mg, 0.192 mmol), Pd(dppf)Cl₂ DCM (8 mg, 0.0096 mmol), and dioxane (1 mL) and water (0.1 mL). The reaction mixture was stirred at 110° C. for 3 h under nitrogen atmosphere. The mixture was filtered off, and the filtrate was diluted with DCM:MeOH (10:1, 60 mL), washed with water (10 mL) and brine (10 mL), dried over anhydrous Na₂SO₄, filtered off, and concentrated in vacuo. The residue was purified by prep-TLC (DCM:MeOH=10:1 and DCM:EtOAc=1:2) to give the title compound (18 mg, 32% yield). ESI-MS (m/z): 590.5 [M+1]⁺. ¹H NMR (400 MHz, CDCl₃) δ 8.62 (s, 1H), 8.33 (s, 2H), 8.22 (s, 1H), 7.79 (d, J=12.3 Hz, 1H), 7.70 (s, 3H), 7.41 (s, 1H), 7.08 (s, 1H), 6.79 (d, J=8.7 Hz, 1H), 3.95 (s, 3H), 2.33 (s, 2H), 2.21 (s, 4H), 2.07 (d, J=12.0 Hz, 2H), 1.99 (d, J=12.8 Hz, 3H), 1.81 (d, J=12.0 Hz, 2H).

### Examples 91

### N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-3-fluoropicolinamide

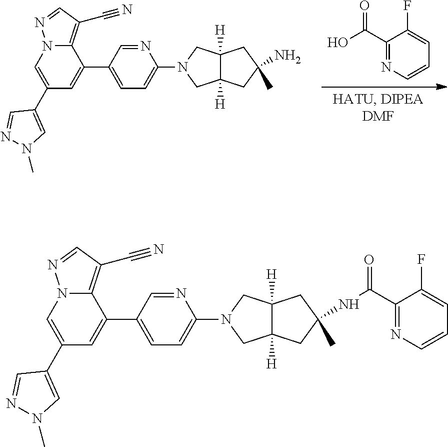 [SMILES: Cn1cc(-c2cc(-c3ccc(N4C[C@@H]5C[C@@](C)(N)C[C@@H]5C4)nc3)c3c(C#N)cnn3c2)cn1 ; O=C(O)c1ncccc1F ; "HATU, DIPEADMF" ; Cn1cc(-c2cc(-c3ccc(N4C[C@@H]5C[C@@](C)(NC(=O)c6ncccc6F)C[C@@H]5C4)nc3)c3c(C#N)cnn3c2)cn1]

To a solution of Intermediate 35 (60 mg, 0.137 mmol), 3-fluoropicolinic acid (21 mg, 0.151 mmol), and HATU (78 mg, 1.5 mmol) in DMF (0.6 mL) was added DIPEA (53 mg, 3.0 mmol) at rt. The mixture was stirred at 70° C. for 2h, cooled to rt, and purified by reverse phase flash column chromatography on C18 (MeOH/H₂O) to give the title compound (38 mg, yield: 51%). ESI-MS (m/z): 562.4 [M+1]⁺. Rotamers: ¹H NMR (400 MHz, DMSO-d₆) δ 9.20 (s, 1H), 8.63 (s, 1H), 8.47-8.29 (m, 3H), 8.19 (s, 1H), 8.10 (s, 1H), 7.89-7.68 (m, 3H), 7.64-7.53 (m, 1H), 6.62 (dd, J=17.4, 8.9 Hz, 1H), 3.86 (s, 3H), 3.62-3.41 (m, 4H), 2.90 (m, 1.6H), 2.71-2.63 (m, 1.6H), 2.19-2.14 (m, 0.4H), 2.05-1.99 (m, 0.4H), 1.50 (s, 3H), 1.47-1.38 (m, 2H).

### Example 92

### 4-(6-((3aR,6aS)-5-((2-chloro-6-fluorophenyl)sulfonyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile

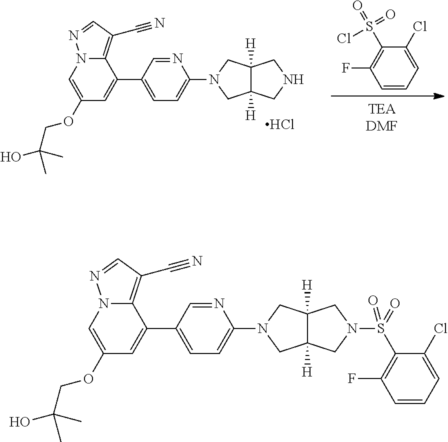

The compound was synthesized following the procedure used to make Example 11 starting from intermediate 11. ESI-MS (m/z): 611.2 [M+1]⁺. ¹H NMR (400 MHz, CDCl₃) δ 8.32 (s, 1H), 8.20 (s, 1H), 8.15 (s, 1H), 7.71 (d, J=8.6 Hz, 1H), 7.37 (t, J=13.8 Hz, 2H), 7.12 (d, J=11.1 Hz, 2H), 6.50 (d, J=8.6 Hz, 1H), 3.86 (s, 3H), 3.82 (m, 4H), 3.47 (m, 4H), 3.15 (s, 2H), 1.39 (s, 6H).

### Example 93

### 2-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-fluorobenzamide

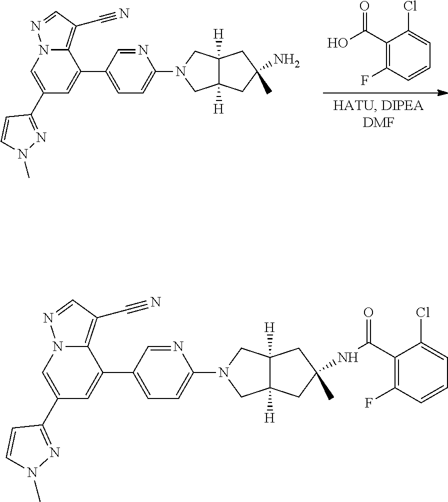

To a solution of Intermediate 35 (88 mg, 0.2 mmol), 2-chloro-6-fluorobenzoic acid (37 mg, 0.24 mmol), and HATU (114 mg, 0.3 mmol) in DMF (5 mL) was added DIPEA (78 mg, 0.6 mmol) at rt. The mixture was stirred at rt for 2h, diluted with EtOAc (100 mL), washed with H₂O (30 mL×2) and brine (30 mL), dried over anhydrous Na₂SO₄, filtered off, and concentrated. The residue was purified by prep-TLC (DCM/MeOH=15/1) to give the title compound (81 mg, yield: 70%). ESI-MS (m/z): 595.4 [M+1]⁺. ¹H NMR (400 MHz, CD₃OD) δ 8.95 (s, 1H), 8.44-8.20 (m, 2H), 8.04 (d, J=84.7 Hz, 1H), 7.85-7.67 (m, 2H), 7.30 (m, 1H), 7.20 (m, 1H), 7.04 (m, 1H), 6.65 (d, J=11.5 Hz, 2H), 3.96 (s, 3H), 3.68-3.46 (m, 4H), 3.06 (m, 1H), 2.71 (m, 1H), 2.31 (d, J=6.5 Hz, 1H), 2.10 (m, 1H), 1.64-1.49 (m, 5H).

### Example 94

### 3-chloro-N-(((1R,5S,6s)-3-(5-(3-cyano-6-ethoxypyrazolo[1,5-a]pyridin-4-yl)pyridin-2-yl)-3-azabicyclo[3.1.0]hexan-6-yl)methyl)picolinamide

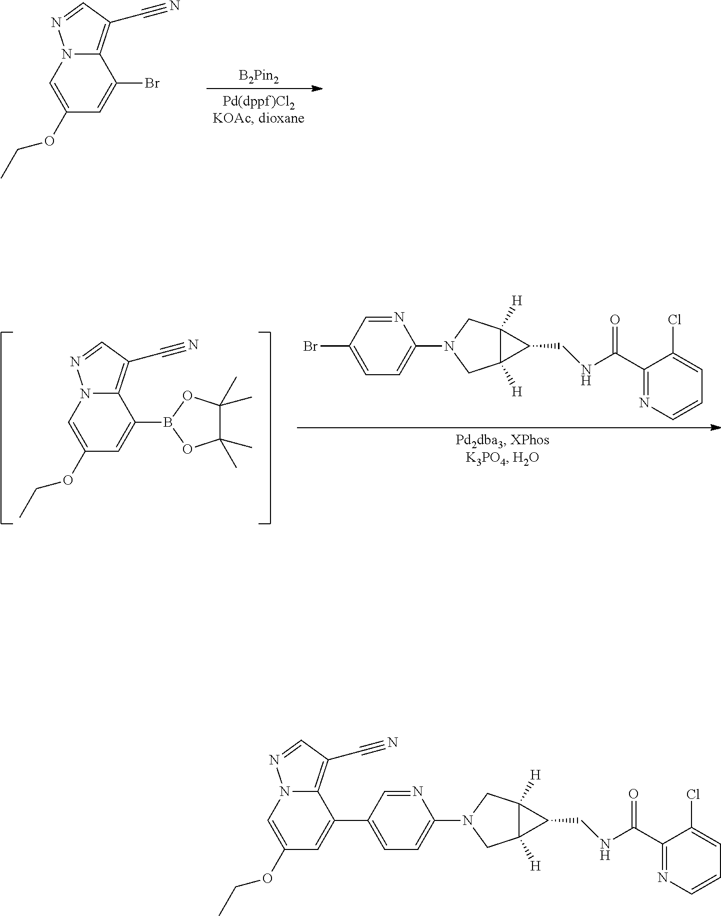

A mixture of Intermediate 4 (58 mg, 0.218 mmol), B₂Pin₂ (58 mg, 0.229 mmol), Pd(dppf)Cl₂ DCM (18 mg, 0.0218 mmol), and KOAc (43 mg, 0.436 mmol) in dioxane (1 mL) was stirred at 100° C. under N₂ for 4h. The mixture was cooled to rt and treated with Intermediate 37 (89 mg, 0.218 mmol), Pd₂dba₃ (10 mg, 0.0109 mmol), XPhos (21 mg, 0.0436 mmol), K₃PO₄ (139 mg, 0.654 mmol) and dioxane/H₂O (4 mL/1 mL). The mixture was stirred at 110° C. under N₂ overnight, cooled to rt, and purified by reverse phase flash column chromatography on C18 (MeOH/H₂O) to give the title compound (4.8 mg, yield: 4.3%). ESI-MS (m/z): 514.3 [M+1]⁺. ¹H NMR (400 MHz, CDCl₃) δ 8.49 (s, 1H), 8.29 (s, 1H), 8.18 (s, 1H), 8.10 (s, 1H), 7.93 (s, 1H), 7.83 (d, J=7.9 Hz, 1H), 7.75-7.66 (m, 1H), 7.38 (dd, J=7.4, 4.2 Hz, 1H), 7.08 (s, 1H), 6.50 (s, 1H), 4.08 (q, J=6.6 Hz, 2H), 3.86 (m, 2H), 3.59 (m, 2H), 3.47 (m, 2H), 1.79 (m, 2H), 1.49 (t, J=6.7 Hz, 3H), 0.92-0.84 (m, 1H).

### Example 95

### 3-chloro-N-(((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)picolinamide

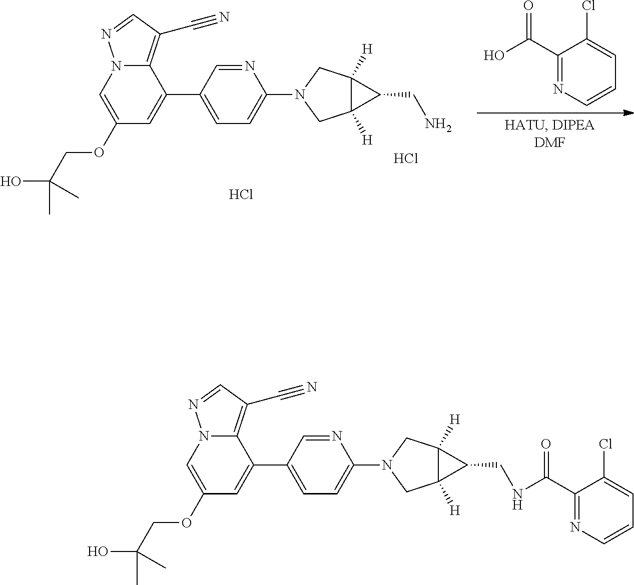

To a solution of Intermediate 38 (70 mg, 0.09 mmol), 3-chloropicolinic acid (14 mg, 0.09 mmol), and HATU (51 mg, 0.135 mmol) in DMF (1 mL) was added DIPEA (58 mg, 0.45 mmol) at rt. The mixture was stirred at 80° C. for 1 h, cooled to rt, and purified by reverse phase flash column chromatography on C18 (MeOH/H₂O) to give the title compound (38 mg, yield: 76%). ESI-MS (m/z): 558.2 [M+1]⁺. ¹H NMR (400 MHz, DMSO-d₆) δ 8.75 (s, 1H), 8.63 (s, 1H), 8.54 (s, 2H), 8.26 (s, 1H), 8.01 (d, J=8.2 Hz, 1H), 7.71 (d, J=7.2 Hz, 1H), 7.53 (d, J=4.6 Hz, 1H), 7.23 (s, 1H), 6.55 (d, J=8.6 Hz, 1H), 4.68 (s, 1H), 3.84 (m, 2H), 3.71 (m, J=10.4 Hz, 2H), 3.42 (m, J=9.8 Hz, 2H), 3.25 (m, 2H), 1.73 (s, 2H), 1.20 (s, 6H), 0.90 (m, 1H).

### Example 96

### 2-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-fluorobenzamide

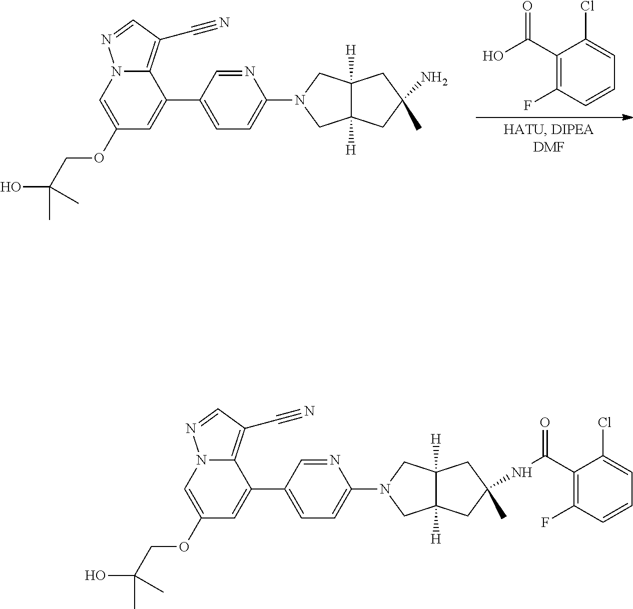

To a solution of Intermediate 41 (30 mg, 0.067 mmol), 2-chloro-6-fluorobenzoic acid (12 mg, 0.067 mmol), and HATU (38 mg, 0.101 mmol) in DMF (3 mL) was added DIPEA (26 mg, 0.201 mmol) at rt. The mixture was stirred at rt overnight and concentrated in vacuo. The residue was taken up in DCM/MeOH (10/1, 50 mL), washed with H₂O (15 mL) and brine (15 mL), dried over anhydrous Na₂SO₄, filtered off, and concentrated. The residue was purified by prep-TLC (DCM/MeOH=10/1) to give the title compound (14 mg, yield: 35%). ESI-MS (m/z): 603.4 [M+1]⁺. ¹H NMR (400 MHz, CDCl₃) δ 8.33 (s, 1H), 8.20 (s, 1H), 8.15 (s, 1H), 7.72 (d, J=7.9 Hz, 1H), 7.30 (d, J=6.5 Hz, 1H), 7.21 (d, J=8.0 Hz, 1H), 7.14 (s, 1H), 7.04 (t, J=8.3 Hz, 1H), 6.58 (d, J=8.7 Hz, 1H), 5.58 (s, 1H), 3.86 (s, 2H), 3.64 (m, 2H), 3.55 (m, 2H), 3.08 (m, 2H), 2.68 (m, 2H), 1.66-1.60 (m, 5H), 1.38 (s, 6H).

### Example 97

### N-((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)acetamide

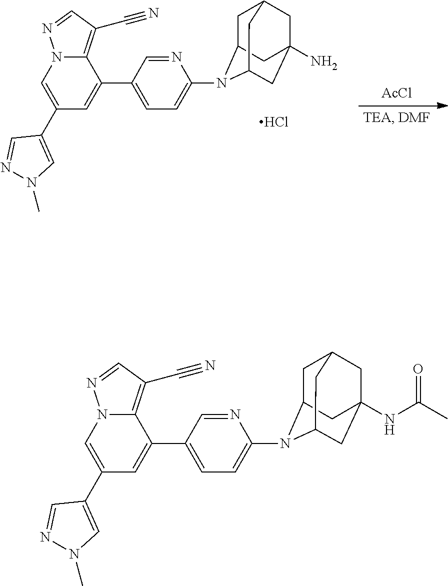 [SMILES: "AcCl" ; Cn1cc(-c2cc(-c3ccc(N4C5CC6CC4CC(N)(C6)C5)nc3)c3c(C#N)cnn3c2)cn1 ; Cl ; "TEA, DMF" ; CC(=O)NC12CC3CC(C1)N(c1ccc(-c4cc(-c5cnn(C)c5)cn5ncc(C#N)c45)cn1)C(C3)C2]

To an ice-water cooled solution of the product of Step 15 in Example 89 (60 mg, 0.107 mmol) in DMF (1 mL) were added TEA (65 mg, 0.642 mmol) and AcCl (17 mg, 0217 mmol) sequentially. The mixture was stirred at rt for 3h and concentrated. The residue was taken up in EtOAc (50 mL), washed with H₂O (10 mL) and brine (10 mL), dried over anhydrous Na₂SO₄, filtered off, and concentrated. The residue was purified by prep-TLC (DCM/MeOH=10/1) to give the title compound (13 mg, yield: 25%). ESI-MS (m/z): 493.4 [M+1]⁺. ¹H NMR (400 MHz, CDCl₃+CD₃OD) δ 8.61 (s, 1H), 8.27 (s, 1H), 8.21 (s, 1H), 7.81-7.60 (m, 3H), 7.26 (s, 1H), 6.74 (d, J=6.0 Hz, 1H), 5.94 (s, 1H), 4.72 (s, 2H), 3.94 (s, 3H), 2.36-1.65 (m, 14H).

### Example 98

### (1R,3 S,5s,7s)-N-(3-chloropyridin-2-yl)-2-(5-(3-cyano-6-(2-hydroxy-2-methylpropoxy)pyrazolo[1,5-a]pyridin-4-yl)pyridin-2-yl)-2-azaadamantane-5-carboxamide

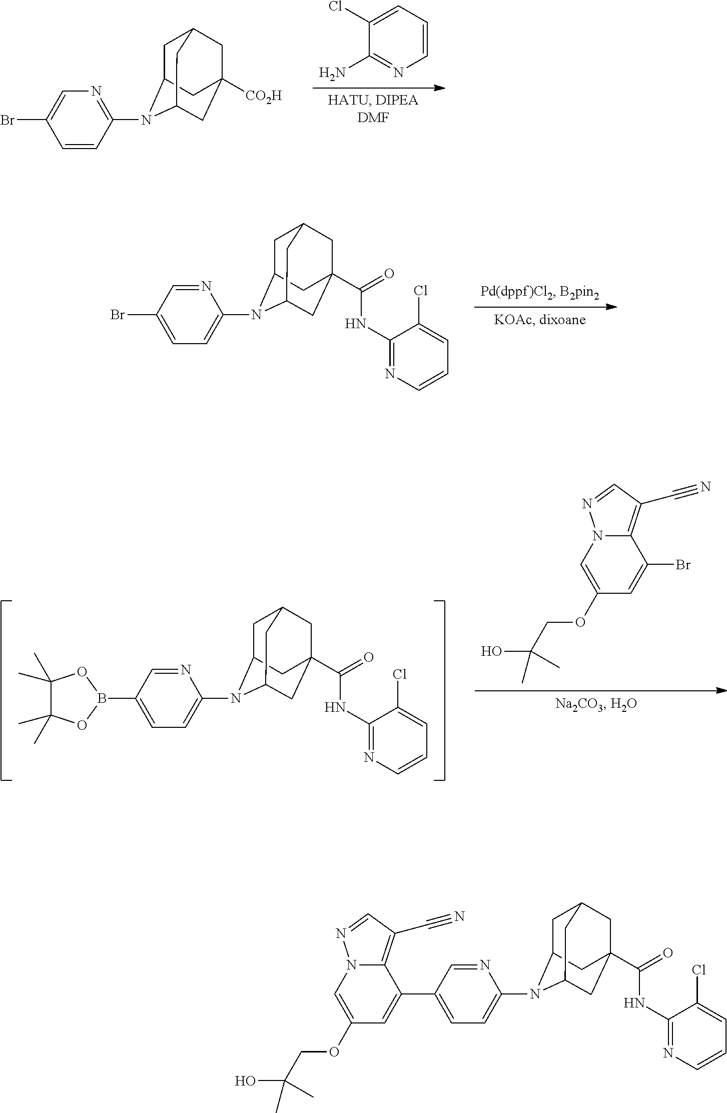

### Step 1. (1R,3S,5s,7s)-2-(5-bromopyridin-2-yl)-N-(3-chloropyridin-2-yl)-2-azaadamantane-5-carboxamide

To a solution of the product of Step 12 in Example 89 (200 mg, 0.593 mmol), 3-chloropyridin-2-amine (114 mg, 0.89 mmol), and HATU (338 mg, 0.89 mmol) in THF (3 mL) was added DIPEA (229 mg, 1.779 mmol) at rt. The mixture was stirred at 50° C. overnight, cooled to rt, and purified by reverse phase flash column chromatography on C18 to give the title compound (86 mg, yield: 32%).

### Step 2. (1R,3S,5s,7s)-N-(3-chloropyridin-2-yl)-2-(5-(3-cyano-6-(2-hydroxy-2-methylpropoxy)pyrazolo[1,5-a]pyridin-4-yl)pyridin-2-yl)-2-azaadamantane-5-carboxamide

A mixture of the product of Step 1 above (43 mg, 0.096 mmol), B₂Pin₂ (24 mg, 0.096 mmol), Pd(dppf)Cl₂ DCM (8 mg, 0.0096 mmol), and KOAc (19 mg, 0.192 mmol) in dioxane (1 mL) was stirred at 100° C. under N₂ for 3h. The mixture was cooled to rt and treated with Intermediate 3 (30 mg, 0.218 mmol), Pd(dppf)Cl₂ DCM (8 mg, 0.0096 mmol), Na₂CO₃ (20 mg, 0.192 mmol) and dioxane/H₂O (1.5 mL/0.15 mL). The mixture was stirred at 110° C. under N₂ for 6h, cooled to rt, filtered, and concentrated. The residue was taken up in EtOAc (50 mL), washed with H₂O (10 mL) and brine (10 mL), dried over anhydrous Na₂SO₄, and concentrated. The residue was purified by prep-TLC (DCM/EtOAc=1/1) to give the title compound (15 mg, yield: 26%). ESI-MS (m/z): 598.3 [M+1]⁺. ¹H NMR (400 MHz, CDCl₃+CD₃OD) δ 8.36-8.29 (m, 1H), 8.26 (s, 1H), 8.14 (s, 2H), 8.13 (s, 1H), 7.71 (d, J=7.9 Hz, 1H), 7.65 (d, J=8.7 Hz, 1H), 7.15 (s, 1H), 7.10-7.02 (m, 1H), 6.76 (d, J=8.9 Hz, 1H), 4.76 (s, 2H), 3.80 (s, 2H), 2.31 (s, 1H), 2.15 (d, J=12.1 Hz, 4H), 2.04 (d, J=12.1 Hz, 2H), 1.96 (d, J=12.2 Hz, 2H), 1.78 (d, J=12.3 Hz, 2H), 1.32 (s, 6H).

### Example 99

### N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-2-hydroxy-3-methylbutanamide

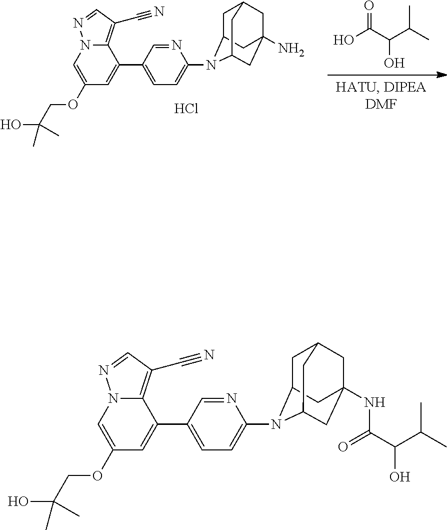

To a solution of Intermediate 42 (35 mg, 0.062 mmol), 2-hydroxy-3-methylbutanoic acid (7 mg, 0.062 mmol), and HATU (35 mg, 0.093 mmol) in DMF (1 mL) was added DIPEA (40 mg, 0.31 mmol) at rt. The mixture was stirred at rt overnight and filtered. The filtrate was purified by reverse phase flash column chromatography on C18 (MeOH/H₂O) to give the title compound (16 mg, yield: 47%). ESI-MS (m/z): 559.4 [M+1]⁺. ¹H NMR (400 MHz, CDCl₃+CD₃OD) δ 8.24 (s, 1H), 8.13 (d, J=7.6 Hz, 2H), 7.63 (d, J=7.2 Hz, 1H), 7.13 (s, 1H), 6.71 (d, J=8.9 Hz, 1H), 6.63 (s, 1H), 4.72 (s, 2H), 3.80 (s, 2H), 3.72 (d, J=2.3 Hz, 1H), 2.26-1.69 (m, 12H), 1.32 (s, 6H), 0.93 (d, J=6.8 Hz, 3H), 0.78 (d, J=6.7 Hz, 3H).

### Example 100

### 2-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)-6-fluorobenzamide

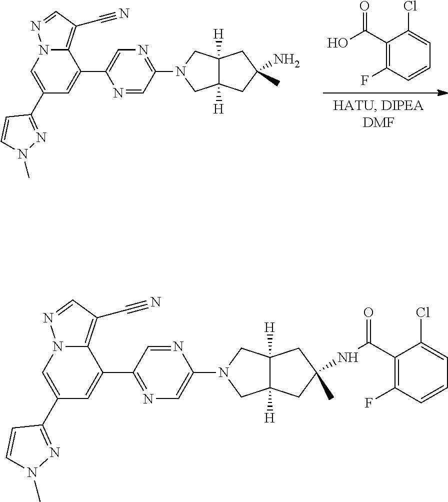

To a solution of Intermediate 44 (44 mg, 0.1 mmol), 2-chloro-6-fluorobenzoic acid (19 mg, 0.11 mmol), and HATU (57 mg, 0.15 mmol) in DMF (3 mL) was added DIPEA (39 mg, 0.30 mmol) at rt. The mixture was stirred at rt overnight and filtered. The filtrate was taken up in DCM/MeOH (10/1, 50 mL), washed with H₂O (15 mL) and brine (15 mL), dried over anhydrous Na₂SO₄, filtered off, and concentrated. The residue was purified by prep-TLC (DCM/MeOH=15/1) to give the title compound (20 mg, yield: 34%). ESI-MS (m/z): 596.3 [M+1]⁺. ¹H NMR (400 MHz, CD₃OD) δ 8.90 (s, 1H), 8.43 (s, 1H), 8.28 (s, 1H), 8.08 (s, 1H), 7.98 (s, 1H), 7.46 (s, 1H), 7.26 (s, 1H), 7.15 (d, J=6.3 Hz, 1H), 7.01 (s, 1H), 6.60 (s, 1H), 3.94 (s, 3H), 3.64 (m, 2H), 3.54 (m, 2H), 3.06 (s, 2H), 2.68 (m, 2H), 1.62-1.51 (m, 5H).

### Example 101

### 4-(5-((3aR,5s,6aS)-5-(((6-methoxypyridin-3-yl)methyl)amino)-5-methylhexahydrocyclopenta[c]pyrrol-2(1H)-yl)pyrazin-2-yl)-6-(1-methyl-1H-pyrazol-4-yl)pyrazolo[1,5-a]pyridine-3-carbonitrile

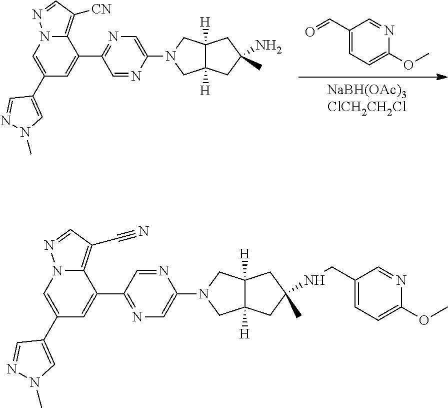

To a solution of Intermediate 45 (30 mg, 0.068 mmol) and 6-methoxynicotinaldehyde (12 mg, 0.082 mmol) in DCM (5 mL) was added NaBH(OAc)₃ (43 mg, 0.204 mmol). The mixture was stirred at 80° C. for 4h, cooled to rt, diluted with DCM/MeOH (10/1, 100 mL), washed with H₂O (30 mL×2) and brine (30 mL), dried over anhydrous Na₂SO₄, filtered off, and concentrated. The residue was purified by reverse phase flash column chromatography on C18 (MeOH/H₂O) to give the title compound (12 mg, yield: 32%). ESI-MS (m/z): 561.4 [M+1]⁺. ¹H NMR (400 MHz, CDCl₃) δ 8.64 (s, 1H), 8.46 (s, 1H), 8.29 (s, 1H), 8.10 (m, 2H), 7.80 (s, 1H), 7.70 (s, 1H), 7.61 (s, 2H), 6.72 (d, J=8.4 Hz, 1H), 3.99 (s, 3H), 3.93 (s, 2H), 3.68 (m, 5H), 3.53 (m, 2H), 3.09 (m, 2H), 2.13 (m, 2H), 1.35 (m, 5H).

### Example 102

### 3-chloro-N-((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)picolinamide

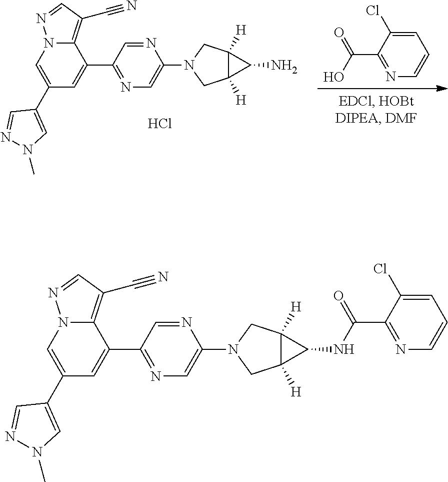

To a solution of Intermediate 47 (100 mg, 0.252 mmol), 3-chloropicolinic acid (39.7 mg, 0.252 mmol), EDCI (72 mg, 0.378 mmol) and HOBt (34 mg, 0.252 mmol) in DMF (1.5 mL) was added DIPEA (0.4 mL, 2.3 mmol) at rt. The mixture was stirred at rt for 2h, diluted with DCM/MeOH (10/1, 30 mL), washed with H₂O (10 mL×2) and brine (10 mL), dried over anhydrous Na₂SO₄, filtered off, and concentrated. The residue was purified by flash column chromatography on silica gel (DCM/MeOH=100/1 to 30/1) to give the title compound (29 mg, yield: 21%). ESI-MS (m/z): 537.4 [M+1]⁺. ¹H NMR (400 MHz, DMSO-d₆) δ 9.23 (s, 1H), 8.86 (s, 1H), 8.63 (d, J=5.4 Hz, 2H), 8.54 (d, J=3.2 Hz, 1H), 8.39 (s, 1H), 8.12 (s, 2H), 8.02 (d, J=8.9 Hz, 2H), 7.58-7.49 (m, 1H), 3.92 (d, J=11.0 Hz, 2H), 3.88 (s, 3H), 3.61 (d, J=9.8 Hz, 2H), 2.66 (s, 1H), 2.03 (s, 2H).

### Example 103

### 3-chloro-N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)picolinamide

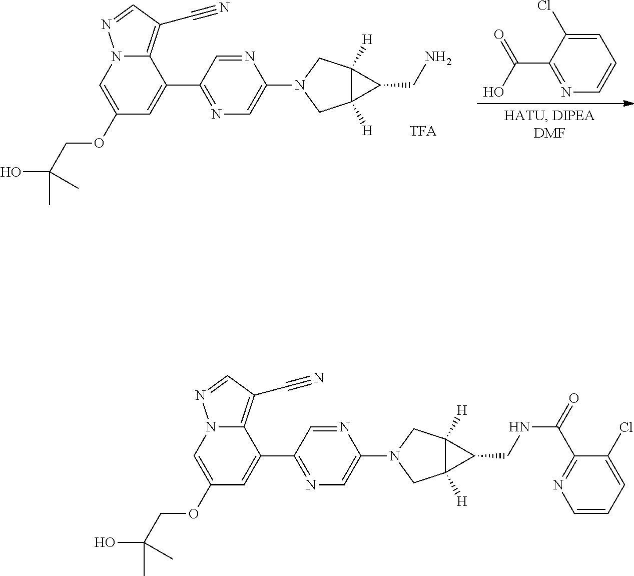

To a solution of Intermediate 49 (50 mg, 0.119 mmol), 3-chloropicolinic acid (19 mg, 0.119 mmol), and HATU (69 mg, 0.179 mmol) in DMF (0.8 mL) was added DIPEA (154 mg, 1.19 mmol) at rt. The mixture was stirred at 70° C. for 2h and purified by reverse phase flash column chromatography on C18 (MeOH/H₂O) to give the title compound (12 mg, yield: 19%). ESI-MS (m/z): 559.3 [M+1]⁺. ¹H NMR (400 MHz, DMSO-d₆) δ 8.78-8.72 (m, 1H), 8.66 (s, 1H), 8.53 (d, J=13.5 Hz, 3H), 8.08-7.97 (m, 2H), 7.53 (s, 2H), 4.68 (s, 1H), 3.86 (s, 2H), 3.78 (d, J=10.6 Hz, 2H), 3.52 (d, J=10.1 Hz, 2H), 3.27-3.22 (m, 2H), 1.77 (s, 2H), 1.21 (s, 6H), 0.91 (s, 1H).

### Example 104

### 4-(5-((1R,3S,5s,7s)-5-hydroxy-2-azaadamantan-2-yl)pyrazin-2-yl)-6-(1-methyl-1H-pyrazol-4-yl)pyrazolo[1,5-a]pyridine-3-carbonitrile

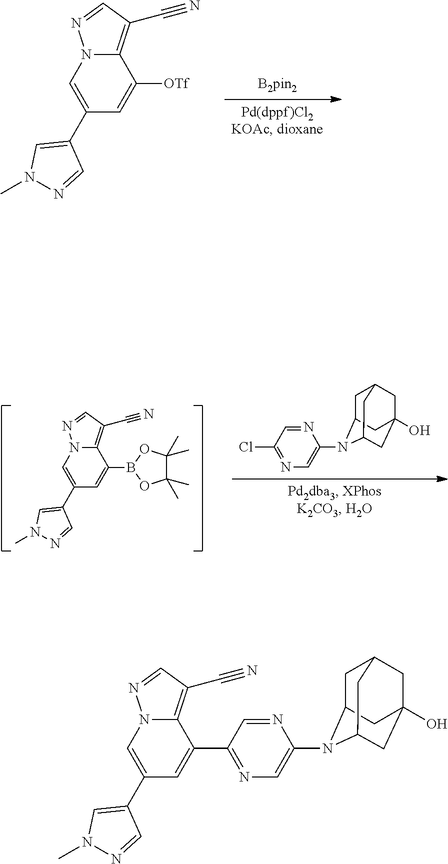

A mixture of Intermediate 1 (100 mg, 0.269 mmol), B₂Pin₂ (68 mg, 0.269 mmol), Pd(dppf)Cl₂ DCM (10 mg, 0.013 mmol), and KOAc (53 mg, 0.538 mmol) in dioxane (0.5 mL) was stirred at 100° C. under N₂ for 4h. The mixture was cooled to rt and treated with the product of Step 1 in Intermediate 54 (71 mg, 0.269 mmol), Pd₂dba₃ (12 mg, 0.013 mmol), XPhos (25 mg, 0.054 mmol), K₂CO₃ (111 mg, 0.807 mmol) and H₂O (0.5 mL). The mixture was stirred at 110° C. for 4h, cooled to rt, and purified by reverse phase flash column chromatography on C18 (MeOH/H₂O) to give the title compound (34 mg, yield: 28%). ESI-MS (m/z): 453.2 [M+1]⁺. ¹H NMR (400 MHz, DMSO-d₆) δ 9.23 (s, 1H), 8.61 (d, J=14.9 Hz, 2H), 8.40 (d, J=5.1 Hz, 2H), 8.12 (s, 1H), 8.02 (s, 1H), 4.90 (s, 2H), 4.69 (s, 1H), 3.87 (s, 3H), 2.26 (m, 1H), 1.86-1.57 (m, 10H).

### Example 105

### 4-(6-((3aR,5r,6aS)-5-hydroxy-5-(pyridin-2-ylmethyl)hexahydrocyclopenta[c]pyrrol-2(1H)-yl)pyridin-3-yl)-6-(1-methyl-1H-pyrazol-3-yl)pyrazolo[1,5-a]pyridine-3-carbonitrile

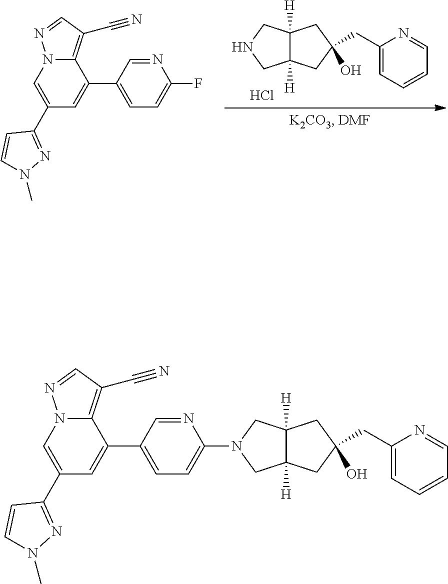

A mixture of the product of Step 1 in Intermediate 34 (50 mg, 0.158 mmol), Intermediate 55 (69 mg, 0.314 mmol), and K₂CO₃ (65 mg, 0.474 mmol) in DMF (5 mL) was stirred at 110° C. under N₂ overnight. The mixture was cooled to rt and concentrated. The residue was taken up in DCM/MeOH (10/1, 100 mL), washed with H₂O (30 mL×2) and brine (30 mL), dried over anhydrous Na₂SO₄, filtered off, and concentrated. The residue was purified by prep-TLC (DCM/MeOH=20/1) to give the title compound (45 mg, yield: 55%). ESI-MS (m/z): 517.4 [M+1]⁺. ¹H NMR (400 MHz, CDCl₃) δ 8.90 (s, 1H), 8.50 (s, 1H), 8.37 (s, 1H), 8.26 (s, 1H), 7.72 (m, 3H), 7.44 (s, 1H), 7.18 (s, 2H), 6.55 (d, J=9.9 Hz, 2H), 3.97 (s, 3H), 3.74 (s, 2H), 3.61 (d, J=10.1 Hz, 2H), 3.04 (s, 2H), 2.88 (s, 2H), 1.99 (m, 2H), 1.82 (m, 2H).

### Example 106

### 3-chloro-N-((3aR,5r,6aS)-2-(5-(3-cyano-6-(2-hydroxy-2-methylpropoxy)pyrazolo[1,5-a]pyridin-4-yl)pyridin-2-yl)octahydrocyclopenta[c]pyrrol-5-yl)picolinamide

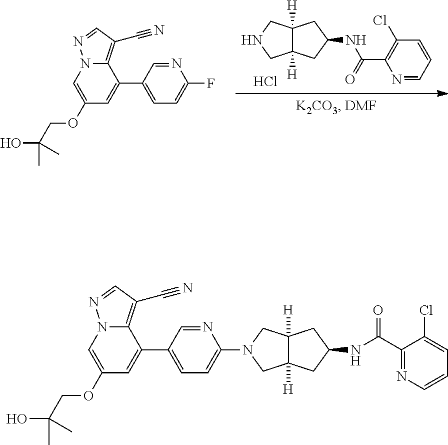

A mixture of Intermediate 29 (25.4 mg, 0.078 mmol), the product of Step 2 in Intermediate 19 (47 mg, 0.156 mmol), and K₂CO₃ (32 mg, 0.4233 mmol) in DMF (1 mL) was stirred at 110° C. under N₂ for 6h. The mixture was cooled to rt and purified by reverse phase flash column chromatography on C18 (MeOH/H₂O) to give the title compound (7 mg, yield: 15%). ESI-MS (m/z): 572.4 [M+1]⁺. ¹H NMR (400 MHz, DMSO-d₆) δ 8.73-8.67 (m, 1H), 8.63 (s, 1H), 8.54 (s, 1H), 8.48 (s, 1H), 8.28 (s, 1H), 7.98 (d, J=7.8 Hz, 1H), 7.72 (d, J=8.7 Hz, 1H), 7.48 (d, 1H), 7.23 (s, 1H), 6.59 (d, J=8.2 Hz, 1H), 4.72 (s, 1H), 4.33 (s, 1H), 3.85 (s, 2H), 3.59-3.50 (m, 2H), 3.48 (m, 2H), 2.77 (s, 2H), 2.33-2.24 (m, 2H), 1.53-1.46 (m, 2H), 1.20 (s, 6H).

### Example 107

### N-((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)formamide

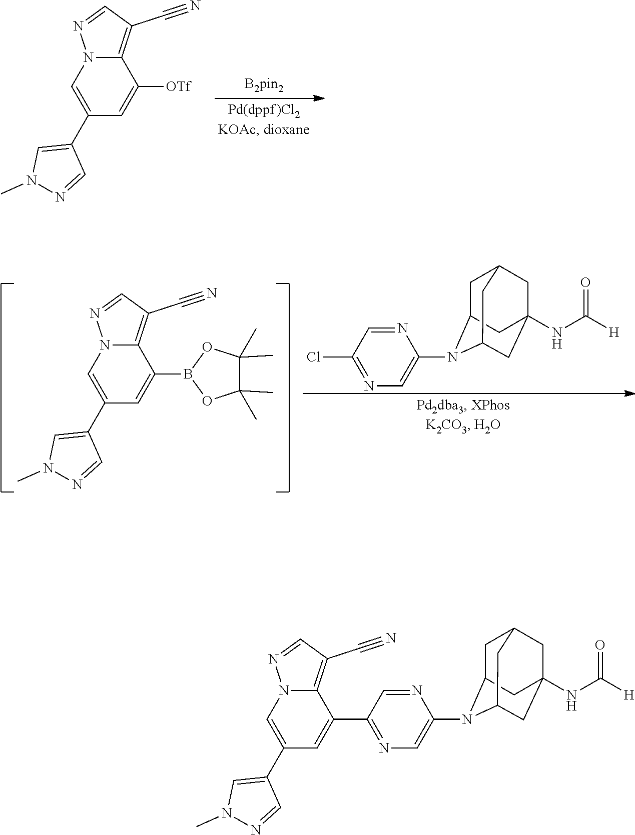

A mixture of Intermediate 1 (633 mg, 1.707 mmol), B₂Pin₂ (433 mg, 1.707 mmol), Pd(dppf)Cl₂ DCM (70 mg, 0.085 mmol), and KOAc (334 mg, 3.41 mmol) in dioxane (6 mL) was stirred at 100° C. under N₂ for 4h. The mixture was cooled to rt and treated with Intermediate 54 (200 mg, 0.687 mmol), Pd₂dba₃ (38 mg, 0.042 mmol), XPhos (87 mg, 0.171 mmol), K₂CO₃ (353 mg, 2.56 mmol) and H₂O (1.0 mL). The mixture was stirred at 110° C. for 4h, cooled to rt, and purified by reverse phase flash column chromatography on C18 (MeOH/H₂O) to give the title compound (220 mg, yield: 67%). ESI-MS (m/z): 480.4 [M+1]⁺. ¹H NMR (400 MHz, DMSO-d₆) δ 9.23 (d, J=1.4 Hz, 1H), 8.63 (s, 1H), 8.60 (dd, J=2.9, 1.4 Hz, 1H), 8.43 (dd, J=6.6, 1.5 Hz, 1H), 8.39 (s, 1H), 8.12 (s, 1H), 8.02 (dd, J=2.9, 1.5 Hz, 1H), 7.86 (d, J=2.1 Hz, 1H), 7.81 (s, 1H), 4.90 (s, 2H), 3.87 (s, 3H), 2.23 (s, 1H), 2.14-1.75 (m, 10H).

### Example 108

### 4-(5-((1R,3S,5s,7s)-5-amino-2-azaadamantan-2-yl)pyrazin-2-yl)-6-(1-methyl-1H-pyrazol-4-yl)pyrazolo[1,5-a]pyridine-3-carbonitrile

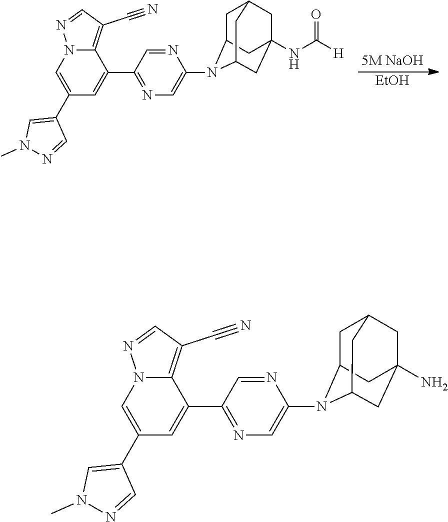 [SMILES: "EtOH" ; Cn1cc(-c2cc(-c3cnc(N4C5CC6CC4CC(NC=O)(C6)C5)cn3)c3c(C#N)cnn3c2)cn1 ; Cn1cc(-c2cc(-c3cnc(N4C5CC6CC4CC(N)(C6)C5)cn3)c3c(C#N)cnn3c2)cn1]

To a solution of Example 107 (200 mg, 0.417 mmol) in EtOH (20 mL) was added aqueous NaOH (5 N, 20 mL). The mixture was stirred at 50° C. for 3h, cooled to rt, diluted with DCM/MeOH (10/1, 100 mL), washed with H₂O (50 mL×2) and brine (50 mL), dried over anhydrous Na₂SO₄, filtered off, and concentrated to give the title compound (190 mg, quantitative). ESI-MS (m/z): 452.2 [M+1]⁺. ¹H NMR (400 MHz, DMSO-d₆) δ 9.23 (s, 1H), 8.61 (m, 2H), 8.39 (s, 2H), 8.12 (s, 1H), 8.02 (s, 1H), 4.84 (s, 2H), 3.87 (s, 3H), 2.19 (s, 1H), 1.80-1.45 (m, 10H).

### Example 109

### tert-butyl ((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)carbamate

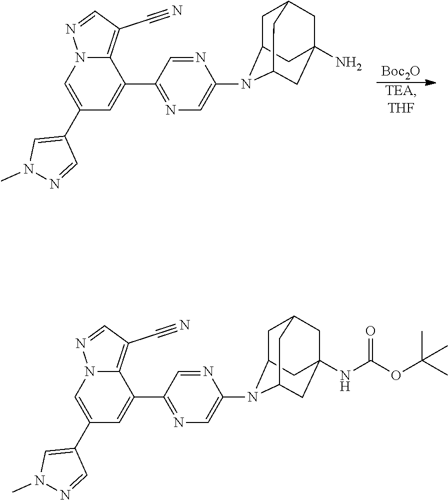 [SMILES: "Boc2O" ; Cn1cc(-c2cc(-c3cnc(N4C5CC6CC4CC(N)(C6)C5)cn3)c3c(C#N)cnn3c2)cn1 ; "TEA,THF" ; Cn1cc(-c2cc(-c3cnc(N4C5CC6CC4CC(NC(=O)OC(C)(C)C)(C6)C5)cn3)c3c(C#N)cnn3c2)cn1]

To a solution of Example 108 (50 mg, 0.11 mmol) and Boc₂O (29 mg, 0.13 mmol) in THF (1 mL) was added TEA at rt. The mixture was stirred at rt for 4h, diluted with DCM/MeOH (10/1, 50 mL), washed with H₂O (10 mL) and brine (10 mL), dried over anhydrous Na₂SO₄, filtered off, and concentrated. The residue was purified by reverse phase flash column chromatography on C18 (MeOH/H₂O) to give the title compound (53 mg, yield: 87%). ESI-MS (m/z): 552.5 [M+1]⁺. ¹H NMR (400 MHz, DMSO-d₆) δ 9.23 (s, 1H), 8.63 (s, 1H), 8.60 (s, 1H), 8.40 (s, 2H), 8.12 (s, 1H), 8.02 (s, 1H), 6.64 (s, 1H), 4.85 (s, 2H), 3.87 (s, 3H), 2.21 (s, 1H), 2.01 (s, 2H), 1.95 (m, 4H), 1.78-1.66 (m, 4H), 1.34 (s, 9H).

### Example 110

### N-((3aR,5r,6aS)-2-(5-(3-cyano-6-(2-hydroxy-2-methylpropoxy)pyrazolo[1,5-a]pyridin-4-yl)pyrazin-2-yl)octahydrocyclopenta[c]pyrrol-5-yl)-6-methoxynicotinamide

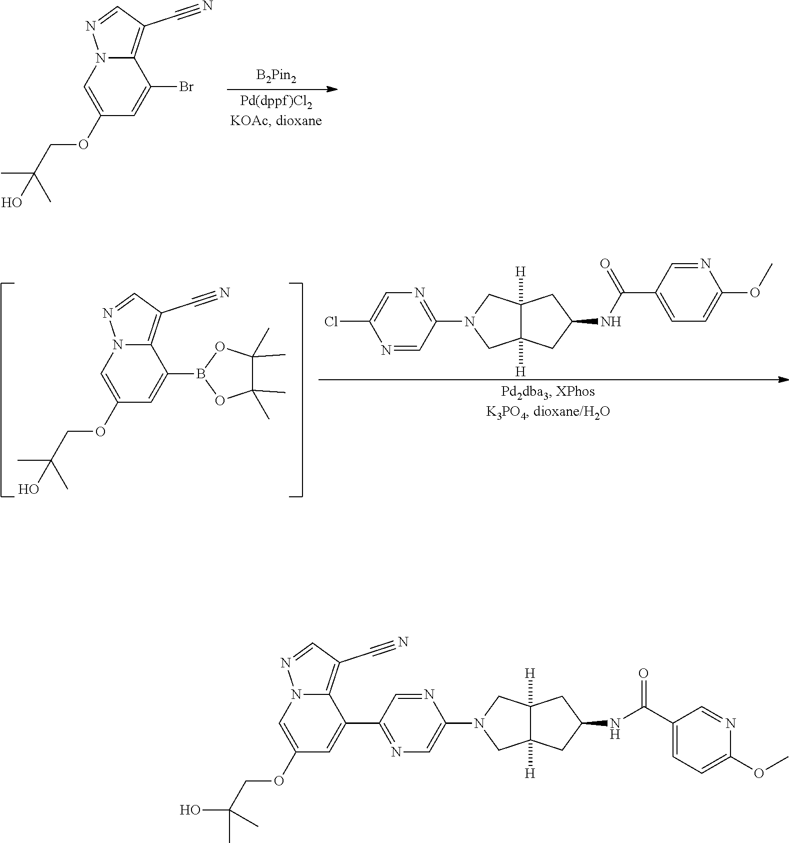

A mixture of Intermediate 3 (62 mg, 0.2 mmol), B₂Pin₂ (53 mg, 0.21 mmol), Pd(dppf)Cl₂ DCM (8.16 mg, 0.01 mmol), and KOAc (39 mg, 0.4 mmol) in dioxane (1 mL) was stirred at 100° C. under N₂ for 7h. The mixture was cooled to rt and treated with Intermediate 56 (60 mg, 0.16 mmol), Pd₂dba₃ (9.18 mg, 0.01 mmol), XPhos (19.2 mg, 0.04 mmol), K₂CO₃ (69 mg, 0.5 mmol) and dioxane/H₂O (1 mL/0.2 mL). The mixture was stirred at 110° C. under N₂ for 7h, cooled to rt, and purified by reverse phase flash column chromatography on C18 (MeOH/H₂O) to give the title compound (11 mg, yield: 12%). ESI-MS (m/z): 569.4 [M+1]⁺. ¹H NMR (400 MHz, DMSO-d₆) δ 8.68-8.65 (m, 1H), 8.61 (d, 1H), 8.57-8.53 (m, 2H), 8.39 (d, J=7.8 Hz, 1H), 8.12 (d, J=1.2 Hz, 1H), 8.08 (dd, J=8.7, 2.4 Hz, 1H), 7.54 (d, J=2.0 Hz, 1H), 6.84 (d, J=8.7 Hz, 1H), 4.70 (s, 1H), 4.37 (m, 1H), 3.87 (s, 3H), 3.87-3.85 (m, 2H), 3.61 m, 4H), 2.82-2.74 (m, 2H), 2.32-2.25 (m, 2H), 1.55-1.50 (m, 2H), 1.22 (s, 6H).

### Example 111

### N-((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)acetamide

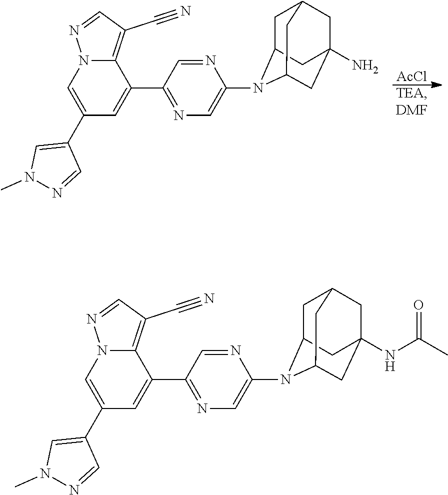 [SMILES: "AcCl" ; Cn1cc(-c2cc(-c3cnc(N4C5CC6CC4CC(N)(C6)C5)cn3)c3c(C#N)cnn3c2)cn1 ; "TEA,DMF" ; CC(=O)NC12CC3CC(C1)N(c1cnc(-c4cc(-c5cnn(C)c5)cn5ncc(C#N)c45)cn1)C(C3)C2]

To an ice-water cooled solution of Example 108 (50 mg, 0.11 mmol) in DMF (1 mL) was added TEA (33 mg, 0.33 mmol) and AcCl (9 mg, 0.11 mmol) sequentially. The mixture was stirred at rt overnight, diluted with DCM/MeOH (10/1, 50 mL), washed with H₂O (10 mL) and brine (10 mL), dried over anhydrous Na₂SO₄, filtered off, and concentrated. The residue was purified by reverse phase flash column chromatography on C18 (MeOH/H₂O) to give the title compound (22 mg, yield: 40%). ESI-MS (m/z): 494.4 [M+1]⁺. ¹H NMR (400 MHz, DMSO-d₆) δ 9.23 (d, J=1.5 Hz, 1H), 8.63 (s, 1H), 8.60 (d, J=1.4 Hz, 1H), 8.43-8.34 (m, 2H), 8.12 (s, 1H), 8.03 (d, J=1.5 Hz, 1H), 7.51 (s, 1H), 4.85 (s, 1H), 3.87 (s, 3H), 2.21 (s, 1H), 2.10-2.05 (m, 2H), 2.02 (m, 4H), 1.74 (m, 7H).

### Example 112

### 3-chloro-N-((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)picolinamide

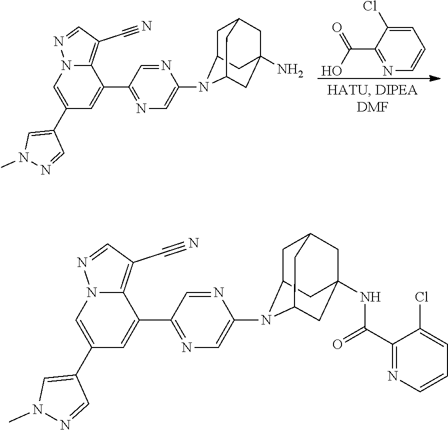 [SMILES: Cn1cc(-c2cc(-c3cnc(N4C5CC6CC4CC(N)(C6)C5)cn3)c3c(C#N)cnn3c2)cn1 ; O=C(O)c1ncccc1Cl ; Cn1cc(-c2cc(-c3cnc(N4C5CC6CC4CC(NC(=O)c4ncccc4Cl)(C6)C5)cn3)c3c(C#N)cnn3c2)cn1]

To a solution of Example 108 (50 mg, 0.11 mmol), 3-chloropicolinic acid (18 mg, 0.11 mmol), and HATU (63 mg, 0.165 mmol) in DMF (0.8 mL) was added DIPEA (71 mg, 0.55 mmol) at rt. The mixture was stirred at rt for 4h and purified by reverse phase flash column chromatography on C18 (MeOH/H₂O) to give the title compound (34 mg, yield: 52%). ESI-MS (m/z): 591.4 [M+1]⁺. ¹H NMR (400 MHz, DMSO-d₆) δ 9.24 (s, 1H), 8.63 (d, J=6.1 Hz, 2H), 8.48 (d, J=4.6 Hz, 1H), 8.45 (s, 1H), 8.40 (s, 1H), 8.26 (s, 1H), 8.13 (s, 1H), 8.04 (s, 1H), 7.97 (d, J=8.2 Hz, 1H), 7.47 (dd, J=8.2, 4.7 Hz, 1H), 4.92 (s, 2H), 3.87 (s, 3H), 2.28 (s, 1H), 2.22 (m, 2H), 2.17 (m, 4H), 1.80 (s, 4H).

### Example 113

### (3aR,5s,6aS)-2-(5-(3-cyano-6-(1-methyl-1H-pyrazol-3-yl)pyrazolo[1,5-a]pyridin-4-yl)pyridin-2-yl)-N-(6-methoxypyridin-3-yl)-5-methyloctahydrocyclopenta[c]pyrrole-5-carboxamide

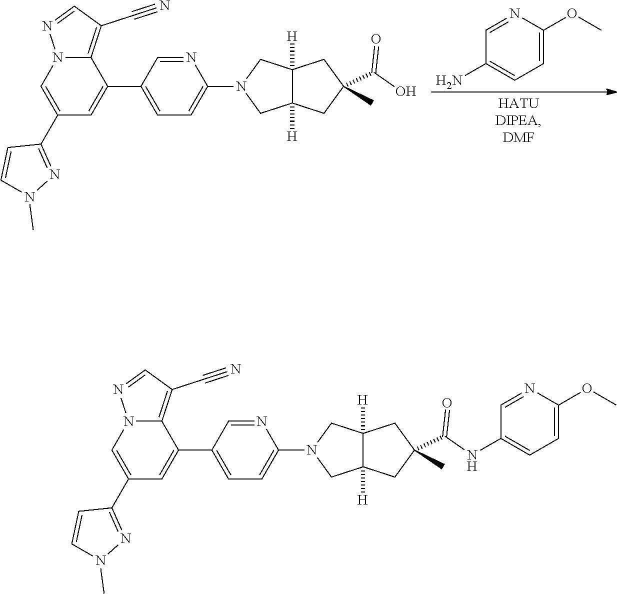

To a solution of Intermediate 36 (30 mg, 0.064 mmol), 6-methoxypyridin-3-amine (10 mg, 0.077 mmol), and HATU (37 mg, 0.096 mmol) in DMF (3 mL) was added DIPEA (25 mg, 0.192 mmol) at rt. The mixture was stirred at rt for 3 h and concentrated. The residue was taken up in DCM/MeOH (10/1, 100 mL), washed with H₂O (30 mL×2) and brine (30 mL), dried over anhydrous Na₂SO₄, filtered off, and concentrated. The residue was purified by prep-TLC (DCM/MeOH=20/1) to give the title compound (22 mg, yield: 59%). ESI-MS (m/z): 574.6 [M+1]⁺. ¹H NMR (400 MHz, CDCl₃) δ 8.65 (s, 1H), 8.37 (s, 1H), 8.26 (s, 1H), 8.16 (s, 1H), 7.90 (d, J=6.9 Hz, 1H), 7.79 (s, 2H), 7.70 (s, 1H), 7.41 (s, 1H), 7.30 (s, 1H), 6.74 (d, J=8.3 Hz, 1H), 6.64 (s, 1H), 3.99 (s, 3H), 3.91 (s, 3H), 3.71 (m, 2H), 3.59 (m, 2H), 3.05 (m, 2H), 2.68 (m, 2H), 1.57 (m, 2H), 1.32 (s, 3H).

### Example 114

### (1R,3S,5s,7s)-2-(5-(3-cyano-6-(1-methyl-1H-pyrazol-4-yl)pyrazolo[1,5-a]pyridin-4-yl)pyrazin-2-yl)-N-(6-methoxypyridin-3-yl)-2-azaadamantane-5-carboxamide

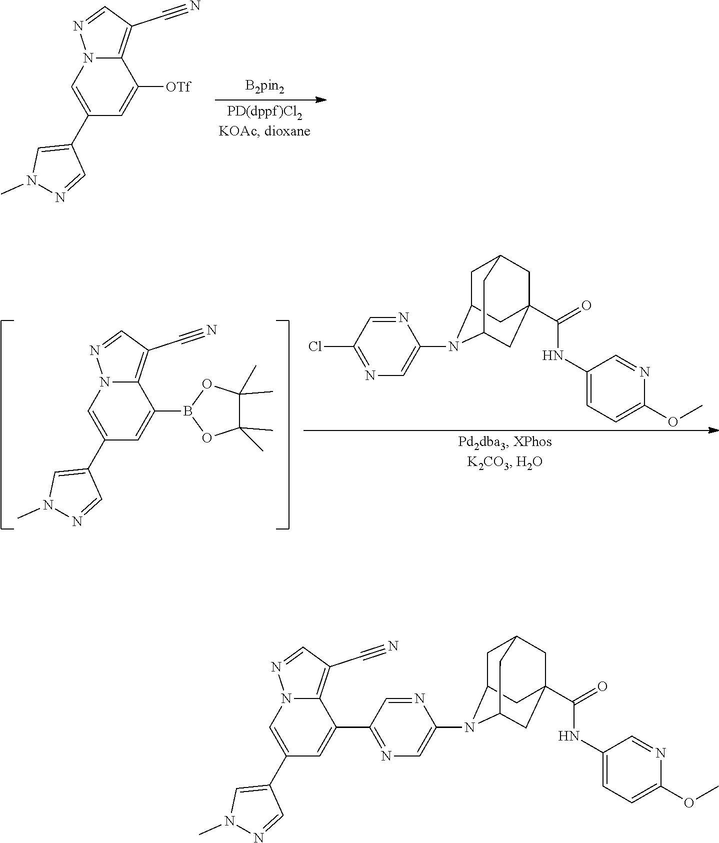

A mixture of Intermediate 1 (74 mg, 0.2 mmol), B₂Pin₂ (51 mg, 0.2 mmol), Pd(dppf)Cl₂ DCM (16 mg, 0.02 mmol), and KOAc (39 mg, 0.4 mmol) in dioxane (0.5 mL) was stirred at 100° C. under N₂ for 4h. The mixture was cooled to rt and treated with Intermediate 58 (80 mg, 0.2 mmol), Pd₂dba₃ (18 mg, 0.02 mmol), XPhos (19 mg, 0.04 mmol), K₃PO₄ (85 mg, 0.4 mmol) and dioxane/H₂O (2 mL/0.5 mL). The mixture was stirred at 110° C. under N₂ for 6h, cooled to rt, and purified by reverse phase flash column chromatography on C18 (MeOH/H₂O) to give the title compound (12 mg, yield: 10%). ESI-MS (m/z): 587.3 [M+1]⁺. ¹H NMR (400 MHz, CDCl₃) δ 8.64 (s, 1H), 8.41 (s, 1H), 8.28 (d, J=7.9 Hz, 2H), 8.15 (s, 1H), 7.98 (s, 1H), 7.91 (d, J=7.6 Hz, 1H), 7.79 (s, 1H), 7.73 (s, 1H), 7.64 (s, 1H), 6.70 (d, J=8.4 Hz, 1H), 4.82 (s, 2H), 3.96 (s, 3H), 3.87 (s, 3H), 2.27-2.22 (m, 1H), 2.13 (s, 4H), 2.00 (d, J=11.3 Hz, 4H), 1.85 (d, J=12.0 Hz, 2H).

### Example 115

### 3-chloro-N-((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide

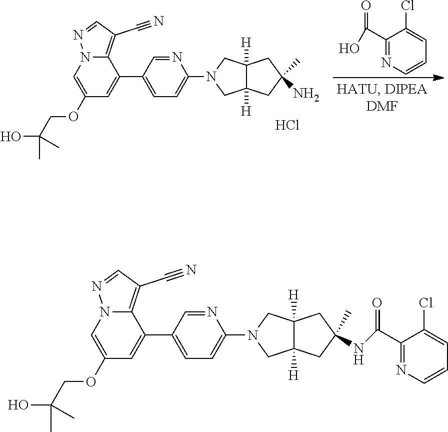 [SMILES: CC(C)(O)COc1cc(-c2ccc(N3C[C@@H]4C[C@](C)(N)C[C@@H]4C3)nc2)c2c(C#N)cnn2c1 ; Cl ; O=C(O)c1ncccc1Cl ; CC(C)(O)COc1cc(-c2ccc(N3C[C@@H]4C[C@](C)(NC(=O)c5ncccc5Cl)C[C@@H]4C3)nc2)c2c(C#N)cnn2c1]

To a solution of Intermediate 61 (75 mg, 0.168 mmol), 3-chloropicolinic acid (40 mg, 0.252 mmol), and HATU (96 mg, 0.252 mmol) in DMF (5 mL) was added DIPEA (65 mg, 0.504 mmol) at rt. The mixture was stirred at rt for 2h, diluted with DCM/MeOH (10/1, 100 mL), washed with H₂O (30 mL×2) and brine (30 mL), dried over anhydrous Na₂SO₄, filtered off, and concentrated. The residue was purified by prep-TLC (DCM/MeOH=15/1) to give the title compound (47 mg, yield: 48%). ESI-MS (m/z): 586.1 [M+1]⁺. ¹H NMR (400 MHz, CDCl₃) δ 8.35 (d, J=3.2 Hz, 1H), 8.29 (s, 1H), 8.19 (s, 1H), 8.13 (s, 1H), 8.04 (s, 1H), 7.76 (d, J=8.0 Hz, 1H), 7.65 (s, 1H), 7.29 (s, 1H), 7.11 (s, 1H), 6.51 (d, J=8.8 Hz, 1H), 3.85 (s, 2H), 3.65 (m, 4H), 2.97 (b, 2H), 2.33 (m, 2H), 2.13 (m, 2H), 1.56 (s, 3H), 1.39 (s, 6H).

### Example 116

### (3aR,5r,6aS)-2-(5-(3-cyano-6-(2-hydroxy-2-methylpropoxy)pyrazolo[1,5-a]pyridin-4-yl)pyridin-2-yl)-N-(6-methoxypyridin-3-yl)-5-methyloctahydrocyclopenta[c]pyrrole-5-carboxamide

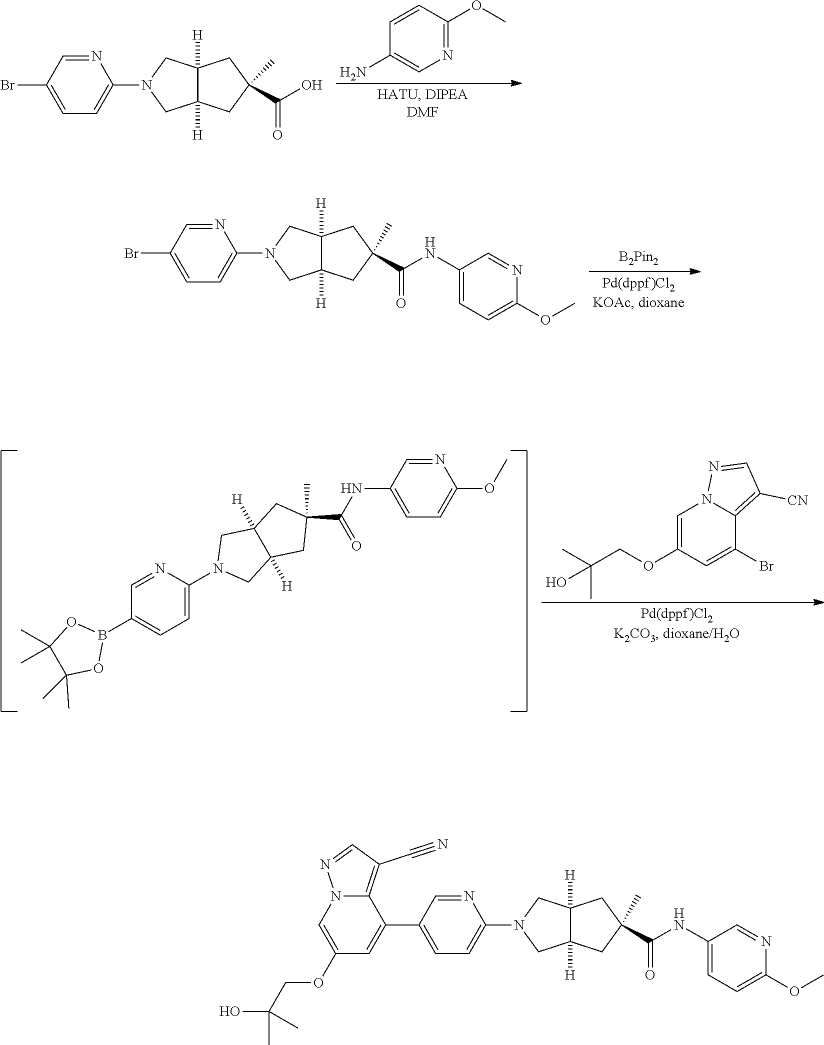

### Step 1. (3aR,5r,6aS)-2-(5-bromopyridin-2-yl)-N-(6-methoxypyridin-3-yl)-5-methyloctahydrocyclopenta[c]pyrrole-5-carboxamide

To a solution of Intermediate 59 (100 mg, 0.31 mmol) in DMF (5 mL) were added 6-methoxypyridin-3-amine (57 mg, 0.46 mmol), HATU (175 mg, 0.46 mmol) and DIPEA (120 mg, 0.93 mmol) sequentially. The reaction mixture was stirred at rt overnight, diluted with EtOAc (100 mL), washed with H₂O (30 mL×2) and brine (30 mL), dried over anhydrous Na₂SO₄, filtered off, and concentrated. The residue was purified via flash column chromatography on silica gel (PE/EtOAc=1/1) to give the title compound (110 mg, yield: 82%).

### Step 2. (3aR,5r,6aS)-2-(5-(3-cyano-6-(2-hydroxy-2-methylpropoxy)pyrazolo[1,5-a]pyridin-4-yl)pyridin-2-yl)-N-(6-methoxypyridin-3-yl)-5-methyloctahydrocyclopenta[c]pyrrole-5-carboxamide

A mixture of the product of Step 1 above (110 mg, 0.26 mmol), B₂Pin₂ (68 mg, 0.27 mmol), AcOK (51 mg, 0.52 mmol) and Pd(dppf)Cl₂DCM (24 mg, 0.03 mmol) in dioxane (3 mL) was stirred at 95° C. for 3h under N₂. The mixture was cooled to rt and treated with Intermediate 3 (73 mg, 0.23 mmol), K₂CO₃ (72 mg, 0.52 mmol), Pd(dppf)Cl₂DCM (24 mg, 0.03 mmol), and dioxane/H₂O (5 mL/1 mL). The reaction mixture was stirred at 100° C. for 3h under N₂, cooled to rt, diluted with DCM/MeOH (10/1, 100 mL), washed with H₂O (30 mL×2) and brine (30 mL), dried over anhydrous Na₂SO₄, filtered off, and concentrated. The residue was purified by flash column chromatography on silica gel (DCM/MeOH=30/1) to give the crude product, which was further purified by perp-TLC (DCM/MeOH=15/1) to give the title compound (46 mg, yield: 34%). ESI-MS (m/z): 582.1 [M+1]⁺. ¹H NMR (400 MHz, CD₃OD) δ 8.24-8.16 (m, 3H), 7.81 (d, J=8.7 Hz, 1H), 7.68 (d, J=8.4 Hz, 1H), 7.57-7.44 (m, 3H), 6.69 (d, J=8.8 Hz, 1H), 6.59 (d, J=7.9 Hz, 1H), 3.84 (s, 5H), 3.62 (m, 2H), 3.51 (m, 2H), 3.02 (b, 2H), 2.08 (m, 4H), 1.40 (s, 3H), 1.34 (s, 6H).

### Example 117

### 6-(2-hydroxy-2-methylpropoxy)-4-(6-((3aR,4S,7R,7aS)-8-((6-methoxypyridin-3-yl)methyl)hexahydro-1H-4,7-epiminoisoindol-2(3H)-yl)pyridin-3-yl)pyrazolo[1,5-a]pyridine-3-carbonitrile

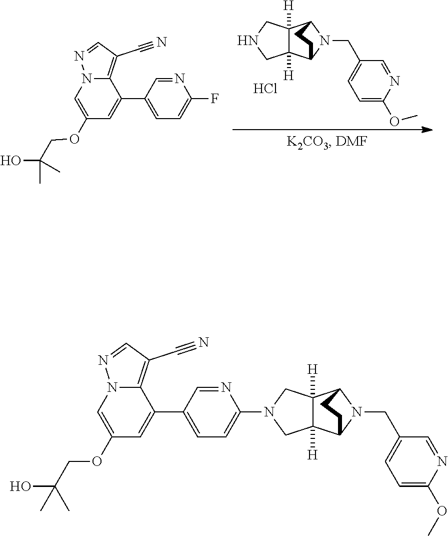

A mixture of Intermediate 29 (75 mg, 0.23 mmol), Intermediate 31 (75 mg, 0.25 mmol), and K₂CO₃ (63 mg, 0.46 mmol) in DMF (1 mL) was stirred at 110° C. under N₂ for 6h. The mixture was cooled to rt and purified by the reverse phase flash column chromatography on C18 (MeOH/H₂O) to give the title compound (11 mg, yield: 8%). ESI-MS (m/z): 566.4 [M+1]⁺. ¹H NMR (400 MHz, CD₃OD) δ 8.44 (s, 1H), 8.33 (s, 1H), 8.28 (s, 1H), 8.13 (s, 1H), 7.78 (d, J=8.3 Hz, 2H), 7.30 (s, 1H), 6.79 (dd, J=14.3, 8.9 Hz, 2H), 3.91 (s, 5H), 3.81 (d, J=11.5 Hz, 2H), 3.73 (s, 2H), 3.40 (s, 2H), 3.15 (s, 2H), 3.02 (s, 2H), 1.73 (s, 2H), 1.60 (d, J=7.4 Hz, 2H), 1.35 (s, 6H).

### Example 118

### 4-(6-((3aR,4S,7R,7aS)-8-((6-methoxypyridin-3-yl)methyl)hexahydro-1H-4,7-epiminoisoindol-2(3H)-yl)pyridin-3-yl)-6-(1-methyl-1H-pyrazol-4-yl)pyrazolo[1,5-a]pyridine-3-carbonitrile

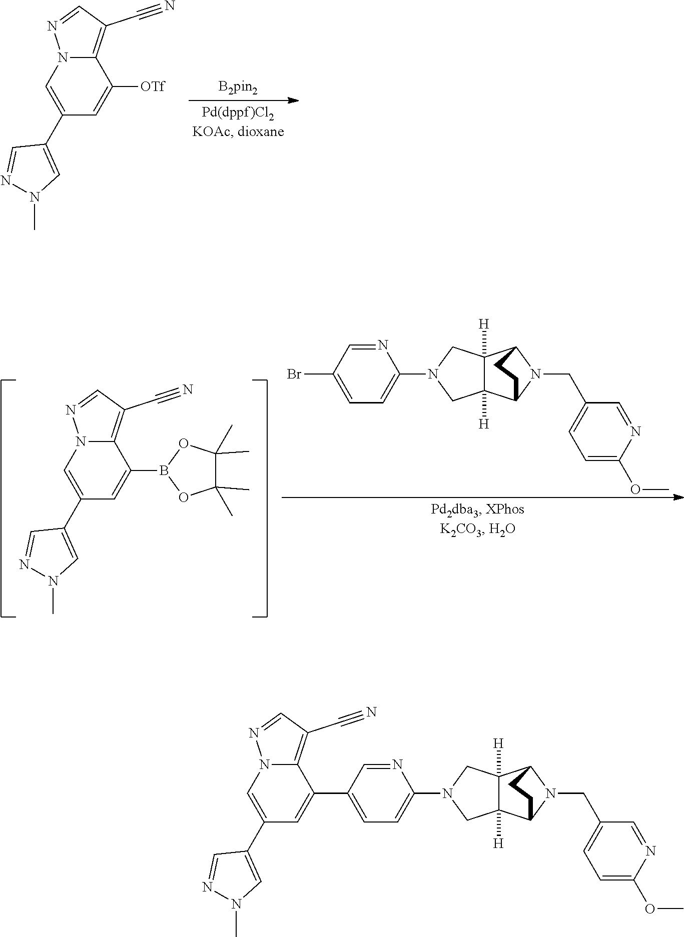

A mixture of Intermediate 1 (50 mg, 0.135 mmol), B₂Pin₂ (36 mg, 0.14 mmol), Pd(dppf)Cl₂ DCM (12 mg, 0.0135 mmol), and KOAc (27 mg, 0.27 mmol) in dioxane (1 mL) was stirred at 110° C. under N₂ for 4h. The mixture was cooled to rt and treated with Intermediate 32(25 mg, 0.06 mmol), Pd₂dba₃ (6 mg, 0.00675 mmol), XPhos (13 mg, 0.027 mmol), K₂CO₃ (56 mg, 0.405 mmol) and H₂O (0.2 mL). The mixture was stirred at 110° C. under N₂ for 4h, cooled to rt, diluted with DCM/MeOH (10/1, 100 mL), washed with H₂O (30 mL×2) and brine (30 mL), dried over anhydrous Na₂SO₄, filtered off, and concentrated. The residue was purified by reverse phase flash column chromatography on C18 (MeOH/H₂O) to give the title compound (20 mg, yield: 59%). ESI-MS (m/z): 558.3 [M+1]⁺. ¹H NMR (400 MHz, CD₃OD) δ 8.88 (s, 1H), 8.34 (d, J=6.8 Hz, 2H), 8.19 (s, 1H), 8.06 (s, 1H), 7.91 (s, 1H), 7.81 (t, J=7.0 Hz, 2H), 7.60 (s, 1H), 6.84 (d, J=8.4 Hz, 1H), 6.78 (d, J=8.5 Hz, 1H), 4.01 (s, 2H), 3.96 (s, 3H), 3.92 (s, 3H), 3.87 (d, J=11.6 Hz, 2H), 3.75 (s, 2H), 3.24 (m, 2H), 3.14 (m, 2H), 1.90-1.75 (m, 4H).

### Example 119

### (1R,5S,6r)-3-(5-(3-cyano-6-(2-hydroxy-2-methylpropoxy)pyrazolo[1,5-a]pyridin-4-yl)pyridin-2-yl)-N-((6-methoxypyridin-3-yl)methyl)-3-azabicyclo[3.1.0]hexane-6-carboxamide

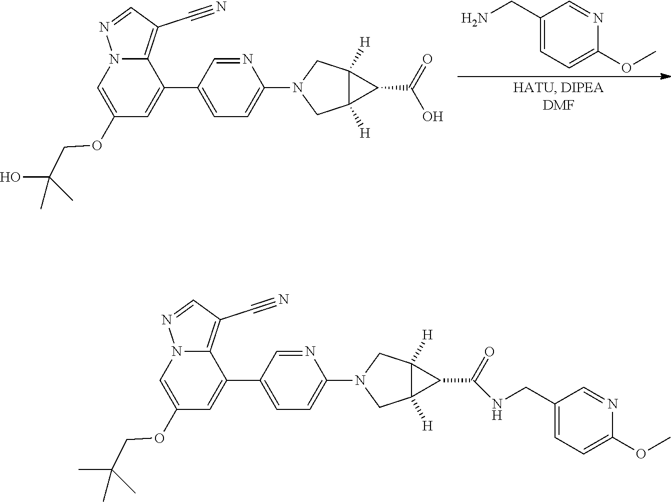

To a solution of Intermediate 63 (12.7 mg, 0.092 mmol), and HATU (52.5 mg, 0.138 mmol) in DMF (0.5 mL) was added DIPEA (35.7 mg, 0.276 mmol). The reaction mixture was stirred at 50° C. for 2 h, cooled to rt, and purified by reverse phase flash column chromatography on C18 (MeOH/H₂O) to give the title compound (20 mg, yield: 31%). ESI-MS (m/z): 554.4 [M+1]⁺. ¹H NMR (400 MHz, CDCl₃) δ 8.12 (d, J=8.3 Hz, 3H), 7.97 (s, 1H), 7.57 (d, J=8.6 Hz, 1H), 7.50 (d, J=8.4 Hz, 1H), 7.09 (s, 1H), 6.65 (d, J=8.3 Hz, 1H), 6.41 (d, J=8.5 Hz, 1H), 4.25 (s, 2H), 3.83 (s, 3H), 3.75 (d, J=10.5 Hz, 4H), 3.52 (d, J=9.9 Hz, 2H), 2.19 (s, 2H), 1.34 (s, 1H), 1.28 (s, 6H).

### Example 120

### 6-(2-hydroxy-2-methylpropoxy)-4-(5-((3aR,6aS)-5-((6-methoxypyridin-3-yl)methyl)hexahydropyrrolo[3,4-c]pyrrol-2(1H)-yl)pyrazin-2-yl)pyrazolo[1,5-a]pyridine-3-carbonitrile

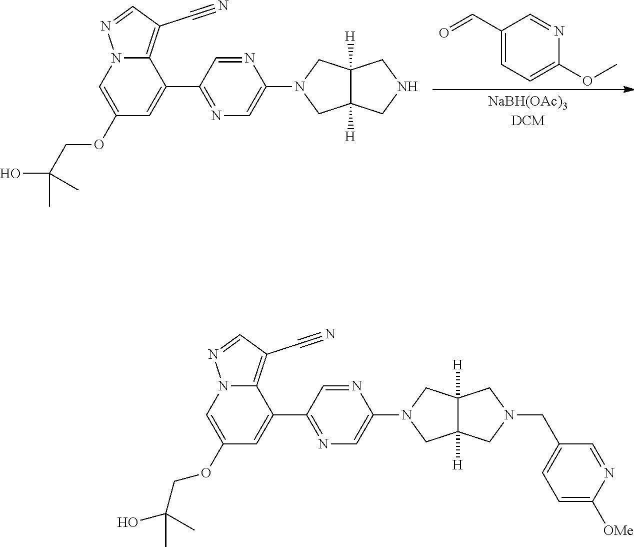

To a solution of Intermediate 53 (50 mg, 0.119 mmol) and 6-methoxynicotinaldehyde (25 mg, 0.178 mmol) in DCM (3 mL) were added NaBH(OAc)₃ (50 mg, 0.238 mmol) and 1 drop of AcOH. The mixture was stirred at rt for 2 h, treated with saturated aqueous Na₂CO₃ (10 mL), diluted with DCM/MeOH (10/1, 50 mL), washed with H₂O (20 mL×2) and brine (20 mL), dried over anhydrous Na₂SO₄, filtered off, and concentrated. The residue was purified by reverse phase flash column chromatography on C18 (MeOH/H₂O) to give the title compound (33 mg, yield: 52%). ESI-MS (m/z): 541.3 [M+1]⁺. ¹H NMR (400 MHz, DMSO-d₆) δ 8.66 (s, 1H), 8.54 (d, J=9.0 Hz, 2H), 8.08 (s, 1H), 8.02 (s, 1H), 7.59 (d, J=7.9 Hz, 1H), 7.53 (s, 1H), 6.73 (d, J=8.3 Hz, 1H), 4.68 (s, 1H), 3.86 (s, 2H), 3.79 (s, 3H), 3.75-3.66 (m, 2H), 3.49 (s, 2H), 3.40 (d, J=10.9 Hz, 2H), 2.94 (s, 2H), 2.57 (b, 2H), 2.48 (b, 2H), 1.21 (s, 6H).

Table 9 lists examples that were prepared according to the procedures as indicated below the structure of each example by using the corresponding intermediates and reagents under appropriate conditions that could be accomplished by the skilled persons.

TABLE 9

| Ex. | Structure | | Mass | |
|---|---|---|---|---|
| # | (Synthetic Method) | Chemical Name | m/z | 1H NMR |
| 121 | 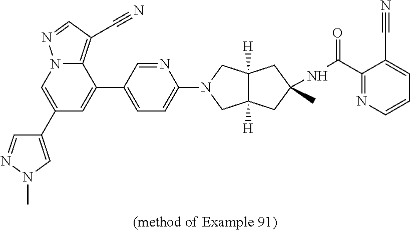 [SMILES: Cn1cc(-c2cc(-c3ccc(N4C[C@@H]5C[C@@](C)(NC(=O)c6ncccc6C#N)C[C@@H]5C4)nc3)c3c(C#N)cnn3c2)cn1] | 3-cyano-N- ((3aR,5s,6aS)-2-(5- (3-cyano-6-(1- methyl-1H-pyrazol- 4-yl)pyrazolo[1,5- a]pyridin-4- yl)pyridin-2-yl)-5- methyloctahydrocyclo- penta[c]pyrrol-5- yl)picolinamide | 569.4 | ¹H NMR (400 MHz, DMSO-d₆) δ 9.19 (d, J = 3.9 Hz, 1H), 8.86 (d, J = 4.6 Hz, 1H), 8.62 (s, 1H), 8.45 (d, J = 7.9 Hz, 1H), 8.40-8.31 (m, 3H), 8.10 (s, 1H), 7.83-7.66 (m, 3H), 6.64 (d, J = 8.8 Hz, 1H), 3.86 (s, 3H), 3.49 (m, 4H), 2.93 (m, 2H), 2.81-2.68 (m, 2H), 1.49 (m, 5H). |
| 122 | 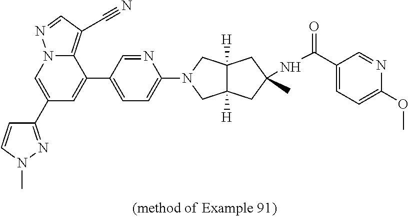 [SMILES: COc1ccc(C(=O)N[C@]2(C)C[C@H]3CN(c4ccc(-c5cc(-c6ccn(C)n6)cn6ncc(C#N)c56)cn4)C[C@H]3C2)cn1] | N-((3aR,5s,6aS)-2- (5-(3-cyano-6-(1- methyl-1H-pyrazol- 3-yl)pyrazolo[1,5- a]pyridin-4- yl)pyridin-2-yl)-5- methyloctahydrocyclo- penta[c]pyrrol-5- yl)-6- methoxynicotinamide | 574.4 | ¹H NMR (400 MHz, CD₃OD) δ 8.98 (s, 1H), 8.54 (m, 1H), 8.37- 8.18 (m, 2H), 8.02 (m, 1H), 7.76 (m, 2H), 7.60 (s, 1H), 6.83-6.56 (m, 3H), 4.01-3.84 (m, 6H), 3.67-3.48 (m, 4H), 3.02 (m, 1H), 2.78-2.68 (m, 1H), 2.30 (m, 1H), 2.10 (m, 1H), 1.55 (m, 5H) |
| 123 | 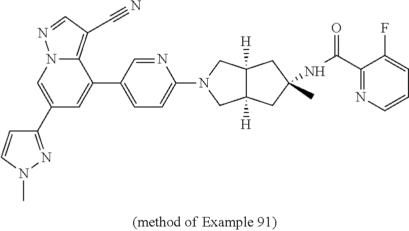 [SMILES: Cn1ccc(-c2cc(-c3ccc(N4C[C@@H]5C[C@@](C)(NC(=O)c6ncccc6F)C[C@@H]5C4)nc3)c3c(C#N)cnn3c2)n1] | N-((3aR,5s,6aS)-2- (5-(3-cyano-6-(1- methyl-1H-pyrazol- 3-yl)pyrazolo[1,5- a]pyridin-4- yl)pyridin-2-yl)-5- methyloctahydrocyclo- penta[c]pyrrol-5- yl)-3- fluoropicolinamide | 562.3 | Rotamers: ¹H NMR (400 MHz, CD₃OD) δ 8.94 (s, 1H), 8.30 (dd, J = 38.4, 26.2 Hz, 3H), 8.04 (s, 1H), 7.82-7.57 (m, 3H), 7.47 (m, 1H), 6.68-6.55 (m, 2H), 3.96 (s, 3H), 3.57 (m, 4H), 3.14- 2.97 (m, 1.4H), 2.72 (m, 1.4H), 2.30 (m, 0.6H), 2.17 (m, 0.6H), 1.62 (m, 5H). |
| 124 | 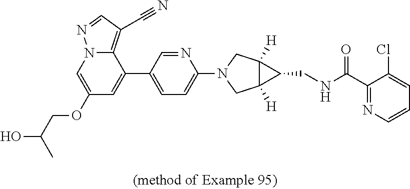 [SMILES: CC(O)COc1cc(-c2ccc(N3C[C@@H]4[C@@H](CNC(=O)c5ncccc5Cl)[C@@H]4C3)nc2)c2c(C#N)cnn2c1] | 3-chloro-N- (((1R,5S,6s)-3-(5-(3- cyano-6-(2- hydroxypropoxy) pyrazolo[1,5-a] pyridin- 4-yl)pyridin-2-yl)-3- azabicyclo[3.1.0] hexan-6- yl)methyl) picolinamide | 544.3 | ¹H NMR (400 MHz, DMSO-d₆) δ 8.75 (s, 1H), 8.64 (s, 1H), 8.55 (s, 2H), 8.26 (s, 1H), 8.01 (d, J = 8.2 Hz, 1H), 7.70 (d, J = 8.1 Hz, 1H), 7.53 (d, J = 4.6 Hz, 1H), 7.22 (s, 1H), 6.54 (d, J = 8.5 Hz, 1H), 4.93 (d, J = 3.9 Hz, 1H), 3.96 (m, 1H), 3.93 (m, 2H), 3.71 (m, 2H), 3.42 m, 2H), 3.25 (m, 2H), 1.72 (s, 1H), 1.15 (d, J = 5.7 Hz, 3H), |
| 125 | 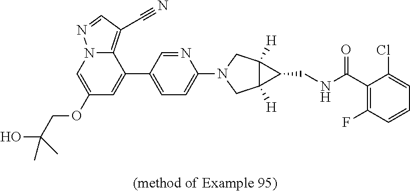 [SMILES: CC(C)(O)COc1cc(-c2ccc(N3C[C@@H]4[C@@H](CNC(=O)c5c(F)cccc5Cl)[C@@H]4C3)nc2)c2c(C#N)cnn2c1] | 2-chloro-N- (((1R,5S,6s)-3-(5-(3- cyano-6-(2-hydroxy- 2- methylpropoxy) pyrazolo [1,5-a]pyridin-4- yl)pyridin-2-yl)-3- azabicyclo[3.1.0] hexan-6-yl)methyl)-6- fluorobenzamide | 575.3 | ¹H NMR (400 MHz, DMSO-d₆) δ 8.86-8.75 (m, 1H), 8.63 (s, 1H), 8.57-8.51 (m, 1H), 8.27 (s, 1H), 7.76-7.65 (m, 1H), 7.52-7.41 (m, 1H), 7.39-7.34 (m, 1H), 7.32-7.26 (m, 1H), 7.23 (s, 1H), 6.59-6.50 (m, 1H), 4.68 (s, 1H), 3.85 (s, 2H), 3.70 (d, J = 10.1 Hz, 2H), 3.43 (d, J = 8.6 Hz, 2H), 3.24 (s, 2H), 1.72 (s, 2H), 1.20 (s, 6H), 0.87 (s, 1H). |
| 126 | 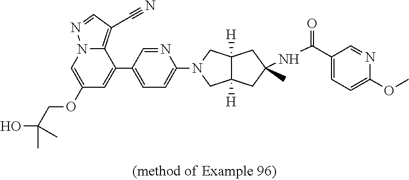 [SMILES: COc1ccc(C(=O)N[C@]2(C)C[C@H]3CN(c4ccc(-c5cc(OCC(C)(C)O)cn6ncc(C#N)c56)cn4)C[C@H]3C2)cn1] | N-((3aR,5s,6aS)-2- (5-(3-cyano-6-(2- hydroxy-2- methylpropoxy) pyrazolo [1,5-a]pyridin-4- yl)pyridin-2-yl)-5- methyloctahydrocyclo- penta[c]pyrrol-5- yl)-6- methoxynicotinamide | 582.1 | ¹H NMR (400 MHz, CDCl₃) δ 8.47 (m, 1H), 8.16 (m, 2H), 7.95 (m, 1H), 7.68 (s, 1H), 7.14 (s, 1H), 6.78-6.65 (m, 1H), 6.56 (m, 1H), 3.91 (m, 3H), 3.79 (m, 2H), 3.61 (m, 4H), 3.01 (m, 2H), 2.64 (m, 2H), 1.37-1.14 (m, 11H). |
| 127 | 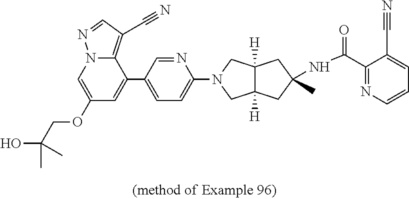 [SMILES: CC(C)(O)COc1cc(-c2ccc(N3C[C@@H]4C[C@@](C)(NC(=O)c5ncccc5C#N)C[C@@H]4C3)nc2)c2c(C#N)cnn2c1] | 3-cyano-N- ((3aR,5s,6aS)-2-(5- (3-cyano-6-(2- hydroxy-2- methylpropoxy) pyrazolo [1,5-a] pyridin-4- yl)pyridin-2-yl)-5- methyloctahydrocyclo- penta[c]pyrrol-5- yl)picolinamide | 577.4 | ¹H NMR (400 MHz, CDCl₃) δ 8.73 (s, 1H), 8.33 (s, 1H), 8.22- 8.11 (m, 3H), 7.94 (s, 1H), 7.72 (d, J = 8.2 Hz, 1H), 7.59 (s, 1H), 7.14 (s, 1H), 6.58 (s, 1H), 3.86 (s, 2H), 3.61 (m, 4H), 3.06 (m, 2H), 2.76 (m, 2H), 1.66 (m, 5H), 1.39 (s, 6H). |
| 128 | 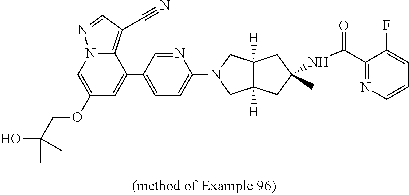 [SMILES: CC(C)(O)COc1cc(-c2ccc(N3C[C@@H]4C[C@@](C)(NC(=O)c5ncccc5F)C[C@@H]4C3)nc2)c2c(C#N)cnn2c1] | N-((3aR,5s,6aS)-2- (5-(3-cyano-6-(2- hydroxy-2- methylpropoxy) pyrazolo [1,5-a]pyridin-4- yl)pyridin-2-yl)-5- methyloctahydrocyclo- penta[c]pyrrol-5- yl)-3- fluoropicolinamide | 570.3 | ¹H NMR (400 MHz, CDCl₃) δ 8.34 (m, 2H), 8.20 (s, 1H), 8.14 (s, 1H), 7.81(s, 1H), 7.71 (d, J = 8.8 Hz, 1H), 7.55 (s, 1H), 7.48 (s, 1H), 7.13 (s, 1H), 6.56 (d, J = 8.6 Hz, 1H), 3.86 (s, 2H), 3.65 (m, 2H), 3.54 (m, 2H), 3.08 (m, 2H), 2.74 (m, 2H), 1.67 (m, 5H), 1.39 (s, 6H). |
| 129 | 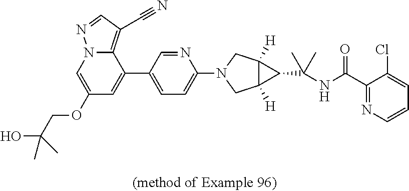 [SMILES: CC(C)(O)COc1cc(-c2ccc(N3C[C@@H]4[C@H](C3)[C@H]4C(C)(C)NC(=O)c3ncccc3Cl)nc2)c2c(C#N)cnn2c1] | 3-chloro-N-(2- ((1R,5S,6r)-3-(5-(3- cyano-6-(2-hydroxy- 2- methylpropoxy) pyrazolo [1,5-a]pyridin-4- yl)pyridin-2-yl)-3- azabicyclo[3.1.0] hexan-6-yl)propan-2- yl)picolinamide | 586.3 | ¹H NMR (400 MHz, CD₃OD) δ 8.48 (d, J = 4.7 Hz, 1H), 8.42 (s, 1H), 8.31 (s, 1H), 8.21 (s, 1H), 7.94 (d, J = 8.2 Hz, 1H), 7.72 (m, 1H), 7.47 (dd, J = 8.1, 4.7 Hz, 1H), 7.27 (s, 1H), 6.61 (d, J = 8.8 Hz, 1H), 3.90 (s, 2H), 3.79 (m, 2H), 3.57-3.50 (m, 2H), 1.96 (s, 2H), 1.44 (s, 6H), 1.34 (s, 6H), 0.97-0.86 (m, 1H). |
| 130 | 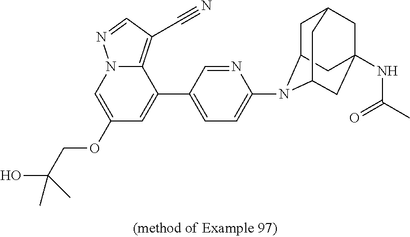 [SMILES: CC(=O)NC12CC3CC(C1)N(c1ccc(-c4cc(OCC(C)(C)O)cn5ncc(C#N)c45)cn1)C(C3)C2] | N-((1R,3S,5s,7s)-2- (5-(3-cyano-6-(2- hydroxy-2- methylpropoxy) pyrazolo [1,5-a]pyridin-4- yl)pyridin-2-yl)-2- azaadamantan-5- yl)acetamide | 501.4 | ¹H NMR (400 MHz, CDCl₃ + CD₃OD) δ 8.23 (s, 1H), 8.14 (s, 1H), 8.12 (s, 1H), 7.62 (d, J = 8.6 Hz, 1H), 7.12 (s, 1H), 6.70 (d, J = 8.9 Hz, 1H), 5.98 (s, 1H), 4.70 (s, 2H), 3.80 (s, 2H), 2.24 (s, 1H), 2.19 (s, 2H), 2.11 (d, J = 11.8 Hz, 2H), 1.91 (d, J = 11.5 Hz, 2H), 1.83 (m, 5H), 1.70 (d, J = 12.1 Hz, 2H), 1.32 (s, 6H). |
| 131 | 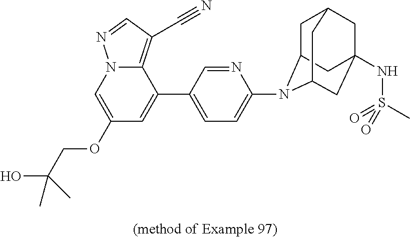 [SMILES: CC(C)(O)COc1cc(-c2ccc(N3C4CC5CC3CC(NS(C)(=O)=O)(C5)C4)nc2)c2c(C#N)cnn2c1] | N-((1R,3S,5s,7s)-2- (5-(3-cyano-6-(2- hydroxy-2- methylpropoxy) pyrazolo [1,5-a]pyidin-4- yl)pyridin-2-yl)-2- azaadamantan-5- yl) methanesulfonamide | 537.4 | ¹H NMR (400 MHz, CDCl₃ + CD₃OD) δ 8.28 (s, 1H), 8.16 (d, J = 6.8 Hz, 2H), 7.66 (d, J = 7.2 Hz, 1H), 7.15 (s, 1H), 6.74 (d, J = 8.9 Hz, 1H), 4.77 (s, 2H), 3.83 (s, 2H), 3.00 (s, 3H), 2.32 (s, 1H), 2.17 (s, 2H), 2.03 (s, 4H), 1.88 (d, J = 12.4 Hz, 2H), 1.71 (d, J = 12.3 Hz, 2H), 1.35 (s, 6H). |
| 132 | 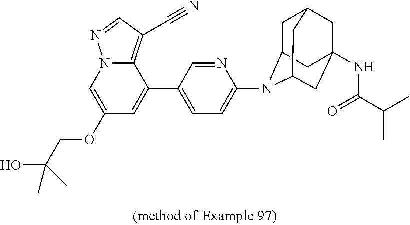 [SMILES: CC(C)C(=O)NC12CC3CC(C1)N(c1ccc(-c4cc(OCC(C)(C)O)cn5ncc(C#N)c45)cn1)C(C3)C2] | N-((1R,3S,5s,7s)-2- (5-(3-cyano-6-(2- hydroxy-2- methylpropoxy) pyrazolo [1,5-a]pyridin-4- yl)pyridin-2-yl)-2- azaadamantan-5- yl)isobutyramide | 528.645 | ¹H NMR (400 MHz, cdcl₃) δ 8.25 (s, 1HX 8.15 (s, 1H), 8.13 (s, 1HX 7.64 (d, J = 8.6 Hz, 1H), 7.14 (s, 1H), 6.73 (d, J = 8.8 Hz, 1H), 5.59 (s, 1H), 4.72 (s, 2H), 3.81 (s, 2H), 3.39 (s, 1H), 2.26 (s, 1H), 2.20 (s, 2H), 2.12 (d, J = 11.7 Hz, 2H), 1.94 (d, J = 11.3 Hz, 2H), 1.85 (d, J = 12.2 Hz, 2H), 1.72 (d, J = 12.1 Hz, 2H), 1.33 (s, 6H), 1.05 (d, J = 6.7 Hz, 6H). |
| 133 | 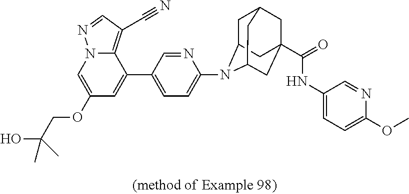 [SMILES: COc1ccc(NC(=O)C23CC4CC(C2)N(c2ccc(-c5cc(OCC(C)(C)O)cn6ncc(C#N)c56)cn2)C(C4)C3)cn1] | (1R,3S,5s,7s)-2-(5- (3-cyano-6-(2- hydroxy-2- methylpropoxy) pyrazolo [1,5-a]pyridin-4- yl)pyridin-2-yl)-N- (6-methoxypyridin- 3-yl)-2- azaadamantane-5- carboxamide | 593.676 | ¹H NMR (400 MHz, CDCl₃) δ 8.28 (s, 1H), 8.15 (d, J = 10.1 Hz, 3H), 7.88 (d, J = 8.5 Hz, 1H), 7.82 (s, 1H), 7.61 (d, J = 8.3 Hz, 1H), 7.16 (s, 1H), 6.77 (d, J = 8.3 Hz, 1H), 6.67 (d, J = 8.4 Hz, 1H), 4.74 (s, 2H), 3.87 (s, 3H), 3.84 (s, 2H), 2.31 (s, 1H), 2.15- 160 (m, 10H), 1.36 (s, 6H). |
| 134 | 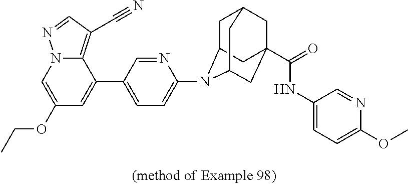 [SMILES: CCOc1cc(-c2ccc(N3C4CC5CC3CC(C(=O)Nc3ccc(OC)nc3)(C5)C4)nc2)c2c(C#N)cnn2c1] | (1R3S,5s,7s)-2-(5- (3-cyano-6- ethoxypyrazolo[1,5- a]pyridin-4- yl)pyridin-2-yl)-N- (6-methoxypyridin- 3-yl)-2- azaadamantane-5- carboxamide | 550.6 | ¹H NMR (400 MHz, CDCl₃) δ 8.25 (d, J = 2.1 Hz, 1H), 8.13 (d, J = 10.1 Hz, 2H), 8.09-8.02 (m, 2H), 7.86 (dd, J = 8.9, 2.6 Hz, 1H), 7.60 (dd, J = 8.8, 2.3 Hz, 1H), 7.08 (d, J = 1.8 Hz, 1H), 6.75 (d, J = 8.9 Hz, 1H), 6.65 (d, J = 8.9 Hz, 1H), 4.71 (s, 2H), 4.04 (q, J = 6.9 Hz, 2H), 3.89-3.79 (m, 3H), 2.28 (s, 1H), 2.12-2.01 (m, 4H), 1.92 (dd, 24.1. 12.3 Hz, |
| | | | | 4H), 1.77 (d, J = 12.0 Hz, |
| | | | | 2H), 1.44 (t, J = 6.9 Hz, 3H). |
| 135 | 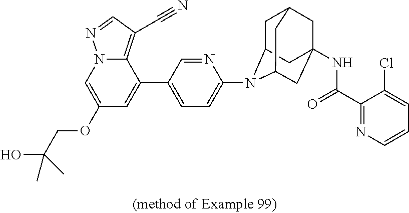 [SMILES: CC(C)(O)COc1cc(-c2ccc(N3C4CC5CC3CC(NC(=O)c3ncccc3Cl)(C5)C4)nc2)c2c(C#N)cnn2c1] | 3-chloro-N- ((1R,3S,5s,7s)-2-(5- (3-cyano-6-(2- hydroxy-2- methylpropoxy) pyrazolo [1,5-a] pyridin-4- yl)pyridin-2-yl)-2- azaadamantan-5- yl)picolinainide | 598.3 | ¹H NMR (400 MHz, CDCl₃ + CD₃OD) δ 8.37 (d, J = 3.5 Hz, 1H), 8.26 (s, 1H), 8.14 (d, J = 8.7 Hz, 2H), 7.75 (d, J = 7.9 Hz, 1H), 7.65 (d, J = 8.0 Hz, 2H), 7.31 (dd, J = 8.0, 4.5 Hz, 1H), 7.13 (s, 1H), 6.74 (d, J = 8.9 Hz, 1H), 4.78 (s, 2H), 3.81 (s, 2H), 2.32 (d, J = 19.1 Hz, 4H), 2.27 (s, 1H), 2.14 (d, J = 11.1 Hz, 2H), 1.89 (d, J = 12.2 Hz, 2H), 1.77 (d, J = 12.3 Hz, 2H), 1.33 (s, 6H). |
| 136 | 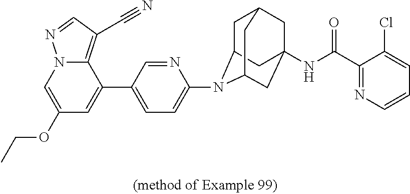 [SMILES: CCOc1cc(-c2ccc(N3C4CC5CC3CC(NC(=O)c3ncccc3Cl)(C5)C4)nc2)c2c(C#N)cnn2c1] | 3-chloro-N- ((1R,3S,5s,7s)-2-(5- (3-cyano-6- ethoxypyrazolo[1,5- a]pyridin-4- yl)pyridin-2-yl)-2- azaadamantan-5- yl)picolinamide | 554.3 | ¹H NMR (400 MHz, CDC1₃ + CD₃OD) δ 8.38 (d, J = 3.4 Hz, 1H), 8.25 (s, 1H), 8.15 (s, 1H), 8.07 (s, 1H), 7.76 (d, J = 8.0 Hz, 1H), 7.66 (d, J = 8.9 Hz, 2H), 7.31 (dd, J = 7.9, 4.5 Hz, 1H), 7.07 (s, 1H), 6.76 (d, J = 8.9 Hz, 1H), 4.79 (s, 2H), 4.05 (q, J = 6.8 Hz, 2H), 2.40-2.26 (m, 5H), 2.13 (d, J = 11.4 Hz, 2H), 1.90 (d, J = 12.3 Hz, 2H), 1.78 (d, J = 12.4 Hz, 2H), 1.44 (t, J = 6.9 Hz, 3H). |
| 137 | 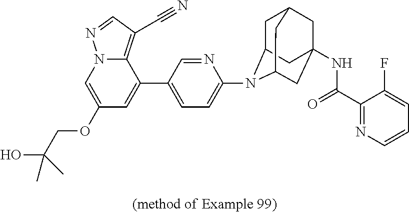 [SMILES: CC(C)(O)COc1cc(-c2ccc(N3C4CC5CC3CC(NC(=O)c3ncccc3F)(C5)C4)nc2)c2c(C#N)cnn2c1] | N-((1R,3S,5s,7s)-2- (5-(3-cyano-6-(2- hydroxy-2- methylpropoxy) pyrazolo [1,5-a] pyridin-4- yl)pyridin-2-yl)-2- azaadamantan-5-yl)- 3-fluoropicolinamide | 582.3 | ¹H NMR (400 MHz, CDCl₃) δ 8.28 (d, J = 8.3 Hz, 2H), 8.14 (d, J = 10.1 Hz, 2H), 7.74 (s, 1H), 7.65 (d, J = 7.1 Hz, 1H), 7.54- 7.46 (m, 1H), 7.45-7.38 (m, 1H), 7.13 (s, 1H), 6.75 (d, J = 8.9 Hz, 1H), 4.79 (s, 2H), 3.82 (s, 2H), 2.34 (s, 3H), 2.28 (d, J = 12.0 Hz, 2H), 2.16 (d, J = 11.3 Hz, 2H), 1.90 (d, J = 12.2 Hz, 2H), 1.77 (d, J = 12.3 Hz, 2H), 1.33 (s, 6H). |
| 138 | 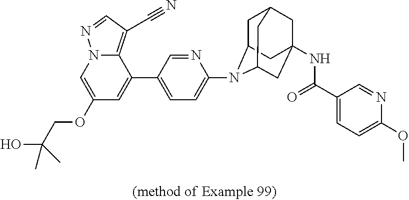 [SMILES: COc1ccc(C(=O)NC23CC4CC(C2)N(c2ccc(-c5cc(OCC(C)(C)O)cn6ncc(C#N)c56)cn2)C(C4)C3)cn1] | N-((1R,3S,5s,7s)-2- (5-(3-cyano-6-(2- hydroxy-2- methylpropoxy) pyrazolo [1,5-a]pyridin-4- yl)pyridin-2-yl)-2- azaadamantan-5-yl)- 6- methoxynicotinamide | 594.4 | ¹H NMR (400 MHz, Methanol-d₄) δ 8.55 (s, 1H), 8.40 (s, 1H), 8.29 (d, J = 6.4 Hz, 2H), 8.00 (d, J = 8.6 Hz, 1H), 7.73 (s, 1H), 7.29 (s, 1H), 6.92 (d, J = 8.9 Hz, 1H), 6.79 (d, J = 8.7 Hz, 1H), 3.94 (s, 3H), 3.90 (s, 2H), 2.41-2.27 (m, 6H), 2.20 (d, J = 12.3 Hz, 2H), 1.93 (d, J = 12.6 Hz, 2H), 1.83 (d, J = 12.8 Hz, 2H), 1.35 (s, 6H). |
| 139 | 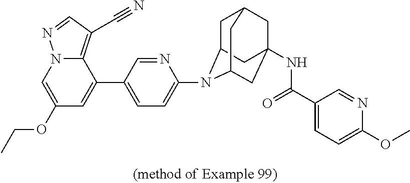 [SMILES: CCOc1cc(-c2ccc(N3C4CC5CC3CC(NC(=O)c3ccc(OC)nc3)(C5)C4)nc2)c2c(C#N)cnn2c1] | N-((1R,3S,5s,7s)-2- (5-(3-cyano-6- ethoxypyrazolo[1,5- a]pyridin-4- yl)pyridin-2-yl)-2- azaadamantan-5-yl)- 6- methoxynicotinamide | 550.5 | ¹H NMR (400 MHz, DMSO-d₆) δ 8.66-8.50 (m, 3H), 8.31 (d, J = 2.6 Hz, 1H), 8.06 (dd, J = 8.6, 2.5 Hz, 1H), 7.83 (s, 1H), 7.74 (dd, J = 8.9, 2.6 Hz, 1H), 7.27 (d, J = 2.2 Hz, 1H), 6.93 (d, J = 9.0 Hz, 1H), 6.82 (d, J = 8.7 Hz, 1H), 4.82 (s, 2H), 4.14 (q, J = 7.0 Hz, 2H), 3.87 (s, 3H), 2.27-2.04 (m, 7H), 1.72 (m, 4H), 1.36 (t, J = 7.0 Hz, 3H). |
| 140 | 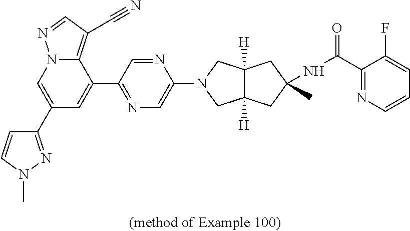 [SMILES: Cn1ccc(-c2cc(-c3cnc(N4C[C@@H]5C[C@@](C)(NC(=O)c6ncccc6F)C[C@@H]5C4)cn3)c3c(C#N)cnn3c2)n1] | N-((3aR,5s,6aS)-2- (5-(3-cyano-6-(1- methyl-1H-pyrazol- 3-yl)pyrazolo[1,5- a]pyridin-4- yl)pyrazin-2-yl)-5- methyloctahydrocyclo- penta[c]pyrrol-5- yl)-3- fluoropicolinamide | 562.6 | ¹H NMR (400 MHz, CDCl₃) δ 8.92 (s, 1H), 8.54-8.23 (m, 3H), 8.08 (m, 1H), 7.99 (m, 1H), 7.82 (s, 1H), 7.57-7.42 (m, 3H), 6.58 (s, 1H), 3.98 (s, 3H), 3.69 (m, 2H), 3.59 (m, 2H), 3.17-3.04 (m, 2H), 2.82-2.69 (m, 2H), 1.58 (m, 5H). |
| 141 | 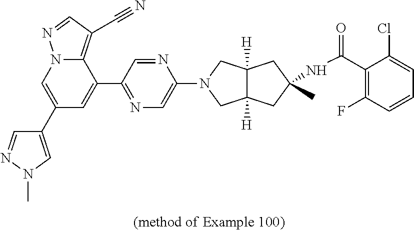 [SMILES: Cn1cc(-c2cc(-c3cnc(N4C[C@@H]5C[C@@](C)(NC(=O)c6c(F)cccc6Cl)C[C@@H]5C4)cn3)c3c(C#N)cnn3c2)cn1] | 2-chloro-N- ((3aR,5s,6aS)-2-(5- (3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-6- fluorobenzamide | 596.3 | ¹H NMR (400 MHz, CD₃OD) δ 8.72 (s, 1H), 8.41 (s, 1H), 8.29 (s, 1H), 8.08 (s, 1H), 7.88 (s, 1H), 7.82 (s, 1H), 7.68 (s, 1H), 7.34- 7.24 (m, 1H), 7.19 (m, 1H), 7.03 (m, 1H), 3.95 (s, 3H), 3.66 (m, 2H), 3.55 (m, 2H), 3.09 (m, 2H), 2.72 (m, 2H), 1.63-1.46 (m, 5H). |
| 142 | 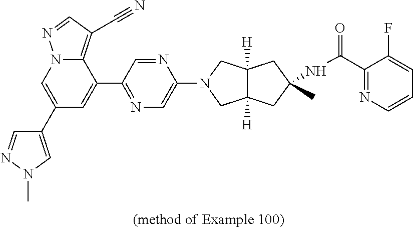 [SMILES: Cn1cc(-c2cc(-c3cnc(N4C[C@@H]5C[C@@](C)(NC(=O)c6ncccc6F)C[C@@H]5C4)cn3)c3c(C#N)cnn3c2)cn1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-3- fluoropicolinamide | 563.4 | ¹H NMR (400 MHz, CDCl₃) δ 8.64 (s, 1H), 8.44 (m, 1H), 8.36 (s, 1H), 8.29 (s, 1H), 8.08 (m, 1H), 7.81 (d, J = 9.5 Hz, 2H), 7.70 (s, 1H), 7.63-7.43 (m, 3H), 3.99 (s, 3H), 3.70 (m, 2H), 3.59 (m, 2H), 3.12 (m, 2H), 2.77 (m, 2H), 1.60 (m, 5H). |
| 143 | 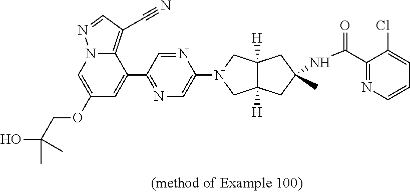 [SMILES: CC(C)(O)COc1cc(-c2cnc(N3C[C@@H]4C[C@@](C)(NC(=O)c5ncccc5Cl)C[C@@H]4C3)cn2)c2c(C#N)cnn2c1] | 3-chloro-N- ((3aR,5s,6aS)-2-(5- (3-cyano-6-(2- hydroxy-2- methylpropoxy) pyrazolo [1,5-a]pyridin-4- yl)pyrazin-2-yl)-5- methyloctahydrocyclo- penta[c]pyrrol-5- yl)picolinamide | 587.3 | ¹H NMR (400 MHz, CDCl₃) δ 8.44 (s, 2H), 8.24 (s, 1H), 8.16 (s, 1H), 8.09 (s, 1H), 7.88-7.70 (m, 2H), 7.37 (s, 2H), 3.87 (s, 2H), 3.76-3.51 (m, 4H), 3.12 (s, 2H), 2.76 (d, J = 7.2 Hz, 2H), 1.39 (s, 6H), 1.26 (m, 5H). |
| 144 | 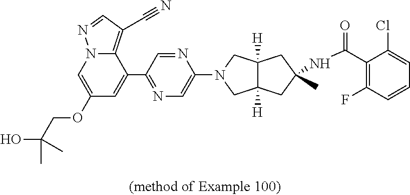 [SMILES: CC(C)(O)COc1cc(-c2cnc(N3C[C@@H]4C[C@@](C)(NC(=O)c5c(F)cccc5Cl)C[C@@H]4C3)cn2)c2c(C#N)cnn2c1] | 2-chloro-N- ((3aR,5s,6aS)-2-(5- (3-cyano-6-(2- hydroxy-2- methylpropoxy) pyrazolo [1,5-a]pyridin-4- yl)pyrazin-2-yl)-5- methyloctahydrocyclo- penta[c]pyrrol-5- yl)-6- fluorobenzainide | 604.5 | ¹H NMR (400 MHz, CDCl₃) δ 8.43 (s, 1H), 8.23 (s, 1H), 8.16 (s, 1H), 8.10 (s, 1H), 7.37 (s, 1H), 7.32-7.28 (m, 1H), 7.21 (d, J = 8.0 Hz, 1H), 7.04 (s, 2H), 3.87 (s, 2H), 3.69 (m, 2H), 3.59 (m, 2H), 3.12 (b, 2H), 2.71(m, 2H), 1.64 (m, 5H), 1.39 (s, 6H). |
| 145 | 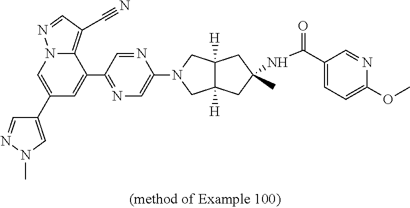 [SMILES: COc1ccc(C(=O)N[C@]2(C)C[C@H]3CN(c4cnc(-c5cc(-c6cnn(C)c6)cn6ncc(C#N)c56)cn4)C[C@H]3C2)cn1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo penta[c]pyrrol-5- yl)-6- methoxynicotinamide | 575.4 | ¹H NMR (400 MHz, CD₃OD) δ 8.76 (d, J = 1.4 Hz, 1H), 8.54 (d, J = 2.3 Hz, 1H), 8.43 (d, J = 1.3 Hz, 1H), 8.31 (d, J = 2.8 Hz, 1H), 8.08 (d, J = 1.2 Hz, 1H), 8.05- 7.98 (m, 1H), 7.94-7.91 (m, 1H), 7.83 (d, J = 12.0 Hz, 1H), 7.70 (dd, J = 6.3, 1.4 Hz, 1H), 6.78 (d, J = 8.7 Hz, 1H), 4.01 (s, 3H), 3.93 (s, 3H), 3.73-3.66 (m, 2H), 3.56 (m, 2H), 3.12-2.98 (m, 2H), 2.80-2.67 (m, 2H), 1.65-1.54 (m, 5H). |
| 146 | 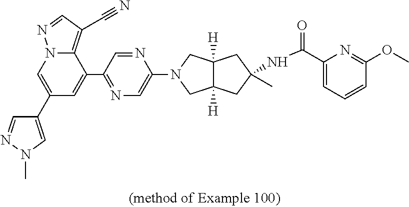 [SMILES: COc1cccc(C(=O)N[C@]2(C)C[C@H]3CN(c4cnc(-c5cc(-c6cnn(C)c6)cn6ncc(C#N)c56)cn4)C[C@H]3C2)n1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-6- methoxypicolinamide | 574.636 | ¹H NMR (400 MHz, CDCl₃) δ 8.63 (s, 1H), 8.46 (s, 1H), 8.29 (s, 1H), 8.10 (s, 1H), 7.84-7.65 (m, 5H), 7.60 (s, 1H), 6.89 (d, J = 8.0 Hz, 1H), 3.98 (s, 6H), 3.73-3.54 (m, 4H), 3.09 (s, 2H), 2.73 (m, 2H), 1.71-1.59 (m, 5H). |
| 147 | 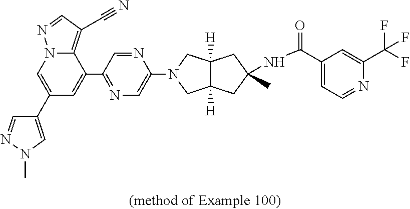 [SMILES: Cn1cc(-c2cc(-c3cnc(N4C[C@@H]5C[C@@](C)(NC(=O)c6ccnc(C(F)(F)F)c6)C[C@@H]5C4)cn3)c3c(C#N)cnn3c2)cn1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-2- (trifluoromethyl)iso- nicotinamide | 613.3 | ¹H NMR (400 MHz, CDCl₃) δ 8.86 (d, J = 4.6 Hz, 1H), 8.64 (s, 1H), 8.47 (s, 1H), 8.29 (s, 1H), 8.11 (s, 1H), 7.96 (s, 1H), 7.80 (s, 2H), 7.66 (d, J = 33.2 Hz, 2H), 6.05 (s, 1H), 3.99 (s, 3H), 3.78- 3.53 (m, 4H), 3.10 (s, 2H), 2.77- 2.63 (m, 2H), 1.77-1.65 (m, 5H). |
| 148 | 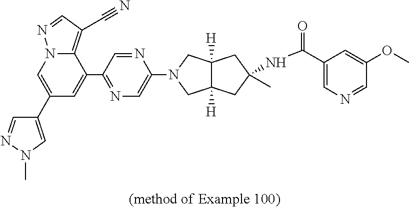 [SMILES: COc1cncc(C(=O)N[C@]2(C)C[C@H]3CN(c4cnc(-c5cc(-c6cnn(C)c6)cn6ncc(C#N)c56)cn4)C[C@H]3C2)c1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-5- methoxynicotinamide | 575.5 | ¹H NMR (400 MHz, CDCl₃) δ 8.64 (s, 1H), 8.43 (d, J = 21.1 Hz, 1H), 8.27 (d, J = 12.0 Hz, 2H), 8.08 (d, J = 16.7 Hz, 1H), 7.79 (s, 1H), 7.71 (d, J = 9.5 Hz, 1H), 7.61 (s, 1H), 7.14 (s, 1H), 6.97 (d, J = 12.2 Hz, 1H), 5.92 (s, 1H), 3.97 (d, J = 7.5 Hz, 6H), 3.79- 3.47 (m, 4H), 3.08 (m, 2H), 2.67 (m, 2H), 1.57 (m, 5H). |
| 149 | 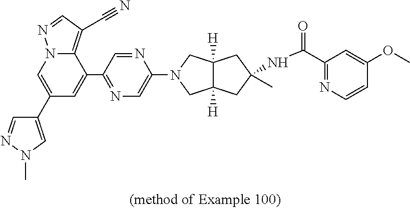 [SMILES: COc1ccnc(C(=O)N[C@]2(C)C[C@H]3CN(c4cnc(-c5cc(-c6cnn(C)c6)cn6ncc(C#N)c56)cn4)C[C@H]3C2)c1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-4- methoxypicolinamide | 575.5 | ¹H NMR (400 MHz, CDCl₃) δ 8.64 (s, 1H), 8.43 (d, J = 23.3 Hz, 1H), 8.34-8.18 (m, 2H), 8.07 (d, J = 23.5 Hz, 2H), 7.80 (s, 1H), 7.72 (d, J = 17.9 Hz, 2H), 7.59 (d, J = 9.9 Hz, 1H), 6.92 (d, J = 3.0 Hz, 1H), 3.99 (s, 3H), 3.90 (d, J = 16.3 Hz, 3H), 3.69 (t, J = 8.8 Hz, 2H), 3.58 (d, J = 10.5 Hz, 2H), 3.11 (s, 2H), 2.84-2.69 (m, 2H), 1.69-1.63 (m, 5H). |
| 150 | 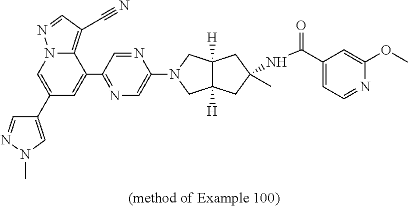 [SMILES: COc1cc(C(=O)N[C@]2(C)C[C@H]3CN(c4cnc(-c5cc(-c6cnn(C)c6)cn6ncc(C#N)c56)cn4)C[C@H]3C2)ccn1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-2- methoxyisonicotin- amide | 575.5 | ¹H NMR (400 MHz, CDCl₃) δ 8.63 (s, 1H), 8.54 (s, 1H), 8.44 (d, J = 19.2 Hz, 2H), 8.28 (s, 1H), 8.10 (s, 1H), 7.80 (s, 1H), 7.69 (s, 2H), 7.61 (s, 1H), 6.12 (s, 1H), 3.99 (s, 3H), 3.89 (d, J = 20.8 Hz, 3H), 3.70 (s, 3H), 3.59 (d, J = 10.8 Hz, 2H), 3.11 (s, 2H), 2.72 (m, 2H), 1.73-1.59 (m, 5H). |
| 151 | 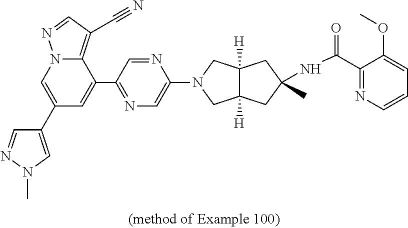 [SMILES: COc1cccnc1C(=O)N[C@]1(C)C[C@H]2CN(c3cnc(-c4cc(-c5cnn(C)c5)cn5ncc(C#N)c45)cn3)C[C@H]2C1] | N-((3aR,5s,6aS)-2- (5-(3-cyano-6-(1- methyl-1H-pyrazol- 4-yl)pyrazolo[1,5- a]pyridin-4- yl)pyrazin-2-yl)-5- methyloctahydrocyclo- penta[c]pyrrol-5- yl)-3- methoxypicolinamide | 575.5 | ¹H NMR (400 MHz, CDCl₃) δ 8.63 (s, 1H), 8.45 (s, 1H), 8.28 (s, 1H), 8.12 (m, 2H), 7.82 (m, 2H), 7.69 (s, 1H). 7.60 (s, 1H), 7.40 (s, 2H), 4.03-3.90 (m, 6H), 3.68 (d, J = 7.3 Hz, 2H), 3.56 (d, J = 10.6 Hz, 2H), 3.11 (s, 2H), 2.76 (m, 2H), 1.59 (m, 5H). |
| 152 | 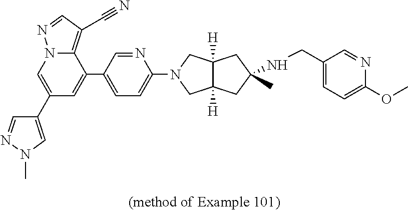 [SMILES: COc1ccc(CN[C@]2(C)C[C@H]3CN(c4ccc(-c5cc(-c6cnn(C)c6)cn6ncc(C#N)c56)cn4)C[C@H]3C2)cn1] | 4-(6-((3aR,5s,6aS)- 5-(((6- methoxypyridin-3- yl)methyl)amino)-5- methylhexahydrocyclo- penta[c]pyrrol- 2(1H)-yl)pyridin-3- yl)-6-(1-methyl-1H- pyrazol-4- yl)pyrazolo[1,5- a]pyridine-3- carbonitrile | 560.3 | ¹H NMR (400 MHz, CDCl₃) δ 8.62 (s, 1H), 8.35 (s, 1H), 8.25 (s, 1H), 8.13 (s, 1H), 7.78 (s, 1H), 7.74-7.61 (m, 3H), 7.39 (d, J = 10.2 Hz, 2H), 6.72 (d, J = 8.2 Hz, 1H), 6.54 (d, J = 8.3 Hz, 1H), 3.99 (s, 3H), 3.89 (m, 3H), 3.63 (m, 4H), 3.48 (m, 2H), 3.05 (s, 2H), 2.07 (m, 2H), 1.35 (m, 5H). |
| 153 | 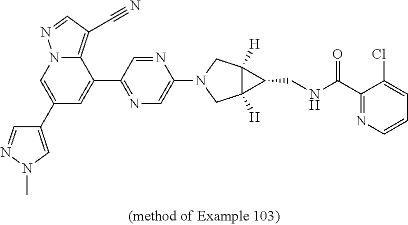 [SMILES: Cn1cc(-c2cc(-c3cnc(N4C[C@@H]5[C@@H](CNC(=O)c6ncccc6Cl)[C@@H]5C4)cn3)c3c(C#N)cnn3c2)cn1] | 3-chloro-N- (((1R,5S,6s)-3-(5-(3- cyano-6-(1-methyl- 1H-pyrazol-4- yl)pyrazolo[1,5- a]pyridin-4- yl)pyrazin-2-yl)-3- azabicyclo[3.1.0] hexan-6- yl)methyl) picolinamide | 551.4 | ¹H NMR (400 MHz, DMSO-d₆) δ 9.21 (s, 1H), 8.76 (d, J = 5.9 Hz, 1H), 8.60 (d, J = 16.2 Hz, 2H), 8.55 (d, J = 4.5 Hz, 1H), 8.38 (s, 1H), 8.09 (d, J = 17.4 Hz, 2H), 8.04-7.95 (m, 2H), 7.53 (dd, J = 8.3, 4.6 Hz, 1H), 3.87 (s, 3H), 3.79 (d, J = 10.7 Hz, 2H), 3.53 (d, J = 10.5 Hz, 2H), 1.78 (s, 2H), 0.96-0.87 (m, 1H). |
| 154 | 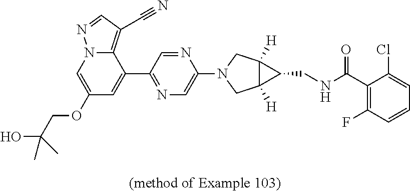 [SMILES: CC(C)(O)COc1cc(-c2cnc(N3C[C@@H]4[C@@H](CNC(=O)c5c(F)cccc5Cl)[C@@H]4C3)cn2)c2c(C#N)cnn2c1] | 2-chloro-N- (((1R,5S,6s)-3-(5-(3- cyano-6-(2-hydroxy- 2- methylpropoxy) pyrazolo [1,5-a]pyridin-4- yl)pyrazin-2-yl)-3- azabicyclo[3.1.0] hexan- 6-yl)methyl)-6- fluorobenzamide | 576.5 | ¹H NMR (400 MHz, DMSO-d₆) δ 8.82 (s, 1H), 8.66 (s, 1H), 8.53 (d, J = 12.6 Hz, 2H), 8.05 (s, 1H), 7.53 (s, 1H), 7.45 (s, 1H), 7.36 (d, J = 7.9 Hz, 1H), 7.29 (s, 1H), 4.69 (s, 1H), 3.86 (s, 2H), 3.77 (d, J = 10.3 Hz, 2H), 3.53 (d, J = 9.4 Hz, 2H), 3.27-3.22 (m, 2H), 1.97 (s, 1H), 1.21 (s, 6H, 0.87 (m, 2H)). |
| 155 | 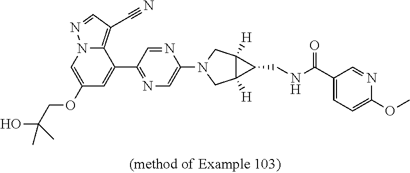 [SMILES: COc1ccc(C(=O)NC[C@@H]2[C@H]3CN(c4cnc(-c5cc(OCC(C)(C)O)cn6ncc(C#N)c56)cn4)C[C@@H]23)cn1] | N-(((1R,5S,6s)-3-(5- (3-cyano-6-(2- hydroxy-2- methylpropoxy) pyrazolo [1,5-a]pyridin-4- yl)pyrazin-2-yl)-3- azabicyclo[3.1.0] hexan- 6-yl)methyl)-6- methoxynicotinamide | 555.4 | ¹H NMR (400 MHz, DMSO-d₆) δ 8.66 (s, 2H), 8.53 (m, 3H), 8.11 (d, J = 7.4 Hz, 1H), 8.04 (s, 1H), 7.52 (s, 1H), 6.87 (d, J = 8.2 Hz, 1H), 4.69 (s, 1H), 3.89 (s, 3H), 3.86 (s, 2H), 3.80 (d, J = 10.1 Hz, 2H), 3.51 (d, J = 9.8 Hz, 2H), 3.27-3.22 (m, 2H), 1.75 (s, 2H), 1.21 (s, 6H), 0.92 (m, 1H). |
| 156 | 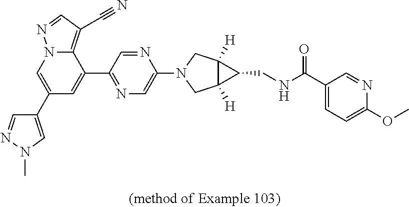 [SMILES: COc1ccc(C(=O)NC[C@@H]2[C@H]3CN(c4cnc(-c5cc(-c6cnn(C)c6)cn6ncc(C#N)c56)cn4)C[C@@H]23)cn1] | N-(((1R,5S,6s)-3-(5- (3-cyano-6-(1- methyl-1H-pyrazol- 4-yl)pyrazolo[1,5- a]pyridin-4- yl)pyrazin-2-yl)-3- azabicyclo[3.1.0] hexan- 6-yl)methyl)-6- methoxynicotinamide | 547.4 | ¹H NMR (400 MHz, DMSO-d₆) δ 9.22 (s, 1H), 8.72-8.51 (m, 4H), 8.38 (s, 1H), 8.17-8.04 (m, 3H), 7.98 (s, 1H), 6.88 (d, J = 8.3 Hz, 1H), 3.99-3.85 (m, 6H), 3.81 (d, J = 10.5 Hz, 2H), 3.52 (d, J = 9.2 Hz, 2H), 3.27-3.21 (m, 2H), 1.76 (s, 2H), 0.93 (s, 1H). |
| 157 | 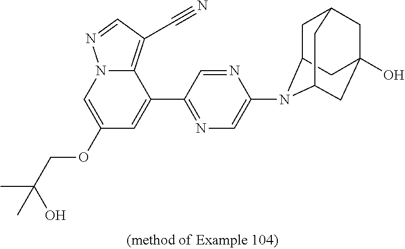 [SMILES: CC(C)(O)COc1cc(-c2cnc(N3C4CC5CC3CC(O)(C5)C4)cn2)c2c(C#N)cnn2c1] | 4-(5-((1R,3S,5s,7s)- 5-hydroxy-2- azaadamantan-2- yl)pyrazin-2-yl)-6- (2-hydroxy-2- methylpropoxy) pyrazolo [1,5-a]pyridine- 3-carbonitrile | 461.3 | ¹H NMR (400 MHz, DMSO-d₆) δ 8.66 (s, 1H), 8.54 (d, J = 9.7 Hz, 2H), 8.38 (s, 1H), 7.56 (s, 1H), 4.88 (s, 2H), 4.68 (s, 2H), 3.87 (s, 2H), 2.25 (s, 1H), 1.75 (s, 2H), 1.66 (d, J = 10.7 Hz, 8H), 1.22 (s, 6H). |
| 158 | 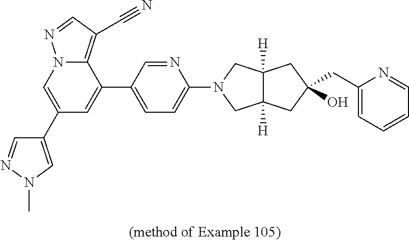 [SMILES: Cn1cc(-c2cc(-c3ccc(N4C[C@@H]5C[C@@](O)(Cc6ccccn6)C[C@@H]5C4)nc3)c3c(C#N)cnn3c2)cn1] | 4-(6-((3aR,5r,6aS)- 5-hydroxy-5- (pyridin-2- ylmethyl)hexahydro cyclopenta[c]pyrrol- 2(1H)-yl)pyridin-3- yl)-6-(1-methyl-1H- pyrazol-4- yl)pyrazolo[1,5- a]pyridine-3- carbonitrile | 517.4 | ¹H NMR (400 MHz, CDCl₃) δ 8.61 (s, 1H), 8.49 (s, 1H), 8.33 (s, 1H), 8.24 (s, 1H), 7.78 (s, 1H), 7.68 (m, 3H), 7.37 (s, 1H), 7.16 (d, J = 7.7 Hz, 2H), 6.54 (d, J = 8.3 Hz, 1H), 3.98 (s, 3H), 3.73 (s, 2H), 3.60 (d, J = 9.5 Hz, 2H), 3.03 (s, 2H), 2.88 (s, 2H), 1.99 (m, 2H), 1.83 (m, 2H). |
| 159 | 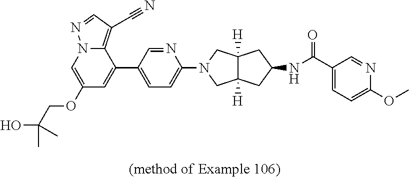 [SMILES: COc1ccc(C(=O)N[C@@H]2C[C@@H]3CN(c4ccc(-c5cc(OCC(C)(C)O)cn6ncc(C#N)c56)cn4)C[C@@H]3C2)cn1] | N-((3aR,5r,6aS)-2- (5-(3-cyano-6-(2- hydroxy-2- methylpropoxy) pyrazolo [1,5-a]pyridin-4- yl)pyridin-2- yl)octahydrocyclo- penta[c]pyrrol-5-yl)- 6- methoxynicotinamide | 568.3 | ¹H NMR (400 MHz, DMSO-d₆) δ 8.63 (d, J = 7.9 Hz, 2H), 8.55 (s, 1H), 8.39 (d, J = 6.7 Hz, 1H), 8.30 (s, 1H), 8.09 (d, J = 7.3 Hz, 1H), 7.73 (d, J = 8.1 Hz, 1H), 7.24 (s, 1H), 6.84 (d, J = 8.4 Hz, 1H), 6.62 (d, J = 8.5 Hz, 1H), 4.68 (s, 1H), 4.42-4.30 (m, 1H), 3.87 (s, 3H), 3.85 (s, 2H), 3.53 (s, 4H), 2.77 (m, 2H), 2.33-2.20 (m, 2H), 1.60-1.43 (m, 2H), 1.21 (s, 6H). |
| 160 | 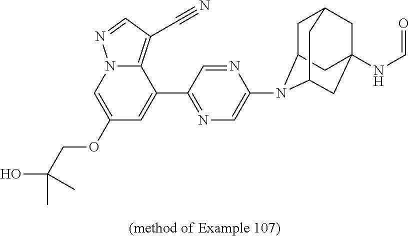 [SMILES: CC(C)(O)COc1cc(-c2cnc(N3C4CC5CC3CC(NC=O)(C5)C4)cn2)c2c(C#N)cnn2c1] | N-((1R,3S,5s,7s)-2- (5-(3-cyano-6-(2- hydroxy-2- methylpropoxy) pyrazolo [1,5-a]pyridin-4- yl)pyrazin-2-yl)-2- azaadamantan-5- yl)formamide | 487.554 | ¹H NMR (400 MHz, DMSO-d₆) δ 8.67 (s, 1H), 8.55 (d, J = 5.4 Hz, 2H), 8.41 (d, J = 7.4 Hz, 1H), 7.85 (s, 1H), 7.80 (s, 1H), 7.58 (s, 1H), 4.86 (s, 2H), 4.69 (s, 1H), 3.87 (s, 2H), 2.23 (s, 1H), 2.08 (m, 2H), 2.03 (m, 2H), 1.97 (m, 2H), 1.74 (s, 4H), 1.22 (s, 6H). |
| 161 | 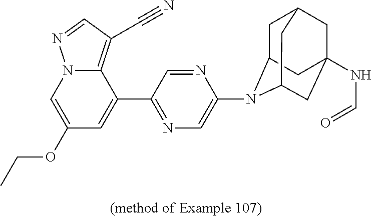 [SMILES: CCOc1cc(-c2cnc(N3C4CC5CC3CC(NC=O)(C5)C4)cn2)c2c(C#N)cnn2c1] | N-((1R,3S,5s,7s)-2- (5-(3-cyano-6- ethoxypyrazolo[1,5- a]pyridin-4- yl)pyrazin-2-yl)-2- azaadamantan-5- yl)formamide | 444.4 | ¹H NMR (400 MHz, DMSO-d₆) δ 8.64 (s, 1H), 8.54 (d, J = 7.9 Hz, 1H), 8.39 (d, J = 7.1 Hz, 1H), 7.82 (d, J = 17.6 Hz, 2H), 7.55 (s, 1H), 4.98-4.78 (m, 2H), 4.27- 4.07 (m, 2H), 2.50-1.70 m, 11H), 1.43-1.33 (m, 3H). |
| 162 | 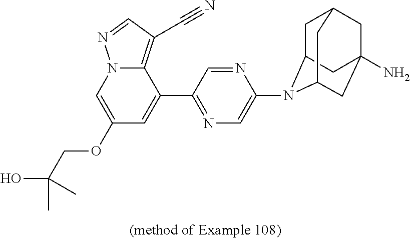 [SMILES: CC(C)(O)COc1cc(-c2cnc(N3C4CC5CC3CC(N)(C5)C4)cn2)c2c(C#N)cnn2c1] | 4-(5-((1R,3S,5s,7s)- 5-amino-2- azaadamantan-2- yl)pyrazin-2-yl)-6- (2-hydroxy-2- methylpropoxy) pyrazolo [1,5-a]pyridine- 3-carbonitrile | 460.4 | ¹H NMR (400 MHz, DMSO-d₆) δ 8.69 (d, J = 1.9 Hz, 1H), 8.58 (s, 1H), 8.56 (s, 1H), 8.44 (s, 1H), 8.00 (s, 2H), 7.58 (d, J = 1.9 Hz, 1H), 4.93 (s, 2H), 4.73-4.65 (m, 1H), 3.93-3.85 (m, 2H), 2.31 (s, 1H), 1.95 (m, 2H), 1.89 (m, 4H), 1.79 (d, J = 12.7 Hz, 2H), 1.70 (d, J = 12.2 Hz, 2H), 1.22 (s. 6H). |
| 163 | 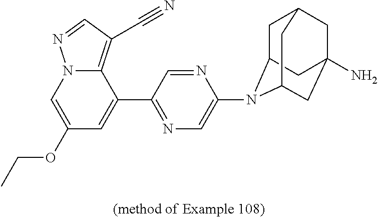 [SMILES: CCOc1cc(-c2cnc(N3C4CC5CC3CC(N)(C5)C4)cn2)c2c(C#N)cnn2c1] | 4-(5-((1R,3S,5s,7s)- 5-amino-2- azaadamantan-2- yl)pyrazin-2-yl)-6- ethoxypyrazolo[1,5- a]pyridine-3- carbonitrile | 416.4 | ¹H NMR (400 MHz, DMSO-d₆) δ 8.67 (s, 1H), 8.57 (s, 2H), 8.43 (s, 1H), 7.84 (s, 2H), 7.57 (s, 1H), 4.92 (s, 2H), 4.16 (d, J = 6.9 Hz, 2H), 2.30 (s, 1H), 1.94 (s, 2H), 1.88 (m, 4H), 1.79 (d, J = 12.0 Hz, 2H), 1.69 (d, J = 12.4 Hz, 2H), 1.35 (t, J = 6.9 Hz, 3H). |
| 164 | 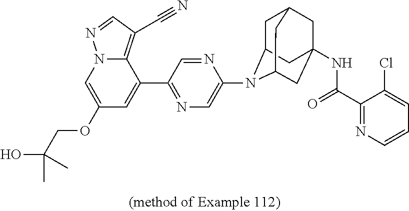 [SMILES: CC(C)(O)COc1cc(-c2cnc(N3C4CC5CC3CC(NC(=O)c3ncccc3Cl)(C5)C4)cn2)c2c(C#N)cnn2c1] | 3-chloro-N- ((1R,3S,5s,7s)-2-(5- (3-cyano-6-(2- hydroxy-2- methylpropoxy) pyrazolo [1,5-a]pyridin-4- yl)pyrazin-2-yl)-2- azaadamantan-5- yl)picolinamide | 599.5 | ¹H NMR (400 MHz, DMSO-d₆) δ 8.67 (d, J = 2.0 Hz, 1H), 8.56 (s, 2H), 8.48 (dd, J = 4.7, 1.2 Hz, 1H), 8.43 (d, J = 1.0 Hz, 1H), 8.26 (s, 1H), 7.97 (dd, J = 8.2, 1.3 Hz, 1H), 7.59 (d, J = 2.0 Hz, 1H), 7.47 (dd, J = 8.2, 4.7 Hz, 1H), 4.91 (s, 2H), 4.69 (s, 1H), 3.88 (s, 2H), 2.28 (s, 1H), 2.18 (m, 6H), 1.78 (s, 4H), 1.22 (s, 6H). |
| 165 | 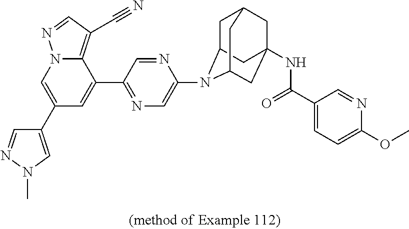 [SMILES: COc1ccc(C(=O)NC23CC4CC(C2)N(c2cnc(-c5cc(-c6cnn(C)c6)cn6ncc(C#N)c56)cn2)C(C4)C3)cn1] | N-((1R,3S,5s,7s)-2- (5-(3-cyano-6-(1- methyl-1H-pyrazol- 4-yl)pyrazolo[1,5- a]pyridin-4- yl)pyrazin-2-yl)-2- azaadamantan-5-yl)- 6- methoxynicotinamide | 587.4 | ¹H NMR (400 MHz, DMSO-d₆) δ 9.23 (d, J = 1.3 Hz, 1H), 8.64- 8.61 (m, 2H), 8.58 (d, J = 2.2 Hz, 1H), 8.44 (s, 1H), 8.40 (s, 1H), 8.13 (s, 1H), 8.04 (t, J = 2.2 Hz, 2H), 7.85 (s, 1H), 6.83 (d, J = 8.7 Hz, 1H), 4.92 (s, 2H), 3.87 (s, 6H), 2.28 (s, 1H), 2.24 (s, 2H), 2.18 (d, J = 6.2 Hz, 4H), 1.79 (s, 4H). |
| 166 | 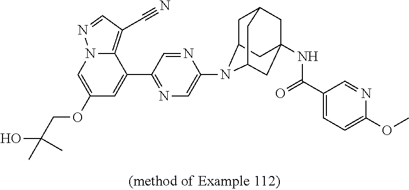 [SMILES: COc1ccc(C(=O)NC23CC4CC(C2)N(c2cnc(-c5cc(OCC(C)(C)O)cn6ncc(C#N)c56)cn2)C(C4)C3)cn1] | N-((1R,3S,5s,7s)-2- (5-(3-cyano-6-(2- hydroxy-2- methylpropoxy) pyrazolo [1,5-a]pyridin-4- yl)pyrazin-2-yl)-2- azaadamantan-5-yl)- 6- methoxynicotinamide | 595.5 | ¹H NMR (400 MHz, DMSO-d₆) δ 8.67 (d, J = 1.9 Hz, 1H). 8.58 (d, J = 2.1 Hz, 1H), 8.56 (s, 2H), 8.42 (s, 1H), 8.05 (dd, J = 8.7, 2.4 Hz, 1H), 7.85 (s, 1H), 7.58 (d, J = 1.9 Hz, 1H), 6.83 (d, J = 8.7 Hz, 1H), 4.90 (s, 2H), 4.69 (s, 1H), 3.87 (s, 5H), 2.27 (s, 1H), 2.24 (s, 2H), 2.19 (s, 2H), 2.13 (m, 2H), 1.76 (m, 4H), 1.22 (s, 6H). |
| 167 | 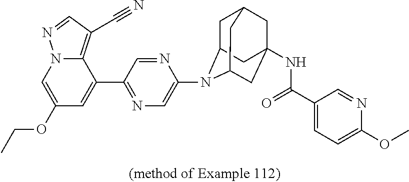 [SMILES: CCOc1cc(-c2cnc(N3C4CC5CC3CC(NC(=O)c3ccc(OC)nc3)(C5)C4)cn2)c2c(C#N)cnn2c1] | N-((1R,3S,5s,7s)-2- (5-(3-cyano-6- cthoxypyrazolo[1,5- a]pyridin-4- yl)pyrazin-2-yl)-2- azaadamantan-5-yl)- 6- methoxynicotinamide | 551.5 | ¹H NMR (400 MHz, DMSO-d₆) δ 8.65 (s, 1H), 8.57 (d, J = 11.0 Hz, 2H), 8.41 (s, 1H), 8.05 (d, J = 8.7 Hz, 1H), 7.85 (s, 1H), 7.56 (s, 1H), 6.83 (d, J = 8.8 Hz, 1H), 4.90 (s, 2H), 4.16 (q, J = 7.1 Hz, 2H), 3.87 (s, 3H), 2.31-2.11 (m, 7H), 1.77 (s, 4H), 1.37 (t, J = 7.1 Hz, 3H). |
| 168 | 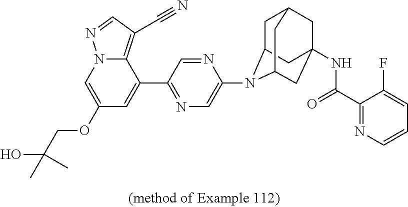 [SMILES: CC(C)(O)COc1cc(-c2cnc(N3C4CC5CC3CC(NC(=O)c3ncccc3F)(C5)C4)cn2)c2c(C#N)cnn2c1] | N-((1R,3S,5s,7s)-2- (5-(3-cyano-6-(2- hydroxy-2- methylpropoxy) pyrazolo [1,5-a]pyridin-4- yl)pyrazin-2-yl)-2- azaadamantan-5-yl)- 3-fluoropicolinamide | 583.4 | ¹H NMR (400 MHz, DMSO-d₆) δ 8.67 (t, J = 6.0 Hz, 1H), 8.61- 8.50 (m, 2H), 8.43 (t, J = 5.9 Hz, 2H), 8.12 (t, J = 6.2 Hz, 1H), 7.88- 7.75 (m, 1H), 7.61 (m, 2H), 4.91 (s, 2H), 4.73-4.65 (m, 1H), 3.95-3.79 (m, 2H), 2.33-2.07 (m, 7H), 1.78 (s, 4H), 1.21 (s, 6H). |
| 169 | 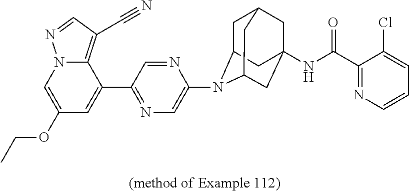 [SMILES: CCOc1cc(-c2cnc(N3C4CC5CC3CC(NC(=O)c3ncccc3Cl)(C5)C4)cn2)c2c(C#N)cnn2c1] | 3-chloro-N- ((1R,3S,5s,7s)-2-(5- (3-cyano-6- ethoxypyrazolo[1,5- a]pyridin-4- yl)pyrazin-2-yl)-2- azaadamantan-5- yl)picolinamide | 555.2 | ¹H NMR (400 MHz, DMSO-d₆) δ 8.70-8.36 (m, 4H), 8.26 (d, J = 7.1Hz, 1H), 7.97 (q, J = 8.9, 7.9 Hz, 1H), 7.63-7.41 (m, 2H), 4.91 (s, 1H), 4.15 (q, J = 7.0 Hz, 2H), 2.20 (m, 7H), 1.78 (s, 4H), 1.38 (q, J = 7.6, 7.1 Hz, 3H). |
| 170 | 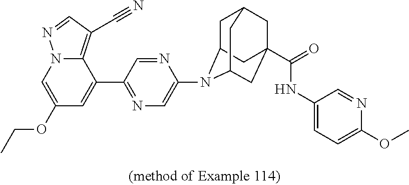 [SMILES: CCOc1cc(-c2cnc(N3C4CC5CC3CC(C(=O)Nc3ccc(OC)nc3)(C5)C4)cn2)c2c(C#N)cnn2c1] | (1R,3S,5s,7s)-2-(5- (3-cyano-6- ethoxypyrazolo[1,5- a]pyridin-4- yl)pyrazin-2-yl)-N- (6-methoxypyridin- 3-yl)-2- azaadamantane-5- carboxamide | 551.1 | ¹H NMR (400 MHz, CDCl₃) δ 8.35 (s, 1H), 8.26 (s, 1H), 8.19 (s, 1H), 8.15 (s, 1H), 8.11 (s, 1H), 7.90 (d, J = 8.7 Hz, 1H), 7.31 (s, 1H), 6.68 (d, J = 8.9 Hz, 1H), 4.81 (s, 2H), 4.07 (dd, J = 13.5, 6.6 Hz, 2H), 3.93-3.77 (m, 3H), 2.34 (s, 1H), 2.12 (s, 4H), 1.98 (d, J = 12.5 Hz, 4H), 1.83 (d, J = 12.4 Hz, 2H), 1.46 (t, J = 6.8 Hz, 3H). |
| 171 | 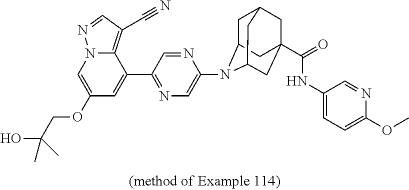 [SMILES: COc1ccc(NC(=O)C23CC4CC(C2)N(c2cnc(-c5cc(OCC(C)(C)O)cn6ncc(C#N)c56)cn2)C(C4)C3)cn1] | (1R,3S,5s,7s)-2-(5- (3-cyano-6-(2- hydroxy-2- methylpropoxy) pyrazolo [1,5-a]pyridin-4- yl)pyrazin-2-yl)-N- (6-methoxypyridin- 3-yl)-2- azaadamantane-5- carboxamide | 595.2 | ¹H NMR (400 MHz, CDCl₃) δ 8.36 (s, 1H), 8.25 (s, 1H), 8.20 (s, 1H), 8.16 (s, 2H), 8.00 (s, 1H), 7.90 (d, J = 8.5 Hz, 1H), 7.37 (s, 1H), 6.68 (d, J = 8.7 Hz, 1H), 4.80 (s, 2H), 3.86 (s, 3H), 3.83 (s, 2H), 2.34 (s, 1H), 2.12 (s, 4H), 1.98 (d, J = 11.9 Hz, 4H), 1.83 (d, J = 12.0 Hz, 2H), 1.35 (s, 6H). |
| 172 | 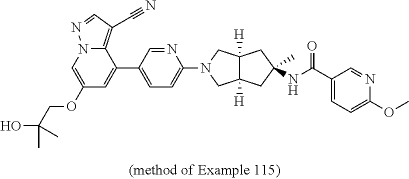 [SMILES: COc1ccc(C(=O)N[C@@]2(C)C[C@H]3CN(c4ccc(-c5cc(OCC(C)(C)O)cn6ncc(C#N)c56)cn4)C[C@H]3C2)cn1] | N-((3aR,5r,6aS)-2- (5-(3-cyano-6-(2- hydroxy-2- methylpropoxy) pyrazolo [1,5-a]pyridin-4- yl)pyridin-2-yl)-5- methyloctahydrocyclo- penta[c]pyrrol-5- yl)-6- methoxynicotinamide | 582.0 | ¹H NMR (400 MHz, CD₃OD) δ 8.50 (s, 1H), 8.30-8.12 (m, 3H), 7.91 (d, J = 8.2 Hz, 1H), 7.76- 7.59 (m, 2H), 7.20 (s, 1H), 6.69 (d, J = 8.6 Hz, 1H), 6.59 (d, J = 8.8 Hz, 1H), 3.88 (s, 3H), 3.84 (s, 2H), 3.71-3.51 (m, 4H), 2.97 (b, 2H), 2.28 (m, 2H), 2.11 (m, 2H), 1.49 (s, 3H), 1.33 (s, 6H). |
| 173 | 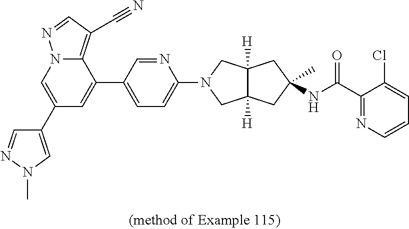 [SMILES: Cn1cc(-c2cc(-c3ccc(N4C[C@@H]5C[C@](C)(NC(=O)c6ncccc6Cl)C[C@@H]5C4)nc3)c3c(C#N)cnn3c2)cn1] | 3-chloro-N- ((3aR,5r,6aS)-2-(5- (3-cyano-6-(1- methyl-1H-pyrazol- 4-yl)pyrazolo[1,5- a]pyridin-4- yl)pyridin-2-yl)-5- methyloctahydrocyclo- penta[c]pyrrol-5- yl)picolinamide | 578.4 | ¹H NMR (400 MHz, CDCl₃) δ 8.61 (s, 1H), 8.37-8.28 (m, 2H), 8.24 (s, 1H), 8.04 (s, 1H), 7.76 (d, J = 8.8 Hz, 2H), 7.68 (d, J = 9.5 Hz, 2H), 7.36 (s, 1H), 7.29 (d, J = 3.6 Hz, 1H), 6.51 (d, J = 8.7 Hz, 1H), 3.98 (s, 3H), 3.67 (d, J = 6.1 Hz, 2H), 3.60 (d, J = 10.3 Hz, 2H), 2.97 (s, 2H), 2.35 (d, J = 6.2 Hz, 2H), 2.17-2.08 (m, 2H), 1.56 (s, 3H). |
| 174 | 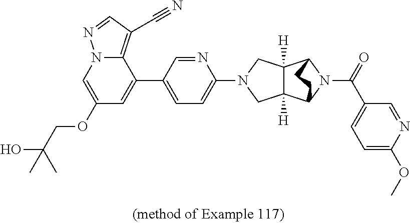 | 6-(2-hydroxy-2- methylpropoxy)-4- (6- ((3aR,4S,7R,7aS)-8- (6- methoxynicotinoyl) hexahydro-1H-4,7- epiminoisoindol- 2(3H)-yl)pyridin-3- yl)pyrazolo[1,5- a]pyridine-3- carbonitrile | 580.4 | ¹H NMR (400 MHz, CD₃OD) δ 8.44 (s, 2H), 8.33 (s, 1H), 8.29 (s, 1H), 7.91 (d, J = 8.3 Hz, 1H), 7.80 (d, J = 8.9 Hz, 1H), 7.30 (s, 1H), 6.87 (d, J = 8.6 Hz, 1H), 6.79 (d, J = 8.8 Hz, 1H), 3.97 (s, 3H), 3.91 (s, 2H), 3.90-3.78 (m, 2H), 3.32 (m, 2H), 3.19 (m, 2H), 3.13 (m, 2H), 1.73 (b. 4H), 1.35 (s, 6H). |
| 175 | 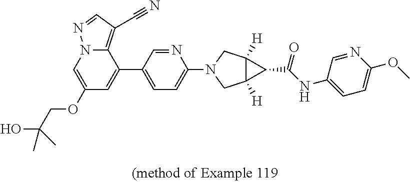 [SMILES: COc1ccc(NC(=O)[C@H]2[C@@H]3CN(c4ccc(-c5cc(OCC(C)(C)O)cn6ncc(C#N)c56)cn4)C[C@@H]32)cn1] | (1R,5S,6r)-3-(5-(3- cyano-6-(2-hydroxy- 2- methylpropoxy) pyrazolo [1,5-a]pyridin-4- yl)pyridin-2-yl)-N- (6-methoxypyridin- 3-yl)-3- azabicyclo[3.1.0] hexane- 6-carboxamide | 540.3 | ¹H NMR (400 MHz, CDCl₃) δ 8.17-8.04 (m, 4H), 7.83 (d, J = 8.7 Hz, 1H), 7.57 (d, J = 8.4 Hz, 1H), 7.09 (s, 1H), 6.61 (d, J =8.8 Hz, 1H), 6.43 (d, J = 8.6 Hz, 1H), 3.78 (s, 3H), 3.76-3.74 (m, 4H), 3.54 (d, J = 10.1 Hz, 2H), 2.23 (s, 2H), 1.51 (s, 1H), 1.25 (s, 6H). |
| 176 | 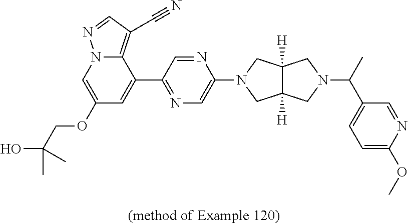 [SMILES: COc1ccc(C(C)N2C[C@H]3CN(c4cnc(-c5cc(OCC(C)(C)O)cn6ncc(C#N)c56)cn4)C[C@H]3C2)cn1] | 6-(2-hydroxy-2- methylpropoxy)-4- (5-((3aR,6aS)-5-(1- (6-methoxypyridin- 3- yl)ethyl)hexahydro- pyrrolo[3,4-c]pyrrol- 2(1H)-yl)pyrazin-2- yl)pyrazolo[1,5- a]pyridine-3- carbonitrile | 555.3 | ¹H NMR (400 MHz, DMSO-d₆) δ 8.66 (s, 1H), 8.54 (d, J = 7.5 Hz, 2H), 8.08 (s, 1H), 8.02 (s, 1H), 7.62 (d, J = 8.0 Hz, 1H), 7.54 (s, 1H), 6.74 (d, J = 8.6 Hz, 1H), 4.69 (s, 1H), 3.86 (s, 2H), 3.80 (s, 3H), 3.71 (d, J = 8.4 Hz, 2H), 3.71 (d, J = 8.4 Hz, 2H), 3.45 (s, 2H), 3.45 (s, 2H), 3.34 (s, 1H), 2.91 (s, 2H), 2.59 (s, 2H), 2.49 (s, 2H), 1.27 (d, 3H), 1.21 (s, 6H). |
| 177 | 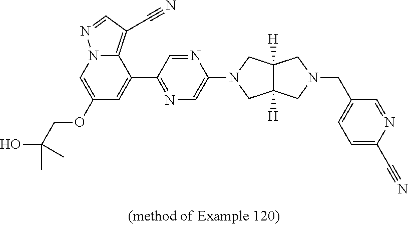 [SMILES: CC(C)(O)COc1cc(-c2cnc(N3C[C@H]4CN(Cc5ccc(C#N)nc5)C[C@H]4C3)cn2)c2c(C#N)cnn2c1] | 4-(5-((3aR,6aS)-5- ((6-cyanopyridin-3- yl)methyl)hexahydro pyrrolo[3,4-c]pyrrol- 2(1H)-yl)pyrazin-2- yl)-6-(2-hydroxy-2- methylpropoxy) pyrazolo [1,5-a]pyridine- 3-carbonitrile | 536.4 | ¹H NMR (400 MHz, DMSO-d₆) δ 8.66 (s, 2H), 8.54 (d, J = 7.3 Hz, 2H), 8.09 (s, 1H), 7.99-7.91 (m, 2H), 7.54 (d, J = 1.9 Hz, 1H), 4.69 (s, 1H), 3.87 (s, 2H), 3.78- 3.68 (m, 4H), 3.43 (d, J = 13.5 Hz, 2H), 2.97 (s, 2H), 2.62 (s, 2H), 2.55 (d, J = 7.9 Hz, 2H), 1.21 (s, 6H). |
| 178 | 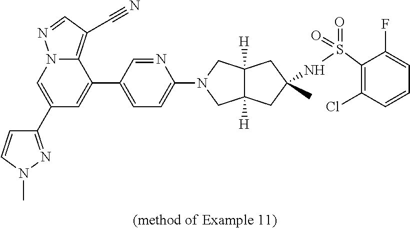 [SMILES: Cn1ccc(-c2cc(-c3ccc(N4C[C@@H]5C[C@@](C)(NS(=O)(=O)c6c(F)cccc6Cl)C[C@@H]5C4)nc3)c3c(C#N)cnn3c2)n1] | 2-chloro-N- ((3aR,5s,6aS)-2-(5- (3-cyano-6-(1- methyl-1H-pyrazol- 3-yl)pyrazolo[1,5- a]pyridin-4- yl)pyridin-2-yl)-5- methyloctahydrocyclo- penta[c]pyrrol-5- yl)-6- fluorobenzenesulfon- amide | 631.5 | ¹H NMR (400 MHz, CD₃OD) δ 8.96 (s, 1H), 8.35-8.20 (m, 2H), 7.77 (m, 2H), 7.58 (s, 1H), 7.53- 7.27 (m, 2H), 7.19 (m, 1H), 6.70- 6.56 (m, 2H), 3.95 (s, 3H), 3.62- 3.35(m, 4H), 2.7(m, 1.5H), 2.49 (m, 1.5H), 2.10 (m, 0.5H), 1.85 (m, 0.5H), 1.50-1.20 (m, 5H). |
| 179 | 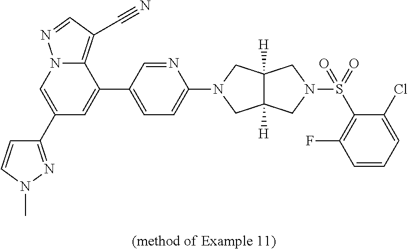 [SMILES: Cn1ccc(-c2cc(-c3ccc(N4C[C@@H]5CN(S(=O)(=O)c6c(F)cccc6Cl)C[C@@H]5C4)nc3)c3c(C#N)cnn3c2)n1] | 4-(6-((3aR,6aS)-5- ((2-chloro-6- fluorophenyl)sulfonyl) hexahydropyrrolo [3,4-c]pyrrol-2(1H)- yl)pyridin-3-yl)-6- (1-methyl-1H- pyrazol-3- yl)pyrazolo[1,5- a]pyridine-3- carbonitrile | 603.2 | ¹H NMR (400 MHz, CD₃OD) δ 8.92 (d, J = 8.2 Hz, 1H), 8.27 (s, 2H), 7.75 (m, 2H), 7.41 (m, 3H), 7.16 (d, J = 9.6 Hz, 1H), 6.67- 6.49 (m, 2H), 3.94 (s, 3H), 3.77 (m, 4H), 3.45 (m, 4H), 3.15 (m, 2H). |
| 180 | 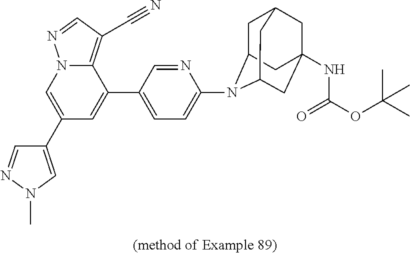 [SMILES: Cn1cc(-c2cc(-c3ccc(N4C5CC6CC4CC(NC(=O)OC(C)(C)C)(C6)C5)nc3)c3c(C#N)cnn3c2)cn1] | ((1R,3S,5s,7s)-2-(5- (3-cyano-6-(1- methyl-1H-pyrazol- 4-yl)pyrazolo[1,5- a]pyridin-4- yl)pyridin-2-yl)-2- azaadamantan-5- yl)carbamate | 551.4 | ¹H NMR (400 MHz, CDCl₃) δ 8.62 (s, 1H), 8.35 (s, 1H), 8.25 (s, 1H), 7.79 (s, 1H), 7.74 (d, J = 8.3 Hz, 1H), 7.68 (s, 1H), 7.40 (s, 1H), 6.77 (d, J = 8.7 Hz, 1H), 4.81 (s, 2H), 4.46 (s, 1H), 3.99 (s, 3H), 2.31 (s, 1H), 2.16 (s, 2H), 2.07 (s, 2H), 2.01 (s, 2H), 1.90 (m, 2H), 1.74 (m, 2H), 1.42 (s, 9H). |
| 181 | 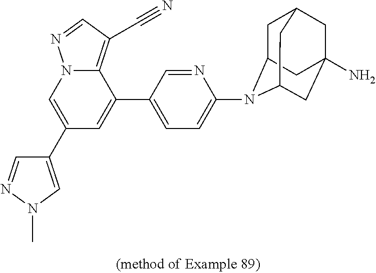 [SMILES: Cn1cc(-c2cc(-c3ccc(N4C5CC6CC4CC(N)(C6)C5)nc3)c3c(C#N)cnn3c2)cn1] | 4-(6-((1R,3S,5s,7s)- 5-amino-2- azaadamantan-2- yl)pyridin-3-yl)-6- (1-methyl-1H- pyrazol-4- yl)pyrazolo[1,5- a]pyridine-3- carbonitrile | 451.3 | ¹H NMR (400 MHz, CDCl₃) δ 8.58 (s, 1H), 8.29 (s, 1H), 8.19 (s, 1H), 7.77 (s, 1H), 7.70 (s, 2H), 7.41 (s, 1H), 6.76 (d, J = 7.1 Hz, 1H), 4.85 (s, 2H), 3.95 (s, 3H), 2.50-2.20 (s, 9H), 1.89 (m, 2H), 1.76 (m, 2H). |
| 182 | 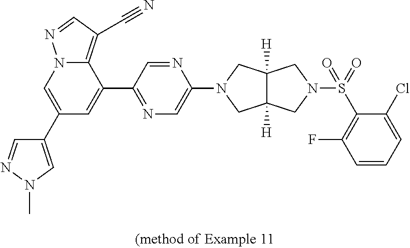 [SMILES: Cn1cc(-c2cc(-c3cnc(N4C[C@@H]5CN(S(=O)(=O)c6c(F)cccc6Cl)C[C@@H]5C4)cn3)c3c(C#N)cnn3c2)cn1] | 4-(5-((3aR,6aS)-5- ((2-chloro-6- fluorophenyl)sulfonyl) hexah dropyrrolo [3,4-c]pyrrol-2(1H)- yl)pyrazin-2-yl)-6- (1-methyl-1H- pyrazol-4- yl)pyrazolo[1,5- a]pyridine-3- carbonitrile | 604.4 | ¹H NMR (400 MHz, DMSO-d₆) δ 9.23 (s, 1H), 8.63 (d, J = 6.6 Hz, 2H), 8.39 (s, 1H), 8.12 (s, 1H), 8.03 (d, J = 17.9 Hz, 2H), 7.70- 7.61 (m, 1H), 7.53 (d, J = 8.1 Hz, 1H), 7.44 (t, J = 9.9 Hz, 1H), 3.88 (s, 3H), 3.78-3.70 (m, 2H), 3.62 (d, J = 8.4 Hz, 2H), 3.44 (d, J = 11.2 Hz, 2H), 3.35 (s, 2H), 3.13 (s, 2H). |
| 183 | 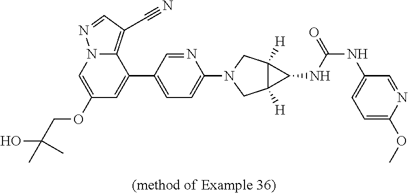 [SMILES: COc1ccc(NC(=O)N[C@H]2[C@@H]3CN(c4ccc(-c5cc(OCC(C)(C)O)cn6ncc(C#N)c56)cn4)C[C@@H]32)cn1] | 1-((1R,5S,6s)-3-(5- (3-cyano-6-(2- hydroxy-2- methylpropoxy) pyrazolo [1,5-a]pyridin-4- yl)pyridin-2-yl)-3- azabicyclo[3.1.0] hexan-6-yl)-3-(6- methoxypyridin-3- yl)urea | 555.5 | ¹H NMR (400 MHz, DMSO-d₆) δ 8.67-8.60 (m, 1H), 8.55 (s, 1H), 8.36-8.26 (m, 2H), 8.14 (d, J = 2.7 Hz, 1H), 7.74 (ddd, J = 11.6, 8.7, 2.6 Hz, 2H), 7.29-7.20 (m, 1H), 6.71 (d, J = 8.9 Hz, 1H), 6.62-6.51 (m, 2H), 3.85 (s, mH), 3.81 (s, 2H), 3.48 (m, 2H), 2.36 (s, 1H), 1.86 (s, 2H), 1.20 (s, 6H). |
| 184 | 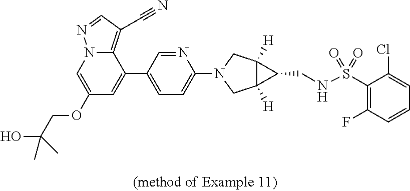 [SMILES: CC(C)(O)COc1cc(-c2ccc(N3C[C@@H]4[C@@H](CNS(=O)(=O)c5c(F)cccc5Cl)[C@@H]4C3)nc2)c2c(C#N)cnn2c1] | 2-chloro-N- (((1R,5S,6r)-3-(5-(3- cyano-6-(2-hydroxy- 2- methylpropoxy) pyrazolo [1,5-a]pyridin-4- yl)pyridin-2-yl)-3- azabicyclo[3.1.0] hexan- 6-yl)methyl)-6- fluorobenzenesulfon- amide | 611.3 | ¹H NMR (400 MHz, CDCl₃) δ 8.33 (s, 1H), 8.19 (d, J = 10.6 Hz, 2H), 7.78 (s, 1H), 7.47 (s, 1H), 7.39 (d, J = 1.6 Hz, 1H), 7.20 (d, J = 9.6 Hz, 2H), 6.52 (s, 1H), 5.57 (s, 1H), 3.87 (m, 2H), 3.70 (m, 2H), 3.58 (m, 2H), 3.14 (m, 2H), 1.69 (m, 2H), 1.39 (s, 6H), 0.88 (s, 1H). |
| 185 | 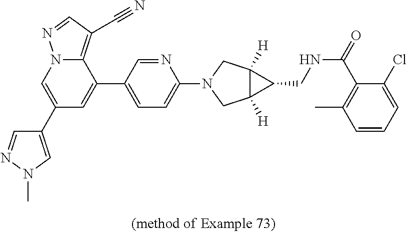 [SMILES: Cc1cccc(Cl)c1C(=O)NC[C@@H]1[C@H]2CN(c3ccc(-c4cc(-c5cnn(C)c5)cn5ncc(C#N)c45)cn3)C[C@@H]12] | 2-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan- 6-yl)methyl)-6- methylbenzamide | 563.2 | ¹H NMR (400 MHz, DMSO-d₆) δ 9.19 (s, 1H), 8.62 (s, 2H), 8.41- 8.27 (m, 2H), 8.09 (s, 1H), 7.79- 7.69 (m, 2H), 7.34-7.25 (m, 2H), 7.21 (s, 1H), 6.57 (d, J = 8.8 Hz, 1H), 3.86 (s, 3H), 3.71 (d, J = 10.6 Hz, 2H), 3.44 (d, J = 10.2 Hz, 2H), 3.21 (d, J = 6.4 Hz, 2H), 2.27 (s, 3H), 1.74 (s, 2H), 0.91 (s, 1H). |
| 186 | 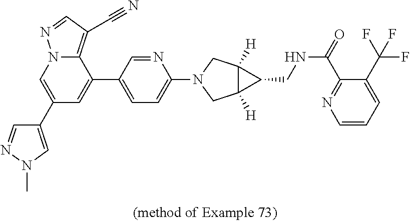 [SMILES: Cn1cc(-c2cc(-c3ccc(N4C[C@@H]5[C@@H](CNC(=O)c6ncccc6C(F)(F)F)[C@@H]5C4)nc3)c3c(C#N)cnn3c2)cn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-3- (trifluoromethyl) picolinamide | 584.4 | ¹H NMR (400 MHz, DMSO-d₆) δ 9.19 (s, 1H), 8.88-8.77 (m, 2H), 8.62 (s, 1H), 8.36 (s, 1H), 8.30 (dd, J = 9.5, 5.4 Hz, 2H), 8.09 (s, 1H), 7.80-7.67 (m, 3H), 6.57 (d, J = 8.8 Hz, 1H), 3.86 (s, 3H), 3.71 (d, J = 10.5 Hz, 2H), 3.43 (d, J = 10.2 Hz, 2H), 3.26 (d, J = 7.1 Hz, 2H), 1.73 (m, 2H), 0.90 (m, 1H). |
| 187 | 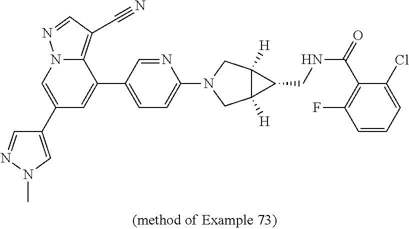 [SMILES: Cn1cc(-c2cc(-c3ccc(N4C[C@@H]5[C@@H](CNC(=O)c6c(F)cccc6Cl)[C@@H]5C4)nc3)c3c(C#N)cnn3c2)cn1] | 2-chloro-N- (((1R,5S,6s)-3-(5-(3- cyano-6-(1-methyl- 1H-pyrazol-4- yl)pyrazolo[1,5- a]pyridin-4- yl)pyridin-2-yl)-3- azabicyclo[3.1.0] hexan- 6-yl)methyl)-6- fluorobenzamide | 567.3 | ¹H NMR (400 MHz, Chloroform- d) δ 8.61 (s, 1H), 8.19 (s, 2H), 7.74 (s, 3H), 7.40 (s, 1H), 7.17- 6.95 (m, 3H), 6.53 (s, 1H), 3.90 (s, 3H), 3.80-3.50 (m, 4H), 3.29 (m, 2H), 1.76 (s, 2H), 0.97 (s, 1H). |
| 188 | 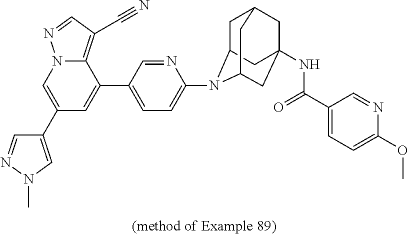 [SMILES: COc1ccc(C(=O)NC23CC4CC(C2)N(c2ccc(-c5cc(-c6cnn(C)c6)cn6ncc(C#N)c56)cn2)C(C4)C3)cn1] | N-((1R,3S,5s,7s)-2- (5-(3-cyano-6-(1- methyl-1H-pyrazol- 4-yl)pyrazolo[1,5- a]pyridin-4- yl)pyridin-2-yl)-2- azaadainantan-5-yl)- 6- methoxynicotinamide | 586.2 | ¹H NMR (400 MHz, Methanol-d₄) δ 8.93 (s, 1H), 8.55 (s, 1H), 8.36 (m, 2H), 8.14 (s, 1H), 8.00 (m, 2H), 7.78 (d, J = 9.1 Hz, 1H), 7.66 (s, 1H), 6.87 (m, 2H), 3.95 (s, 6H), 2.40-2.27 (m, 5H), 2.21 m, 2H), 1.98-1.78 (m. 4H), 1.38 (m, 2H). |
| 189 | 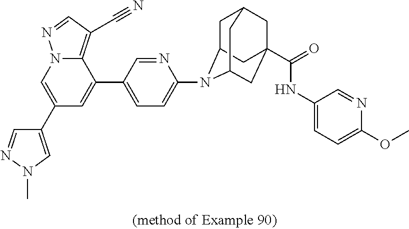 [SMILES: COc1ccc(NC(=O)C23CC4CC(C2)N(c2ccc(-c5cc(-c6cnn(C)c6)cn6ncc(C#N)c56)cn2)C(C4)C3)cn1] | (1R,3S,5s,7s)-2-(5- (3-cyano-6-(1- methyl-1H-pyrazol- 4-yl)pyrazolo[1,5- a]pyridin-4- yl)pyridin-2-yl)-N- (6-methoxypyridin- 3-yl)-2- azaadamantane-5- carboxamide | 586.3 | ¹H NMR (400 MHz, CDCl₃) δ 8.62 (s, 1H), 8.31 (s, 1H), 8.23 (s, 1H), 8.17 (s, 1H), 7.89 (d, J = 8.0 Hz, 1H), 7.79 (d, J = 8.9 Hz, 2H), 7.70 (s, 1H), 7.65 (d, J = 7.7 Hz, 1H), 7.42 (s, 1H), 6.79 (d, J = 7.9 Hz, 1H), 6.68 (d, J = 8.1 Hz, 1H), 4.75 (s, 2H), 3.96 (s, 3H), 3.87 (s, 3H), 2.32 (s, 1H), 2.10-1.80 (m, 10H). |
| 190 | 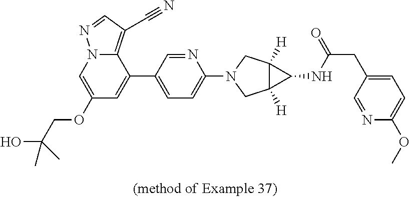 [SMILES: COc1ccc(CC(=O)N[C@H]2[C@@H]3CN(c4ccc(-c5cc(OCC(C)(C)O)cn6ncc(C#N)c56)cn4)C[C@@H]32)cn1] | N-((1R,5S,6s)-3-(5- (3-cyano-6-(2- hydroxy-2- methylpropoxy) pyrazolo [1,5-a]pyridin-4- yl)pyridin-2-yl)-3- azabicyclo[3.1.0] hexan- 6-yl)-2-(6- methoxypyridin-3- yl)acetamide | 554.3 | ¹H NMR (400 MHz, DMSO-d₆) δ 8.63 (d, J = 2.1 Hz, 1H), 8.54 (s, 1H), 8.28 (dd, J = 6.8, 3.2 Hz. 2H), 7.98 (d, J = 2.5 Hz, 1H), 7.72 (dd, J = 8.7, 2.5 Hz, 1H), 7.56 (dd, J = 8.5, 2.5 Hz, 1H), 7.23 (d, J = 2.1 Hz, 1H), 6.74 (d, J = 8.5 Hz, 1H), 6.56 (d, J = 8.7 Hz, 1H), 4.68 (s, 1H), 3.84 (s, 2H), 3.80 (s, 3H), 3.75 (s, 1H), 3.72 (s, 1H), 3.51-3.41 (m, 2H), 2.43-2.37 (m, 1H), 1.82 (d, J = 3.2 Hz, 2H), 1.21 (d, J = 5.6 Hz, 6H). |
| 191 | 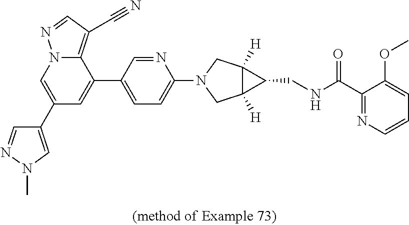 [SMILES: COc1cccnc1C(=O)NC[C@@H]1[C@H]2CN(c3ccc(-c4cc(-c5cnn(C)c5)cn5ncc(C#N)c45)cn3)C[C@@H]12] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-3- methoxypicolinamide | 546.4 | ¹H NMR (400 MHz, DMSO-d₆) δ 9.18 (s, 1H), 8.61 (s, 1H), 8.42 (t, 1H), 8.36 (s, 1H), 8.31 (s, 1H), 8.12 (d, J = 4.0 Hz, 1H), 8.09 (s, 1H), 7.76-7.70 (m, 2H), 7.55 (d, J = 8.4 Hz, 1H), 7.44 (dd, J = 8.2, 4.5 Hz, 1H), 6.56 (d, J = 8.7 Hz, 1H), 3.85 (s, 3H), 3.80 (s, 3H), 3.72 (d, J = 10.4 Hz, 2H), 3.42 (d, J = 9.6 Hz, 2H), 3.22 (t, 2H), 1.71 (s, 2H), 0.88 (s, 1H). |
| 192 | 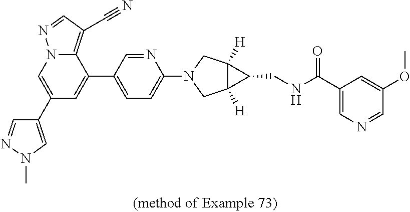 [SMILES: COc1cncc(C(=O)NC[C@@H]2[C@H]3CN(c4ccc(-c5cc(-c6cnn(C)c6)cn6ncc(C#N)c56)cn4)C[C@@H]23)c1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-5- methoxynicotinainide | 546.3 | ¹H NMR (400 MHz, DMSO-d₆) δ 9.19 (s, 1H), 8.79-8.72 (m, 1H), 8.61 (s, 2H), 8.40 (d, J = 1.4 Hz, 1H), 8.36 (s, 1H), 8.30 (s, 1H), 8.09 (s, 1H), 7.73 (t, J = 8.1 Hz, 3H), 6.55 (d, J = 8.7 Hz, 1H), 3.86 (s, 3H), 3.85 (s, 3H), 3.74 (d, J = 10.4 Hz, 2H), 3.42 (d, J = 9.9 Hz, 2H), 3.27 (d, J = 6.0 Hz, 2H), 1.72 (s, 2H), 0.94 (s, 1H). |
| 193 | 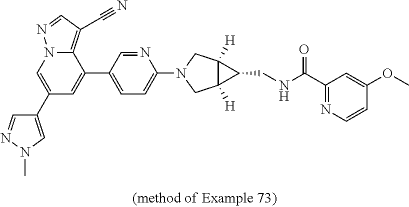 [SMILES: COc1ccnc(C(=O)NC[C@@H]2[C@H]3CN(c4ccc(-c5cc(-c6cnn(C)c6)cn6ncc(C#N)c56)cn4)C[C@@H]23)c1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-4- methoxypicolinamide | 546.3 | ¹H NMR (400 MHz, DMSO-d₆) δ 9.18 (s, 1H), 8.91 (t, J = 5.2 Hz, 1H), 8.61 (s, 1H), 8.44 (d, J = 5.5 Hz, 1H), 8.36 (s, 1H), 8.30 (s, 1H), 8.09 (s, 1H), 7.73 (m, 2H), 7.54 (d, J = 0.9 Hz, 1H), 7.14 (dd, J = 4.0, 1.0 Hz, 1H), 6.54 (d, J = 8.7 Hz, 1H), 3.88 (s, 3H), 3.85 (s, 3H), 3.70 (d, J = 10.4 Hz, 2H), 3.40 (d, J = 9.5 Hz, 2H), 3.26 (d, J = 5.9 Hz, 2H), 1.73 (s, 2H), 0.96 (s, 1H). |
| 194 | 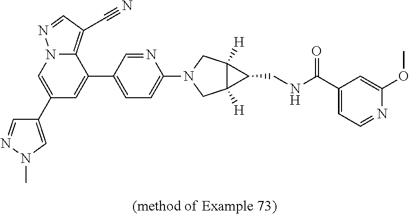 [SMILES: COc1cc(C(=O)NC[C@@H]2[C@H]3CN(c4ccc(-c5cc(-c6cnn(C)c6)cn6ncc(C#N)c56)cn4)C[C@@H]23)ccn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-2- methoxyiso- nicotinamide | 546.3 | ¹H NMR (400 MHz, DMSO-d₆) δ 9.19 (s, 1H), 8.77 (t, J = 6.9 Hz, 1H), 8.61 (s, 1H), 8.36 (s, 1H), 8.30 (s, 1H), 8.27 (d, J = 5.2 Hz, 1H), 8.09 (s, 1H), 7.80-7.69 (m, 2H), 7.34 (d, J = 4.9 Hz, 1H), 7.18 (s, 1H), 6.55 (d, J = 8.6 Hz, 1H), 3.87 (s, 3H), 3.85 (s, 3H), 3.73 (d, J = 10.5 Hz, 2H), 3.41 (d, J = 9.6 Hz, 2H), 3.26-3.21 (m, 2H), 1.71 (s, 2H), 0.92 (s, 1H). |
| 195 | 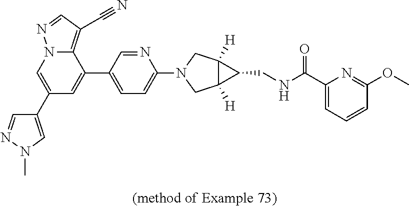 [SMILES: COc1cccc(C(=O)NC[C@@H]2[C@H]3CN(c4ccc(-c5cc(-c6cnn(C)c6)cn6ncc(C#N)c56)cn4)C[C@@H]23)n1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-6- methoxypicolinamide | 546.3 | ¹H NMR (400 MHz, DMSO-d₆) δ 9.18 (s, 1H), 8.71-8.65 (m, 1H), 8.61 (s, 1H), 8.36 (s, 1H), 8.30 (s, 1H), 8.09 (s, 1H), 7.84 (t, J = 7.7 Hz, 1H), 7.78-7.69 (m, 2H), 7.61 (d, J = 7.2 Hz, 1H), 6.99 (d, J = 8.2 Hz, 1H), 6.55 (d, J = 8.7 Hz, 1H), 3.97 (s, 3H), 3.85 (s, 3H), 3.71 (d, J = 10.4 Hz, 2H), 3.41 (d, J = 9.5 Hz, 2H), 3.29- 3.25 (m, 2H), 1.75 (s, 2H), 1.00 (s, 1H). |
| 196 | 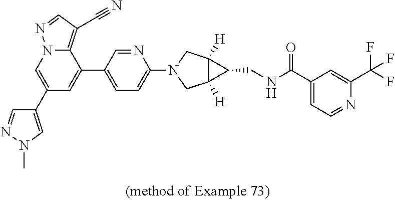 [SMILES: Cn1cc(-c2cc(-c3ccc(N4C[C@@H]5[C@@H](CNC(=O)c6ccnc(C(F)(F)F)c6)[C@@H]5C4)nc3)c3c(C#N)cnn3c2)cn1] | N-(((1R,5S,6s)-3-(5- (3-cyano-6-(1- methyl-1H-pyrazol- 4-yl)pyrazolo[1,5- a]pyridin-4- yl)pyridin-2-yl)-3- azabicyclo[3.1.0] hexan- 6-yl)methyl)-2- (trifluoromethyl)iso- nicotinamide | 584.3 | ¹H NMR (400 MHz, DMSO-d₆) δ 9.19 (s, 1H), 9.16-9.08 (m, 1H), 8.92 (d, J = 4.5 Hz, 1H), 8.61 (s, 1H), 8.36 (s, 1H), 8.30 (s, 1H), 8.25 (s, 1H), 8.09 (s, 2H), 7.79- 7.68 (m, 2H), 6.56 (d, J = 8.7 Hz, 1H), 3.85 (s, 3H), 3.75 (d, J = 10.5 Hz, 2H), 3.42 (d, J = 9.9 Hz, 2H), 3.29-3.25 (m, 2H), 1.73 (s, 2H), 0.95 (s. 1H). |
| 197 | 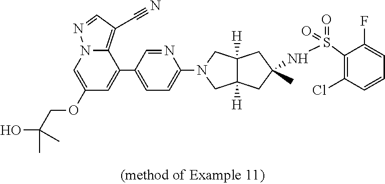 [SMILES: CC(C)(O)COc1cc(-c2ccc(N3C[C@@H]4C[C@@](C)(NS(=O)(=O)c5c(F)cccc5Cl)C[C@@H]4C3)nc2)c2c(C#N)cnn2c1] | 2-chloro-N- ((3aR,5s,6aS)-2-(5- (3-cyano-6-(2- hydroxy-2- methylpropoxy) pyrazolo [1,5-a]pyridin-4- yl)pyridin-2-yl)-5- methyloctahydrocyclo- penta[c]pyrrol-5- yl)-6- fluorobenzenesulfon- amide | 639.3 | |
| 198 | 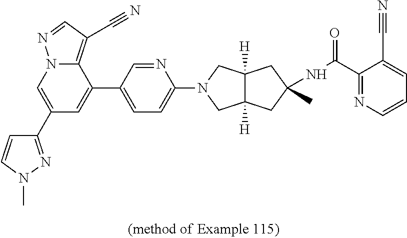 [SMILES: Cn1ccc(-c2cc(-c3ccc(N4C[C@@H]5C[C@@](C)(NC(=O)c6ncccc6C#N)C[C@@H]5C4)nc3)c3c(C#N)cnn3c2)n1] | 3-cyano-N- ((3aR,5s,6aS)-2-(5- (3-cyano-6-(1- methyl-1H-pyrazol- 3-yl)pyrazolo[1,5- a]pyridin-4- yl)pyridin-2-yl)-5- methyloctahydrocyclo- penta[c]pyrrol-5- yl)picolinamide | 569.4 | |
| 199 | 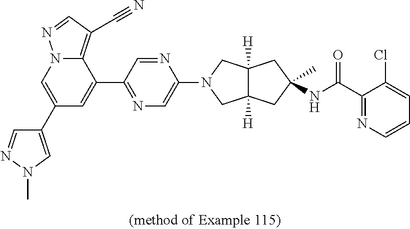 [SMILES: Cn1cc(-c2cc(-c3cnc(N4C[C@@H]5C[C@](C)(NC(=O)c6ncccc6Cl)C[C@@H]5C4)cn3)c3c(C#N)cnn3c2)cn1] | 3-chloro-N- ((3aR,5r,6aS)-2- (5-(3-cyano-6-(1- methyl-1H- pyrazol-4- yl)pyrazolo[1,5- a]pyridin-4- yl)pyrazin-2-yl)- 5- methyloctahydro cyclopenta[c] pyrrol-5- yl)picolinamide | 579.4 | |

### RET Kinase Assay

Compounds were tested in a LanthaScreen™ time-resolved fluorescence energy transfer (TR-FRET) enzymatic assay from Invitrogen. The assay used human RET kinase (Carna 08-159). Test compounds were prepared and diluted in DMSO in 3-fold serial dilutions to 50× of the final testing concentrations. The compounds were then further diluted to 5× by the kinase reaction buffer (50 mM HEPES pH 7.5, 0.0015% Brij-35). The enzymatic reaction for compound testing was performed in a white 384-well polypropylene plate (Corning 3573) with a total reaction volume of 25 μl containing 7 nM RET, 3 μM peptide substrate FAM-P2 (GL Biochem 112394), and 23 μM ATP (Sigma A7699-1G). The assay started with loading RET diluted in kinase reaction buffer to wells, followed by addition of equal volume of 5× compounds for 15-min incubation at the room temperature for pre-treatment. The enzymatic reaction was initiated by addition of mixture of the substrate and ATP prepared in kinase reaction buffer. After incubation at 28° C. for one hour, 25 μl of stopper buffer (a mixture of 100 mM HEPES pH 7.5 buffer, 0.015% Brij-35, 50 mM EDTA and 0.2% of coating reagent 3 (Cliper Lifesciences)) and produce TR-FRET signals. After 30 minutes of incubation at room temperature, the plate was read in a Caliper with the following settings: Excitation 340 nm (30)/Emission1 495 nm (10)/Emission2 520 nm (25). The TR-FRET values were dimensionless numbers that were calculated as the ratio of the acceptor (Green Fluorescent Protein) signal to the donor (Terbium) signal. Percent of control was calculated as the percentage of compound-treated vs 1% DMSO vehicle-treated. The dose-response curves were generated and the IC₅₀s were calculated by nonlinear sigmoid curve fitting using XLFit.

The IC₅₀ values of RET biochemical activity for the examples disclosed herein are listed in Table 10, A: ≤10 nM; B: >10 nM and ≤50 nM; C: >50 nM and ≤100 nM; D.>100 nM.

### KDR Kinase Assay

Compounds were tested in a LanthaScreen™ time-resolved fluorescence energy transfer (TR-FRET) enzymatic assay from Invitrogen. The assay used human KDR kinase (Carna 08-191). Test compounds were prepared and diluted in DMSO in 3-fold serial dilutions to 50× of the final testing concentrations. The compounds were then further diluted to 5× by the kinase reaction buffer (50 mM HEPES pH 7.5, 0.0015% Brij-35). The enzymatic reaction for compound testing was performed in a white 384-well polypropylene plate (Corning 3573) with a total reaction volume of 25 μl containing 1.2 nM KDR, 3 μM peptide substrate FAM-P22 (GL Biochem 112393), and 92 μM ATP (Sigma A7699-1G). The assay started with loading RET diluted in kinase reaction buffer to wells, followed by addition of equal volume of 5× compounds for 15-min incubation at the room temperature for pre-treatment. The enzymatic reaction was initiated by addition of mixture of the substrate and ATP prepared in kinase reaction buffer. After incubation at 28° C. for one hour, 25 μl of stopper buffer (a mixture of 100 mM HEPES pH 7.5 buffer, 0.015% Brij-35, 50 mM EDTA and 0.2% of coating reagent 3 (Cliper Lifesciences)) and produce TR-FRET signals. After 30 minutes of incubation at room temperature, the plate was read in a Caliper with the following settings: Excitation 340 nm (30)/Emission1 495 nm (10)/Emission2 520 nm (25). The TR-FRET values were dimensionless numbers that were calculated as the ratio of the acceptor (Green Fluorescent Protein) signal to the donor (Terbium) signal. Percent of control was calculated as the percentage of compound-treated vs 1% DMSO vehicle-treated. The dose-response curves were generated and the IC₅₀s were calculated by nonlinear sigmoid curve fitting using XLFit.

The IC₅₀ values of RET biochemical activity for the examples disclosed herein are listed in Table 10, A: ≤10 nM; B: >10 nM and ≤50 nM; C: >50 nM and ≤100 nM; D.>100 nM.

### Cellular Assay

### TT Cell Proliferation Assay

Compounds disclosed herein were tested for the inhibition of RET by a cancer cell proliferation assay commonly known as MTT assay. In this assay, a complete media was prepared by adding 10% fetal bovine serum to RPMI-1640 medium (Life technology). TT cells were added to each of 88 wells of a 96 well plate at a seeding density of 6,000 cells/well/90 μL. The cells were allowed to attach to the plate by incubating at 37° C. for 24 hours. The compound was dissolved in DMSO (SIGMA). A solution of test compound was prepared in complete media by serial dilution to obtain the following concentrations: 50 μM, 15 μM, 5 μM, 1.5 μM, 0.5 μM, 0.15 μM, 0.05 μM, 0.015 μM and 0.005 μM. The test compound solution (10 μL) was added to each of 80 cell-containing wells. The final concentrations of the compound were following: 5 μM, 1.5 μM, 0.5 μM, 0.15 μM, 0.05 μM, 0.015 μM, 0.005 μM, 0.0015 μM and 0.0005 μM. The final concentration of DMSO is 0.5%. To the 8 remaining cell-containing wells, only complete media (containing 0.5% DMSO) was added to form a control group in order to measure maximal proliferation. To the remaining 8 empty wells, complete media was added to for a vehicle control group in order to measure background. The plates were incubated at 37° C. for 8 days. 10 μL WST-8 solution (DOJINDO, Cell Counting KIT-8) was added to each well. The plates were further incubated at 37° C. for 5 hours, and then read for the absorbance using a microplate reader at 450 nm. The IC₅₀ was calculated using GraphPad Prism.

The IC₅₀ values of growth inhibition in TT cells for Compounds disclosed are listed in Table 10, A: ≤10 nM; B: >10 nM and ≤50 nM; C: >50 nM and ≤100 nM; D.>100 nM.

### BAF3-KIF5B-RET Cell proliferation assay

Compounds disclosed herein were tested for the inhibition of RET by a cancer cell proliferation assay commonly known as CellTiter-Glo assay. In this assay, a complete media was prepared by adding 10% fetal bovine serum to RPMI-1640 medium (Life technology) for BAF3-FIF5B-RET cells. BAF3-KIF5B-RET cells were added to each of 88 wells of a 96 well plate at a seeding density of 2,000 cells/well/95 μL. The cells were allowed to attach to the plate by incubating at 37° C. for 24 hours. The compound was dissolved in DMSO (SIGMA). A solution of test compound was prepared in complete media by serial dilution to obtain the following concentrations: 20 μM, 6.67 μM, 2.22 μM, 0.74 μM, 0.25 μM, 0.082 μM, 0.027 μM, 0.0091 μM and 0.0030 μM. The test compound solution (5 μL) was added to each of 80 cell-containing wells. The final concentrations of the compound were following: 1 μM, 0.33 μM, 0.11 μM, 0.037 μM, 0.012 μM, 0.0041 μM, 0.0014 μM, 0.00046 μM and 0.00015 μM. The final concentration of DMSO is 0.1%. To the 8 remaining cell-containing wells, only complete media (containing 0.1% DMSO) was added to form a control group in order to measure maximal proliferation. To the remaining 8 empty wells, complete media was added to for a vehicle control group in order to measure background. The plates were incubated at 37° C. for 72 hours. 50 μl of CellTiter-Glo® Reagent was added to each well. Mix contents for 2 minutes on an orbital shaker to induce cell lysis. Incubate at room temperature for 10 minutes to stabilize luminescent signal. Record luminescence on Paradigm. Cell viability (CV %) was calculated relative to vehicle (DMSO) treated control wells. The IC₅₀ was calculated using GraphPad Prism.

The IC₅₀ values of growth inhibition in TT cells for Compounds disclosed are listed in Table 10, A: ≤10 nM; B: >10 nM and ≤50 nM; C: >50 nM and ≤100 nM; D.>100 nM.

TABLE 10*

| | | | | BA/F3- |
|---|---|---|---|---|
| | | | | KIF5B- |
| | RET | KDR | TT | RET |
| Example | Enzymatic | Enzymatic | Cellular | Cellular |
| # | Activity | Activity | Activity | activity |
| 1 | A | B | B | |
| 2 | A | B | B | |
| 3 | A | B | B | |
| 4 | A | B | A | A |
| 5 | A | A | A | A |
| 6 | A | D | B | |
| 7 | A | D | A | C |
| 8 | A | D | B | |
| 9 | A | C | A | B |
| 10 | A | B | B | |
| 11 | A | A | A | A |
| 12 | A | B | B | A |
| 13 | A | B | B | |
| 14 | A | D | B | |
| 15 | B | C | B | |
| 16 | A | B | B | |
| 17 | A | B | B | |
| 18 | A | B | B | |
| 19 | A | B | B | |
| 20 | A | B | B | B |
| 21 | A | A | B | |
| 22 | A | B | C | |
| 23 | A | A | A | |
| 24 | A | A | B | |
| 25 | A | B | B | |
| 26 | A | A | B | |
| 27 | A | B | B | |
| 28 | A | A | B | |
| 29 | A | B | C | |
| 30 | A | B | B | |
| 31 | A | B | C | |
| 32 | A | D | A | |
| 33 | A | B | B | |
| 34 | A | B | B | |
| 35 | A | B | B | |
| 36 | A | A | B | |
| 37 | A | D | C | D |
| 38 | B | D | D | |
| 39 | C | D | D | |
| 40 | B | D | C | |
| 41 | D | D | D | |
| 42 | C | D | D | |
| 43 | B | D | D | |
| 44 | C | D | D | |
| 45 | A | A | C | |
| 46 | A | A | A | A |
| 47 | A | B | B | |
| 48 | A | B | A | A |
| 49 | A | B | B | |
| 50 | A | D | B | |
| 51 | A | B | B | |
| 52 | A | C | B | |
| 53 | A | B | A | A |
| 54 | A | D | B | B |
| 55 | A | A | A | A |
| 56 | A | B | A | A |
| 57 | B | D | A | |
| 58 | A | A | B | |
| 59 | A | A | A | |
| 60 | A | D | B | |
| 61 | A | B | A | A |
| 62 | A | C | B | |
| 63 | A | C | B | |
| 64 | A | B | C | |
| 65 | A | C | B | |
| 66 | A | C | B | |
| 67 | A | D | C | |
| 68 | A | C | B | |
| 69 | A | D | A | |
| 70 | A | B | B | A |
| 71 | A | B | A | |
| 72 | A | A | A | |
| 73 | A | A | A | A |
| 74 | A | B | B | |
| 75 | A | B | A | A |
| 76 | A | A | B | |
| 77 | A | A | A | |
| 78 | A | A | A | |
| 79 | A | A | A | |
| 80 | A | A | A | |
| 81 | A | B | A | |
| 82 | A | A | A | |
| 83 | A | B | A | |
| 84 | A | A | A | |
| 85 | A | A | A | |
| 86 | A | B | A | |
| 87 | A | B | A | |
| 88 | A | B | A | |
| 89 | A | A | A | |
| 90 | A | A | A | A |
| 91 | A | A | A | |
| 92 | A | B | B | |
| 93 | A | B | B | |
| 94 | A | C | B | |
| 95 | A | D | B | |
| 96 | A | B | A | A |
| 97 | A | A | A | A |
| 98 | A | B | A | A |
| 99 | A | B | B | |
| 100 | A | B | A | |
| 101 | A | C | C | C |
| 102 | A | C | | B |
| 103 | A | D | | B |
| 104 | A | | | B |
| 105 | A | | | B |
| 106 | A | | | A |
| 107 | A | | | B |
| 108 | A | | | B |
| 109 | A | | | A |
| 110 | C | | | D |
| 111 | A | | | A |
| 112 | A | | | A |
| 113 | A | | | A |
| 114 | A | | | |
| 115 | A | | | B |
| 116 | B | | | D |
| 117 | B | | | D |
| 118 | A | | | B |
| 119 | A | | | C |
| 120 | A | | | B |
| 121 | A | A | A | A |
| 122 | A | B | | |
| 123 | A | B | A | |
| 124 | A | D | B | |
| 125 | A | | | A |
| 126 | B | B | C | |
| 127 | A | D | B | |
| 128 | A | B | B | |
| 129 | B | | | C |
| 130 | A | D | | |
| 131 | A | C | B | |
| 132 | A | D | A | B |
| 133 | A | | | B |
| 134 | A | | | |
| 135 | A | B | A | A |
| 136 | A | B | A | A |
| 137 | A | C | A | A |
| 138 | A | A | | B |
| 139 | A | | | C |
| 140 | A | B | B | |
| 141 | A | A | A | |
| 142 | A | B | A | |
| 143 | A | D | | B |
| 144 | A | | | A |
| 145 | A | | | A |
| 146 | A | | | A |
| 147 | A | | | B |
| 148 | A | | | A |
| 149 | A | | | A |
| 150 | A | | | A |
| 151 | A | | | A |
| 152 | A | B | | C |
| 153 | A | B | | A |
| 154 | A | | | B |
| 155 | C | | | D |
| 156 | A | | | B |
| 157 | B | | | D |
| 158 | A | | | B |
| 159 | B | | | B |
| 160 | B | | | D |
| 161 | A | | | D |
| 162 | B | | | D |
| 163 | B | | | D |
| 164 | A | | | A |
| 165 | A | | | A |
| 166 | A | | | B |
| 167 | A | | | C |
| 168 | A | | | B |
| 169 | A | | | A |
| 170 | A | | | B |
| 171 | A | | | B |
| 172 | B | | | D |
| 173 | A | | | A |
| 174 | A | | | B |
| 175 | A | | | |
| 176 | C | | | D |
| 177 | C | | | D |
| 178 | A | B | A | |
| 179 | B | B | A | |
| 180 | A | A | A | A |
| 181 | A | A | A | B |
| 182 | A | B | A | |
| 183 | B | D | D | |
| 184 | B | D | C | |
| 185 | A | A | B | |
| 186 | A | A | A | |
| 187 | A | A | | |
| 188 | A | | | A |
| 189 | A | | | A |
| 190 | C | | | D |
| 191 | A | | | A |
| 192 | A | | | B |
| 193 | A | | | A |
| 194 | A | | | B |
| 195 | A | | | A |
| 196 | A | | | C |
| 197 | A | B | B | |
| 198 | B | C | B | |
| 199 | A | | | B |

A: ≤10 nM; B: >10 nM and ≤50 nM; C: >50 nM and ≤100 nM; D. >100 nM.

### INDUSTRIAL APPLICABILITY

The compound of the present invention can be applied to the field of medicine.

What is claimed is:

1. A compound of Formula I: 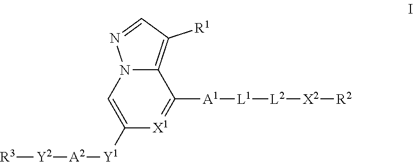
and/or stereoisomers, stable isotopes, or pharmaceutically acceptable salts or solvates thereof, wherein R¹, R², R³, A¹, A², L¹, L², X¹, X², Y¹, and Y² are defined below:
R¹ is selected from H, —CN, ethynyl, halo, —CF₃, —CH₃, —CH₂CH₃, cyclopropyl, —CH₂CN, and —CH(CN)CH₃;
R² is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R² is 1-4 substituents independently selected from R⁴, wherein each R⁴ is independently selected from halo, —OH, NH₂, ═O, —CN, OC(O)R⁵, —CO₂R¹, —C(O)N(R^{6a}R^{6b}), —C(═NR⁷)N(R^{6a}R^{6b}), —C(O)R⁵, —S(O)₀₋₂R⁸, —S(O)(═NR⁷)R⁸, —S(O)₁₋₂N(R^{6a}R^{6b}), —N(R^{6a}R^{6b}), —N(R^{6a})C(O)R⁸, —N(R^{6a})C(═NR⁷)R⁸, —N(R^{6a})S(O)₁₋₂R⁸, —N(R^{6c})C(O)N(R^{6a}R^{6b}), —N(R^{6c})C(═NR⁷)N(R^{6a}R^{6b}), —N(R^{6c})S(O)₁₋₂N(R^{6a}R^{6b}), —N(R^{6a})CO₂R⁸, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated and unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R⁵, R^{6a}, R^{6b} and R^{6c} are independently selected from H, C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; R⁷ is independently selected from H, —CN, —OH, C1-C4 alkyl and C1-C4 alkoxy; R⁸ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring members; wherein each of R⁵, R^{6a}, R^{6b}, R^{6c}, R⁷, and R⁸ is optionally substituted with 1-3 groups independently selected from halo, —OH, NH₂, ═O, —CN, —S(O)₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl;
wherein two substituents on the same or adjacent carbon atoms of R² can optionally be taken together to form a 4-6 membered ring that can be saturated or aromatic and optionally contains 1-2 heteroatoms selected from N, O and S and can optionally be substituted with 1-2 groups independently selected from R⁴;
R³ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, saturated 7-8 membered bridged heterocyclyl containing 1-2 heteroatoms selected from N, O, and S as ring members, saturated 7-11 membered spiroheterocyclyl containing 1-2 heteroatoms selected from N, O, and S as ring members, and 5-membered heteroaryl containing 1-3 heteroatoms selected from N, O, and S as ring members; and wherein the optional substituents for R³ is 1-4 substituents independently selected from R⁴;
A¹ is an optionally substituted group selected from para-attached benzene, para-attached 6-membered heteroarene containing 1-2 N as ring members, 2,5-attached thiophene, and 2,5-attached thiazole, wherein the optional substituents are 1-3 substituents selected from F, Cl, CN, CH₃, and CF₃;
A² is a bond or an optionally substituted C1-C6 alkylenyl, wherein the optional substituents are 1-3 substituents selected from R⁴;
L¹ is selected from 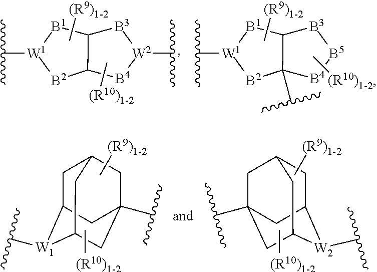
wherein W¹ is N or 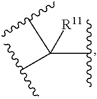
wherein R¹¹ is selected from H, OH, CN, F, and an optionally substituted group selected from C1-C6 alkyl, and C1-C6 alkoxy, and wherein the optional substituents are 1-3 groups independently selected from halo, OH, CN, C1-C3 alkyl, C1-C3 haloalkyl, C1-C3 alkoxy, C1-C3 haloalkoxy, C3-C6 cycloalkyl, and C3-C6 cycloalkyloxy;
wherein W² is N or 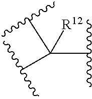
or, wherein R¹² is selected from H, F, OH, —CO₂H and an optionally substituted group selected from C1-C6 alkyl and C1-C6 alkoxy, and wherein the optional substituents are 1-3 groups independently selected from R⁴;
wherein the left wavy line indicates the point of attachment of L¹ to A¹; wherein the right wavy line indicates the point of attachment of L¹ to L²;
wherein B¹, B², B³, and B⁴ are independently selected from a bond, —O—, and an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein zero, one, or two of B¹, B², B³ and B⁴ is a bond or —O—;
wherein B5 is —O—, or an optionally substituted C1-C3 alkylenyl wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein when B⁵ is —O—, B³ and B⁴ cannot be —O—, or zero or one of B³ and B⁴ is a bond;
wherein R⁹ and R¹⁰ are independently selected from R⁴;
L² is a bond or an optionally substituted C1-C4 alkylenyl wherein the optional substituents are 1-3 groups independently selected from R⁴; wherein L² and W² via R¹² together optionally form 3-6 membered spirocycloalkyl or 4-6 membered spiroheterocycles containing 1-2 heteroatoms independently selected from N, O, and S as ring members;
X¹ is —C(H)— or N;
X² is selected from a bond, —O—, —N(R¹³)—, —C(O)—, —C(O)O—, C(O)N(R¹³)—, —N(R¹³)C(O)—, —N(R¹³)C(O)N(R¹⁴)—, —N(R¹³)C(O)O—, —S(O)₀₋₂—, —S(O)₁₋₂NR¹³—, —N(R¹³)S(O)₁₋₂—, —S(O)(═NR¹⁵)—, —S(O)(═NR¹⁵)NR—, —NR¹³S(O)(═NR¹⁵)—, N(R¹³)S(O)₂N(R¹⁴)—, and an optionally substituted group selected from C1-C3 alkylenyl and C3-C6 cycloalkylidenyl; wherein R¹³ and R¹⁴ are independently selected from H and an optionally substituted group independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, heteroaryl containing 1-4 heteroatoms selected from N, O, and S as ring member, and the optional substituents are 1-3 groups independently selected from R⁴; R¹⁵ is selected from H, —CN, —OH, and an optionally submitted group selected from C1-C4 alkyl and C1-C4 alkoxy, and the optional substituents are 1-3 groups independently selected from R⁴;
Y¹ is selected from a bond, O, —N(R¹³)—, and an optionally substituted C1-C3 alkylenyl, wherein the optional substituents are 1-3 groups independently selected R⁴; and
Y² is selected from a bond, —O—, and —N(R¹³)—.

2. The compound of claim 1, and/or a stereoisomer, a stable isotope, or a pharmaceutically acceptable salt or solvate thereof, where L¹ is selected from 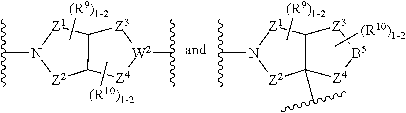
wherein the left wavy line indicates the point of attachment of L¹ to A¹; wherein the right wavy line indicates the point of attachment of L¹ to L²;
wherein Z¹, Z², Z³, and Z⁴ are independently selected from a bond and an optionally substituted C1-C3 alkylenyl, wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein zero or one of Z¹ and Z² is bond, and zero, one, or two of Z¹, Z², Z³, and Z⁴ are bonds;
wherein B5 is —O—, or an optionally substituted C1-C3 alkylenyl, wherein the optional substituents are 1-3 substituent each independently selected from halo, —OH, NH₂, =0, C1-C4 alkyl, C1-C4 haloalkyl, C1-C4 alkoxy, C1-C4 haloalkoxy, C3-C6 cycloalkyl, C3-C6 cycloalkylidenyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, C3-C6 cycloalkylaminosulfonyl, and (C1-C6 alkyl)₁₋₂amino; wherein when B⁵ is —O—, Z³ and Z⁴ cannot be —O—, or zero or one of Z³ and Z⁴ is a bond; and
wherein R⁹, R¹⁰, and W² are as defined in claim 1.

3. A compound of claim 1, and/or a stereoisomer, a stable isotope, or a pharmaceutically acceptable salt or solvate thereof, where L¹ is 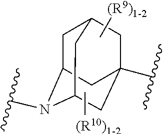
wherein the left wavy line indicates the point of attachment of L¹ to A¹; wherein the right wavy line indicates the point of attachment of L¹ to L²; and
wherein R⁹ and R¹⁰ are as defined in claim 1.

4. The compound of any one of claims 1-2, and/or a stereoisomer, a stable isotope, or a pharmaceutically acceptable salt or solvate thereof, wherein L¹ is 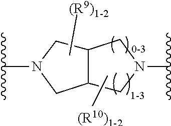
wherein the left wavy line indicates the point of attachment of L¹ to A¹; wherein the right wavy line indicates the point of attachment of L¹ to L²; and
wherein R⁹ and R¹⁰ are as defined in claim 1.

5. The compound of any one of claims 1-2, and/or a stereoisomer, a stable isotope, or a pharmaceutically acceptable salt or solvate thereof, wherein L¹ is selected from 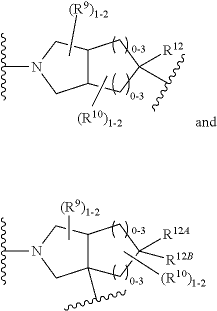
wherein the left wavy line indicates the point of attachment of L¹ to A¹; wherein the right wavy line indicates the point of attachment of L¹ to L²;
wherein R^{12A} and R^{12B} are independently selected from H, F, OH, —CO₂H, and an optionally substituted group selected from C1-C6 alkyl and C1-C6 alkoxy, and wherein the optional substituents are 1-3 groups independently selected from R⁴; and
wherein R⁹, R¹⁰, and R¹² are as defined in claim 1.

6. The compound of any one of claims 1-2, and/or a stereoisomer, a stable isotope, or a pharmaceutically acceptable salt or solvate thereof, wherein L¹ is selected from 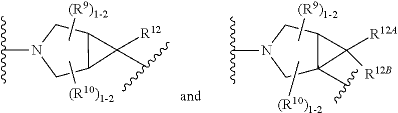
wherein the left wavy line indicates the point of attachment of L¹ to A¹; wherein the right wavy line indicates the point of attachment of L¹ to L²;
wherein R^{12A} and R^{12B} are independently selected from H, F, OH, —CO₂H, and an optionally substituted group selected from C1-C6 alkyl and C1-C6 alkoxy, and wherein the optional substituents are 1-3 groups independently selected from R⁴; and
wherein R⁹, R¹⁰, and R¹² are as defined in claim 1.

7. The compound of any one of claims 1-6, and/or a stereoisomer, a stable isotope, or a pharmaceutically acceptable salt or solvate thereof, wherein L² is a bond.

8. The compound of any one of claims 1-6, and/or a stereoisomer, a stable isotope, or a pharmaceutically acceptable salt or solvate thereof, wherein L² is an optionally substituted C1-C4 alkylenyl, wherein the optional substituents are 1-3 groups independently selected R⁴.

9. The compound of any one of claims 1, 2, and 5, and/or a stereoisomer, a stable isotope, or a pharmaceutically acceptable salt or solvate thereof, wherein L¹ and L² together form 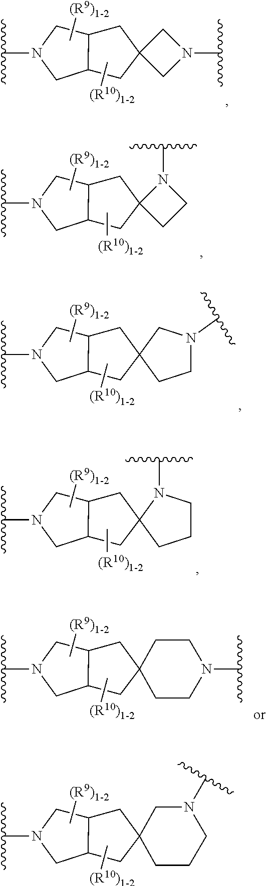
wherein the left wavy line indicates the point of attachment of L¹ to A¹; wherein the right wavy line indicates the point of attachment of L¹ to X²; and
wherein R⁹ and R¹⁰are as defined in claim 1.

10. The compound of any one of claims 1-9, and/or a stereoisomer, a stable isotope, or a pharmaceutically acceptable salt or solvate thereof, wherein A¹ is 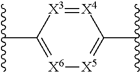
wherein X³, X⁴, X⁵ and X⁶ are independently selected from CH, —C(CH₃)—, CF, and N, wherein zero, one, or two of X³, X⁴, X⁵, and X⁶ is N.

11. The compound of any one of claims 1-10, and/or a stereoisomer, a stable isotope, or a pharmaceutically acceptable salt or solvate thereof, wherein X² is selected from —N(R¹³)C(O)—, C(O)N(R¹³)—, —N(R¹³)C(O)N(R¹⁴)—, —N(R¹³)C(O)O—, —N(R¹³)S(O)₂—, C1-C3 alkylenyl, and C3-C6 cycloalkylidenyl; and
wherein R¹³ and R¹⁴ are as defined in claim 1.

12. The compound of any one of claims 1-3, 5-8, and 10-11, and/or a stereoisomer, a stable isotope, or a pharmaceutically acceptable salt or solvate thereof, wherein -L¹-L²-X²—R² is selected from 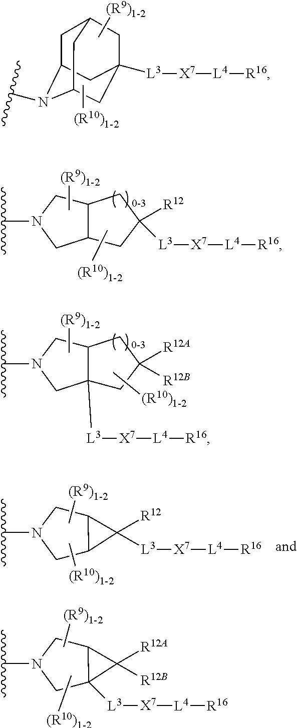
wherein L³ and L⁴ are independently selected from a bond and a C1-C3 alkylenyl group optionally substituted by 1-3 substituents independently selected from R⁴; X⁷ is selected from a bond, —O—, —N(R¹³)—, —N(R¹³)C(O)—, —N(R¹³)S(O)₂—, —C(O)N(R¹³)—, —S(O)₂N(R¹³)—, —N(R¹³)C(O)N(R¹⁴)—, —N(R¹³)C(O)O—, —OC(O)N(R¹³)—, and —N(R¹³)S(O)₂N(R¹⁴)—; R¹⁶ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁶ is 1-4 substituents independently selected from R⁴;
wherein R^{12A} and R^{12B} are independently selected from H, F, OH, —CO₂H, and an optionally substituted group selected from C1-C6 alkyl and C1-C6 alkoxy, and wherein the optional substituents are 1-3 groups independently selected from R⁴; and
wherein R⁴, R⁹, R¹⁰, R¹², and R¹³ are as defined in claim 1.

13. The compound of any one of claims 1-2, 4, and 7-11, and/or a stereoisomer, a stable isotope, or a pharmaceutically acceptable salt or solvate thereof, wherein -L¹-L²-X²-R² is selected from 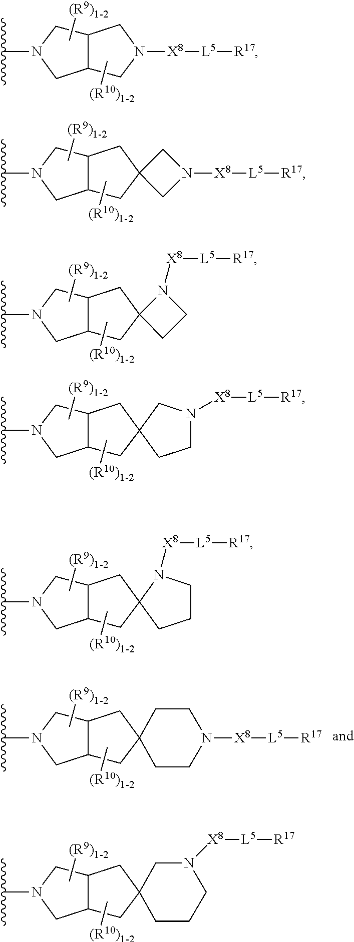
wherein L⁵ is selected from a bond or a C1-C3 alkylenyl group optionally substituted by 1-3 substituents independently selected from R⁴; X⁸ is selected from a bond, —C(O)—, and —S(O)₂—; R⁷ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁷ is 1-4 substituents independently selected from R⁴;
wherein R⁴, R⁹, and R¹⁰ are as defined in claim 1.

14. The compound of any one of claims 1-13, and/or a stereoisomer, a stable isotope, or a pharmaceutically acceptable salt or solvate thereof, wherein R³ is a saturated or unsaturated 4-7 membered heterocyclyl containing 1-2 heteroatoms selected from N, O, and S as ring members.

15. The compound of any one of claims 1-13, and/or a stereoisomer, a stable isotope, or a pharmaceutically acceptable salt or solvate thereof, wherein R³ is saturated 7-8 membered bridged heterocyclyl containing 1-2 heteroatoms selected from N, O, and S as ring members.

16. The compound of any one of claims 1-13, and/or a stereoisomer, a stable isotope, or a pharmaceutically acceptable salt or solvate thereof, wherein A², Y¹, and Y² are bonds; R³ is an optionally substituted group selected from saturated and unsaturated 4-6 membered heterocyclyl containing 1-2 heteroatoms selected from N, O, and S as ring members, and 5-membered heteroaryl containing 1-4 heteroatoms selected from N, O, and S as ring members; and wherein the optional substituents for R³ is 1-4 substituents independently selected from R⁴, wherein R⁴ is as defined in claim 1.

17. The compound of any one of claims 1-13, and/or a stereoisomer, a stable isotope, or a pharmaceutically acceptable salt or solvate thereof, wherein Y¹ is selected from a bond, —O—, and —N(R¹³)—; A² is a an optionally substituted C1-C6 alkylenyl, wherein the optional substituents are 1-3 substituents selected from R⁴; Y² is selected from a bond, —O—, and —N(R¹³)—; and wherein R⁴ and R¹³ are as defined in claim 1.

18. The compound of any one of claims 1-13 and 15, and/or a stereoisomer, a stable isotope, or a pharmaceutically acceptable salt or solvate thereof, wherein Y¹ is selected from O; A² is a an optionally substituted C1-C6 alkylenyl, wherein the optional substituents are 1-3 substituents selected from R⁴; Y² is selected from a bond and —O—; and wherein R⁴ is as defined in claim 1.

19. The compound of claims 1-13 and 15-16, and/or a stereoisomer, a stable isotope, or a pharmaceutically acceptable salt or solvate thereof, wherein R³—Y²-A²-Y¹— is 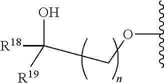
wherein n is 1, 2 or 3; R¹⁸ and R¹⁹ are independently selected from H and an optionally substituted group selected from C1-C6 alkyl, C3-C6 cycloalkyl, and saturated and unsaturated 4-7 membered heterocyclyl containing 1-2 heteroatoms selected from N, O, and S as ring members; and wherein the optional substituents are 1-4 substituents independently selected from R⁴; wherein R¹⁸ and R¹⁹ together optionally form 3-6 membered cycloalkyl or 4-6 membered heterocycles containing 1-2 heteroatoms independently selected from N, O, and S as ring members.

20. The compound of any one of claims 1-19, and/or a stereoisomer, a stable isotope, or a pharmaceutically acceptable salt or solvate thereof, wherein R¹ is CN; X¹ is CH.

21. The compound of any one of claims 1-14 and 20, and/or a stereoisomer, a stable isotope, or a pharmaceutically acceptable salt or solvate thereof, wherein Y¹, A² and Y² are bonds; R³ is selected from 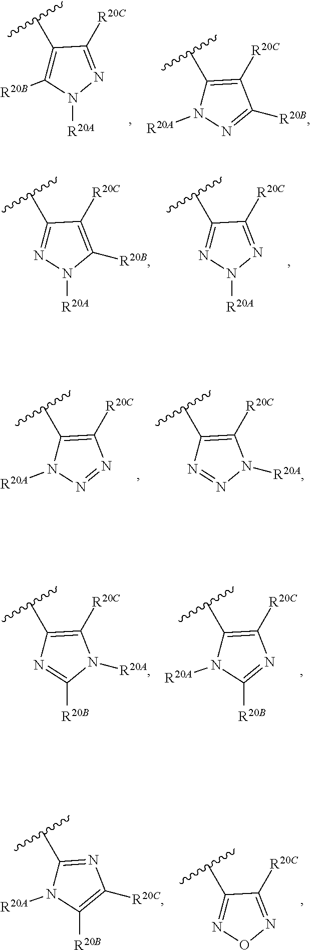
wherein R^{20A} is independently selected from H, Me, Et, propyl, isopropyl, butyl, isobutyl, sec-butyl, t-butyl, —CH₂F, —CF₂H, —CF₃, and cyclopropyl; and R^{20B} and R^{20C} are independently selected from H, Me, Et, propyl, isopropyl, butyl, isobutyl, sec-butyl, t-butyl, —CH₂F —CF₂H, —CF₃, cyclopropyl, —OMe, —OEt, —OPr, —OⁱPr, —OBu, —OⁱBu, —O^{s}Bu, —O^{t}Bu, —OCF₃, —O(cycloproyl), —CN, Cl, and F.

22. The compound of claim 1, which is of the Formula IA, and/or a stereoisomer, a stable isotope, or a pharmaceutically acceptable salt or solvate thereof: 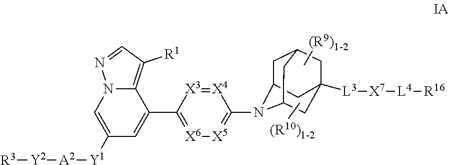
wherein L³ and L⁴ are independently selected from a bond and a C1-C3 alkylenyl group optionally substituted by 1-3 substituents independently selected from R⁴; X⁷ is selected from a bond, —O—, —N(R¹³)—, —N(R¹³)C(O)—, —N(R¹³)S(O)₂—, —C(O)N(R¹³)—, —S(O)₂N(R¹³)—, —N(R¹³)C(O)N(R¹⁴)—, —N(R¹³)C(O)O—, —OC(O)N(R¹³)—, and —N(R¹³)S(O)₂N(R¹⁴)—; R¹⁶ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁶ is 1-4 substituents independently selected from R⁴;
wherein X³, X⁴, X⁵, and X⁶ are independently selected from CH, —C(CH₃)—, CF, and N, wherein zero, one, or two of X³, X⁴, X⁵, and X⁶ is N; and wherein R¹, R³, R⁴, R⁹, R¹⁰, R¹³, R¹⁴, A², Y¹, and Y² are as defined in claim 1.

23. The compound of claim 1, which is of the Formula IB, and/or a stereoisomer, a stable isotope, or a pharmaceutically acceptable salt or solvate thereof: 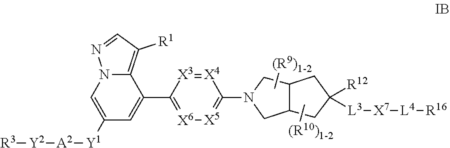
wherein L³ and L⁴ are independently selected from a bond and a C1-C3 alkylenyl group optionally substituted by 1-3 substituents independently selected from R⁴; X⁷ is selected from a bond, —O—, —N(R¹³)—, —N(R¹³)C(O)—, —N(R¹³)S(O)₂—, —C(O)N(R¹³)—, —S(O)₂N(R¹³)—, —N(R¹³)C(O)N(R¹⁴)—, —N(R¹³)C(O)O—, —OC(O)N(R¹³)—, and —N(R¹³)S(O)₂N(R¹⁴)—; R¹⁶ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁶ is 1-4 substituents independently selected from R⁴;
wherein X³, X⁴, X⁵, and X⁶ are independently selected from CH, —C(CH₃)—, CF, and N, wherein zero, one, or two of X³, X⁴, X⁵, and X⁶ is N; and
wherein R¹, R³, R⁴, R⁹, R¹⁰, R¹³, R¹⁴, A², Y¹, and Y² are as defined in claim 1.

24. The compound of claim 1, which is of the Formula IC, and/or a stereoisomer, a stable isotope, or a pharmaceutically acceptable salt or solvate thereof: 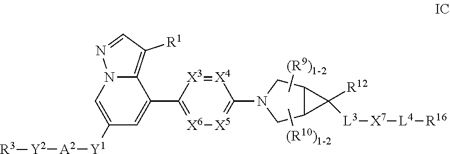
wherein L³ and L⁴ are independently selected from a bond and a C1-C3 alkylenyl group optionally substituted by 1-3 substituents independently selected from R⁴; X⁷ is selected from a bond, —O—, —N(R¹³)—, —N(R¹³)C(O)—, —N(R¹³)S(O)₂—, —C(O)N(R¹³)—, —S(O)₂N(R¹³)—, —N(R¹³)C(O)N(R¹⁴)—, —N(R¹³)C(O)O—, —OC(O)N(R¹³)—, and —N(R¹³)S(O)₂N(R¹⁴)—; R¹⁶ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁶ is 1-4 substituents independently selected from R⁴;
wherein X³, X⁴, X⁵, and X⁶ are independently selected from CH, —C(CH₃)—, CF, and N, wherein zero, one, or two of X³, X⁴, X⁵, and X⁶ is N; and
wherein R¹, R³, R⁴, R⁹, R¹⁰, R¹³, R¹⁴, A², Y¹, and Y² are as defined in claim 1.

25. The compound of claim 1, which is of the Formula ID, and/or a stereoisomer, a stable isotope, or a pharmaceutically acceptable salt or solvate thereof: 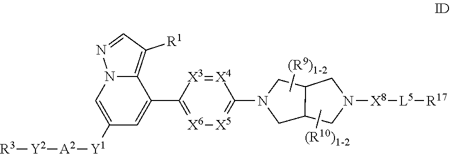
wherein L⁵ is independently selected from a bond and a C1-C3 alkylenyl group optionally substituted by 1-3 substituents independently selected from R⁴; X⁸ is selected from a bond, —C(O)—, and —S(O)₂—; R¹⁷ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁷ is 1-4 substituents independently selected from R⁴;
wherein X³, X⁴, X⁵, and X⁶ are independently selected from CH, —C(CH₃)—, CF, and N, wherein zero, one, or two of X³, X⁴, X⁵, and X⁶ is N; and
wherein R¹, R³, R⁴, R⁹, R¹⁰, R¹³, R¹⁴, A², Y¹, and Y² are as defined in claim 1.

26. The compound of claim 1, which is of the Formula IE, and/or a stereoisomer, a stable isotope, or a pharmaceutically acceptable salt or solvate thereof: 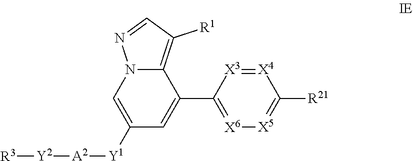
wherein R²¹ is selected from 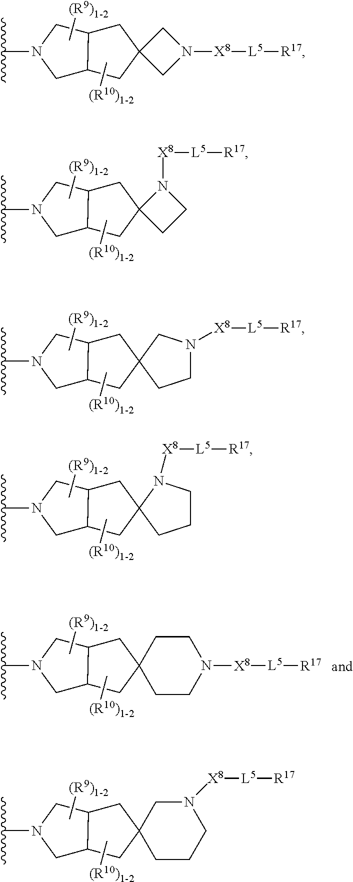
wherein L⁵ is selected from a bond or a C1-C3 alkylenyl group optionally substituted by 1-3 substituents independently selected from R⁴; X⁸ is selected from a bond, —C(O)—, and —S(O)₂—; R¹⁷ is selected from H and an optionally substituted group selected from C1-C6 alkyl, C3-C6 cycloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members; and wherein the optional substituents for R¹⁷ is 1-4 substituents independently selected from R⁴;
wherein X³, X⁴, X⁵, and X⁶ are independently selected from CH, —C(CH₃)—, CF, and N, wherein zero, one, or two of X³, X⁴, X⁵, and X⁶ is N; and
wherein R¹, R³, R⁴, R⁹, R¹⁰, R¹³, R¹⁴, A², Y¹ and Y² are as defined in claim 1.

27. The compound of claim 1, which is selected from the following compounds, and/or a stereoisomer, a stable isotope, or a pharmaceutically acceptable salt or solvate thereof:
4-(6-((3aR,6aS)-5-(6-methoxynicotinoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-hydroxy-3-methylbutanoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-hydroxy-2-phenylacetyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-chloro-6-fluorobenzoyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-ethoxypyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
4-(6-((3aR,6aS)-5-(3-chloropicolinoyl)hexahydropyrrolo[3,4-c]pyrrol-2(1H)-yl)pyridin-3-yl)-6-(2-hydroxypropoxy)pyrazolo[1,5-a]pyridine-3-carbonitrile,
4-(6-((3aR,6aS)-5-(2-chloro-6-fluorobenzoyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
4-(6-((3aR,6aS)-5-isobutyrylhexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-(2-chloro-6-fluorophenylsulfonyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-((6-methoxypyridin-3-yl)methyl)hexahydropyrrolo[3,4-c]pyrrol-2(1H)-yl)pyridin-3-yl)-6-(1-methyl-1H-pyrazol-4-yl)pyrazolo[1,5-a]pyridine-3-carbonitrile,
4-(6-((3aR,6aS)-5-((6-methoxypyridin-3-yl)methyl)hexahydropyrrolo[3,4-c]pyrrol-2(1H)-yl)pyridin-3-yl)-6-(1-methyl-1H-pyrazol-3-yl)pyrazolo[1,5-a]pyridine-3-carbonitrile,
6-(2-hydroxy-2-methylpropoxy)-4-(6-((3aR,6aS)-5-((6-methoxypyridin-3-yl)methyl)hexahydropyrrolo[3,4-c]pyrrol-2(1H)-yl)pyridin-3-yl)pyrazolo[1,5-a]pyridine-3-carbonitrile,
N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-6-methoxynicotinamide,
N-((1R,5S,6s)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-2-hydroxy-3-methylbutanamide,
N-((1R,5S,6r)-3-(4-(3-cyano-6-(1-methyl-1H-pyrazol-4-yl)pyrazolo[1,5-a]pyridin-4-yl)phenyl)-3-azabicyclo[3.1.0]hexan-6-yl)-2-hydroxy-3-methylbutanamide,
(R)-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-hydroxy-2-phenylacetamide,
(R)-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-hydroxy-3-methylbutanamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)picolinamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-(trifluoromethyl)picolinamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluoropicolinamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-methylbenzamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-methylbutanamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluoro-2-methylbenzamide,
3-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-methylpicolinamide,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-5-fluorobenzamide,
3-chloro-N-((1R,5S,6s)-3-(5-(3-cyano-6-(1-methyl-1H-pyrazol-3-yl)pyrazolo[1,5-a]pyridin-4-yl)pyridin-2-yl)-3-azabicyclo[3.1.0]hexan-6-yl)picolinamide,
3-chloro-N-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)picolinamide,
N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)isobutyramide,
2-amino-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-2-phenylacetamide,
4-(6-((1R,5S,6s)-6-(((6-methoxypyridin-3-yl)methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile,
4-(6-((1R,5S,6s)-6-(((6-methoxypyridin-3-yl)methyl)(methyl)amino)-3-azabicyclo[3.1.0]hexan-3-yl)pyridin-3-yl)-6-(1-methyl-1H-pyrazol-4-yl)pyrazolo[1,5-a]pyridine-3-carbonitrile,
2-chloro-N-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzenesulfonamide,
1-((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-phenylurea,
3-chloro-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)picolinamide,
3-chloro-N-((1R,5S,6s)-3-(5-(3-cyano-6-ethoxypyrazolo[1,5-a]pyridin-4-yl)pyridin-2-yl)-3-azabicyclo[3.1.0]hexan-6-yl)picolinamide,
3-chloro-N-((1R,5S,6s)-3-(5-(3-cyano-6-methoxypyrazolo[1,5-a]pyridin-4-yl)pyridin-2-yl)-3-azabicyclo[3.1.0]hexan-6-yl)picolinamide,
(R)-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-2-hydroxy-2-phenylacetamide,
3-chloro-N-((1R,5S,6r)-3-(5-(3-cyano-6-ethoxypyrazolo[1,5-a]pyridin-4-yl)pyrazin-2-yl)-3-azabicyclo[3.1.0]hexan-6-yl)picolinamide,
3-chloro-N-((1R,5S,6s)-3-(5-(3-cyano-6-ethoxypyrazolo[1,5-a]pyridin-4-yl)pyrazin-2-yl)-3-azabicyclo[3.1.0]hexan-6-yl)picolinamide,
3-chloro-N-((1R,5S,6r)-3-(5-(3-cyano-6-ethoxypyrazolo[1,5-a]pyridin-4-yl)pyridin-2-yl)-3-azabicyclo[3.1.0]hexan-6-yl)picolinamide,
3-chloro-N-((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)picolinamide,
1-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-3-(6-methoxypyridin-3-yl)urea,
2-chloro-N-((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)-6-fluorobenzenesulfonamide,
2-chloro-N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-6-fluorobenzenesulfonamide,
3-chloro-N-((3aR,5r,6aS)-2-(5-(3-cyano-6-ethoxypyrazolo[1,5-a]pyridin-4-yl)pyridin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyridin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyrazin-2-yl)octahydrocyclopenta[c]pyrrol-5-yl)picolinamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-methoxypyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(morpholin-2-ylmethoxy)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)picolinamide,
2-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-methoxynicotinamide,
2-chloro-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-6-fluorobenzenesulfonamide,
3-chloro-N-((3aR,5s,6aS)-2-(5-(3-cyano-6-ethoxypyrazolo[1,5-a]pyridin-4-yl)pyrazin-2-yl)-5-methyloctahydrocyclopenta[c]pyrrol-5-yl)picolinamide,
3-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)picolinamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-3-methylbutanamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-methylbenzamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluoropicolinamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-3-(trifluoromethyl)picolinamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-fluorobenzamide,
3-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-6-methylpicolinamide,
2-chloro-N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluorobenzamide,
N-((3aR,5s,6aS)-2-(5-(3-cyano-6-ethoxypyrazolo[1,5-a]pyridin-4-yl)pyridin-2-yl)-5-methyloctahydrocyclopenta[c]pyrrol-5-yl)-5-fluoro-2-methylbenzamide,
3-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)picolinamide,
tert-butyl (((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate,
tert-butyl (((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)carbamate,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)picolinamide,
2-chloro-N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzenesulfonamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-3-methylbutanamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-2-phenylacetamide,
N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-3-methylbutanamide,
3-chloro-N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)picolinamide,
N-(((1R,5S,6r)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-hydroxy-2-phenylacetamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluorobenzamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluoro-2-methylbenzamide,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylpicolinamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylbenzamide,
3-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-fluoropicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-(trifluoromethyl)picolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)pivalamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-methylbutanamide.
3-chloro-N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)picolinamide,
(1R,3 S,5s,7s)-N-(3-chloropyridin-2-yl)-2-(5-(3-cyano-6-(1-methyl-1H-pyrazol-4-yl)pyrazolo[1,5-a]pyridin-4-yl)pyridin-2-yl)-2-azaadamantane-5-carboxamide,
N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)-3-fluoropicolinamide,
4-(6-((3aR,6aS)-5-((2-chloro-6-fluorophenyl)sulfonyl)hexahydropyrrolo[3,4-c]pyrrol-2(1H)-yl)pyridin-3-yl)-6-(2-hydroxy-2-methylpropoxy)pyrazolo[1,5-a]pyridine-3-carbonitrile,
2-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-fluorobenzamide,
3-chloro-N-(((1R,5S,6s)-3-(5-(3-cyano-6-ethoxypyrazolo[1,5-a]pyridin-4-yl)pyridin-2-yl)-3-azabicyclo[3.1.0]hexan-6-yl)methyl)picolinamide,
3-chloro-N-(((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)picolinamide,
2-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-fluorobenzamide,
N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)acetamide,
(1R,3 S,5s,7s)-N-(3-chloropyridin-2-yl)-2-(5-(3-cyano-6-(2-hydroxy-2-methylpropoxy)pyrazolo[1,5-a]pyridin-4-yl)pyridin-2-yl)-2-azaadamantane-5-carboxamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-2-hydroxy-3-methylbutanamide,
2-chloro-N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)-6-fluorobenzamide,
4-(5-((3aR,5s,6aS)-5-(((6-methoxypyridin-3-yl)methyl)amino)-5-methylhexahydrocyclopenta[c]pyrrol-2(1H)-yl)pyrazin-2-yl)-6-(1-methyl-1H-pyrazol-4-yl)pyrazolo[1,5-a]pyridine-3-carbonitrile,
3-chloro-N-((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)picolinamide,
3-chloro-N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)picolinamide,
4-(5-((1R,3 S,5s,7s)-5-hydroxy-2-azaadamantan-2-yl)pyrazin-2-yl)-6-(1-methyl-1H-pyrazol-4-yl)pyrazolo[1,5-a]pyridine-3-carbonitrile,
4-(6-((3aR,5r,6aS)-5-hydroxy-5-(pyridin-2-ylmethyl)hexahydrocyclopenta[c]pyrrol-2(1H)-yl)pyridin-3-yl)-6-(1-methyl-1H-pyrazol-3-yl)pyrazolo[1,5-a]pyridine-3-carbonitrile,
3-chloro-N-((3aR,5r,6aS)-2-(5-(3-cyano-6-(2-hydroxy-2-methylpropoxy)pyrazolo[1,5-a]pyridin-4-yl)pyridin-2-yl)octahydrocyclopenta[c]pyrrol-5-yl)picolinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)formamide,
4-(5-((1R,3 S,5s,7s)-5-amino-2-azaadamantan-2-yl)pyrazin-2-yl)-6-(1-methyl-1H-pyrazol-4-yl)pyrazolo[1,5-a]pyridine-3-carbonitrile,
tert-butyl ((1R,3S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)carbamate,
N-((3aR,5r,6aS)-2-(5-(3-cyano-6-(2-hydroxy-2-methylpropoxy)pyrazolo[1,5-a]pyridin-4-yl)pyrazin-2-yl)octahydrocyclopenta[c]pyrrol-5-yl)-6-methoxynicotinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)acetamide,
3-chloro-N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)picolinamide,
(3aR,5s,6aS)-2-(5-(3-cyano-6-(1-methyl-1H-pyrazol-3-yl)pyrazolo[1,5-a]pyridin-4-yl)pyridin-2-yl)-N-(6-methoxypyridin-3-yl)-5-methyloctahydrocyclopenta[c]pyrrole-5-carboxamide,
(1R,3 S,5s,7s)-2-(5-(3-cyano-6-(1-methyl-1H-pyrazol-4-yl)pyrazolo[1,5-a]pyridin-4-yl)pyrazin-2-yl)-N-(6-methoxypyridin-3-yl)-2-azaadamantane-5-carboxamide,
3-chloro-N-((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
(3aR,5r,6aS)-2-(5-(3-cyano-6-(2-hydroxy-2-methylpropoxy)pyrazolo[1,5-a]pyridin-4-yl)pyridin-2-yl)-N-(6-methoxypyridin-3-yl)-5-methyloctahydrocyclopenta[c]pyrrole-5-carboxamide,
6-(2-hydroxy-2-methylpropoxy)-4-(6-((3aR,4S,7R,7aS)-8-((6-methoxypyridin-3-yl)methyl)hexahydro-1H-4,7-epiminoisoindol-2(3H)-yl)pyridin-3-yl)pyrazolo[1,5-a]pyridine-3-carbonitrile,
4-(6-((3aR,4S,7R,7aS)-8-((6-methoxypyridin-3-yl)methyl)hexahydro-1H-4,7-epiminoisoindol-2(3H)-yl)pyridin-3-yl)-6-(1-methyl-1H-pyrazol-4-yl)pyrazolo[1,5-a]pyridine-3-carbonitrile,
(1R,5S,6r)-3-(5-(3-cyano-6-(2-hydroxy-2-methylpropoxy)pyrazolo[1,5-a]pyridin-4-yl)pyridin-2-yl)-N-((6-methoxypyridin-3-yl)methyl)-3-azabicyclo[3.1.0]hexane-6-carboxamide,
6-(2-hydroxy-2-methylpropoxy)-4-(5-((3aR,6aS)-5-((6-methoxypyridin-3-yl)methyl)hexahydropyrrolo[3,4-c]pyrrol-2(1H)-yl)pyrazin-2-yl)pyrazolo[1,5-a]pyridine-3-carbonitrile,
3-cyano-N-((3aR,5s,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-methoxynicotinamide,
N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-3-fluoropicolinamide,
3-chloro-N-(((1R,5S,6s)-3-(5-(3-cyano-6-(2-hydroxypropoxy)pyrazolo[1,5-a]pyridin-4-yl)pyridin-2-yl)-3-azabicyclo[3.1.0]hexan-6-yl)methyl)picolinamide,
2-chloro-N-(((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)-6-fluorobenzamide,
N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-methoxynicotinamide,
3-cyano-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)picolinamide,
N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-3-fluoropicolinamide,
3-chloro-N-(2-((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)propan-2-yl)picolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)acetamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)methanesulfonamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)isobutyramide,
(1R,3 S,5s,7s)-2-(5-(3-cyano-6-(2-hydroxy-2-methylpropoxy)pyrazolo[1,5-a]pyridin-4-yl)pyridin-2-yl)-N-(6-methoxypyridin-3-yl)-2-azaadamantane-5-carboxamide,
(1R,3 S,5s,7s)-2-(5-(3-cyano-6-ethoxypyrazolo[1,5-a]pyridin-4-yl)pyridin-2-yl)-N-(6-methoxypyridin-3-yl)-2-azaadamantane-5-carboxamide,
3-chloro-N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)picolinamide,
3-chloro-N-((1R,3 S,5s,7s)-2-(5-(3-cyano-6-ethoxypyrazolo[1,5-a]pyridin-4-yl)pyridin-2-yl)-2-azaadamantan-5-yl)picolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-3-fluoropicolinamide,
N-((1R,3 S,5s,7s)-2-(5-(3-cyano-6-(2-hydroxy-2-methylpropoxy)pyrazolo[1,5-a]pyridin-4-yl)pyridin-2-yl)-2-azaadamantan-5-yl)-6-methoxynicotinamide,
N-((1R,3 S,5s,7s)-2-(5-(3-cyano-6-ethoxypyrazolo[1,5-a]pyridin-4-yl)pyridin-2-yl)-2-azaadamantan-5-yl)-6-methoxynicotinamide,
N-((3aR,5s,6aS)-2-(5-(3-cyano-6-(1-methyl-1H-pyrazol-3-yl)pyrazolo[1,5-a]pyridin-4-yl)pyrazin-2-yl)-5-methyloctahydrocyclopenta[c]pyrrol-5-yl)-3-fluoropicolinamide,
2-chloro-N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-3-fluoropicolinamide,
3-chloro-N-((3aR,5s,6aS)-2-(5-(3-cyano-6-(2-hydroxy-2-methylpropoxy)pyrazolo[1,5-a]pyridin-4-yl)pyrazin-2-yl)-5-methyloctahydrocyclopenta[c]pyrrol-5-yl)picolinamide,
2-chloro-N-((3aR,5s,6aS)-2-(5-(3-cyano-6-(2-hydroxy-2-methylpropoxy)pyrazolo[1,5-a]pyridin-4-yl)pyrazin-2-yl)-5-methyloctahydrocyclopenta[c]pyrrol-5-yl)-6-fluorobenzamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-methoxynicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-6-methoxypicolinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-2-(trifluoromethyl)isonicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-5-methoxynicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-4-methoxypicolinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-2-methoxyisonicotinamide,
N-((3aR,5s,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)-3-methoxypicolinamide,
4-(6-((3aR,5s,6aS)-5-(((6-methoxypyridin-3-yl)methyl)amino)-5-methylhexahydrocyclopenta[c]pyrrol-2(1H)-yl)pyridin-3-yl)-6-(1-methyl-1H-pyrazol-4-yl)pyrazolo[1,5-a]pyridine-3-carbonitrile,
3-chloro-N-(((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)methyl)picolinamide,
2-chloro-N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)-6-fluorobenzamide,
N-(((1R,5S,6s)-3-(5-(3-cyano-6-(2-hydroxy-2-methylpropoxy)pyrazolo[1,5-a]pyridin-4-yl)pyrazin-2-yl)-3-azabicyclo[3.1.0]hexan-6-yl)methyl)-6-methoxynicotinamide,
N-(((1R,5S,6s)-3-(5-(3-cyano-6-(1-methyl-1H-pyrazol-4-yl)pyrazolo[1,5-a]pyridin-4-yl)pyrazin-2-yl)-3-azabicyclo[3.1.0]hexan-6-yl)methyl)-6-methoxynicotinamide,
4-(5-((1R,3 S,5s,7s)-5-hydroxy-2-azaadamantan-2-yl)pyrazin-2-yl)-6-(2-hydroxy-2-methylpropoxy)pyrazolo[1,5-a]pyridine-3-carbonitrile,
4-(6-((3aR,5r,6aS)-5-hydroxy-5-(pyridin-2-ylmethyl)hexahydrocyclopenta[c]pyrrol-2(1H)-yl)pyridin-3-yl)-6-(1-methyl-1H-pyrazol-4-yl)pyrazolo[1,5-a]pyridine-3-carbonitrile,
N-((3aR,5r,6aS)-2-(5-(3-cyano-6-(2-hydroxy-2-methylpropoxy)pyrazolo[1,5-a]pyridin-4-yl)pyridin-2-yl)octahydrocyclopenta[c]pyrrol-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)formamide,
N-((1R,3 S,5s,7s)-2-(5-(3-cyano-6-ethoxypyrazolo[1,5-a]pyridin-4-yl)pyrazin-2-yl)-2-azaadamantan-5-yl)formamide,
4-(5-((1R,3 S,5s,7s)-5-amino-2-azaadamantan-2-yl)pyrazin-2-yl)-6-(2-hydroxy-2-methylpropoxy)pyrazolo[1,5-a]pyridine-3-carbonitrile,
4-(5-((1R,3 S,5s,7s)-5-amino-2-azaadamantan-2-yl)pyrazin-2-yl)-6-ethoxypyrazolo[1,5-a]pyridine-3-carbonitrile,
3-chloro-N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)picolinamide,
N-((1R,3 S,5s,7s)-2-(5-(3-cyano-6-(1-methyl-1H-pyrazol-4-yl)pyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-ethoxypyrazolo[1,5-a]pyridin-4-yl)pyrazin-2-yl)-2-azaadamantan-5-yl)-6-methoxynicotinamide,
N-((1R,3 S,5s,7s)-2-(5-(3-cyano-6-(2-hydroxy-2-methylpropoxy)pyrazolo[1,5-a]pyridin-4-yl)pyrazin-2-yl)-2-azaadamantan-5-yl)-3-fluoropicolinamide,
3-chloro-N-((1R,3 S,5s,7s)-2-(5-(3-cyano-6-ethoxypyrazolo[1,5-a]pyridin-4-yl)pyrazin-2-yl)-2-azaadamantan-5-yl)picolinamide,
(1R,3 S,5s,7s)-2-(5-(3-cyano-6-ethoxypyrazolo[1,5-a]pyridin-4-yl)pyrazin-2-yl)-N-(6-methoxypyridin-3-yl)-2-azaadamantane-5-carboxamide,
(1R,3 S,5s,7s)-2-(5-(3-cyano-6-(2-hydroxy-2-methylpropoxy)pyrazolo[1,5-a]pyridin-4-yl)pyrazin-2-yl)-N-(6-methoxypyridin-3-yl)-2-azaadamantane-5-carboxamide,
N-((3aR,5r,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-methoxynicotinamide,
3-chloro-N-((3aR,5r,6aS)-2-(5-(3-cyano-6-(1-methyl-1H-pyrazol-4-yl)pyrazolo[1,5-a]pyridin-4-yl)pyridin-2-yl)-5-methyloctahydrocyclopenta[c]pyrrol-5-yl)picolinamide,
6-(2-hydroxy-2-methylpropoxy)-4-(6-((3aR,4S,7R,7aS)-8-(6-methoxynicotinoyl)hexahydro-1H-4,7-epiminoisoindol-2(3H)-yl)pyridin-3-yl)pyrazolo[1,5-a]pyridine-3-carbonitrile,
(1R,5S,6r)-3-(5-(3-cyano-6-(2-hydroxy-2-methylpropoxy)pyrazolo[1,5-a]pyridin-4-yl)pyridin-2-yl)-N-(6-methoxypyridin-3-yl)-3-azabicyclo[3.1.0]hexane-6-carboxamide,
6-(2-hydroxy-2-methylpropoxy)-4-(5-((3aR,6aS)-5-(1-(6-methoxypyridin-3-yl)ethyl)hexahydropyrrolo[3,4-c]pyrrol-2(1H)-yl)pyrazin-2-yl)pyrazolo[1,5-a]pyridine-3-carbonitrile,
4-(5-((3aR,6aS)-5-((6-cyanopyridin-3-yl)methyl)hexahydropyrrolo[3,4-c]pyrrol-2(1H)-yl)pyrazin-2-yl)-6-(2-hydroxy-2-methylpropoxy)pyrazolo[1,5-a]pyridine-3-carbonitrile,
2-chloro-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)-6-fluorobenzenesulfonamide,
4-(6-((3aR,6aS)-5-((2-chloro-6-fluorophenyl)sulfonyl)hexahydropyrrolo[3,4-c]pyrrol-2(1H)-yl)pyridin-3-yl)-6-(1-methyl-1H-pyrazol-3-yl)pyrazolo[1,5-a]pyridine-3-carbonitrile,
tert-butyl ((1R,3S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)carbamate,
4-(6-((1R,3 S,5s,7s)-5-amino-2-azaadamantan-2-yl)pyridin-3-yl)-6-(1-methyl-1H-pyrazol-4-yl)pyrazolo[1,5-a]pyridine-3-carbonitrile,
4-(5-((3aR,6aS)-5-((2-chloro-6-fluorophenyl)sulfonyl)hexahydropyrrolo[3,4-c]pyrrol-2(1H)-yl)pyrazin-2-yl)-6-(1-methyl-1H-pyrazol-4-yl)pyrazolo[1,5-a]pyridine-3-carbonitrile,
1-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-3-(6-methoxypyridin-3-yl)urea,
2-chloro-N-(((1R,5S,6r)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)methyl)-6-fluorobenzenesulfonamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methylbenzamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-(trifluoromethyl)picolinamide,
2-chloro-N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-fluorobenzamide,
N-((1R,3 S,5s,7s)-2-(5-(3-cyano-6-(1-methyl-1H-pyrazol-4-yl)pyrazolo[1,5-a]pyridin-4-yl)pyridin-2-yl)-2-azaadamantan-5-yl)-6-methoxynicotinamide,
(1R,3 S,5s,7s)-2-(5-(3-cyano-6-(1-methyl-1H-pyrazol-4-yl)pyrazolo[1,5-a]pyridin-4-yl)pyridin-2-yl)-N-(6-methoxypyridin-3-yl)-2-azaadamantane-5-carboxamide,
N-((1R,5S,6s)-3-(5-(3-cyano-6-(2-hydroxy-2-methylpropoxy)pyrazolo[1,5-a]pyridin-4-yl)pyridin-2-yl)-3-azabicyclo[3.1.0]hexan-6-yl)-2-(6-methoxypyridin-3-yl)acetamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-3-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-5-methoxynicotinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-4-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-methoxyisonicotinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-6-methoxypicolinamide,
N-(((1R,5S,6s)-3-(5-(3-cyano-6-(1-methyl-1H-pyrazol-4-yl)pyrazolo[1,5-a]pyridin-4-yl)pyridin-2-yl)-3-azabicyclo[3.1.0]hexan-6-yl)methyl)-2-(trifluoromethyl)isonicotinamide,
2-chloro-N-((3aR,5s,6aS)-2-(5-(3-cyano-6-(2-hydroxy-2-methylpropoxy)pyrazolo[1,5-a]pyridin-4-yl)pyridin-2-yl)-5-methyloctahydrocyclopenta[c]pyrrol-5-yl)-6-fluorobenzenesulfonamide,
3-cyano-N-((3aR,5s,6aS)-2-(5-(3-cyano-6-(1-methyl-1H-pyrazol-3-yl)pyrazolo[1,5-a]pyridin-4-yl)pyridin-2-yl)-5-methyloctahydrocyclopenta[c]pyrrol-5-yl)picolinamide, and
3-chloro-N-((3aR,5r,6aS)-2-(5-(3-cyano-6-(1-methyl-1H-pyrazol-4-yl)pyrazolo[1,5-a]pyridin-4-yl)pyrazin-2-yl)-5-methyloctahydrocyclopenta[c]pyrrol-5-yl)picolinamide.

28. A pharmaceutical composition comprising a compound of any one of claims 1-27, and/or a stereoisomer, a stable isotope, or a pharmaceutically acceptable salt or solvate thereof, admixed with at least one pharmaceutically acceptable carrier.

29. The pharmaceutical composition of claim 28, further comprising at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, another RET kinase inhibitor, and kinase inhibitors.

30. The pharmaceutical composition of claim 29, wherein the at least one therapeutic co-agent or co-treatment is combined with the compound in a single dosage form, or the at least one therapeutic co-agent is administered simultaneously or sequentially as separate dosage forms.

31. A method to treat disease in a patient in need thereof whose disease is a RET-associated disease, comprising administering to the subject in need of such treatment a therapeutically effective amount of a compound of any one of claims 1-27, and/or a stereoisomer, a stable isotope, or a pharmaceutically acceptable salt or solvate thereof, or a pharmaceutical composition of any one of claims 28-30.

32. The method of claim 31, wherein the method comprises determining if the disease in the patient is a RET-associated disease, and administering to a subject in need of such treatment a therapeutically effective RET inhibiting amount of a compound of any one of claims 1-27, and/or a stereoisomer, a stable isotope, or a pharmaceutically acceptable salt or solvate thereof, or a pharmaceutical composition of any one of claims 28-30.

33. The method of any one of claims 31-32, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

34. The method of any one of claims 31-32, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

35. The method of claim 33, wherein the treatment comprises administering at least one therapeutic co-agent or co-treatment selected from chemotherapeutics or other anti-cancer agents, apoptosis modulators, immune enhancers, agents for immunotherapy, immune checkpoint inhibitors, radiation, anti-tumor vaccines, agents for cytokine therapy, signal transduction inhibitors and kinase inhibitors.

36. The method of claim 35, wherein the administering the compound is conducted simultaneously or serially with the administering the therapeutic co-agent.

37. The method of claim 36, wherein administering the therapeutic co-agent comprises another RET inhibitor, an immunotherapy, or combination thereof.

38. The method of claim 33, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

39. The method of any one of claims 31-38, wherein the compound of any one of claims 1-27, and/or a stereoisomer, a stable isotope, or a pharmaceutically acceptable salt or solvate thereof, or a pharmaceutical composition of any one of claims 28-30, is orally administered.

40. A use of a compound of any one of claims 1-27, and/or a stereoisomer, a stable isotope, or a pharmaceutically acceptable salt or solvate thereof, or a pharmaceutical composition according to any one of claims 28-30 as a medicament, in the manufacture of a medicament, or in medicine for treatment of a RET-associated disease.

41. The use of claim 40, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

42. The use of claim 41, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

43. The use of any one of claims 41-42, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

44. The use of any one of claims 42-43, wherein the medicament is formulated for oral administration.

45. A compound of any one of claims 1-27, and/or a stereoisomer, a stable isotope, or a pharmaceutically acceptable salt or solvate thereof, or a pharmaceutical composition of claim 28-30 for use in treating a RET-associated disease.

46. The compound of claim 45, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

47. The compound of claim 45, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

48. A compound of claim 46 for use in treating RET-associated cancer in a patient, the method comprising determining if the cancer in the patient is RET-associated cancer, and administering to a subject in need of such treatment a therapeutically effective amount of the compound.

49. The compound of any one of claims 46 and 48, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

50. A method of inhibiting RET kinase activity in vitro or in vivo for a RET-associated cancer cell having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein, with a compound of any one of claims 1-27, and/or a stereoisomer, a stable isotope, or a pharmaceutically acceptable salt or solvate thereof.

51. A method of treating RET-associated cancer in a patient who has developed resistance to a RET inhibitor, comprising administering to a subject in need of such treatment a therapeutically effective RET inhibiting amount of a compound that is active against the RET kinase with RET mutations resistant to the prior treatment of any one of claims 1-27, and/or a stereoisomer, a stable isotope, or a pharmaceutically acceptable salt or solvate thereof, or a pharmaceutical composition of any one of claims 28-30.

52. The method of claim 51, wherein the method comprises (a) determining the RET-mutations of a cancer cell in a sample from a patient who developed resistance to prior treatment of a RET inhibitor; and (b) administering a compound that is active against the RET kinase with RET mutations resistant to the prior treatment of any one of claims 1-27, and/or a stereoisomer, a stable isotope, or a pharmaceutically acceptable salt or solvate thereof, or a pharmaceutical composition of any one of claims 28-30.

53. The method of any one of claims 51-52, wherein the treatment comprises administering at least one therapeutic co-agent or co-treatment selected from chemotherapeutics or other anti-cancer agents, apoptosis modulators, immune enhancers, agents for immunotherapy, immune checkpoint inhibitors, radiation, anti-tumor vaccines, agents for cytokine therapy, signal transduction inhibitors and kinase inhibitors.

54. The method of claim 53, wherein administering the therapeutic co-agent comprises another RET inhibitor, an immunotherapy, or combination thereof.

55. A kit comprising a compound of any one of claims 1-27 or a pharmaceutically acceptable salt thereof, or a pharmaceutical composition according to any one of claims 28-30, and a therapeutic co-agent.

56. A process for preparing compounds of Formula 22, wherein Z₃ is Cl, Br, OTf, OMe, or OR; wherein R is H or an optionally substituted C1-C3 alkyl, wherein the optional substituents are 1-3 groups independently selected from H, halogen, C1-C3 alkoxy, C1-C3 alkanoyloxy, and aryl; X³ and X⁶ are independently —CH— or N; R⁹ is H, OH, F, CF₃, OCF₃, CN, or an optionally substituted group selected from C1-C3 alkyl, C1-C3 alkoxy, C3-C6 cycloalkyl, and C3-C6 cycloalkoxy; and P is an amino protecting group. [chemical structure]
